(19) United States
(12) Patent Application Publication
(10) Pub. No.: US 2008/0249130 A1
(43) Pub. Date: Oct. 9, 2008

(54) Gut microsomal triglyceride transport protein inhibitors

(75) Inventor: Chi B. Vu, Arlington, MA (US)

Correspondence Address: ROPES & GRAY LLP, PATENT DOCKETING 39/41, ONE INTERNATIONAL PLACE, BOSTON, MA, 02110-2624, US

(73) Assignee: Sirtris Pharmaceuticals, Inc., Cambridge, MA (US)

(21) Appl. No.: 12/069,429

(22) Filed: Feb. 8, 2008

Related U.S. Application Data
(60) Provisional application No. 60/900,491, filed on Feb. 9, 2007.
(60) Provisional application No. 60/958,301, filed on Jul. 2, 2007.

(51) Int. Cl. A61K 31/47; C07D 277/62; C07D 215/38; A61K 31/428; A61P 3/00; A61K 31/165; C07C 233/11

(52) U.S. Cl. 514/313; 548/168; 546/162; 564/180; 514/620; 514/367

(57) ABSTRACT

Compounds represented by formula (I):

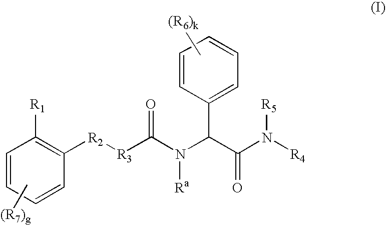

are inhibitors of gut microsomal triglyceride transfer protein. Such compounds are useful in treating diseases or conditions such as diabetes and obesity, along with patients are risk for developing such diseases or conditions.

### RELATED APPLICATIONS

This application claims the benefit of U.S. Provisional Application Nos. 60/900,491, filed Feb. 9, 2007, and 60/958,301, filed Jul. 2, 2007, the contents of which are incorporated by reference in their entirety.

### BACKGROUND

Triglycerides are one of the most efficient storage forms of free energy. Because of their insolubility in biological fluids, their transport between cells and tissues requires that they be assembled into lipoprotein particles. Genetic disruption of the lipoprotein assembly/secretion pathway leads to several human disorders associated with malnutrition and developmental abnormalities. In contrast, patients displaying inappropriately high rates of lipoprotein production display increased risk for the development of atherosclerotic cardiovascular disease (Davis, Biochim. Biophys. Acta, 1999, 1440, 1-31).

The mammalian lipoprotein assembly/secretion pathway requires two components: apolipoprotein B (ApoB, an amphipathic protein) and microsomal triglyceride transfer protein (MTP), a lipid transfer protein. MTP binds and shuttles individual lipid molecules between membranes. In particular, MTP accelerates the transport of triglycerides, cholesteryl ester, and phospholipid from the ER membrane, where lipid molecules are synthesized, to developing lipoproteins within the lumen of the ER (Berriot-Varoqueaux et al., Annu. Rev. Nutr., 2000, 20, 663-697). In vitro analyses show that MTP has a preference for transferring triglycerides and cholesteryl esters (Gordon and Jamil, Biochim. Biophys. Acta, 2000, 1486, 72-83).

MTP is a heterodimeric neutral lipid transfer protein found in the lumen of the endoplasmic reticulum of ApoB lipoprotein-secreting cells, predominantly hepatocytes and intestinal enterocytes, and has been recently detected in the human heart (Herrmann et al., J. Lipid Res., 1998, 39, 2432-2435). The smaller 55 kiloDalton (kDa) subunit of MTP has been identified as protein disulfide isomerase (PDI). The isomerase activity is not required for the complex to transfer lipid. The larger 97 kDa subunit is a unique polypeptide responsible for the in vitro binding and transfer of lipids (Gordon and Jamil, Biochim. Biophys. Acta, 2000, 1486, 72-83).

Elevated plasma lipid levels cause premature atherosclerosis. Studies of the role of MTP in lipoproteinemia demonstrate that the protein is required for both hepatic and intestinal apoB-containing lipoprotein production. An increase in microsomal triglyceride transfer protein in relation to very low density lipoprotein (VLDL) production and secretion is thought to cause hyperlipoproteinemia, which is an underlying cause of cardiovascular disease (Kuriyama et al., Hepatology, 1998, 27, 557-562). These studies suggest that inhibition of MTP function may be an effective strategy to prevent very low density lipoprotein (VLDL) and chylomicron assembly and to lower plasma lipid levels (Jamil et al., Proc. Natl. Acad. Sci. U.S.A., 1996, 93, 11991-11995).

In addition, MTP inhibitors are believed to be useful for treating obesity (Li et al., Bioorg. Med. Chem. Lett., 2006, 16, 3039-3042).

Many small molecule inhibitors of MTP function have been reported to date. However, due to their toxicity, these molecules are generally unfit for pharmaceutical use. Thus there exists a need for more effective and safer inhibitors that can be used to treat human disease.

### SUMMARY

The invention discloses compounds that inhibit MTP.

In one aspect, the invention provides a compound of formula (I):

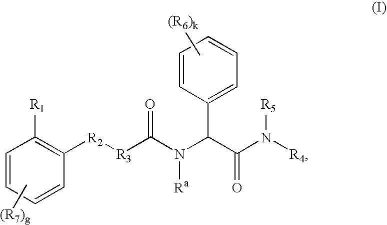

or a pharmaceutically acceptable salt thereof, wherein:

R₁ is selected from phenyl, non-aromatic heterocyclyl, or partially or fully aromatic heterocyclyl;

R₂ is selected from —C(O)—N(R^{a})— or —CH(R^{a})—N(R^{a})—;

R₃ is -L₁-R₂₀-L₂-;

R₄ is selected from H, (C₁-C₆)alkyl, (C₃-C₈)cycloalkyl, —C(O)R₁₅, —C(S)R₁₅, —(CR^{a}R^{b})_{q}O(C₁-C₆ alkyl), —(CR^{a}R^{b})_{q}S(C₁-C₆ alkyl), —(CR^{a}R^{b})ᵣC(O)R₁₅, —(CR^{a}R^{b})ᵣR₁₅ or —SO₂R₁₅;

R₅ is selected from (C₁-C₆)alkyl, (C₂-C₆)alkenyl, (C₂-C₆)alkynyl, —(CR^{a}R^{b})_{q}O(C₁-C₆ alkyl), —(CR^{a}R^{b})_{q}S(C₁-C₆ alkyl); (C₃-C₈)cycloalkyl, —C(O)R₁₅, —C(S)R₅, —(CR^{a}R^{b})ᵣC(O)R₁₅, —(CR^{a}R^{b})ᵣC(S)R₁₅, —(CR^{a}R^{b})ᵣR₁₅, —SO₂R₁₅, phenyl, pyridyl, phenyl-Z₁- or pyridyl-Z₁-;

or R₄ and R₅ taken together with the nitrogen atom to which they are attached form a 4-10 membered heterocyclyl, wherein:
each R^{a} and R^{b} is independently H or (C₁-C₆)alkyl;
L₁ is a direct bond or —CH₂—;
L₂ is a direct bond, —CH₂—, —S—, or —O—, wherein at least one of L₁ and L₂ is a direct bond;
R₂₀ is a carbocyclic or heterocyclic ring;
Z₁ is —SO₂— or —(CR^{a}R^{b})ᵥ—;
each R₁₅ is independently H, (C₁-C₆)alkyl, or (C₃-C₈)cycloalkyl;
k is an integer from 0 to 5;
g is an integer from 0 to 4;
each j is independently 0, 1 or 2;
each q is independently an integer from 0 to 6;
each r is independently an integer from 1 to 5;
each v is independently an integer from 1 to 6; and

R₆ is selected from halo, C₁-C₄ alkyl, or O—C₁-C₄ alkyl; and

R₇ is selected from R₆ or —X—R₁₆, wherein:
X is selected from a bond, —O—, —S—, —N(R^{a})—, —C(O)N(R^{a})—, or —N(R^{a})C(O)—; and
R₁₆ is selected from C₁-C₄ alkyl, —CH₂C(O)N(R₁₇)(R₁₈), cycloalkyl, or —(CH₂)ⱼ-heterocyclyl, wherein R₁₇ and R₁₈ are independently selected from hydrogen, alkyl, carbocyclyl, heterocyclyl, heteroalkyl, aminocarbonyl, alkylcarbonyl, alkoxycarbonyl, arylcarbonyl, or aryloxycarbonyl;
   provided that:

a) when R₂ is —C(O)—N(R^{a})— and R₃ is quinolinyl, R₁ is not phenyl, pyridyl, naphthyl, benzofuranyl, benzodioxolyl, tetrahydroquinoline or dihydrobenzofuranyl;

b) when R₁ is phenyl or pyridyl and R₂ is —C(O)—N(R^{a})—; R₃ is not benzofuranyl, benzo[b]thienyl, indolyl, pyrrolyl, phenyl, pyridyl or phenyl-CH₂; and

c) when R₂ is —C(O)—N(R^{a})— and R₃ is benzofuranyl, benzo[b]thienyl, or indolyl; R₅ is not pyridyl, -Z₁-pyridyl, phenyl, or -Z₁-phenyl.

In another aspect, the invention provides a compound of formula (II):

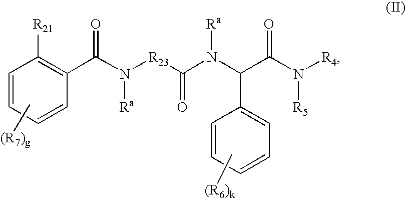

or a pharmaceutically acceptable salt thereof, wherein:

R₂₁ is selected from non-aromatic heterocyclyl; monocyclic heteroaryl, wherein said monocyclic heteroaryl comprises either: (i) at least one ring heteroatom selected from O or S, or (ii) at least two ring atoms independently selected from O, N or S; or bicyclic heteroaryl, wherein said bicyclic heteroaryl comprises a ring heteroatom selected from N or S;

R₂₃ is quinolinyl;

R₄ is selected from H, (C₁-C₆)alkyl, (C₃-C₈)cycloalkyl, —C(O)R₁₅, —C(S)R₁₅, —(CR^{a}R^{b})_{q}O(C₁-C₆ alkyl), —(CR^{a}R^{b})_{q}S(C₁-C₆ alkyl), —(CR^{a}R^{b})ᵣC(O)R₁₅, —(CR^{a}R^{b})ᵣR₁₅ or —SO₂R₁₅;

R₅ is selected from (C₁-C₆)alkyl, (C₂-C₆)alkenyl, (C₂-C₆)alkynyl, —(CR^{a}R^{b})_{q}O(C₁-C₆ alkyl), (CR^{a}R^{b})_{q}S(C₁-C₆ alkyl), (C₃-C₈)cycloalkyl, —C(O)R₁₅, —C(S)R₁₅, —(CR^{a}R^{b})ᵣC(O)R₁₅, —(CR^{a}R^{b})ᵣC(S)R₁₅, —(CR^{a}R^{b})ᵣR₁₅, —SO₂R₁₅, phenyl, pyridyl, phenyl-Z₁- or pyridyl-Z₁-;

or R₄ and R₅ taken together with the nitrogen atom to which they are attached form a 4-10 membered heterocyclyl;
each R^{a} and R^{b} is independently H or (C₁-C₆)alkyl;
Z₁ is —SO₂— or —(CR^{a}R^{b})ᵥ—;
each R₁₅ is independently H, (C₁-C₆)alkyl, or (C₃-C₈)cycloalkyl;
k is an integer from 0 to 5;
g is an integer from 0 to 4;
each j is 0, 1 or 2;
each q is independently an integer from 0 to 6;
each r is independently an integer from 1 to 5;
each v is independently an integer from 1 to 6; and

R₆ is selected from halo, C₁-C₄ alkyl, or O—C₁-C₄ alkyl; and

R₇ is selected from R₆ or —X—R₁₆, wherein:
X is selected from a bond, —O—, —S—, —N(R^{a})—, —C(O)N(R^{a})—, or —N(R^{a})C(O)—; and
R₁₆ is selected from C₁-C₄ alkyl, —CH₂C(O)N(R₁₇)(R₁₈), cycloalkyl, or —(CH₂)ⱼ-heterocyclyl, wherein R₁₇ and R₁₈ are independently selected from hydrogen, alkyl, carbocyclyl, heterocyclyl, heteroalkyl, aminocarbonyl, alkylcarbonyl, alkoxycarbonyl, arylcarbonyl, or aryloxycarbonyl.

In yet another aspect, the invention provides a compound of formula (III):

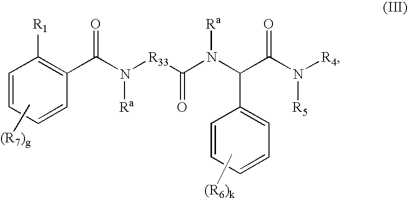

or a pharmaceutically acceptable salt thereof, wherein:

R₁ is selected from phenyl, a non-aromatic heterocyclyl, or partially or fully aromatic heterocyclic;

R₃₃ is selected from a bicyclic heterocycle comprising at least one aromatic ring and comprising at least two ring heteroatoms independently selected from N, O or S; -(bicyclic heteroaryl)-CH₂—; -bicyclic aryl-; -(bicyclic aryl)-CH₂—; —CH₂-(bicyclic non-aromatic carbocyclic)-; -(bicyclic non-aromatic carbocyclic)-; -(bicyclic non-aromatic carbocyclic)-CH₂; —CH₂-(bicyclic non-aromatic heterocyclyl)-; -(bicyclic non-aromatic heterocyclyl)-CH₂—; -phenyl-S—; -phenyl-O—; -(monocyclic saturated heterocyclyl)-CH₂—; -monocyclic non-aromatic carbocyclic-; or -(monocyclic non-aromatic carbocyclic)-CH₂—;

R₄ is selected from H, (C₁-C₆)alkyl, (C₃-C₈)cycloalkyl, —C(O)R₁₅, —C(S)R₁₅, —(CR^{a}R^{b})_{q}O(C₁-C₆ alkyl), —(CR^{a}R^{b})_{q}S(C₁-C₆ alkyl), —(CR^{a}R^{b})ᵣC(O)R₁₅, —(CR^{a}R^{b})ᵣR₁₅ or —SO₂R₁₅;

R₅ is selected from (C₁-C₆)alkyl, (C₂-C₆)alkenyl, (C₂-C₆)alkynyl, —(CR^{a}R^{b})_{q}O(C₁-C₆ alkyl), —(CR^{a}R^{b})_{q}S(C₁-C₆ alkyl); (C₃-C₈)cycloalkyl, —C(O)R₁₅, —C(S)R₁₅, —(CR^{a}R^{b})ᵣC(O)R₁₅, —(CR^{a}R^{b})ᵣC(S)R₅, —(CR^{a}R^{b})ᵣR₁₅, —SO₂R₁₅, phenyl, pyridyl, phenyl-Z₁- or pyridyl-Z₁-;

or R₄ and R₅ taken together with the nitrogen atom to which they are attached form a 4-10 membered heterocyclyl, wherein:
each R^{a} and R^{b} is independently H or (C₁-C₆)alkyl;
Z₁ is —SO₂— or —(CR^{a}R^{b})ᵥ—;
each R₁₅ is independently H, (C₁-C₆)alkyl, or (C₃-C₈)cycloalkyl;
k is an integer from 0 to 5;
g is an integer from 0 to 4;
each j is independently 0, 1 or 2;
each q is independently an integer from 0 to 6;
each r is independently an integer from 1 to 5;
each v is independently an integer from 1 to 6; and

R₆ is selected from halo, C₁-C₄ alkyl, or O—C₁-C₄ alkyl; and

R₇ is selected from R₆, or —X—R₁₆, wherein:
X is selected from a bond, —O—, —S—, —N(R^{a})—, —C(O)N(R^{a})—, or —N(R^{a})C(O)—; and
R₁₆ is selected from C₁-C₄ alkyl, —CH₂C(O)N(R₁₇)(R₁₈), cycloalkyl, or —(CH₂)ⱼ-heterocyclyl, wherein R₁₇ and R₁₈ are independently selected from hydrogen, alkyl, carbocyclyl, heterocyclyl, heteroalkyl, aminocarbonyl, alkylcarbonyl, alkoxycarbonyl, arylcarbonyl, or aryloxycarbonyl.

In a further aspect, the invention provides a compound of formula (IV):

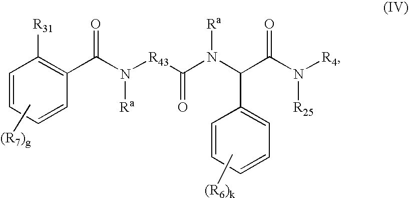

or a pharmaceutically acceptable salt thereof, wherein:

R₃₁ is selected from non-aromatic heterocyclyl, or partially or fully aromatic heterocyclic, wherein when said partially or fully aromatic heterocyclic is monocyclic it comprises either (i) at least one ring heteroatom selected from O or S, or (ii) at least two ring atoms independently selected from O, N or S;

R₄₃ is selected from benzofuranyl, dihydrobenzofuranyl, tetrahydrobenzofuranyl, benzothienyl, dihydrobenzothienyl, tetrahydrobenzothienyl, indolyl, dihydroindolyl or tetrahydroindolyl;

each of R₄ and R₂₅ is independently selected from (C₁-C₆)alkyl, (C₂-C₆)alkenyl, (C₂-C₆)alkynyl, —(CR^{a}R^{b})_{q}O(C₁-C₆ alkyl), (CR^{a}R^{b})_{q}S(C₁-C₆ alkyl); (C₃-C₈)cycloalkyl, —C(O)R₁₅, —C(S)R₁₅, —(CR^{a}R^{b})ᵣC(O)R₅, —(CR^{a}R^{b})ᵣC(S)R₅, —(CR^{a}R^{b})ᵣR₁₅, or —SO₂R₁₅;

or R₄ and R₂₅ taken together with the nitrogen atom to which they are attached together form a 4-10 membered heterocyclyl, wherein:
each R^{a} and R^{b} is independently H or (C₁-C₆)alkyl;
each R₁₅ is independently H, (C₁-C₆)alkyl, or (C₃-C₈)cycloalkyl;
k is an integer from 0 to 5;
g is an integer from 0 to 4;
each j is independently 0, 1 or 2;
each q is independently an integer from 0 to 6;
each r is independently an integer from 1 to 5; and

R₆ is selected from halo, C₁-C₄ alkyl, or O—C₁-C₄ alkyl; and

R₇ is selected from R₆, or —X—R₁₆, wherein:
X is selected from a bond, —O—, —S—, —N(R^{a})—, —C(O)N(R^{a})—, or —N(R^{a})C(O)—; and
R₁₆ is selected from C₁-C₄ alkyl, —CH₂C(O)N(R₁₇)(R₁₈), cycloalkyl, or —(CH₂)ⱼ-heterocyclyl, wherein R₁₇ and R₁₈ are independently selected from hydrogen, alkyl, carbocyclyl, heterocyclyl, heteroalkyl, aminocarbonyl, alkylcarbonyl, alkoxycarbonyl, arylcarbonyl, or aryloxycarbonyl.

The invention additionally provides a compound of formula (V):

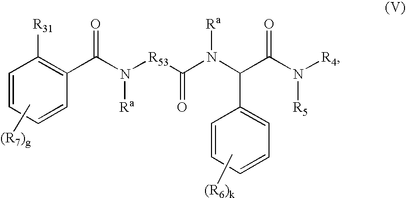

or a pharmaceutically acceptable salt thereof, wherein:

R₃₁ is selected from non-aromatic heterocyclyl, or partially or fully aromatic heterocyclic, wherein when said partially or fully aromatic heterocyclic is monocyclic it comprises either (i) at least one ring heteroatom selected from O or S, or (ii) at least two ring atoms independently selected from O, N or S;

R₅₃ is selected from phenyl or —CH₂— (phenyl)-;

R₄ is selected from H, (C₁-C₆)alkyl, (C₃-C₈)cycloalkyl, —C(O)R₁₅, —C(S)R₁₅, —(CR^{a}R^{b})_{q}O(C₁-C₆ alkyl), —(CR^{a}R^{b})_{q}S(C₁-C₆ alkyl), —(CR^{a}R^{b})ᵣC(O)R₁₅, —(CR^{a}R^{b})ᵣR₁₅ or —SO₂R₁₅;

R₅ is selected from (C₁-C₆)alkyl, (C₂-C₆)alkenyl, (C₂-C₆)alkynyl, —(CR^{a}R^{b})_{q}O(C₁-C₆ alkyl), —(CR^{a}R^{b})_{q}S(C₁-C₆ alkyl); (C₃-C₈)cycloalkyl, —C(O)R₁₅, —C(S)R₁₅, —(CR^{a}R^{b})ᵣC(O)R₁₅, —(CR^{a}R^{b})ᵣC(S)R₁₅, —(CR^{a}R^{b})ᵣR₁₅, —SO₂R₁₅, phenyl, pyridyl, phenyl-Z₁- or pyridyl-Z₁-;

or R₄ and R₅ taken together with the nitrogen atom to which they are attached form a 4-10 membered monocyclic heterocyclyl, wherein:
each R^{a} and R^{b} is independently H or (C₁-C₆)alkyl;
Z₁ is —SO₂— or —(CR^{a}R^{b})ᵥ—;
each R₁₅ is independently H, (C₁-C₆)alkyl, or (C₃-C₈)cycloalkyl;
k is an integer from 0 to 5;
g is an integer from 0 to 4;
each j is independently 0, 1 or 2;
each q is independently an integer from 0 to 6;
each r is independently an integer from 1 to 5;
each v is independently an integer from 1 to 6; and

R₆ is selected from halo, C₁-C₄ alkyl, or O—C₁-C₄ alkyl; and

R₇ is selected from R₆ or —X—R₁₆, wherein:
X is selected from a bond, —O—, —S—, —N(R^{a})—, —C(O)N(R^{a})—, or —N(R^{a})C(O)—; and
R₁₆ is selected from C₁-C₄ alkyl, —CH₂C(O)N(R₁₇)(R₁₈), cycloalkyl, or —(CH₂)ⱼ-heterocyclyl, wherein R₁₇ and R₁₈ are independently selected from hydrogen, alkyl, carbocyclyl, heterocyclyl, heteroalkyl, aminocarbonyl, alkylcarbonyl, alkoxycarbonyl, arylcarbonyl, or aryloxycarbonyl.

In another aspect, the invention provides a compound of formula (VI):

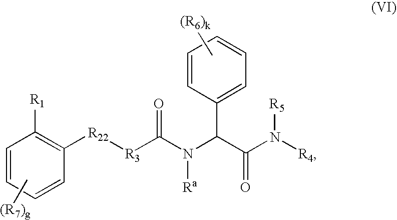

or a pharmaceutically acceptable salt thereof, wherein:

R₁ is selected from phenyl, non-aromatic heterocyclyl, or partially or fully aromatic heterocyclyl;

R₂₂ is —CH(R^{a})—N(R^{a})—;

R₃ is selected from -bicyclic partially or fully aromatic heterocyclyl-, -(bicyclic partially or fully aromatic heterocyclyl)-CH₂—, -bicyclic aryl-, -(bicyclic aryl)-CH₂—, —CH₂-(bicyclic non-aromatic cycloalkyl), —CH₂-(bicyclic non-aromatic heterocyclyl), -phenyl-S—, -phenyl-O—, -phenyl-CH₂—, -phenyl-, -(monocyclic non-aromatic heterocyclyl)-CH₂—, -monocyclic non-aromatic cycloalkyl-, or -(monocyclic non-aromatic cycloalkyl)-CH₂—;

R₄ is selected from H, (C₁-C₆)alkyl, (C₃-C₈)cycloalkyl, —C(O)R₁₅, —C(S)R₁₅, —(CR^{a}R^{b})_{q}O(C₁-C₆ alkyl), —(CR^{a}R^{b})_{q}S(C₁-C₆ alkyl), —(CR^{a}R^{b})ᵣC(O)R₁₅, —(CR^{a}R^{b})ᵣR₁₅ or —SO₂R₁₅;

R₅ is selected from (C₁-C₆)alkyl, (C₂-C₆)alkenyl, (C₂-C₆)alkynyl, —(CR^{a}R^{b})_{q}O(C₁-C₆ alkyl), —(CR^{a}R^{b})_{q}S(C₁-C₆ alkyl); (C₃-C₈)cycloalkyl, —C(O)R₁₅, —C(S)R₁₅, —(CR^{a}R^{b})ᵣC(O)R₁₅, —(CR^{a}R^{b})ᵣC(S)R₁₅, —(CR^{a}R^{b})ᵣR₁₅, —SO₂R₁₅, phenyl, pyridyl, phenyl-Z₁- or pyridyl-Z₁-;

or R₄ and R₅ taken together with the nitrogen atom to which they are attached form a 4-10 membered monocyclic heterocyclyl, wherein:
each R^{a} and R^{b} is independently H or (C₁-C₆)alkyl;
Z₁ is —SO₂— or —(CR^{a}R^{b})ᵥ—;
each R₁₅ is independently H, (C₁-C₆)alkyl, or (C₃-C₈)cycloalkyl;
k is an integer from 0 to 5;
g is an integer from 0 to 4;
each j is independently 0, 1 or 2;
each q is independently an integer from 0 to 6;
each r is independently an integer from 1 to 5;
each v is independently an integer from 1 to 6; and

R₆ is selected from halo, C₁-C₄ alkyl, or O—C₁-C₄ alkyl; and

R₇ is selected from R₆, or —X—R₁₆, wherein:
X is selected from a bond, —O—, —S—, —N(R^{a})—, —C(O)N(R^{a})—, or —N(R^{a})C(O)—; and
R₁₆ is selected from C₁-C₄ alkyl, —CH₂C(O)N(R₁₇)(R₁₈), cycloalkyl, or —(CH₂)ⱼ-heterocyclyl, wherein R₁₇ and R₁₈ are independently selected from hydrogen, alkyl, carbocyclyl, heterocyclyl, heteroalkyl, aminocarbonyl, alkylcarbonyl, alkoxycarbonyl, arylcarbonyl, or aryloxycarbonyl.

In certain embodiments, the invention also includes pharmaceutical compositions containing one or more compounds of the invention, along with pyrogen-free compositions that include one or more compounds of the invention.

In addition, certain embodiments of the invention include methods for treating one or more of the diseases or conditions described herein. The invention also includes the use of compounds disclosed herein in treating the diseases and conditions disclosed herein and the use of compounds described herein in the manufacture of a medicament for the treatment of a disorder or condition disclosed herein.

### DETAILED DESCRIPTION

### 1. Definitions

As used herein, the following terms and phrases shall have the meanings set forth below. Unless defined otherwise, all technical and scientific terms used herein have the same meaning as commonly understood to one of ordinary skill in the art.

The singular forms “a,” “an,” and “the” include plural reference unless the context clearly dictates otherwise.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject.

The term “bioavailable” when referring to a compound is art-recognized and refers to a form of a compound that allows for it, or a portion of the amount of compound administered, to be absorbed by, incorporated to, or otherwise physiologically available to a subject or patient to whom it is administered.

“Diabetes” refers to high blood sugar or ketoacidosis, as well as chronic, general metabolic abnormalities arising from a prolonged high blood sugar status or a decrease in glucose tolerance. “Diabetes” encompasses both the type I and type II (Non Insulin Dependent Diabetes Mellitus or NIDDM) forms of the disease. The risk factors for diabetes include the following factors: waistline of more than 40 inches for men or 35 inches for women, blood pressure of 130/85 mmHg or higher, triglycerides above 150 mg/dl, fasting blood glucose greater than 100 mg/dl or high-density lipoprotein of less than 40 mg/dl in men or 50 mg/dl in women.

The term “ED₅₀” is art-recognized. In certain embodiments, ED₅₀ means the dose of a drug which produces 50% of its maximum response or effect, or alternatively, the dose which produces a pre-determined response in 50% of test subjects or preparations. The term “LD₅₀” is art-recognized. In certain embodiments, LD₅₀ means the dose of a drug which is lethal in 50% of test subjects. The term “therapeutic index” is an art-recognized term which refers to the therapeutic index of a drug, defined as LD₅₀/ED₅₀.

The term “hyperinsulinemia” refers to a state in an individual in which the level of insulin in the blood is higher than normal.

The term “including” is used to mean “including but not limited to”. “Including” and “including but not limited to” are used interchangeably.

The term “insulin resistance” refers to a state in which a normal amount of insulin produces a subnormal biologic response relative to the biological response in a subject that does not have insulin resistance.

An “insulin resistance disorder,” as discussed herein, refers to any disease or condition that is caused by or contributed to by insulin resistance. Examples include: diabetes, obesity, metabolic syndrome, insulin-resistance syndromes, syndrome X, insulin resistance, high blood pressure, hypertension, high blood cholesterol, dyslipidemia, hyperlipidemia, dyslipidemia, atherosclerotic disease including stroke, coronary artery disease or myocardial infarction, hyperglycemia, hyperinsulinemia and/or hyperproinsulinemia, impaired glucose tolerance, delayed insulin release, diabetic complications, including coronary heart disease, angina pectoris, congestive heart failure, stroke, cognitive functions in dementia, retinopathy, peripheral neuropathy, nephropathy, glomerulonephritis, glomerulosclerosis, nephrotic syndrome, hypertensive nephrosclerosis some types of cancer (such as endometrial, breast, prostate, and colon), complications of pregnancy, poor female reproductive health (such as menstrual irregularities, infertility, irregular ovulation, polycystic ovarian syndrome (PCOS)), lipodystrophy, cholesterol related disorders, such as gallstones, cholescystitis and cholelithiasis, gout, obstructive sleep apnea and respiratory problems, osteoarthritis, and prevention and treatment of bone loss, e.g. osteoporosis.

The term “livestock animals” refers to domesticated quadrupeds, which includes those being raised for meat and various byproducts, e.g., a bovine animal including cattle and other members of the genus Bos, a porcine animal including domestic swine and other members of the genus Sus, an ovine animal including sheep and other members of the genus Ovis, domestic goats and other members of the genus Capra; domesticated quadrupeds being raised for specialized tasks such as use as a beast of burden, e.g., an equine animal including domestic horses and other members of the family Equidae, genus Equus.

The term “mammal” is known in the art, and exemplary mammals include humans, primates, livestock animals (including bovines, porcines, etc.), companion animals (e.g., canines, felines, etc.) and rodents (e.g., mice and rats).

“Obese” individuals or individuals suffering from obesity are generally individuals having a body mass index (BMI) of at least 25 or greater. Obesity may or may not be associated with insulin resistance.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrasternal injection and infusion.

A “patient”, “subject”, “individual” or “host” refers to either a human or a non-human animal.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “prophylactic” or “therapeutic” treatment is art-recognized and refers to administration of a drug to a host. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The term “pyrogen-free”, with reference to a composition, refers to a composition that does not contain a pyrogen in an amount that would lead to an adverse effect (e.g., irritation, fever, inflammation, diarrhea, respiratory distress, endotoxic shock, etc.) in a subject to which the composition has been administered. For example, the term is meant to encompass compositions that are free of, or substantially free of, an endotoxin such as, for example, a lipopolysaccharide (LPS).

The terms “systemic administration,” “administered systemically,” “peripheral administration” and “administered peripherally” are art-recognized and refer to the administration of a subject composition, therapeutic or other material other than directly into the central nervous system, such that it enters the patient's system and, thus, is subject to metabolism and other like processes.

The term “therapeutic agent” is art-recognized and refers to any chemical moiety that is a biologically, physiologically, or pharmacologically active substance that acts locally or systemically in a subject. The term also means any substance intended for use in the diagnosis, cure, mitigation, treatment or prevention of disease or in the enhancement of desirable physical or mental development and/or conditions in an animal or human.

The term “therapeutic effect” is art-recognized and refers to a local or systemic effect in animals, particularly mammals, and more particularly humans caused by a pharmacologically active substance. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art. For example, certain compositions described herein may be administered in a sufficient amount to produce a desired effect at a reasonable benefit/risk ratio applicable to such treatment.

“Treating” a condition or disease refers to curing as well as ameliorating at least one symptom of the condition or disease.

### 2. Compounds of the Invention

In one aspect, the invention provides a compound of formula (I):

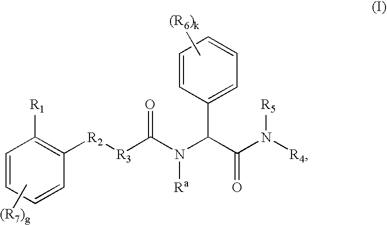

or a pharmaceutically acceptable salt thereof, wherein:

R₁ is selected from phenyl, non-aromatic heterocyclyl, or partially or fully aromatic heterocyclic;

R₂ is selected from —C(O)—N(R³)— or —CH(R^{a})—N(R^{a})—;

R₃ is -L₁-R₂₀-L₂-;

R₄ is selected from H, (C₁-C₆)alkyl, (C₃-C₈)cycloalkyl, —C(O)R₁₅, —C(S)R₁₅, —(CR^{a}R^{b})_{q}O(C₁-C₆ alkyl), —(CR^{a}R^{b})_{q}S(C₁-C₆ alkyl), —(CR^{a}R^{b})ᵣC(O)R₁₅, —(CR^{a}R^{b})ᵣR₁₅ or —SO₂R₁₅;

R₅ is selected from (C₁-C₆)alkyl, (C₂-C₆)alkenyl, (C₂-C₆)alkynyl, —(CR^{a}R^{b})_{q}O(C₁-C₆ alkyl), —(CR^{a}R^{b})_{q}S(C₁-C₆ alkyl); (C₃-C₈)cycloalkyl, —C(O)R₁₅, —C(S)R₁₅, —(CR^{a}R^{b})ᵣC(O)R₁₅, —(CR^{a}R^{b})ᵣC(S)R₁₅, —(CR^{a}R^{b})ᵣR₁₅, —SO₂R₁₅, phenyl, pyridyl, phenyl-Z₁- or pyridyl-Z₁-;

or R₄ and R₅ taken together with the nitrogen atom to which they are attached form a 4-10 membered heterocyclyl, wherein:
each R^{a} and R^{b} is independently H or (C₁-C₆)alkyl, such as CF₃;
L₁ is a direct bond or —CH₂—;
L₂ is a direct bond, —CH₂—, —S—, or —O—, wherein at least one of L₁ and L₂ is a direct bond;
R₂₀ is a carbocyclic or heterocyclic ring;
Z₁ is —SO₂— or —(CR^{a}R^{b})ᵥ—;
each R₁₅ is independently H, (C₁-C₆)alkyl, or (C₃-C₈)cycloalkyl;
k is an integer from 0 to 5;
g is an integer from 0 to 4;
each j is independently 0, 1 or 2;
each q is independently an integer from 0 to 6;
each r is independently an integer from 1 to 5;
each v is independently an integer from 1 to 6; and

R₆ is selected from halo, C₁-C₄ alkyl, or O—C₁-C₄ alkyl; and

R₇ is selected from R₆ or —X—R₁₆, wherein:
X is selected from a bond, —O—, —S—, —N(R^{a})—, —C(O)N(R^{a})—, or —N(R^{a})C(O)—; and
R₁₆ is selected from C₁-C₄ alkyl, —CH₂C(O)N(R₁₇)(R₁₈), cycloalkyl, or —(CH₂)ⱼ-heterocyclyl, wherein R₁₇ and R₁₈ are independently selected from hydrogen, alkyl, carbocyclyl, heterocyclyl, heteroalkyl, aminocarbonyl, alkylcarbonyl, alkoxycarbonyl, arylcarbonyl, or aryloxycarbonyl;
   provided that:

a) when R₂ is —C(O)—N(R^{a})— and R₃ is quinolinyl, R₁ is not phenyl, pyridyl, naphthyl, benzofuranyl, benzodioxolyl, tetrahydroquinoline or dihydrobenzofuranyl;

b) when R₁ is phenyl or pyridyl and R₂ is —C(O)—N(R^{a})—; R₃ is not benzofuranyl, benzo[b]thienyl, indolyl, pyrrolyl, phenyl, pyridyl or phenyl-CH₂; and

c) when R₂ is —C(O)—N(R^{a})— and R₃ is benzofuranyl, benzo[b]thienyl, or indolyl; R₅ is not pyridyl, -Z₁-pyridyl, phenyl, or -Z₁-phenyl.

In certain embodiments, R₂₀ is a monocyclic ring, particularly a monocyclic aromatic ring.

In certain embodiments, R₂₀ is a bicyclic ring, particularly a bicyclic ring wherein at least one ring is aromatic.

In certain embodiments, R₃ is selected from -bicyclic heteroaryl-, -(bicyclic partially or fully aromatic heterocyclic)-CH₂—, —CH₂-(bicyclic partially or fully aromatic heterocyclic)-, -(bicyclic partially or fully aromatic heterocyclic)-S—, -(bicyclic partially or fully aromatic heterocyclic)-O—, -bicyclic aryl-, -(bicyclic aryl)-CH₂—, —CH₂-(bicyclic aryl)-, -(bicyclic aryl)-O—, -(bicyclic aryl)-S—, -(bicyclic non-aromatic carbocyclic)-, —CH₂-(bicyclic non-aromatic carbocyclic)-, -(bicyclic non-aromatic carbocyclic)-CH₂, -(bicyclic non-aromatic carbocyclic)-O—, -(bicyclic non-aromatic carbocyclic)-S—, -(bicyclic non-aromatic heterocyclyl)-, —CH₂-(bicyclic non-aromatic heterocyclyl)-, -(bicyclic non-aromatic heterocyclyl)-CH₂—, -(bicyclic non-aromatic heterocyclyl)-S—, -(bicyclic non-aromatic heterocyclyl)-O—, -phenyl-S—, -phenyl-O—, -phenyl-CH₂—, -phenyl-, -(monocyclic non-aromatic heterocyclyl)-, —CH₂-(monocyclic non-aromatic heterocyclyl)-, -(monocyclic non-aromatic heterocyclyl)-CH₂—, -(monocyclic non-aromatic heterocyclyl)-S—, -(monocyclic non-aromatic heterocyclyl)-O—, -monocyclic non-aromatic carbocyclic-, —CH₂-(monocyclic non-aromatic carbocyclic)-, -(monocyclic non-aromatic carbocyclic)-CH₂—, -(monocyclic non-aromatic carbocyclic)-S—, or -(monocyclic non-aromatic carbocyclic)-O—.

In certain such embodiments, R₃ is selected from -bicyclic partially or fully aromatic heterocyclic-, -(bicyclic partially or fully aromatic heterocyclic)-CH₂—, -bicyclic aryl-, -(bicyclic aryl)-CH₂—, —CH₂-(bicyclic non-aromatic carbocyclic)-, —CH₂-(bicyclic non-aromatic heterocyclyl)-, -(bicyclic non-aromatic carbocyclic)-, -(bicyclic non-aromatic heterocyclyl)-, -phenyl-S—, -phenyl-O—, -phenyl-CH₂—, -phenyl-, -(monocyclic non-aromatic heterocyclyl)-CH₂—, -monocyclic non-aromatic carbocyclic-, or -(monocyclic non-aromatic carbocyclic)-CH₂—;

R₄ is selected from H, (C₁-C₆)alkyl, (C₃-C₈)cycloalkyl, —C(O)R₁₅, —C(S)R₁₅, —(CR^{a}R^{b})_{q}O(C₁-C₆ alkyl), —(CR^{a}R^{b})_{q}S(C₁-C₆ alkyl), —(CR^{a}R^{b})ᵣC(O)R₁₅, —(CR^{a}R^{b})ᵣR₁₅ or —SO₂R₁₅;

R₅ is selected from (C₁-C₆)alkyl, (C₂-C₆)alkenyl, (C₂-C₆)alkynyl, —(CR^{a}R^{b})_{q}O(C₁-C₆ alkyl), (CR^{a}R^{b})_{q}S(C₁-C₆ alkyl); (C₃-C₈)cycloalkyl, —C(O)R₁₅, —C(S)R₁₅, —(CR^{a}R^{b})ᵣC(O)R₁₅, —(CR^{a}R^{b})ᵣC(S)R₁₅, —(CR^{a}R^{b})ᵣR₁₅, —SO₂R₁₅, phenyl, pyridyl, phenyl-Z₁- or pyridyl-Z₁-, wherein said phenyl or pyridyl is optionally substituted with one to five independently selected R₁₂;

or R₄ and R₅ taken together with the nitrogen atom to which they are attached form a 4-10 membered heterocyclyl (e.g., monocyclic heterocyclyl), wherein:
R₁₂ is selected from halo, cyano, nitro, azido, amino, hydroxy, (C₁-C₆)alkyl, (C₂-C₆)alkoxy, methoxy, (C₁-C₆)alkoxy(C₁-C₆)alkyl, mono-, di- or tri-halo(C₂-C₆)alkyl, perfluoro(C₂-C₄)alkyl, trifluoromethyl, trifluoromethyl(C₁-C₅)alkyl, mono-, di- or tri-halo(C₂-C₆)alkoxy, trifluoromethyl(C₁-C₅)alkoxy, (C₁-C₆)alkylthio, hydroxy(C₁-C₆)alkyl, (C₃-C₈)cycloalkyl(CR^{a}R^{b})_{q}—, (C₂-C₆)alkenyl, (C₂-C₆)alkynyl, (C₁-C₆)alkylamino-, (C₁-C₆)dialkylamino, amino(C₁-C₆)alkyl-, —(CR^{a}R^{b})_{q}NR^{a}R₁₄, —C(O)NR^{a}R₁₄, —NR₁₄C(O)R₁₅, —NR₁₄OR₁₅, —CH═NOR₁₅, —NR₁₄C(O)OR₁₅, —NR₁₄S(O)ⱼR₁₅, —C(O)R₁₅, —C(S)R₁₅, —C(O)OR₁₅, —OC(O)R₁₅, —SO₂NR^{a}R₁₄, —S(O)ⱼR₁₅, or —(CR^{a}R^{b})_{q}S(O)ⱼR₁₅;
each R₁₄ is independently H, (C₁-C₆)alkyl, (C₃-C₈)cycloalkyl, —C(O)R₁₅, —C(S)R₁₅, —(CR^{a}R^{b})ₜO(C₁-C₆ alkyl), —(CR^{a}R^{b})ₜS(C₁-C₆ alkyl), —(CR^{a}R^{b})ᵣC(O)R₁₅, —(CR^{a}R^{b})ₜR₁₅ or —SO₂R₁₅;
each R₁₅ is independently H, (C₁-C₆)alkyl or (C₃-C₈)cycloalkyl, wherein the alkyl moieties of the foregoing R₁₅ groups are independently optionally substituted with 1 to 3 substituents independently selected from C₁-C₆ alkyl, C₁-C₆ alkoxy, amino, hydroxy, halo, cyano, nitro, trifluoromethyl and trifluoromethoxy;
each j is independently 0, 1 or 2;
each q is independently an integer from 0 to 6;
each r is independently an integer from 1 to 5;
each t is independently an integer from 1 to 6; and
each v is independently an integer from 1 to 6;

wherein any alkyl, alkenyl, alkynyl or cyclic moieties of R₁, R₂, R₃, R₄, R₅, R₆, or R₇ groups are independently optionally substituted with 1 to 3 substituents independently selected from halo, cyano, nitro, trifluoromethyl, trifluoromethoxy, azido, —OR₁₅, —C(O)R₁₅, —C(O)OR₁₅, —OC(O)R₁₅, —NR₁₄C(O)R₁₅, —C(O)NR^{a}R₁₄, NR^{a}R₁₄, and —NR₁₄OR₁₅, C₁-C₆ alkyl, C₂-C₆ alkenyl, and C₂-C₆ alkynyl.

In certain embodiments, g is 0 or 1 and k is 0 or 1.

In another aspect, the invention provides a compound of formula (II):

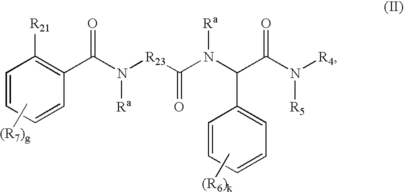

or a pharmaceutically acceptable salt thereof, wherein:

R₂₁ is selected from non-aromatic heterocyclyl; monocyclic heteroaryl, wherein said monocyclic heteroaryl comprises either: (i) at least one ring heteroatom selected from O or S, or (ii) at least two ring atoms independently selected from O, N or S; or bicyclic heteroaryl, wherein said bicyclic heteroaryl comprises a ring heteroatom selected from N or S;

R₂₃ is quinolinyl;

R₄ is selected from H, (C₁-C₆)alkyl, (C₃-C₈)cycloalkyl, —C(O)R₁₅, —C(S)R₁₅, —(CR^{a}R^{b})_{q}O(C₁-C₆ alkyl), —(CR^{a}R^{b})_{q}S(C₁-C₆ alkyl), —(CR^{a}R^{b})ᵣC(O)R₁₅, —(CR^{a}R^{b})ᵣR₁₅ or —SO₂R₁₅, such as —CF₃;

R₅ is selected from (C₁-C₆)alkyl, (C₂-C₆)alkenyl, (C₂-C₆)alkynyl, —(CR^{a}R^{b})_{q}O(C₁-C₆ alkyl), (CR^{a}R^{b})_{q}S(C₁-C₆ alkyl), (C₃-C₈)cycloalkyl, —C(O)R₁₅, —C(S)R₁₅, —(CR^{a}R^{b})ᵣC(O)R₁₅, —(CR^{a}R^{b})ᵣC(S)R₁₅, —(CR^{a}R^{b})ᵣR₁₅, —SO₂R₁₅, phenyl, pyridyl, phenyl-Z₁- or pyridyl-Z₁-;

or R₄ and R₅ taken together with the nitrogen atom to which they are attached form a 4-10 membered heterocyclyl;

each R^{a} and R^{b} is independently H or (C₁-C₆)alkyl, such as —CF₃;

Z₁ is —SO₂— or —(CR^{a}R^{b})ᵥ—;

each R₁₅ is independently H, (C₁-C₆)alkyl, or (C₃-C₈)cycloalkyl;

k is an integer from 0 to 5;

g is an integer from 0 to 4;

each j is 0, 1 or 2;

each q is independently an integer from 0 to 6;

each r is independently an integer from 1 to 5;

each v is independently an integer from 1 to 6;

R₆ is selected from halo, C₁-C₄ alkyl, or O—C₁-C₄ alkyl; and

R₇ is selected from R₆ or —X—R₁₆, wherein:
X is selected from a bond, —O—, —S—, —N(R^{a})—, —C(O)N(R^{a})—, or —N(R^{a})C(O)—; and
R₁₆ is selected from C₁-C₄ alkyl, —CH₂C(O)N(R₁₇)(R₁₈), cycloalkyl, or —(CH₂)ⱼ-heterocyclyl, wherein R₁₇ and R₁₈ are independently selected from hydrogen, alkyl, carbocyclyl, heterocyclyl, heteroalkyl, aminocarbonyl, alkylcarbonyl, alkoxycarbonyl, arylcarbonyl, or aryloxycarbonyl.

In certain embodiments, R₄ is selected from H, (C₁-C₆)alkyl, (C₃-C₈)cycloalkyl, —C(O)R₁₅, —C(S)R₁₅, —(CR^{a}R^{b})_{q}O(C₁-C₆ alkyl), —(CR^{a}R^{b})_{q}S(C₁-C₆ alkyl), —(CR^{a}R^{b})ᵣC(O)R₁₅, —(CR^{a}R^{b})ᵣR₁₅ or —SO₂R₁₅;

R₅ is selected from (C₁-C₆)alkyl, (C₂-C₆)alkenyl, (C₂-C₆)alkynyl, —(CR^{a}R^{b})_{q}O(C₁-C₆ alkyl), (CR^{a}R^{b})_{q}S(C₁-C₆ alkyl); (C₃-C₈)cycloalkyl, —C(O)R₁₅, —C(S)R₁₅, —(CR^{a}R^{b})ᵣC(O)R₁₅, —(CR^{a}R^{b})ᵣC(S)R₁₅, —(CR^{a}R^{b})ᵣR₁₅, —SO₂R₁₅, phenyl, pyridyl, phenyl-Z₁- or pyridyl-Z₁-, wherein said phenyl or pyridyl is optionally substituted with one to five independently selected R₁₂;

or R₄ and R₅ taken together with the nitrogen atom to which they are attached form a 4-10 membered heterocyclyl (e.g., monocyclic heterocyclyl), wherein:
R₁₂ is selected from halo, cyano, nitro, azido, amino, hydroxy, (C₁-C₆)alkyl, (C₂-C₆)alkoxy, methoxy, (C₁-C₆)alkoxy(C₁-C₆)alkyl, mono-, di- or tri-halo(C₂-C₆)alkyl, perfluoro(C₂-C₄)alkyl, trifluoromethyl, trifluoromethyl(C₁-C₅)alkyl, mono-, di- or tri-halo(C₂-C₆)alkoxy, trifluoromethyl(C₁-C₅)alkoxy, (C₁-C₆)alkylthio, hydroxy(C₁-C₆)alkyl, (C₃-C₈)cycloalkyl(CR^{a}R^{b})_{q}—, (C₂-C₆)alkenyl, (C₂-C₆)alkynyl, (C₁-C₆)alkylamino-, (C₁-C₆)dialkylamino, amino(C₁-C₆)alkyl-, —(CR^{a}R^{b})_{q}NR^{a}R₁₄, —C(O)NR^{a}R₁₄, —NR₁₄C(O)R₁₅, —NR₁₄OR₁₅, —CH═NOR₁₅, —NR₁₄C(O)OR₁₅, —NR₁₄S(O)ⱼR₁₅, —C(O)R₁₅, —C(S)R₁₅, —C(O)OR₁₅, —OC(O)R₁₅, —SO₂NR^{a}R₁₄, —S(O)ⱼR₁₅, or —(CR^{a}R^{b})_{q}S(O)ⱼR₁₅;
each R₁₄ is independently H, (C₁-C₆)alkyl, (C₃-C₈)cycloalkyl, —C(O)R₁₅, —C(S)R₁₅, —(CR^{a}R^{b})ₜO(C₁-C₆ alkyl), —(CR^{a}R^{b})ₜS(C₁-C₆ alkyl), —(CR^{a}R^{b})ᵣC(O)R₁₅, —(CR^{a}R^{b})ₜR₁₅ or —SO₂R₁₅;
each R₁₅ is independently H, (C₁-C₆)alkyl or (C₃-C₈)cycloalkyl, wherein the alkyl moieties of the foregoing R₁₅ groups are independently optionally substituted with 1 to 3 substituents independently selected from C₁-C₆ alkyl, C₁-C₆ alkoxy, amino, hydroxy, halo, cyano, nitro, trifluoromethyl and trifluoromethoxy;
each j is independently 0, 1 or 2;
each q is independently an integer from 0 to 6;
each r is independently an integer from 1 to 5;
each t is independently an integer from 1 to 6; and
each v is independently an integer from 1 to 6;

wherein any alkyl, alkenyl, alkynyl or cyclic moieties of R₂₁, R₂, R₂₃, R₄, R₅, R₆, or R₇ groups are optionally substituted independently with 1 to 3 substituents independently selected from halo, cyano, nitro, trifluoromethyl, trifluoromethoxy, azido, —OR₁₅, —C(O)R₁₅, —C(O)OR₁₅, —OC(O)R₁₅, —NR₁₄C(O)R₁₅, —C(O)NR^{a}R₁₄, NR^{a}R₁₄, and —NR₁₄OR₁₅, C₁-C₆ alkyl, C₂-C₆ alkenyl, and C₂-C₆ alkynyl.

In certain embodiments, g is 0 or 1 and k is 0 or 1.

In yet another aspect, the invention provides a compound of formula (III):

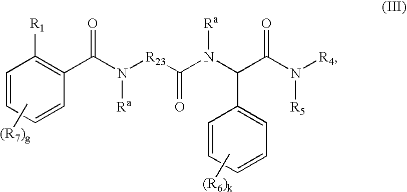

or a pharmaceutically acceptable salt thereof, wherein:

R₁ is selected from phenyl, a non-aromatic heterocyclyl, or partially or fully aromatic heterocyclic;

R₃₃ is selected from a bicyclic heterocycle comprising at least one aromatic ring and comprising at least two ring heteroatoms independently selected from N, O or S; -(bicyclic heteroaryl)-CH₂—; -bicyclic aryl-; -(bicyclic aryl)-CH₂—; —CH₂-(bicyclic non-aromatic carbocyclic)-; -(bicyclic non-aromatic carbocyclic)-; -(bicyclic non-aromatic carbocyclic)-CH₂; —CH₂-(bicyclic non-aromatic heterocyclyl)-; -(bicyclic non-aromatic heterocyclyl)-CH₂—; -phenyl-S—; -phenyl-O—; -(monocyclic saturated heterocyclyl)-CH₂—; -monocyclic non-aromatic carbocyclic-; or -(monocyclic non-aromatic carbocyclic)-CH₂—;

R₄ is selected from H, (C₁-C₆)alkyl, (C₃-C₈)cycloalkyl, —C(O)R₁₅, —C(S)R₁₅, —(CR^{a}R^{b})_{q}O(C₁-C₆ alkyl), —(CR^{a}R^{b})_{q}S(C₁-C₆ alkyl), —(CR^{a}R^{b})ᵣC(O)R₁₅, —(CR^{a}R^{b})ᵣR₁₅ or —SO₂R₁₅, such as —CF₃;

R₅ is selected from (C₁-C₆)alkyl, (C₂-C₆)alkenyl, (C₂-C₆)alkynyl, —(CR^{a}R^{b})_{q}O(C₁-C₆ alkyl), —(CR^{a}R^{b})_{q}S(C₁-C₆ alkyl); (C₃-C₈)cycloalkyl, —C(O)R₁₅, —C(S)R₁₅, —(CR^{a}R^{b})ᵣC(O)R₁₅, —(CR^{a}R^{b})ᵣC(S)R₁₅, —(CR^{a}R^{b})ᵣR₁₅, —SO₂R₁₅, phenyl, pyridyl, phenyl-Z₁- or pyridyl-Z₁-;

or R₄ and R₅ taken together with the nitrogen atom to which they are attached form a 4-10 membered heterocyclyl (e.g., monocyclic heterocyclyl), wherein:
each R^{a} and R^{b} is independently H or (C₁-C₆)alkyl, such as —CF₃;
Z₁ is —SO₂— or —(CR^{a}R^{b})ᵥ—;
each R₁₅ is independently H, (C₁-C₆)alkyl, or (C₃-C₈)cycloalkyl;
k is an integer from 0 to 5;
g is an integer from 0 to 4;
each j is independently 0, 1 or 2;
each q is independently an integer from 0 to 6;
each r is independently an integer from 1 to 5;
each v is independently an integer from 1 to 6; and

R₆ is selected from halo, C₁-C₄ alkyl, or O—C₁-C₄ alkyl; and

R₇ is selected from R₆, or —X—R₁₆, wherein:
X is selected from a bond, —O—, —S—, —N(R^{a})—, —C(O)N(R^{a})—, or —N(R^{a})C(O)—; and

R₁₆ is selected from C₁-C₄ alkyl, —CH₂C(O)N(R₁₇)(R₁₈), cycloalkyl, or —(CH₂)ⱼ-heterocyclyl, wherein R₁₇ and R₁₈ are independently selected from hydrogen, alkyl, carbocyclyl, heterocyclyl, heteroalkyl, aminocarbonyl, alkylcarbonyl, alkoxycarbonyl, arylcarbonyl, or aryloxycarbonyl.

In certain embodiments, R₄ is selected from H, (C₁-C₆)alkyl, (C₃-C₈)cycloalkyl, —C(O)R₁₅, —C(S)R₁₅, —(CR^{a}R^{b})_{q}O(C₁-C₆ alkyl), —(CR^{a}R^{b})_{q}S(C₁-C₆ alkyl), —(CR^{a}R^{b})ᵣC(O)R₁₅, —(CR^{a}R^{b})ᵣR₁₅ or —SO₂R₁₅;

R₅ is selected from (C₁-C₆)alkyl, (C₂-C₆)alkenyl, (C₂-C₆)alkynyl, —(CR^{a}R^{b})_{q}O(C₁-C₆ alkyl), (CR^{a}R^{b})_{q}S(C₁-C₆ alkyl), (C₃-C₈)cycloalkyl, —C(O)R₁₅, —C(S)R₁₅, —(CR^{a}R^{b})ᵣC(O)R₁₅, —(CR^{a}R^{b})ᵣC(S)R₁₅, —(Cr^{a}R^{b})ᵣR₁₅, —SO₂R₁₅, phenyl, pyridyl, phenyl-Z₁- or pyridyl-Z₁-, wherein said phenyl or pyridyl is optionally substituted with one to five independently selected R₁₂;

or R₄ and R₅ taken together with the nitrogen atom to which they are attached form a 4-10 membered monocyclic heterocyclyl, wherein:
R₁₂ is selected from halo, cyano, nitro, azido, amino, hydroxy, (C₁-C₆)alkyl, (C₂-C₆)alkoxy, methoxy, (C₁-C₆)alkoxy(C₁-C₆)alkyl, mono-, di- or tri-halo(C₂-C₆)alkyl, perfluoro(C₂-C₄)alkyl, trifluoromethyl, trifluoromethyl(C₁-C₅)alkyl, mono-, di- or tri-halo(C₂-C₆)alkoxy, trifluoromethyl(C₁-C₅)alkoxy, (C₁-C₆)alkylthio, hydroxy(C₁-C₆)alkyl, (C₃-C₈)cycloalkyl(CR^{a}R^{b})_{q}—, (C₂-C₆)alkenyl, (C₂-C₆)alkynyl, (C₁-C₆)alkylamino-, (C₁-C₆)dialkylamino, amino(C₁-C₆)alkyl-, —(CR^{a}R^{b})_{q}NR^{a}R₁₄, —C(O)NR^{a}R₁₄, —NR₁₄C(O)R₁₅, —NR₁₄OR₁₅, —CH═NOR₁₅, —NR₁₄C(O)OR₁₅, —NR₁₄S(O)ⱼR₁₅, —C(O)R₁₅, —C(S)R₁₅, —C(O)OR₁₅, —OC(O)R₁₅, —SO₂NR^{a}R₁₄, —S(O)ⱼR₁₅, or —(CR^{a}R^{b})_{q}S(O)ⱼR₁₅;
each R₁₄ is independently H, (C₁-C₆)alkyl, (C₃-C₈)cycloalkyl, —C(O)R₁₅, —C(S)R₁₅, —(CR^{a}R^{b})ₜO(C₁-C₆ alkyl), —(CR^{a}R^{b})ₜS(C₁-C₆ alkyl), —(CR^{a}R^{b})ᵣC(O)R₁₅, —(CR^{a}R^{b})ₜR₁₅ or —SO₂R₁₅;
each R₁₅ is independently H, (C₁-C₆)alkyl or (C₃-C₈)cycloalkyl, wherein the alkyl moieties of the foregoing R₁₅ groups are independently optionally substituted with 1 to 3 substituents independently selected from C₁-C₆ alkyl, C₁-C₆ alkoxy, amino, hydroxy, halo, cyano, nitro, trifluoromethyl and trifluoromethoxy;
each j is independently 0, 1 or 2;
each q is independently an integer from 0 to 6;
each r is independently an integer from 1 to 5;
each t is independently an integer from 1 to 6; and
each v is independently an integer from 1 to 6;

wherein any alkyl, alkenyl, alkynyl or cyclic moieties of R₁, R₂, R₃₃, R₄, R₅, R₆, or R₇ groups are optionally substituted independently with 1 to 3 substituents independently selected from halo, cyano, nitro, trifluoromethyl, trifluoromethoxy, azido, —OR₁₅, —C(O)R₁₅, —C(O)OR₁₅, —OC(O)R₁₅, —NR₁₄C(O)R₁₅, —C(O)NR^{a}R₁₄, NR^{a}R₁₄, and —NR₁₄OR₁₅, C₁-C₆ alkyl, C₂-C₆ alkenyl, and C₂-C₆ alkynyl.

In certain embodiments, g is 0 or 1 and k is 0 or 1.

In a further aspect, the invention provides a compound of formula (IV):

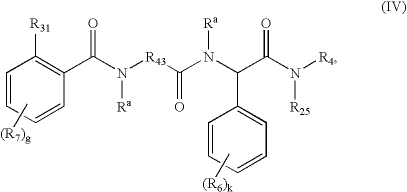

or a pharmaceutically acceptable salt thereof, wherein:

R₃₁ is selected from non-aromatic heterocyclyl, or partially or fully aromatic heterocyclic, wherein when said partially or fully aromatic heterocyclic is monocyclic it comprises either (i) at least one ring heteroatom selected from O or S, or (ii) at least two ring atoms independently selected from O, N or S;

R₄₃ is selected from benzofuranyl, dihydrobenzofuranyl, tetrahydrobenzofuranyl, benzothienyl, dihydrobenzothienyl, tetrahydrobenzothienyl, indolyl, dihydroindolyl or tetrahydroindolyl;

each of R₄ and R₂₅ is independently selected from (C₁-C₆)alkyl, (C₂-C₆)alkenyl, (C₂-C₆)alkynyl, —(CR^{a}R^{b})_{q}O(C₁-C₆ alkyl), (CR^{a}R^{b})_{q}S(C₁-C₆ alkyl); (C₃-C₈)cycloalkyl, —C(O)R₁₅, —C(S)R₁₅, —(CR^{a}R^{b})ᵣC(O)R₁₅, —(CR^{a}R^{b})ᵣC(S)R₁₅, —(CR^{a}R^{b})₁₅, or —SO₂R₁₅;

or R₄ and R₂₅ taken together with the nitrogen atom to which they are attached together form a 4-10 membered heterocyclyl (e.g., monocyclic heterocyclyl), wherein:
each R^{a} and R^{b} is independently H or (C₁-C₆)alkyl;
each R₁₅ is independently H, (C₁-C₆)alkyl, or (C₃-C₈)cycloalkyl;
k is an integer from 0 to 5;
g is an integer from 0 to 4;
each j is independently 0, 1 or 2;
each q is independently an integer from 0 to 6;
each r is independently an integer from 1 to 5; and

R₆ is selected from halo, C₁-C₄ alkyl, or O—C₁-C₄ alkyl; and

R₇ is selected from R₆, or —X—R₁₆, wherein:
X is selected from a bond, —O—, —S—, —N(R^{a})—, —C(O)N(R^{a})—, or —N(R^{a})C(O)—; and

R₁₆ is selected from C₁-C₄ alkyl, —CH₂C(O)N(R₁₇)(R₁₈), cycloalkyl, or —(CH₂)ⱼ-heterocyclyl, wherein R₁₇ and R₁₈ are independently selected from hydrogen, alkyl, carbocyclyl, heterocyclyl, heteroalkyl, aminocarbonyl, alkylcarbonyl, alkoxycarbonyl, arylcarbonyl, or aryloxycarbonyl.

In certain embodiments, each R₁₅ is independently H, (C₁-C₆)alkyl or (C₃-C₈)cycloalkyl, wherein the alkyl moieties of the foregoing R₁₅ groups are independently optionally substituted with 1 to 3 substituents independently selected from C₁-C₆ alkyl, C₁-C₆ alkoxy, amino, hydroxy, halo, cyano, nitro, trifluoromethyl and trifluoromethoxy;
each j is independently 0, 1 or 2;
each q is independently an integer from 0 to 6; and
each r is independently an integer from 1 to 5;

wherein any alkyl, alkenyl, alkynyl or cyclic moieties of R₃₁, R₄₃, R₄, R₂₅, R₆, or R₇ groups are optionally substituted independently with 1 to 3 substituents independently selected from halo, cyano, nitro, trifluoromethyl, trifluoromethoxy, azido, —OR₁₅, —C(O)R₁₅, —C(O)OR₁₅, —OC(O)R₁₅, —NR₁₄C(O)R₁₅, —C(O)NR^{a}R₁₄, NR^{a}R₁₄, and —N₁₄OR₁₅, C₁-C₆ alkyl, C₂-C₆ alkenyl, and C₂-C₆ alkynyl.

In certain embodiments, g is 0 or 1 and k is 0 or 1.

The invention additionally provides a compound of formula (V):

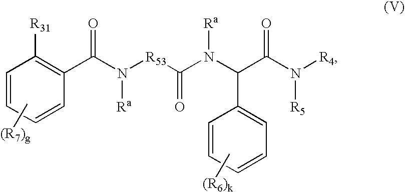

or a pharmaceutically acceptable salt thereof, wherein:

R₃₁ is selected from non-aromatic heterocyclyl, or partially or fully aromatic heterocyclic, wherein when said partially or fully aromatic heterocyclic is monocyclic it comprises either (i) at least one ring heteroatom selected from O or S, or (ii) at least two ring atoms independently selected from O, N or S;

R₅₃ is selected from phenyl or —CH₂-(phenyl)-;

R₄ is selected from H, (C₁-C₆)alkyl, (C₃-C₈)cycloalkyl, —C(O)R₁₅, —C(S)R₁₅, —(CR^{a}R^{b})_{q}O(C₁-C₆ alkyl), —(CR^{a}R^{b})_{q}S(C₁-C₆ alkyl), —(CR^{a}R^{b})ᵣC(O)R₁₅, —(CR^{a}R^{b})ᵣR₁₅ or —SO₂R₁₅;

R₅ is selected from (C₁-C₆)alkyl, (C₂-C₆)alkenyl, (C₂-C₆)alkynyl, —(CR^{a}R^{b})_{q}O(C₁-C₆ alkyl), —(CR^{a}R^{b})_{q}S(C₁-C₆ alkyl); (C₃-C₈)cycloalkyl, —C(O)R₁₅, —C(S)R₁₅, —(CR^{a}R^{b})ᵣC(O)R₁₅, —(CR^{a}R^{b})ᵣC(S)R₁₅, —(CR^{a}R^{b})ᵣR₁₅, —SO₂R₁₅, phenyl, pyridyl, phenyl-Z₁- or pyridyl-Z₁-;

or R₄ and R₅ taken together with the nitrogen atom to which they are attached form a 4-10 membered heterocyclyl (e.g., monocyclic heterocyclyl), wherein:
each R^{a} and R^{b} is independently H or (C₁-C₆)alkyl;
Z₁ is —SO₂— or —(CR^{a}R^{b})ᵥ—;
each R₁₅ is independently H, (C₁-C₆)alkyl, or (C₃-C₈)cycloalkyl;
k is an integer from 0 to 5;
g is an integer from 0 to 4;
each j is independently 0, 1 or 2;
each q is independently an integer from 0 to 6;
each r is independently an integer from 1 to 5;
each v is independently an integer from 1 to 6; and

R₆ is selected from halo, C₁-C₄ alkyl, or O—C₁-C₄ alkyl; and

R₇ is selected from R₆ or —X—R₁₆, wherein:
X is selected from a bond, —O—, —S—, —N(R^{a})—, —C(O)N(R^{a})—, or —N(R^{a})C(O)—; and

R₁₆ is selected from C₁-C₄ alkyl, —CH₂C(O)N(R₁₇)(R₁₈), cycloalkyl, or —(CH₂)ⱼ-heterocyclyl, wherein R₁₇ and R₁₈ are independently selected from hydrogen, alkyl, carbocyclyl, heterocyclyl, heteroalkyl, aminocarbonyl, alkylcarbonyl, alkoxycarbonyl, arylcarbonyl, or aryloxycarbonyl.

In certain embodiments, R₄ is selected from H, (C₁-C₆)alkyl, (C₃-C₈)cycloalkyl, —C(O)R₁₅, —C(S)R₁₅, —(CR^{a}R^{b})_{q}O(C₁-C₆ alkyl), —(CR^{a}R^{b})_{q}S(C₁-C₆ alkyl), —(CR^{a}R^{b})ᵣC(O)R₁₅, —(CR^{a}R^{b})ᵣR₁₅ or —SO₂R₅;

R₅ is selected from (C₁-C₆)alkyl, (C₂-C₆)alkenyl, (C₂-C₆)alkynyl, —(CR^{a}R^{b})_{q}O(C₁-C₆ alkyl), (CR^{a}R^{b})_{q}S(C₁-C₆ alkyl), (C₃-C₈)cycloalkyl, —C(O)R₁₅, —C(S)R₁₅, —(CR^{a}R^{b})ᵣC(O)R₁₅, —(CR^{a}R^{b})ᵣC(S)R₁₅, —(CR^{a}R^{b})ᵣR₁₅, —SO₂R₁₅, phenyl, pyridyl, phenyl-Z₁- or pyridyl-Z₁-, wherein said phenyl or pyridyl is optionally substituted with one to five independently selected R₁₂;

or R₄ and R₅ taken together with the nitrogen atom to which they are attached form a 4-10 membered heterocyclyl (e.g., monocyclic heterocyclyl), wherein:
R₁₂ is selected from halo, cyano, nitro, azido, amino, hydroxy, (C₁-C₆)alkyl, (C₂-C₆)alkoxy, methoxy, (C₁-C₆)alkoxy(C₁-C₆)alkyl, mono-, di- or tri-halo(C₂-C₆)alkyl, perfluoro(C₂-C₄)alkyl, trifluoromethyl, trifluoromethyl(C₁-C₅)alkyl, mono-, di- or tri-halo(C₂-C₆)alkoxy, trifluoromethyl(C₁-C₅)alkoxy, (C₁-C₆)alkylthio, hydroxy(C₁-C₆)alkyl, (C₃-C₈)cycloalkyl(CR^{a}R^{b})_{q}—, (C₂-C₆)alkenyl, (C₂-C₆)alkynyl, (C₁-C₆)alkylamino-, (C₁-C₆)dialkylamino, amino(C₁-C₆)alkyl-, —(CR^{a}R^{b})_{q}NR^{a}R₁₄, —C(O)NR^{a}R₁₄, —NR₁₄C(O)R₁₅, —NR₁₄OR₁₅, —CH═NOR₁₅, —NR₁₄C(O)OR₁₅, —NR₁₄S(O)ⱼR₁₅, —C(O)R₁₅, —C(S)R₁₅, —C(O)OR₁₅, —OC(O)R₁₅, —SO₂NR^{a}R₁₄, —S(O)ⱼR₁₅, or —(CR^{a}R^{b})_{q}S(O)ⱼR₁₅;
each R₁₄ is independently H, (C₁-C₆)alkyl, (C₃-C₈)cycloalkyl, —C(O)R₁₅, —C(S)R₁₅, —(CR^{a}R^{b})ₜO(C₁-C₆ alkyl), —(CR^{a}R^{b})ₜS(C₁-C₆ alkyl), —(CR^{a}R^{b})ᵣC(O)R₁₅, —(CR^{a}R^{b})ₜR₁₅ or —SO₂R₁₅;
each R₁₅ is independently H, (C₁-C₆)alkyl or (C₃-C₈)cycloalkyl, wherein the alkyl moieties of the foregoing R₁₅ groups are independently optionally substituted with 1 to 3 substituents independently selected from C₁-C₆ alkyl, C₁-C₆ alkoxy, amino, hydroxy, halo, cyano, nitro, trifluoromethyl and trifluoromethoxy;
each j is independently 0, 1 or 2;
each q is independently an integer from 0 to 6;
each r is independently an integer from 1 to 5;
each t is independently an integer from 1 to 6; and
each v is independently an integer from 1 to 6;

wherein any alkyl, alkenyl, alkynyl or cyclic moieties of R₃₁, R₅₃, R₄, R₅, R₆, or R₇ groups are optionally substituted independently with 1 to 3 substituents independently selected from halo, cyano, nitro, trifluoromethyl, trifluoromethoxy, azido, —OR₁₅, —C(O)R₁₅, —C(O)OR₁₅, —OC(O)R₁₅, —NR₁₄C(O)R₁₅, —C(O)NR^{a}R₁₄, NR^{a}R₁₄, and —NR₁₄OR₁₅, C₁-C₆ alkyl, C₂-C₆ alkenyl, and C₂-C₆ alkynyl.

In certain embodiments, g is 0 or 1 and k is 0 or 1.

In another aspect, the invention provides a compound of formula (VI):

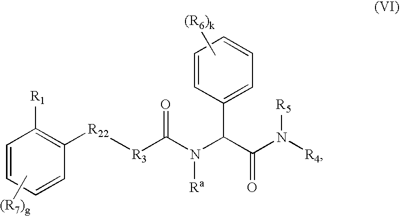

or a pharmaceutically acceptable salt thereof, wherein:

R₁ is selected from phenyl, non-aromatic heterocyclyl, or partially or fully aromatic heterocyclyl;

R₂₂ is —CH(R^{a})—N(R^{a})—;

R₃ is selected from -bicyclic partially or fully aromatic heterocyclyl-, -(bicyclic partially or fully aromatic heterocyclyl)-CH₂—, -bicyclic aryl-, -(bicyclic aryl)-CH₂—, —CH₂-(bicyclic non-aromatic cycloalkyl), —CH₂-(bicyclic non-aromatic heterocyclyl), -phenyl-S—, -phenyl-O—, -phenyl-CH₂—, -phenyl-, -(monocyclic non-aromatic heterocyclyl)-CH₂—, -monocyclic non-aromatic cycloalkyl-, or -(monocyclic non-aromatic cycloalkyl)-CH₂—;

R₄ is selected from H, (C₁-C₆)alkyl, (C₃-C₈)cycloalkyl, —C(O)R₁₅, —C(S)R₁₅, —(CR^{a}R^{b})_{q}O(C₁-C₆ alkyl), —(CR^{a}R^{b})_{q}S(C₁-C₆ alkyl), —(CR^{a}R^{b})ᵣC(O)R₁₅, —(CR^{a}R^{b})ᵣR₁₅ or —SO₂R₁₅;

R₅ is selected from (C₁-C₆)alkyl, (C₂-C₆)alkenyl, (C₂-C₆)alkynyl, —(CR^{a}R^{b})_{q}O(C₁-C₆ alkyl), —(CR^{a}R^{b})_{q}S(C₁-C₆ alkyl); (C₃-C₈)cycloalkyl, —C(O)R₁₅, —C(S)R₁₅, —(CR^{a}R^{b})ᵣC(O)R₁₅, —(CR^{a}R^{b})ᵣC(S)R₁₅, —(CR^{a}R^{b})ᵣR₁₅, —SO₂R₁₅, phenyl, pyridyl, phenyl-Z₁- or pyridyl-Z₁-;

or R₄ and R₅ taken together with the nitrogen atom to which they are attached form a 4-10 membered heterocyclyl (e.g., monocyclic heterocyclyl), wherein:
each R^{a} and R^{b} is independently H or (C₁-C₆)alkyl;
Z₁ is —SO₂— or —(CR^{a}R^{b})ᵥ;
each R₁₅ is independently H, (C₁-C₆)alkyl, or (C₃-C₈)cycloalkyl;
k is an integer from 0 to 5;
g is an integer from 0 to 4;
each j is independently 0, 1 or 2;
each q is independently an integer from 0 to 6;
each r is independently an integer from 1 to 5;
each v is independently an integer from 1 to 6; and

R₆ is selected from halo, C₁-C₄ alkyl, or O—C₁-C₄ alkyl; and

R₇ is selected from R₆, or —X—R₁₆, wherein:
X is selected from a bond, —O—, —S—, —N(R^{a})—, —C(O)N(R^{a})—, or —N(R^{a})C(O)—; and
R₁₆ is selected from C₁-C₄ alkyl, —CH₂C(O)N(R₁₇)(R₁₈), cycloalkyl, or —(CH₂)ⱼ-heterocyclyl, wherein R₁₇ and R₁₈ are independently selected from hydrogen, alkyl, carbocyclyl, heterocyclyl, heteroalkyl, aminocarbonyl, alkylcarbonyl, alkoxycarbonyl, arylcarbonyl, or aryloxycarbonyl.

In certain embodiments, R₄ is selected from H, (C₁-C₆)alkyl, (C₃-C₈)cycloalkyl, —C(O)R₁₅, —C(S)R₁₅, —(CR^{a}R^{b})_{q}O(C₁-C₆ alkyl), —(CR^{a}R^{b})_{q}S(C₁-C₆ alkyl), —(CR^{a}R^{b})ᵣC(O)R₁₅, —(CR^{a}R^{b})ᵣR₁₅ or —SO₂R₁₅;

R₅ is selected from (C₁-C₆)alkyl, (C₂-C₆)alkenyl, (C₂-C₆)alkynyl, —(CR^{a}R^{b})_{q}O(C₁-C₆ alkyl), (CR^{a}R^{b})_{q}S(C₁-C₆ alkyl); (C₃-C₈)cycloalkyl, —C(O)R₁₅, —C(S)R₁₅, —(CR^{a}R^{b})ᵣC(O)R₁₅, —(CR^{a}R^{b})ᵣC(S)R₁₅, —(CR^{a}R^{b})ᵣR₁₅, —SO₂R₁₅, phenyl, pyridyl, phenyl-Z₁- or pyridyl-Z₁-, wherein said phenyl or pyridyl is optionally substituted with one to five independently selected R₁₂;

or R₄ and R₅ taken together with the nitrogen atom to which they are attached form a 4-10 membered monocyclic heterocyclyl, wherein:
R₁₂ is selected from halo, cyano, nitro, azido, amino, hydroxy, (C₁-C₆)alkyl, (C₂-C₆)alkoxy, methoxy, (C₁-C₆)alkoxy(C₁-C₆)alkyl, mono-, di- or tri-halo(C₂-C₆)alkyl, perfluoro(C₂-C₄)alkyl, trifluoromethyl, trifluoromethyl(C₁-C₅)alkyl, mono-, di- or tri-halo(C₂-C₆)alkoxy, trifluoromethyl(C₁-C₅)alkoxy, (C₁-C₆)alkylthio, hydroxy(C₁-C₆)alkyl, (C₃-C₈)cycloalkyl(CR^{a}R^{b})_{q}—, (C₂-C₆)alkenyl, (C₂-C₆)alkynyl, (C₁-C₆)alkylamino-, (C₁-C₆)dialkylamino, amino(C₁-C₆)alkyl-, —(CR^{a}R^{b})_{q}NR^{a}R₁₄, —C(O)NR^{a}R₁₄, —NR₁₄C(O)R₁₅, —NR₁₄OR₁₅, —CH═NOR₁₅, —NR₁₄C(O)OR₁₅, —NR₁₄S(O)ⱼR₁₅, —C(O)R₁₅, —C(S)R₁₅, —C(O)OR₁₅, —OC(O)R₁₅, —SO₂NR^{a}R₁₄, —S(O)ⱼR₁₅, or —(CR^{a}R^{b})_{q}S(O)ⱼR₁₅;
each R₁₄ is independently H, (C₁-C₆)alkyl, (C₃-C₈)cycloalkyl, —C(O)R₁₅, —C(S)R₁₅, —(CR^{a}R^{b})ₜO(C₁-C₆ alkyl), —(CR^{a}R^{b})ₜS(C₁-C₆ alkyl), —(CR^{a}R^{b})ᵣC(O)R₁₅, —(CR^{a}R^{b})ₜR₁₅ or —SO₂R₁₅;
each R₁₅ is independently H, (C₁-C₆)alkyl or (C₃-C₈)cycloalkyl, wherein the alkyl moieties of the foregoing R₁₅ groups are independently optionally substituted with 1 to 3 substituents independently selected from C₁-C₆ alkyl, C₁-C₆ alkoxy, amino, hydroxy, halo, cyano, nitro, trifluoromethyl and trifluoromethoxy;
each j is independently 0, 1 or 2;
each q is independently an integer from 0 to 6;
each r is independently an integer from 1 to 5;
each t is independently an integer from 1 to 6; and
each v is independently an integer from 1 to 6;
wherein any alkyl, alkenyl, alkynyl or cyclic moieties of R₃₁, R₅₃, R₄, R₅, R₆, or R₇ groups are optionally substituted independently with 1 to 3 substituents independently selected from halo, cyano, nitro, trifluoromethyl, trifluoromethoxy, azido, —OR₁₅, —C(O)R₁₅, —C(O)OR₁₅, —OC(O)R₁₅, —NR₁₄C(O)R₁₅, —C(O)NR^{a}R₁₄, NR^{a}R₁₄, and —NR₁₄OR₁₅, C₁-C₆ alkyl, C₂-C₆ alkenyl, and C₂-C₆ alkynyl.

In certain embodiments, g is 0 or 1 and k is 0 or 1.

In certain embodiments, R₁ in compounds of formulas (I), (III) and (VI) is phenyl.

In certain such embodiments, R₁ is a phenyl substituted with a C₁-C₆ alkyl group, such as 4-trifluoromethyl, 4-isopropylphenyl or 4-t-butylphenyl.

In certain embodiments, R₃ in compounds of formulas (I) or (VI) is quinolinyl, particularly as follows:

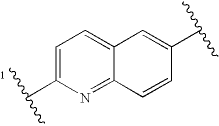

and is oriented such that R₃ is bound to R₂ or R₂₂ through the bond indicated by “1”.

In certain embodiments, R₃ or R₃₃ in compounds of formula (I), (III) or (VI) is selected from:

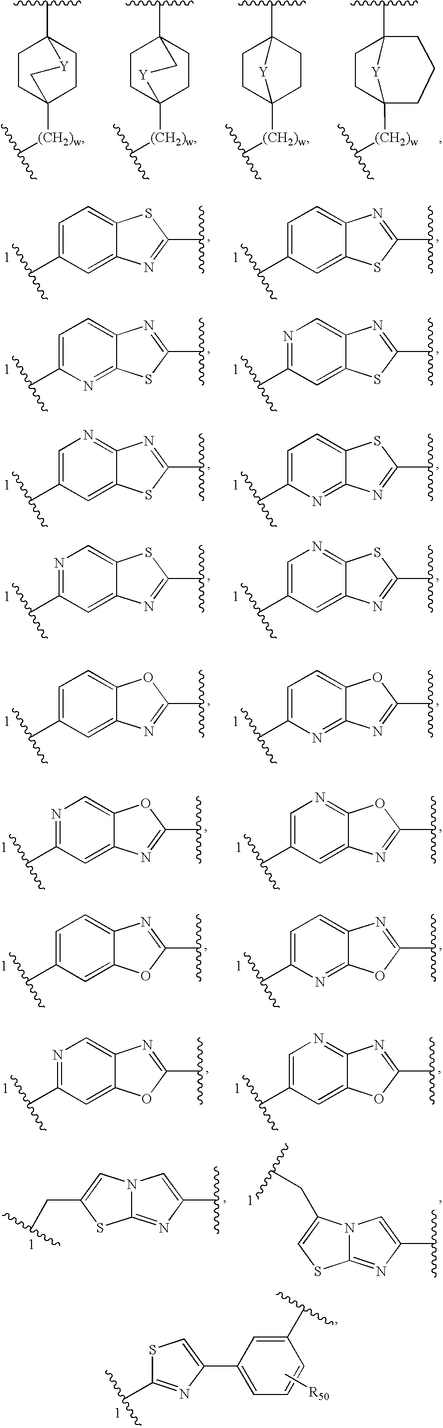

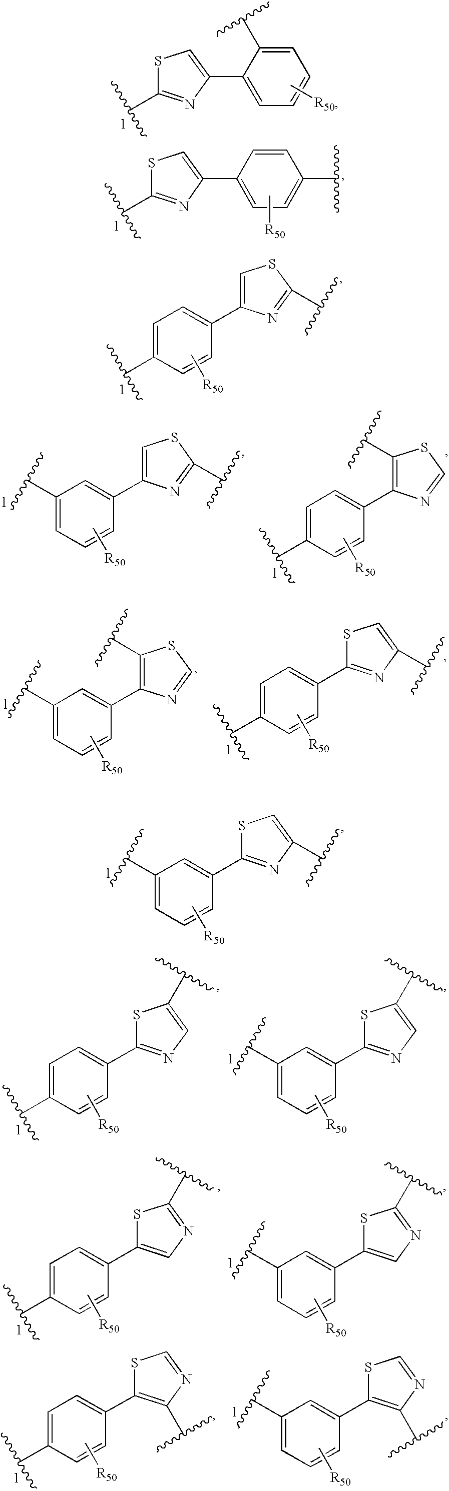

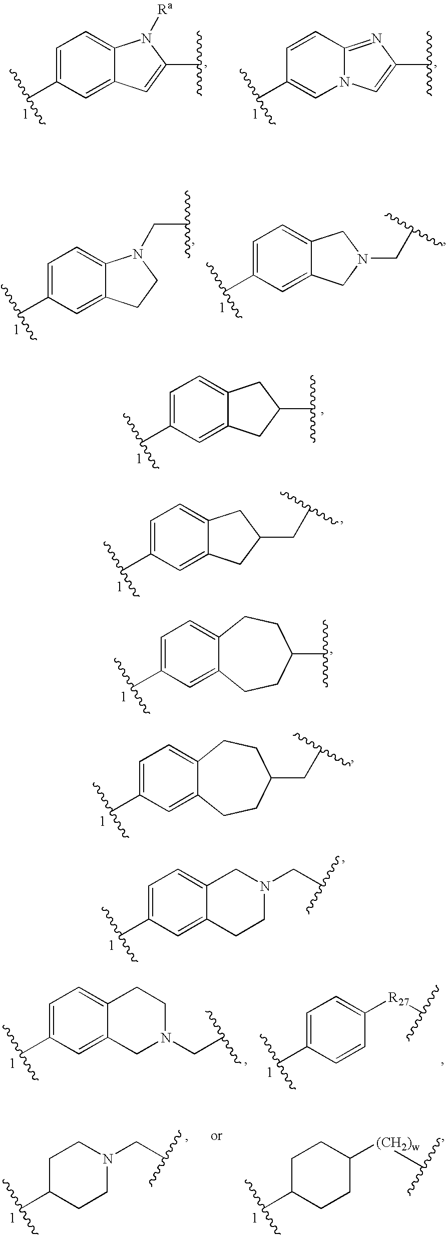

wherein:

Y is selected from —S—, —O—, —N— or —CH₂—;

R₂₇ is selected from a direct bond, —S—, —O—, or —CH₂—;

R₅₀ is —H or —O—(C₁-C₄ alkyl);

w is 0 or 1; and

said R₃ or R₃₃ is bound to R₂, R₂₂ or —N(R^{a}) through the bond indicated by “1” or through either bond if no bond is labeled by to “1”.

In certain such embodiments, R₃ or R₃₃ is

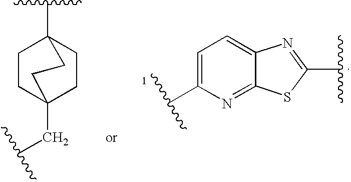

In certain embodiments, R₁, R₂₁ or R₃₁ is not phenyl, such as a heterocyclic group. In certain such embodiments, R₁, R₂₁ or R₃₁ is substituted by a trifluoromethyl or t-butyl group.

In certain embodiments, R₁, R₂₁ or R₃₁ in compounds of the invention is selected from:

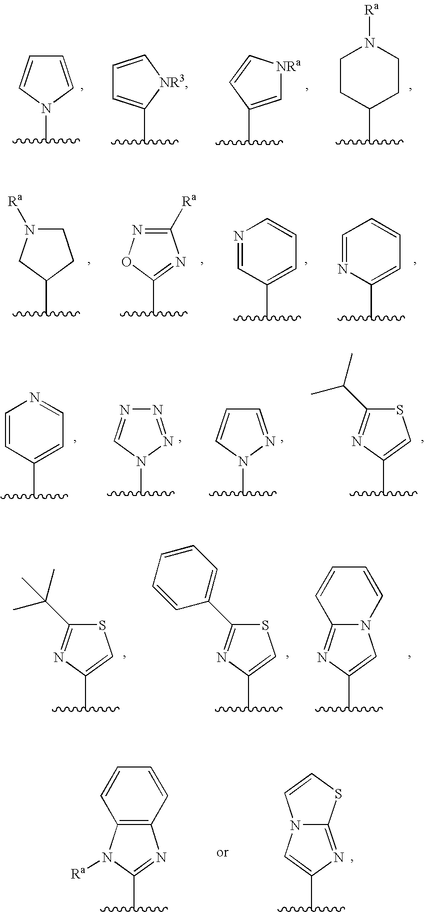

wherein any ring carbon is optionally substituted with a C₁-C₄ straight or branched alkyl group.

In certain embodiments, R₁ is a phenyl, such as a phenyl optionally substituted with one or more of a C₁-C₄ alkyl, —O—(C₁-C₄ alkyl) or —S—(C₁-C₄ alkyl) group. Such substituents may be typically in the ortho position (relative to the bond linking R₁ to the remainder of the compound) distal to the remainder of the molecule.

In certain embodiments, R₂ or R₂₂ in compounds of formula (I) or (VI) is —CH(CH₃)—NH—.

In certain embodiments for compounds of formula (I), (II), (III), (V) or (VI), R₄ is hydrogen or (C₁-C₆)alkyl; and R₅ is (C₁-C₆)alkyl or —(CR^{a}R^{b})ᵥ-phenyl, wherein said phenyl is optionally substituted with a C₁-C₄ alkyl or halo; or wherein R₄ and R₅ taken together with the nitrogen atom to which they are attached form a 5- to 7-membered monocyclic saturated heterocyclyl moiety or an 8- to 10-membered bicyclic heterocyclyl moiety.

In certain such embodiments, N(R₄)(R₅) is selected from

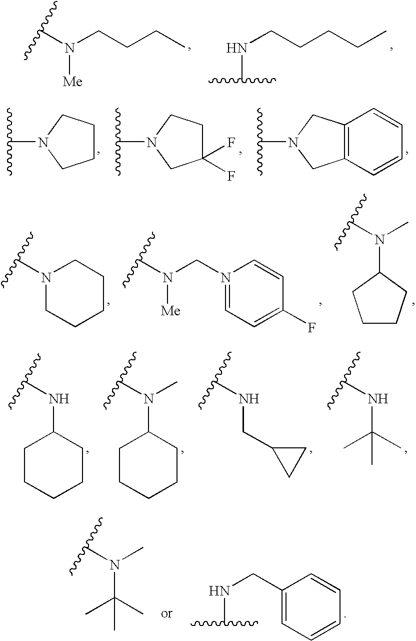

In certain embodiments, R₇ in compounds of the invention is —H, a C₁-C₄ alkyl (e.g, methyl) or a C₁-C₄ alkoxy (e.g, methoxy), particularly —H or —CH₃. In certain embodiments, R₆ is —H. Typically, R₆ is —H and R₇ is —H, a C₁-C₄ alkyl (e.g, methyl) or a C₁-C₄ alkoxy (e.g, methoxy).

In certain embodiments, R₁ in compounds of formula (I) or (III) is phenyl, including a phenyl substituted with one or more alkyl groups, such as iso-propyl, tert-butyl, sec-butyl, iso-pentyl, methyl, ethyl, propyl, n-butyl or phenyl. In certain embodiments, R₁ in compounds of formula (I) and (III) is 4-iso-propylphenyl, or 4-tert-butylphenyl.

In certain embodiments, R₁ in compounds of formula (I) or (III) is a substituted phenyl, e.g., 4-iso-propylphenyl, or 4-tert-butylphenyl, and R₃ or R₃₃ is benzothiazole. In other embodiments, R₁ in compounds of formula (I) or (III) is a substituted phenyl, e.g., 4-iso-propylphenyl, or 4-tert-butylphenyl and R₃ and R₃₃ are

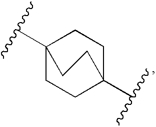

i.e., 1,4-bicyclo[2,2,2]octane. In particular embodiments, R₁ and R₃ or R₃₃ have the values above and R₇ is selected from —H, (C₁-C₄)alkyl or (C₁-C₄)alkoxy, particularly —H, -Me or —OMe. Further, R₆ and R^{a} in certain of these embodiments is —H.

In certain embodiments, —NR₄R₅ of compounds of formula (I) or (III) is a cyclic amine, e.g.,

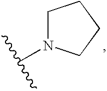

i.e., pyrrolidine. In certain embodiments, —NR₄R₅ of compounds of formula (I) or (III) is

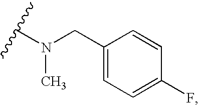

i.e. N-(4-fluorobenzyl)-N-methyl-1-amine. In certain embodiments, R₁ in compounds of formula (I) and (III) is substituted phenyl, e.g., 4-iso-propylphenyl, or 4-tert-butylphenyl, and —NR₄R₅ is pyrrolidine or N-(4-fluorobenzyl)-N-methyl-1-amine. In certain embodiments, R₃ or R₃₃ of compounds of formula (I) or (III) is benzothiazole or 1,4-bicyclo[2,2,2]octane and —NR₄R₅ is pyrrolidine or N-(4-fluorobenzyl)-N-methyl-1-amine. In certain embodiments, R₁ in compounds of formula (I) and (III) is substituted phenyl, e.g., 4-iso-propylphenyl, or 4-tert-butylphenyl, R₃ or R₃₃ is benzothiazole or 1,4-bicyclo[2,2,2]octane, and —NR₄R₅ is pyrrolidine or N-(4-fluorobenzyl)-N-methyl-1-amine.

Other exemplary compounds of the invention are represented by one of the following formulas:

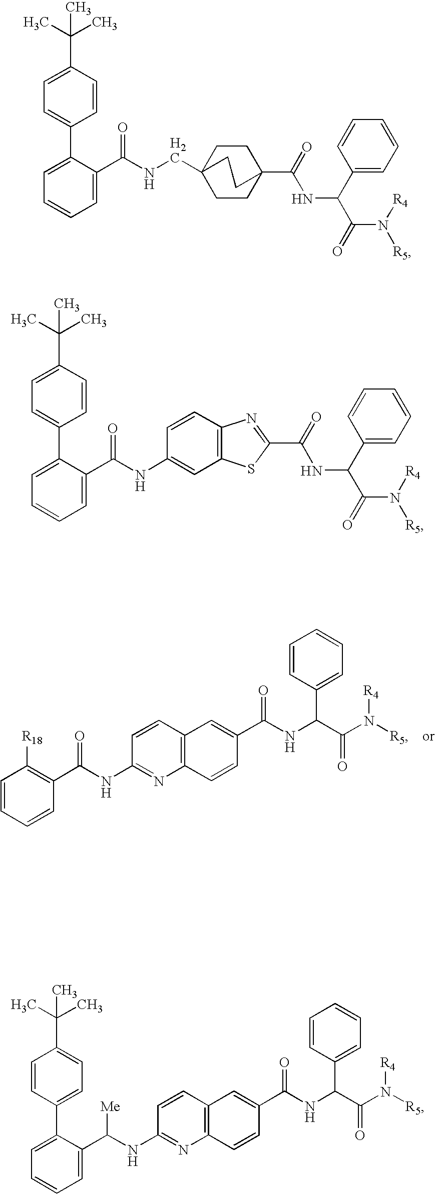

wherein:

R₁₈ is selected from

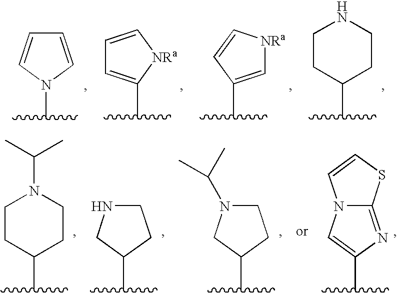

In certain embodiments for compounds of formula (IV), R₄ is hydrogen or (C₁-C₆)alkyl; and R₂₅ is (C₁-C₆)alkyl; or wherein R₄ and R₂₅ taken together with the nitrogen atom to which they are attached form a 5- to 7-membered monocyclic saturated heterocyclyl moiety.

In certain such embodiments, N(R₄)(R₂₅) is selected from

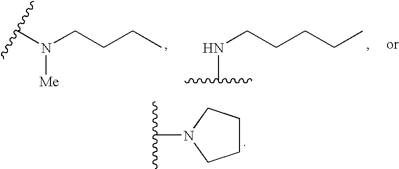

Exemplary compounds of the invention include the following:

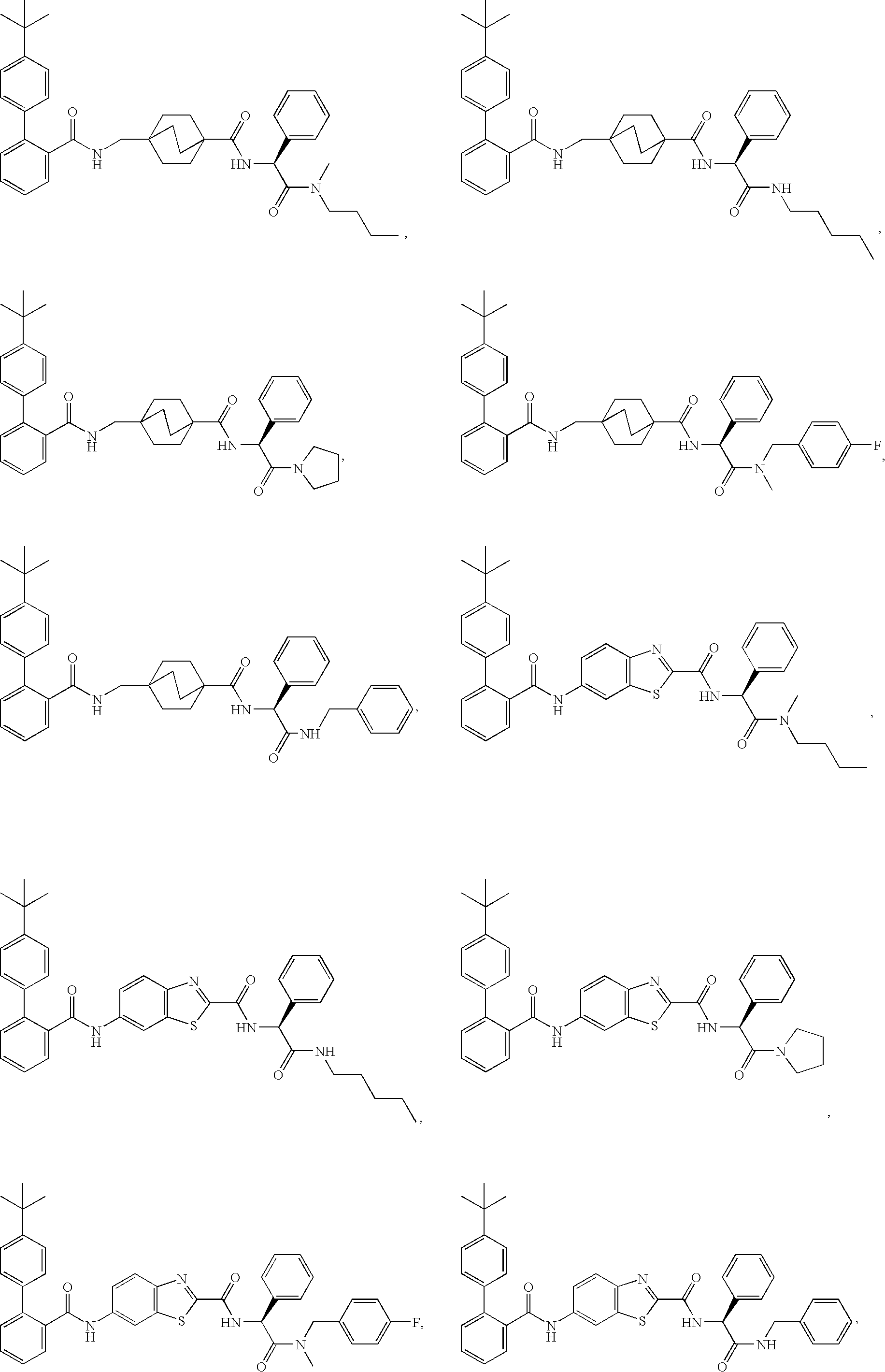

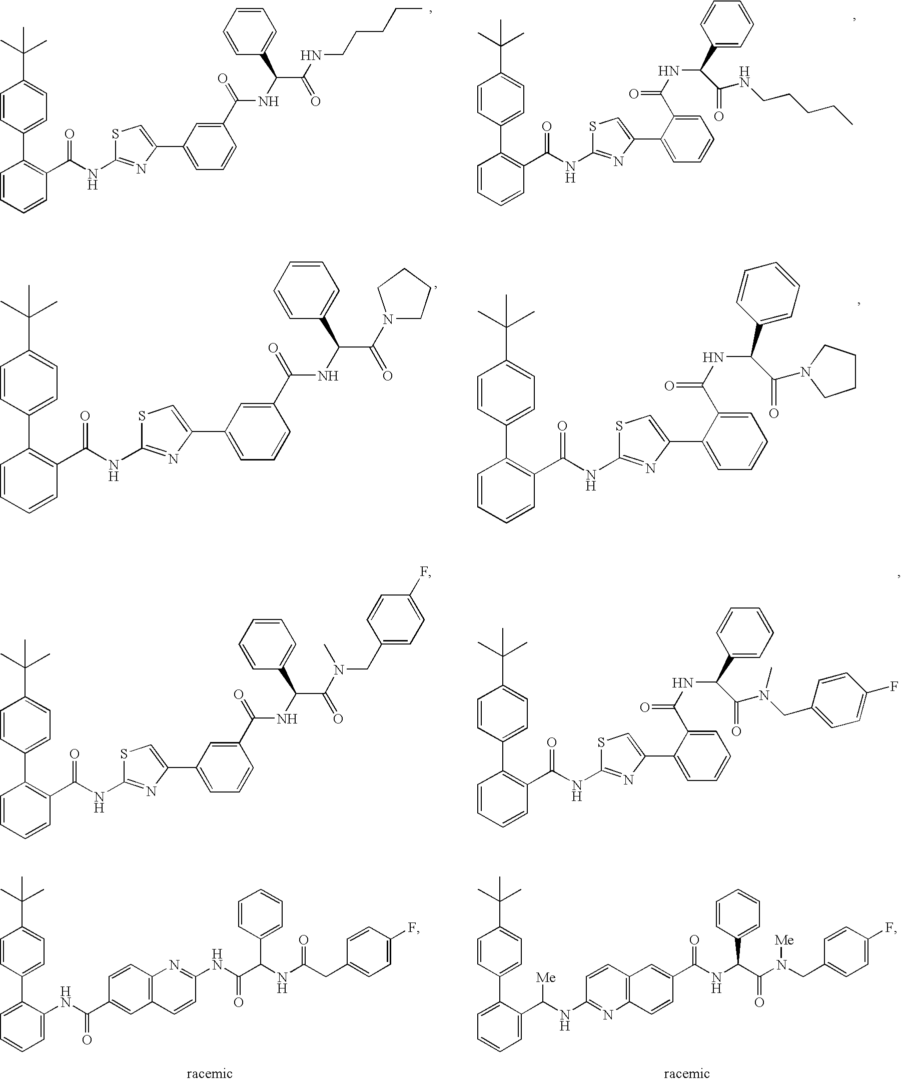

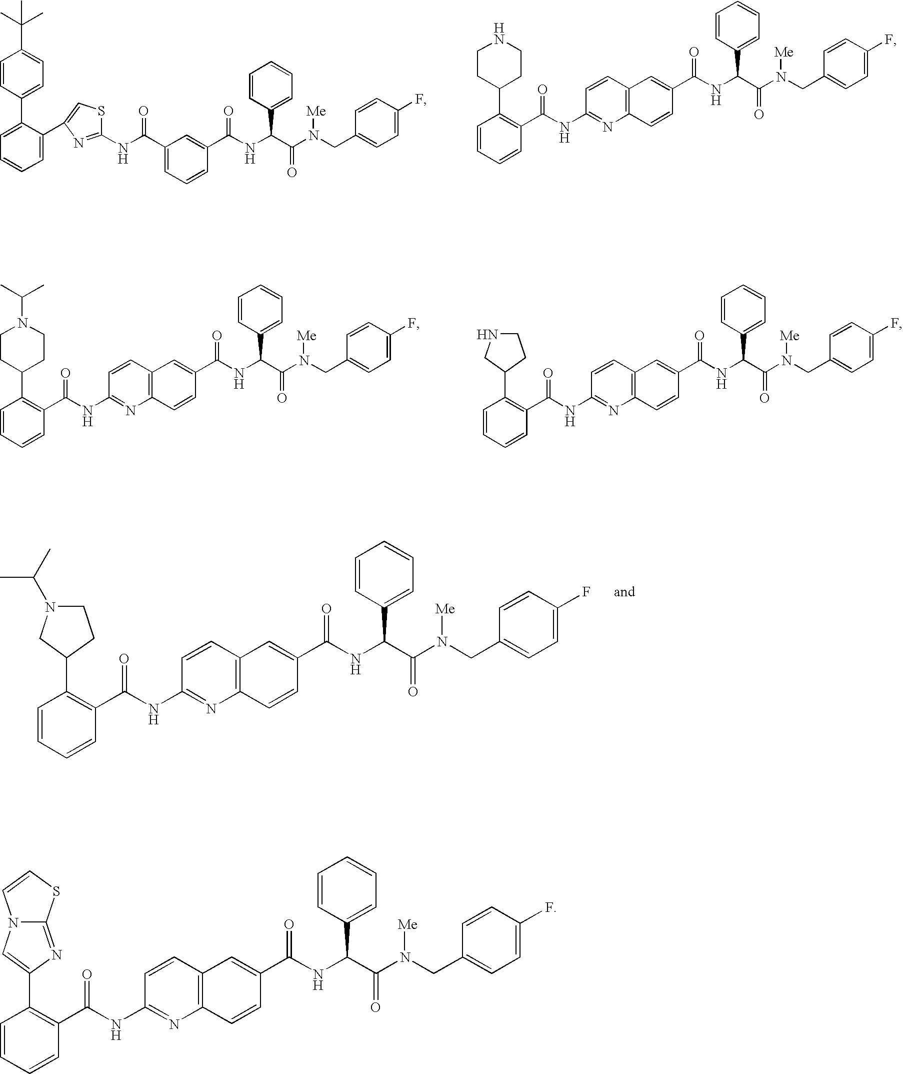

Unless otherwise specified, alkyl, alkenyl, alkynyl and all cyclic (including heterocyclic) groups can be substituted with one or more suitable substituents.

An alkyl group is a straight chained, branched or cyclic non-aromatic hydrocarbon which is completely saturated. Typically, a straight chained or branched alkyl group has from 1 to about 20 carbon atoms, preferably from 1 to about 10, and a cyclic alkyl group has from 3 to about 10 carbon atoms, preferably from 3 to about 8. Examples of straight chained and branched alkyl groups include methyl, ethyl, n-propyl, iso-propyl, n-butyl, sec-butyl, tert-butyl, pentyl, hexyl, pentyl and octyl. A C1-C4 straight chained or branched alkyl group is also referred to as a “lower alkyl” group.

A heteroalkyl group is an alkyl group that is interrupted by one or more heteroatoms (e.g., N, S, O, P, Si). The heteroatom may not be located at a terminus of a heteroalkyl group.

An alkenyl group is a straight chained, branched or cyclic non-aromatic hydrocarbon which contains one or more double bonds. Typically, the double bonds are not located at the terminus of the alkenyl group, such that the double bond is not adjacent to another functional group.

An alkynyl group is a straight chained, branched or cyclic non-aromatic hydrocarbon which contains one or more triple bonds. Typically, the triple bonds are not located at the terminus of the alkynyl group, such that the triple bond is not adjacent to another functional group.

A ring (e.g., 5- to 7-membered ring) or cyclic group includes carbocyclic and heterocyclic rings. Such rings can be saturated or unsaturated, including aromatic and partially aromatic. Heterocyclic rings typically contain 1 to 4 heteroatoms, although oxygen and sulfur atoms cannot be adjacent to each other.

A monocyclic group consists of one ring.

A bicyclic group consists of two rings that a) share a common bond (fused rings), b) share at least two common atoms (bridged rings), c) share a common atom (spiro-fused rings) or d) are connected by a bond.

A carbocyclic group is a monocyclic or polycyclic ring system that contains only carbon atoms.

Aromatic (aryl) groups are fully carbocyclic aromatic groups having one or more rings such as phenyl, naphthyl, and anthracyl. The terms “aryl” and “fully aromatic carbocyclic” are used interchangeably in this application.

Heteroaryl groups consist of one or more rings, where each ring is aromatic and at least one ring includes at least one heteroatom. Examples include imidazolyl, thienyl, furyl, pyridyl, pyrimidyl, pyranyl, pyrazolyl, pyrroyl, pyrazinyl, thiazolyl, oxazolyl, and tetrazolyl.

Heteroaryl groups also include fused polycyclic aromatic ring systems in which a carbocyclic aromatic ring or heteroaryl ring is fused to one or more other heteroaryl rings. Examples include benzothienyl, benzofuryl, indolyl, quinolinyl, benzothiazole, benzoxazole, benzimidazole, quinolinyl, isoquinolinyl and isoindolyl. The terms “heteroaryl” and “fully aromatic heterocyclyl” are used interchangeably in this application.

Partially aromatic heterocyclic groups are polycyclic (including bicyclic) ring systems where at least one ring is aromatic, at least one ring is not aromatic and at least one ring includes a heteroatom. Examples of partially aromatic heterocyclic groups include dihydrobenzofuranyl, tetrahydrobenzofuranyl, dihydrobenzothienyl, tetrahydrobenzothienyl, dihydroindolyl and tetrahydroindolyl. In contrast, all rings within a fully aromatic heterocyclic group have aromatic character.

Non-aromatic heterocyclic groups consist of one or more rings, where each ring is non-aromatic and at least rings contains one or more heteroatoms such as nitrogen, oxygen or sulfur. Non-aromatic heterocyclic group include fully saturated ring systems and ring systems having one or more degrees of unsaturation, provided that the ring system does not have aromatic character. Each ring in the ring system can be five, six, seven or eight-membered. Examples include tetrahydrofuryl, tetrahyrothiophenyl, dihydropyridine, dihydropyran, thiopyran, hexahydrochromene, hexahydroquinoline, terahydroquinone, morpholino, thiomorpholino, pyrrolidinyl, piperazinyl, oxabicyclooctane, oxabicycloheptane, and thiabicyclooctane, azaspirodecane, piperidinyl, and thiazolidinyl, along with the cyclic form of sugars.

Non-aromatic carbocyclic groups consist of one or more rings, where each ring is non-aromatic and each ring atom is a carbon atom. Non-aromatic carbocyclic groups include fully saturated ring systems and ring systems having one or more degrees of unsaturation. Each ring in a non-aromatic heterocyclic group ring can be five, six, seven or eight-membered. Examples include cyclohexadiene, cyclohexene, cyclohexane, cyclopentane, cyclooctane, cyclopentadiene, hexahydronaphthalene, octahydroindene, bicycloheptane, bicyclooctane, spirodecane and spirononadiene.

Suitable substituents on an alkyl, alkenyl, alkynyl, aryl, non-aromatic heterocyclic or partially or fully aryl group (carbocyclic and heteroaryl) are those which do not substantially interfere with the ability of the disclosed compounds to have one or more of the properties disclosed herein. A substituent substantially interferes with the properties of a compound when the magnitude of the property is reduced by more than about 50% in a compound with the substituent compared with a compound without the substituent. Examples of suitable substituents include —OH, halogen (—Br, —Cl, —I and —F), —OR^{a}, —O—COR^{a}, —COR^{a}, —C(O)R^{a}, —CN, —NO², —COOH, —COOR^{a}, —OCO₂R^{a}, —C(O)NR^{a}R^{b}, —OC(O)NR^{a}R^{b}, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NRCOR^{a}, —NHCONH₂, —NHCONR^{a}H, —NHCON(R^{a}R^{b}), —NR^{c}CONH₂, —NR^{c}CONR^{a}H, —NR^{c}CON(R^{a}R^{b}), —C(═NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c})—N(R^{a}R^{b}), —NR^{d}H—C(═NH)—NH₂, —NR^{d}—C(═NH)—NHR^{a}, —NR^{d}—C(═NH)—N(R^{a}R^{b}), —NR^{d}—C(═NR^{c})—NH₂, —NR^{d}—C(═NR^{c})—NHR^{a}, —NR^{d}—C(═NR^{c})—N(R^{a}R^{b}), —NHNH₂, —NHNHR^{a}, —NHR^{a}R^{b}, —SO₂NH₂, —SO₂NHRₐ, —SO₂NR^{a}R^{b}, —CH═CHR^{a}, —CH═CR^{a}R^{b}, —CR^{c}═CR^{a}R^{b}, CR^{c}═CHR^{a}, —CR^{c}═CR^{a}R^{b}, —CCR^{a}, —SH, —SOₖR^{a} (k is 0, 1 or 2), —S(O)ₖOR^{a} (k is 0, 1 or 2) and —NH—C(═NH)—NH₂. R^{a}-R^{d} are each independently an aliphatic, substituted aliphatic, benzyl, substituted benzyl, aromatic or substituted aromatic group, preferably an alkyl, benzylic or aryl group. In addition, —NR^{a}R^{b}, taken together, can also form a substituted or unsubstituted non-aromatic heterocyclic group. A non-aromatic heterocyclic group, benzylic group or aryl group can also have an aliphatic or substituted aliphatic group as a substituent. A substituted aliphatic group can also have a non-aromatic heterocyclic ring, a substituted a non-aromatic heterocyclic ring, benzyl, substituted benzyl, aryl or substituted aryl group as a substituent. A substituted aliphatic, non-aromatic heterocyclic group, substituted aryl, or substituted benzyl group can have more than one substituent.

Combinations of substituents and variables envisioned by this invention are only those that result in the formation of stable compounds. As used herein, the term “stable” refers to compounds that possess stability sufficient to allow manufacture and that maintain the integrity of the compound for a sufficient period of time to be useful for the purposes detailed herein.

Also included in the present invention are salts, particularly pharmaceutically acceptable salts, of the compounds described herein. The compounds of the present invention that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion (e.g., a halide such as bromide, chloride, or fluoride, particularly bromide).

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

The compounds disclosed herein also include partially and fully deuterated variants. In certain embodiments, one or more deuterium atoms are present for kinetic studies. One of ordinary skill in the art can select the sites at which such deuterium atoms are present.

According to another embodiment, the present invention provides methods of producing the above-defined compounds. The compounds may be synthesized using conventional techniques. Advantageously, these compounds are conveniently synthesized from readily available starting materials.

### 3. Exemplary Uses

MTP inhibitors can be employed to decrease LDL-c and triglyceride plasma levels, along with intestinal lipid absorption. MTP inhibitors may therefore be used in the treatment and/or prophylaxis of disorders associated with lipid metabolism, including non-insulin dependent diabetes mellitus, coronary heart disease, pancreatitis, mixed dyslipidemia, hypercholesterolemia, hypertriglyceridemia, hyperlipemia, post-prandial hyperlipemia, atherosclerosis and obesity. Additionally, compounds of the invention may be used to treat neuropathological changes in the brain and their sequelae, which includes treating forms of neurodegeneration such as those associated with Alzheimer's disease, progressive atrophy of the brain, morphological changes in the brain during the normal aging process (presenile dementia), impairment of the cortical cholinergic system, memory impairments, orientation impairments, aphasia, wordfinding impairments, agnosia, apraxia, euphoria, depression, Binswanger's disease, Pick's disease, Niemann-Pick disease and cerebrovascular insufficiency.

The invention provides methods of treating patients in need of MTP inhibition using a compound of the invention. A patient in need of MTP inhibition is a patient having a disease or condition in which MTP plays a role in the disease or condition. Examples of patients in need of MTP inhibition include patients having or at risk of having diabetes (including Type I and Type II, impaired glucose tolerance, insulin resistance, and diabetic complications, such as nephropathy, retinopathy, neuropathy and cataracts), atherosclerosis, obesity, hyperlipidemia, hyperlipoproteinemia, hypercholesterolemia, hypertriglyceridemia, hypoalphalipoproteinemia, pancreatitis, myocardial infarction, stroke, restenosis, or Syndrome X. In certain embodiments, the invention includes methods of treating a patient suffering from diabetes. In certain embodiments, the invention includes methods of treating a patient suffering from obesity or a patient who is overweight.

The term “diabetes,” as used herein, includes both insulin-dependent diabetes mellitus (i.e., IDDM, also known as type I diabetes) and non-insulin-dependent diabetes mellitus (i.e., NIDDM, also known as Type II diabetes). Type I diabetes, or insulin-dependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type II diabetics are also obese. The compositions of the present invention are useful for treating both Type I and Type II diabetes. The compositions are especially effective for treating Type II diabetes. The compounds or combinations of the present invention are also useful for treating and/or preventing gestational diabetes mellitus.

The invention also provides methods of treating patients at risk of having atherosclerosis using a compound of the invention. The characteristics of patients at risk of having atherosclerosis are well known to those in the art and include patients who have a family history of cardiovascular disease, including hypertension and atherosclerosis, obese patients, patients who exercise infrequently, patients with hypercholesterolemia, hyperlipidemia and/or hypertriglyceridemia, patients having high levels of LDL or Lp(a), patients having low levels of HDL (hypoalphalipoproteinemia), and the like.

The invention additionally provides methods of treating patients at risk of developing diabetes using a compound of the invention. Patients at risk of developing diabetes include patients who have a family history of diabetes, obese patients, patients who exercise infrequently, patients who have polycystic ovary syndrome, impaired glucose tolerance or exhibit insulin resistance, and patients who have or have had gestational diabetes. The preferred type of diabetes to be treated by the compounds of the present invention is non-insulin dependent diabetes mellitus, also known as Type II diabetes or NIDDM. It is also noted that the complications associated with diabetes can be treated or prevented through the methods disclosed herein.

The invention further provides methods of treating patients at risk of developing restenosis using a compound of the invention. Patients who are at risk of developing restenosis include patients who have undergone angioplasty procedures, or who have had bypass surgery. In general restenosis can occur whenever a blood vessel has been damaged or stressed. Balloon angioplasty is the most common type of angioplasty.

In another aspect, the present invention provides methods of treating patients at risk of having myocardial infarction using a compound of the invention. Patients who are at risk of having myocardial infarction are patients who are obese, have cardiovascular diseases, such as atherosclerosis, high cholesterol, or hypertension, and the like. In addition, patients having diabetes are at risk of developing cardiovascular diseases to a higher extent than persons not having diabetes. Such development of cardiovascular diseases can result in myocardial infarction.

The invention also provides methods of treating patients at risk of having a stroke using a compound of the invention. Patients who are at risk of having a stoke include patients having atherosclerosis, hypercholesterolemia, hyperlipidemia, hypertriglyceridemia, hypoalphalipoproteinemia, diabetes, patients undergoing angioplasty procedures, bypass surgery or any other form of surgery, obese patients, and the like. Treating or preventing atherosclerosis, helps to lower the probability of having a stroke.

Compounds of the invention are also useful in increasing endogenous levels of various gastrointestinal hormones, such as PYY, GLP-1, GLP-2 and GIP. Accordingly, compounds of the invention are useful for treating conditions or disorders related to a deficiency in one or more of these hormones or where it would be useful to increase levels of one or more of these hormones.

PYY

The peptides called NPY, PYY, and PP are hormones often said to belong to the pancreatic polypeptide family. Neuropeptide Y (NPY) is the most abundant peptide in central and peripheral nervous system in mammals. It stimulates food intake, affects blood pressure, enhances memory retention, and affects circadian rhythms. Human pancreatic polypeptide (PP), as isolated from the pancreas, has 36 amino acid residues with an amidated C-terminal tyrosine. PP is released into the plasma when stimulated by the ingestion of food and inhibits the stimulation of gastric and pancreatic exocrine secretions. A related peptide was discovered in extracts of intestine and named Peptide YY (PYY) because of its N- and C-terminal tyrosine (Y) residues.

Peptide YY (PYY) is a 36-residue peptide amide isolated originally from porcine intestine, and localized in the endocrine cells of the gastrointestinal tract and pancreas (Tatemoto et al. Proc. Natl. Acad. Sci. 79:2514, 1982). PYY shares a number of central and peripheral regulatory roles with its homologous peptide Neuropeptide Y (NPY), which was originally isolated from porcine brain (Tatemoto, Proc. Natl. Acad. Sci. 79:5485, 1982). PYY is localized in intestinal cells; NPY, in contrast, is present in the submucous and myenteric neurons which innervate the mucosal and smooth muscle layers, respectively (Ekblad et al. Neuroscience 20:169, 1987). Both PYY and NPY are believed to inhibit gut motility and blood flow (Laburthe, Trends Endocrinol. Metab. 1:168, 1990), and they are also thought to attenuate basal (Cox et al. Br. J. Pharmacol. 101:247, 1990; Cox et al. J. Physiol. 398:65, 1988; Cox et al. Peptides 12:323, 1991; Friel et al. Br. J. Pharmacol. 88:425, 1986) and secretagogue-induced intestinal secretion in rats (Lundberg et al. Proc. Natl. Acad. Sci. USA 79:4471, 1982; Playford et al. Lancet 335:1555, 1990) and humans (Playford et al., supra), as well as stimulate net absorption (MacFadyen et al. Neuropeptides 7:219, 1986). Elevated plasma PYY levels have been reported in individuals suffering from several conditions that cause diarrhea (Adrian et al. Gastroenterology 89:1070, 1985). Taken together, these observations suggest that PYY and NPY are released into the circulation after a meal (Adrian et al. Gastroenterology 89:1070, 1985; Balasubramaniam et al. Neuropeptides 14:209, 1989), and, thus, may play a physiological role in regulating intestinal secretion and absorption, serving as natural inhibitors of diarrhea.

Structure-activity studies using several partial sequences have led to the identification of PYY(22-36) as the active site for interacting with intestinal PYY receptors (Balsubramaniam et al. Pept. Res. 1:32, 1988). PYY[3-36] is reportedly a selective ligand at the Y2 and Y5 receptors, which appear pharmacologically unique in preferring N-terminally truncated (i.e. C-terminal fragments of) NPY analogs.

PYY has been implicated in a number of physiological activities including nutrient uptake (see, e.g., Bilcheik et al. Digestive Disease Week 506:623, 1993), cell proliferation (see, e.g., Laburthe, Trends Endocrinol. Metab. 1:168, 1990; Voisin et al. J. Biol. Chem, 1993), lipolysis (see, e.g., Valet et al., J. Clin. Invest. 85:291, 1990), and vasoconstriction (see, e.g., Lundberg et al., Proc. Natl. Acad. Sci., USA 79:4471, 1982). Recently it has been suggested that infusion of normal postprandial concentrations of PYY(3-36) significantly reduces appetite and food intake in humans (see Batterham et al., Nature 418:656-654, 2002; Batterham et al., N Engl J Med. 349:941, 2003). Administration of compounds of the invention is believed to cause similar physiological activities.

Peripheral administration of PYY reportedly reduces gastric acid secretion, gastric motility, exocrine pancreatic secretion (Yoshinaga, Mochizuki et al. Am J Physiol 263: G695-701, 1992) (Guan, Maouyo et al. Endocrinology 128: 911-6, 1991) (Pappas, Debas et al. Gastroenterology 91: 1386-9, 1986), gallbladder contraction and intestinal motility (Savage, Adrian et al. Gut 28: 166-70, 1987).

The compounds of the invention are especially useful in the treatment of any number of gastrointestinal disorders that are associated with excess intestinal electrolytes and water secretion as well as decreased absorption, e.g., infectious (e.g., viral or bacterial) diarrhea, inflammatory diarrhea, short bowel syndrome, or the diarrhea which typically occurs following surgical procedure, e.g., ileostomy (see e.g. Harrison's principles of Internal Medicine, McGraw Hill Inc., New York, 12th ed.). Examples of infectious diarrhea include, without limitation, acute viral diarrhea, acute bacterial diarrhea (e.g., salmonella, campylobacter, and clostridium) or diarrhea due to protozoal infections, or travellers' diarrhea (e.g., Norwalk virus or rotavirus). Examples of inflammatory diarrhea include, without limitation, malabsorption syndrome, tropical spue, chronic pancreatitis, Crohn's disease, diarrhea, and irritable bowel syndrome. It has also been discovered that the compounds of the invention can be used to treat an emergency or life-threatening situation involving a gastrointestinal disorder, e.g., after surgery or due to cholera. Furthermore, the compounds of the invention can be used to treat intestinal dysfunction in patients with Acquired Immune Deficiency Syndrome (AIDS), especially during cachexia.

The compounds of the invention are also useful for inhibiting small intestinal fluid and electrolyte secretion, and augmenting nutrient transport, as well as increasing cell proliferation in the gastrointestinal tract, regulating lipolysis in, e.g., adipase tissue and regulating blood flow in a mammal.

Compounds of the invention can also be used to treat ulcerative colitis. Ulcerative colitis is a comparatively common inflammatory bowel disease (“IBD”) with a prevalence of about 70-150 cases in a population of 100,000. There are estimated to be 380,000-480,000 persons in the United States with inflammatory bowel disease. (Ward F M, et al., “Clinical economics review: medical management of inflammatory bowel disease.” Aliment Pharmacol Ther. 1999; 13(1):15-25). Ulcerative colitis typically exhibits a bimodal age distribution. For example, it usually appears in white males in their twenties and thirties and peaks at ages 20-29. In females, it peaks at about ages 30-39. Once affected, the disease is usually recurring with 75% of patients who suffered an initial attack continue to suffer repeated attacks. The second peak for the disease in recurring patients is usually between the ages of 70-79 in both genders. (Garland C F, et al., “Incidence rates of ulcerative colitis and Crohn's disease in fifteen areas of the United States.” Gastroenterology. 1981; 81(6):1115-24).

Surgical removal of the colon is eventually required in 20-25% of ulcerative colitis patients, and radical surgical intervention contributes substantially to the excess mortality in ulcerative colitis. (Cucino C. and Sonnenberg A., “Cause of death in patients with inflammatory bowel disease.” Inflamm Bowel Dis. 2001; 7(3):250-5). An additional contributor to the increased risk of mortality is a propensity to develop colorectal cancer for patients who suffered from ulcerative colitis. The cumulative probability of such development of colorectal cancer has been estimated to be about 18% after 30 years of the disease. Eaden J A, et al., The risk of colorectal cancer in ulcerative colitis: a meta-analysis. Gut. 2001; 48(4):526-35.

Compounds of the invention are also useful for treating inflammatory bowel disease. The major symptoms associated with local inflammatory bowel disease are bloody diarrhea and abdominal pain with blood- and pus-containing stools. The disease may also manifest systemically due to chronic malabsorption (e.g., dehydration, anemia, and hypokalemia) and inflammation (e.g. fever and weight loss). Systemic manifestations of the disease outside of the colon include arthritis, skin changes, and liver changes. Moreover, complications of the disease may include abscesses, fistulas, an increased risk of cancer, toxic megacolon (which carries a mortality of 50%), and bowel perforation with ensuing peritonitis and septicemia, (which is a significant cause of death). Overall, for people affected with inflammatory bowel disease, the quality of life is markedly decreased.

Presently, the etiology of ulcerative colitis is unknown. Some have proposed and described a genetic predisposition to the disease, while others have proposed infective etiopathogenesis. There have also been reports of association between the onset of the disease with periods of emotional stress. Nevertheless, the conventional therapies for inflammatory bowel disease have focused on reducing inflammation of the colon. For example, present drug therapies for ulcerative colitis are either steroid-based (ACTH or glucocorticoids) or directed against prostaglandins such as 5-aminosalicylic acid (5-ASA) or sulfasalazine, which liberates 5-ASA after bacterial breakdown in the colon. But steroid therapies are often associated with major side effects including risk of infection and bone loss with the steroid doses required to control disease. Robinson R J, et al., “Rectal steroids suppress bone formation in patients with colitis.” Aliment Pharmacol Ther. 1997; 11(1):201-4. Nevertheless, the present invention includes combinations of compounds of the invention with present drug therapies for ulcerative colitis and/or inflammatory bowel disease, either administered at the same time or separated in time.

Other experimental agents for the treatment of Crohn's disease, a form of inflammatory bowel disease, are similarly focused on suppressing the immune system of the patient suffering from the disease and may promote opportunistic infections. Some of these experimental drugs include (a) immunomodulators such as tacrolimus and mycophenolate; (b) biologics such as anti-tumor necrosis factor antibody, anti-interleukin-12 (IL-12) antibody, anti-alpha-4 integrin antibody, interleukin-10 (IL-10), interleukin-11 (IL-11), and antisense RNA (ISIS-2302) against ICAM-1. In addition to promoting opportunistic infections, the immunomodulators are further limited by their toxicities at high doses or when used for long periods of time. In certain embodiments, the invention includes combinations of compounds of the invention with immunomodulators, preferably where the dose of immunomodulators is reduced over the standard dose.

Other experimental drugs that are not anti-inflammatory have also been proposed but they are similarly limited by the major side effects associated with them. For example, thalidomide has also been studied as a drug for treating Crohn's disease, but the drug, originally released as a sedative and anti-nausea medication, was discontinued in the 1960s because it caused a high incidence of birth defects. Nicotine has also been used in clinical trials of ulcerative colitis and may work to inhibit production of interleukin-12 (IL-12) and tumor necrosis factor. However, randomized trials in active ulcerative colitis have shown only a modest benefit that is less than that of steroids, and it has relatively frequent side effects. The invention contemplates combinations of these experimental drugs with compounds of the invention.

PYY administration has been shown to prevent bowel mucosal protein loss during total parenteral nutrition and increased the weight and DNA content of the duodenum significantly in nursing rats and adult mice, such that the secretion of PYY caused by compound of the invention is believed to have a similar effect.

GLP-2

GLP-2 is a 33 amino acid peptide expressed in a tissue-specific manner from the pleiotropic proglucagon gene, and thus part of the glucagon super-family of peptide hormones. The major action of GLP-2 involves stimulation of cell growth, and the mechanism coupling GLP-2 receptor activation, directly or indirectly, to cell proliferation has not been examined. GLP-2, as a natural intestinal-derived peptide, has been demonstrated to have a significant reparative activity for the mucosal epithelium of the small and large intestine. It has also been demonstrated to increase the ability of the intestine to digest and absorb nutrients, suggesting a potential therapeutic role in the treatment of intestinal insufficiency. Indeed, several studies have now confirmed that GLP-2 administration reduces or prevents intestinal damage in rodent models of colitis, enteritis, total parenteral nutrition and massive resection. Phase 2 clinical trials of GLP-2 have also been reported, in which patients with short bowel syndrome were demonstrated to exhibit an enhanced ability to absorb enteral nutrients after 30 days of GLP-2 administration, with apparently no undesirable side effects.

The principal metabolic pathway for GLP-2 clearance is through enzymatic degradation. GLP-2 has been shown to be rapidly degraded through the removal of its two N-terminal amino acids by dipeptidylpeptidase-IV (DPP-IV), which represents a major limitation because it leads to the complete inactivation of the peptide. As a result, the half-life of GLP-2 is thus quite short, and current GLP-2 treatment necessitates infusion or frequent injections. It has been shown that peptide analogs of native GLP-2 possess enhanced trophic activity at the small intestine as GLP-2 receptor agonists (see for example U.S. Pat. No. 5,990,077). Although very useful, a critical disadvantage of GLP-2 peptides and analogs, as stated above, is their very short half-lives in vivo, which is typically not more than 2 minutes. Compounds of the invention may allow for a more convenient dosing regimen, such as once or twice daily dosing regimens.

When administered exogenously, GLP-2 can produce a marked increase in the proliferation of small intestinal epithelium in mice, with no apparent side effects (Drucker et al., 1996, Proc. Natl. Acad. Sci. 93:7911-7916). Moreover, GLP-2 increases maximal transport rate of D-glucose across the intestinal basolateral membrane (Cheeseman and Tseng, 1996, Am. J. Phys. 271:G477-G482).

U.S. Pat. No. 5,789,379 teaches GLP-2 analogs among which one has been developed as a long-acting compound (ALX-600) and is currently in clinical trials. Such compounds can be administered together with compounds of the invention.

A chimeric antibody (Remicade) has been developed to bind specifically to human tumor necrosis factor alpha (TNFa) for the short-term treatment of Crohn's. This antibody is indicated for the reduction of the symptoms of moderate to severe Crohn's disease in patients who have had an inadequate response to conventional therapy with corticosteroids, other immunosuppressants and/or antibiotics. Nevertheless, serious side effects are observed with such treatment. For example, it has been associated with hypersensitivity reaction, serious infections including sepsis, as well as fatal infections. In certain embodiments, the dosage of such treatments could be lowered when adminstered in conjunction with compounds of the invention.

Conditions treatable according to the invention include but are not limited to osteoporosis, hypercalcemia of malignancy, osteopenia due to bone metastases, periodontal disease, hyperparathyroidism, periarticular erosions in rheumatoid arthritis, Paget's disease, osteodystrophy, myositis ossificans, Bechterew's disease, malignant hypercalcemia, osteolytic lesions produced by bone metastasis, bone loss due to immobilization, bone loss due to sex steroid hormone deficiency, bone abnormalities due to steroid hormone treatment, bone abnormalities caused by cancer therapeutics, osteomalacia, hyperostosis, osteopetrosis, metastatic bone disease, immobilization-induced osteopenia, or glucocorticoid-induced osteoporosis.

Osteoporosis is the most common form of metabolic bone disease. It affects more than causes bone fractures, including approximately spine, hip and wrist fractures. Hip fractures are the most serious consequence of osteoporosis, with 5-20% of patients dying within one year of the fracture and over 50% of survivors being incapacitated.

Osteoporosis is commonly observed in post-menopausal women, but it also occurs in elderly and young individuals. The disease is characterized by low bone mass and a deterioration of bone tissue, with a consequent increase in bone fragility and susceptibility to fracture. Although the etiology of osteoporosis is not known, its onset is associated with several factors such as increased age, decreased hormone level, and decreased calcium levels. Osteoporosis may occur in elderly men as androgen levels fall. Osteoporosis may also be due to increased secretion of parathyroid hormone, which reduces bone formation and enhances bone absorption. Osteoporosis can also be caused by kidney degeneration, which reduces the activity of hydroxylase-activating vitamin D, decreasing intestinal calcium absorption, and precipitating the loss of bone matrix. Mobilization of nutrient stores in bone can be achieved by stimulating osteoclastic bone resorption. Likewise, resorptive activity can be reversed by increasing dietary availability of nutrients.

In certain embodiments, compounds of the invention can be used to treat certain conditions not at first sight related to bone, but give rise to excessive bone resorption and net bone loss by mechanisms that have not previously been explained. Patients who have suffered damage to the spinal cord at a high level and who are paralyzed suffer bone mass loss which is not explicable on the basis of immobilization alone. Patients who have had bowel resection or who have other bowel disorders often show symptoms of excessive bone resorption with net bone mass loss in a manner which is not explicable by say poor calcium absorption from diet.

Another condition leading to bone loss is hypercalcaemia, which may be produced by a number of causes. These causes may have significantly different underlying mechanisms. Hypercalcemia accompanying malignancy is due to local osteolysis involving stimulation of osteoclast formation and activity by the different soluble factors tumor cells can produce. These factors (eicosanoids, cytokines, growth factors and hormones) act on osteoblatic stromal cells that can produce RANK-L, which can bind to RANK on osteclastic precursor cells and in the presence of macrophage-colony stimulating factor, enhance the differentiation and fusion of these cells to produce functioning multinucleated osteoclasts. Parathyroid related hormone (PTH) is often released by cancer cells, especially by breast cancer cells. Some tumor cells can produce soluble RANKL. Additionally, they can also produce various proteases, which are also important for the invasion of the tumor into bone tissue.

In certain embodiments, compounds of the present invention are administered to treat patients that would benefit from gastrointestinal tissue growth. In one aspect, patient candidates are those who would benefit from growth of small bowel tissue. The effects of compounds of the present invention on this tissue, is dramatic and would clearly benefit those patients suffering from diseases or conditions marked by abnormalities in the small intestinal tract mucosa, including: ulcers and inflammatory disorders; congenital or acquired digestion and absorption disorders including malabsorption syndromes; and diseases and conditions caused by loss of bowel mucosal function particularly in patients undergoing extended parenteral feeding or who, as a result of surgery, have undergone resection of the bowel and suffer from short-gut syndrome and cul-de-sac syndrome. Therapeutic treatment with compounds of the present invention administered so as to reduce or eliminate the disease symptoms in these patients associated with their reduced intestinal tract mucosal function. For example, compounds of the present invention are administrated to a patient with an inflammatory bowel condition in an amount sufficient to ameliorate the intestinal discomfort and diarrhea caused by the condition. Additionally, compounds of the present invention may be administered to patients with malabsorption disorders so as to enhance the nutritional absorption and thereby improve the nutritional status of such patients.

According to the present invention, the compounds of the present invention can be administered to patients that would benefit from growth of the tissue of the upper gastrointestinal tract. In addition, patients who would benefit from increased upper gastrointestinal tract tissue function, whether as a result of increased tissue growth or not, are candidates for treatment with the invention. In general, patients who would benefit from either increased upper gastrointestinal tract mass and/or increased upper gastrointestinal tract mucosal function are candidates for treatment with the compounds of the present invention. Particular conditions that may be treated with the compounds of the present invention include the various forms of inflammatory diseases of the stomach or esophagus, as well as patients who have undergone partial or sub-total resection of the upper gastrointestinal tract. A non-exhaustive list of conditions of the upper gastrointestinal tract including the stomach and esophagus, that may be treated by the compounds of the invention, comprises disorders of the stomach like acute gastritis, acute hemorrhagic gastritis, acute stress gastritis, viral gastritis, parasitic gastritis, fungal gastritis, gastropathy (acute), hemorrhagic gastropathy, acute Helicobacter pylori gastritis, type A, B or C gastritis, hypersecretory gastritis, non specific gastritis secondary to Helicobacter pylori, Helicobacter pylori-associated gastritis, chemical gastritis, reactive gastritis, reflux gastritis, bile gastritis, metaplastic atrophic gastritis and environmental metaplastic atrophic gastritis, idiopathic pangastritis, diffuse corporal gastritis, autoimmune chronic gastritis and autoimmune-associated gastritis, bacterial gastritis other than Helicobacter pilori (Gastrospirillum hominis, phlegmonous, mycobacterial, syphilitic), postantrectomy atrophic gastitis, eosinophilic gastritis, and any other acute infectious gastritis; Crohn's disease, sarcoidosis, isolated granulomatous gastritis, lymphocylic gastritis, Menetriere's disease, etc., and disorders of the esophagus like infectious esophagitis from fungi like Candida species (esp. albicans), Aspergillus sp., Histoplasma capsulatum, Blastomyces dermatitides, or from viruses like herpes simplex virus (type 1), cytomegalovirus, Varicella-zoster virus, or from bacteria like Mycobacterium tuberculosis, Actinomyces Israelii, Streptococcus viridans, Lactobacillus acidophilus, and Treponema pallidum. Other disorders of the esophagus include, without limitation, non-infectious esophagitis, acid reflux, bile reflux, chemical injury (caused by medicines, toxins, acids, alkali etc.), sarcoidosis, Crohn's disease, Behcet's disease, Graft-versus-host disease, AIDS Related Infections (Cryptosporidium sp., Microsporidium sp., Isospora beill, Glardia Lamblia, Salmonella sp., Shigella sp., Campylobacter sp., Mycobacterium tuberculosis, Mycobacterium avium complex, Clostridium difficile, Cytomeglavorius and Herpes simplex).

Other diseases or conditions that can be treated with the compounds of the present invention include abnormalities in the small intestinal tract mucosa, which include ulcers and inflammatory disorders; congenital or acquired digestion and absorption disorders including malabsorption syndromes; and diseases and conditions caused by loss of small intestine mucosal function particularly in patients undergoing extended parenteral feeding or who, as a result of surgery, have undergone resection of the small intestine and suffer from short-gut syndrome and cul-de-sac syndrome. In general, patients who would benefit from either increased small intestinal mass and consequent increased small intestine mucosal function are candidates for treatment with compounds of the present invention. Particular conditions that may be treated with the compounds of the present invention include the various forms of sprue including celiac sprue which results from a toxic reaction to gliadin from wheat, and is marked by a tremendous loss of villae of the small intestine; tropical sprue which results from infection and is marked by partial flattening of the villae; hypogammaglobulinemic sprue which is observed commonly in patients with common variable immunodeficiency or hypogammaglobulinemia and is marked by significant decrease in villus height. The therapeutic efficacy of the treatment may be monitored by enteric biopsy to examine the villus morphology, by biochemical assessment of nutrient absorption, by patient weight gain, or by amelioration of the symptoms associated with these conditions. Other conditions that may be treated with the present derivatives, or for which they may be useful prophylactically, include radiation enteritis, infectious or post-infectious enteritis, regional enteritis (Crohn's disease), small intestinal damage due to toxic or other chemotherapeutic agents, and patients with short bowel syndrome.

In another aspect, patient candidates for treatment with the compounds of the present invention are those who would benefit from growth of pancreatic islets, and particularly from proliferation or regeneration of pancreatic islets. Such patients include those suffering from diseases or conditions marked by the absence or reduction of pancreatic islets or by reduced pancreatic islet function. Particular patient candidates are those suffering from type 1 or type 2 diabetes, as well as patients with secondary forms of diabetes due to infiltration, inflammation or destruction of the pancreas. In certain embodiments, a compound of the present invention is administered to these patients in an amount sufficient to restore at least partial pancreatic function, increase the level of endogenous insulin, and/or ameliorate their symptoms.

The estimated prevalence of short bowel syndrome (SBS) patients with non-malignant disease requiring home parenteral nutrition (HPN) is at least 40 per million of the U.S. population. SBS usually results from surgical resection of some or most of the small intestine for conditions such as Crohn's disease, mesenteric infarction, volvulus, trauma, congenital anomalies, and multiple strictures due to adhesions or radiation. Surgical resection may also include resection of all or part of the colon. SBS patients suffer from malabsorption that may lead to malnutrition, dehydration and weight loss. Some patients can maintain their protein and energy balance through hyperphagia; more rarely they can sustain fluid and electrolyte requirements to become independent from parenteral fluid. In certain embodiments, administration of compounds of the invention allows for a decrease in the amount of HPN required, including a total cessation of HPN.

The endogenous meal-stimulated hormone, glucagon-like peptide-2 (GLP-2), raises considerable interest for SBS patients. GLP-2 functions to slow gastric emptying, reduce gastric secretions, increase intestinal blood-flow and stimulate growth of the small and large intestine. In animal studies, GLP-2 administration induces mucosal epithelial proliferation in the stomach and small and large intestine by stimulation of crypt cell proliferation and inhibition of enterocyte apoptosis.

SBS patients with end-jejunostomy and no colon have low basal GLP-2 levels and limited meal-stimulated GLP-2 secretion due to removal of GLP-2 secreting L-cells, which are located primarily in the terminal ileum and colon. This GLP-2 deficiency results in a minimal adaptive response following resection and could explain the gastric hypersecretion, rapid intestinal transit and lack of intestinal adaptation observed in these SBS patients. Jeppesen et al. (Gastroenterology 2001; 120:806-815) have described positive benefit in an open-label study using pharmacologic doses of native GLP-2 in SBS jejunostomy patients. There was significant improvement in intestinal wet weight absorption and a more modest improvement in energy absorption that led to an increase in body weight, lean body mass and a rise in urinary creatinine excretion. Thus, it is believed that administration of compounds of the invention have similar benefits.

The invention, in certain embodiments, relates to therapeutic and related uses of compounds of the present invention, particularly for promoting the growth and proliferation of gastrointestinal tissue, most particularly small bowel tissue or pancreatic islets. With respect to small bowel tissue, such growth is measured conveniently as a GLP-2-mediated increase in small bowel mass and length, relative to an untreated control. The effect of GLP-2 on small bowel also manifests as an increase in the height of the crypt plus villus axis. Such activity is referred to herein as an “intestinotrophic” activity. Also detectable in response to GLP-2 is an increase in crypt cell proliferation and/or a decrease in small bowel epithelium apoptosis. These cellular effects are noted most significantly in relation to the jejunum, including the distal jejunum and particularly the proximal jejunum, and are also noted in the distal ileum. A compound is considered to have “intestinotrophic effect” if test animals of at least one vertebrate species which responds to a reference GLP-2 peptide exhibit significantly increased small bowel weight, increased height of the crypt plus villus axis, or increased crypt cell proliferation or decreased small bowel epithelium apoptosis when treated with the compound.

In a further aspect, the invention relates to a method for the treatment of one or more of small bowel syndrome, Inflammatory bowel syndrome, Crohn's disease, colitis including collagen colitis, radiation colitis, ulcerative colitis, chronic radiation enteritis, non-tropical (gluten intolerance) and tropical sprue, Celiac disease (gluten sensitive enteropathy), damaged tissue after vascular obstruction or trauma, diarrhea e.g. tourist diarrhea and post-infective diarrhea, chronic bowel dysfunction, dehydration, bacteremia, sepsis, anorexia nervosa, damaged tissue after chemotherapy e.g. chemotherapy-induced intestinal mucositis, premature infants including intestinal failure in premature infants, preborn infants including intestinal failure in preborn infants, scleroderma, gastritis including atrophic gastritis, postantrectomy atrophic gastritis and Helicobacter pylori gastritis, pancreatitis, general septic shock ulcers, enteritis, cul-de-sac, lymphatic obstruction, vascular disease and graft-versus-host, healing after surgical procedures, post radiation atrophy and chemotherapy, weight loss in Parkinson's Disease, intestinal adaptation after surgical procedure, parenteral nutrition-induced mucosal atrophy, e.g. total parenteral nutrition (TPN)-induced mucosal atrophy, and bone-related disorders including osteoporosis, hypercalcemia of malignancy, osteopenia due to bone metastases, periodontal disease, hyperparathyroidism, periarticular erosions in rheumatoid arthritis, Paget's disease, osteodystrophy, myositis ossificans, Bechterew's disease, malignant hypercalcemia, osteolytic lesions produced by bone metastasis, bone loss due to immobilization, bone loss due to sex steroid hormone deficiency, bone abnormalities due to steroid hormone treatment, bone abnormalities caused by cancer therapeutics, osteomalacia, Bechet's disease, hyperostosis, osteopetrosis, metastatic bone disease, immobilization-induced osteopenia, or glucocorticoid-induced osteoporosis, the method comprising administering a therapeutically or prophylactically effective amount of a compound of the present invention.

GLP-1

Human GLP-1 is a 37 amino acid residue peptide originating from preproglucagon which is synthesized in the L-cells in the distal ileum, in the pancreas and in the brain. GLP-1 is an important gut hormone with regulatory function in glucose metabolism and gastrointestinal secretion and metabolism. PCT publications WO 98/08871 and WO 99/43706 disclose stable derivatives of GLP-1 analogues, which have a lipophilic substituent. These stable derivatives of GLP-1 analogues have a protracted profile of action compared to the corresponding GLP-1 analogues. Small non-peptidyl organic molecules are also known to be GLP-1 agonists. The invention includes combinations of these analogues and agonists with compounds of the invention.

GLP-1 agonists have earlier been described to be useful for treating hyperglycemia (WO 98/08871), for treating dyslipidemia (WO 01/66135), for reducing morbidity and mortality after myocardial infarct (MI) (U.S. Pat. No. 6,277,819), for treating acute coronary syndrome (ACS), unstable angina (IJA), non-Q-wave cardiac necrosis (NQCN) and Q-wave MI (QMI) (WO 01/89554), for reducing morbidity and mortality after stroke (WO 00/16797), as well as for increasing urine flow (WO 99/40788).

Subjects said to “be in need of treatment with compounds of the present invention” with respect to GLP-1 include subjects with non-insulin dependent diabetes, insulin dependent diabetes, stress-induced hyperglycemia, stroke (see WO 00/16797), myocardial infarction (see WO 98/08531), obesity (see WO 98/19698), catabolic changes after surgery (see U.S. Pat. No. 6,006,753), functional dyspepsia and irritable bowel syndrome (see WO 99/64060). Also included are subjects requiring prophylactic treatment with a GLP-1 compound, e.g., subjects at risk for developing non-insulin dependent diabetes (see WO 00/07617). Subjects with impaired glucose tolerance or impaired fasting glucose, subjects whose body weight is about 25% above normal body weight for the subject's height and body build, subjects with a partial pancreatectomy, subjects having one or more parents with non-insulin dependent diabetes, subjects who have had gestational diabetes and subjects who have had acute or chronic pancreatitis are at risk for developing non-insulin dependent diabetes.

For example, one embodiment of the present invention is a method for treating a patient suffering from or susceptible to diabetes, particularly non-insulin dependent diabetes mellitus (NIDDM) by administering a compound of the present invention and at least one of the compounds of the present invention. Another embodiment of the present invention is a method for treating a subject for obesity by administering a compound of the present invention and at least one of the compounds of the present invention.

Embodiments of the present invention also relate to the prophylactic treatment of subjects who are at risk for non-insulin dependent diabetes. Individuals at risk for non-insulin dependent diabetes are known to those or ordinary skill in the art, and include subjects with impaired glucose tolerance, impaired fasting glucose, overweight subjects, subjects with a partial pancreatectomy, subjects having one or more parents with non-insulin dependent diabetes, subjects who have had gestational diabetes, and subjects who have had acute or chronic pancreatitis.

Embodiments of the present invention further relate to the prophylactic treatment of subjects who are at risk for obesity. Individuals at risk for obesity are known to those or ordinary skill in the art, and include subjects who are already overweight, subjects who have parents or family members who are overweight, subjects who have undergone lifestyle changes such that they are now prone to weight gain (e.g. quitting smoking, the cessation of chronic alcohol or drug use), or subjects who have become incapacitated and/or unable to maintain previous levels of activity.

The compounds of the present invention can be used to normalize blood glucose levels, prevent pancreatic beta-cell deterioration, induce beta-cell proliferation, stimulate insulin gene transcription, up-regulate IDX-1/PDX-1 or other growth factors, improve beta-cell function, activate dormant beta-cells, differentiate cells into beta-cells, stimulate beta-cell replication, inhibit beta-cell apoptosis, regulate body weight, and induce weight loss.

In another aspect the present invention relates to a method for the treatment or prevention of an early cardiac or early cardiovascular disease, which method comprises administration of an effective amount of a compound of the present invention or a pharmaceutically acceptable salt thereof to a patient in need thereof.

In one embodiment of the method the early cardiac or early cardiovascular disease is selected from the group consisting of left ventricular hypertrophy, coronary artery disease, essential hypertension, acute hypertensive emergency, cardiomyopathy, heart insufficiency, exercise tolerance, chronic heart failure, arrhythmia, cardiac dysrhythmia, syncopy, atheroschlerosis, mild chronic heart failure, angina pectoris, cardiac bypass reocclusion, intermittent claudication (atheroschlerosis oblitterens), diastolic dysfunction and systolic dysfunction.

In another aspect the invention relates to a method for reducing the level of BNP in plasma and/or in heart tissue, which method comprises administration of an effective amount of a compound of the present invention or a pharmaceutically acceptable salt thereof to a patient in need thereof.

In one embodiment the patient suffers from a disease selected from the group consisting of left ventricular hypertrophy, coronary artery disease, essential hypertension, acute hypertensive emergency, cardiomyopathy, heart insufficiency, exercise tolerance, chronic heart failure, arrhythmia, cardiac dysrhythmia, syncopy, atheroschlerosis, mild chronic heart failure, angina pectoris, cardiac bypass reocclusion, intermittent claudication (atheroschlerosis oblitterens), diastolic dysfunction and systolic dysfunction.

In another embodiment the patient suffers from a disease selected from the group consisting of myocardial infarct, acute coronary syndrome, unstable angina, non-Q-wave cardiac necrosis, Q-wave myocardial infarct and morbidity after stroke.

In another embodiment of the methods the patient is a diabetic patient.

In yet another embodiment of the methods the patient is a non-diabetic patient.

According to a further aspect, the present invention relates to method of treating a critically ill patient and/or a critical illness polyneuropathy (CIPNP)-patient and/or a potential CIPNP-patient so that the patient is no longer in need of vital organ system support or to treat a critically ill patient and/or a CIPNP-patient and/or a potential CIPNP-patient so that it is considered sufficient for the patient to receive at least about two third of the caloric need through the normal enteral route to reduce the risk or likelihood of multiple organ failure, to reduce the risk or likelihood of multiple organ failure with a proven septic focus on post-mortem examination, to reduce mortality, for example, in-hospital mortality, to reduce the use of mechanical ventilatory support, to reduce the likelihood of renal replacement therapy and/or renal failure, to reduce the likelihood of disturbed kidney function parameters, to reduce the likelihood of hyperbilirubinemia, to reduce the likelihood for blood stream infections, to reduce the likelihood of disturbance in markers of inflammations and/or inflammatory responses, to reduce the use of antibiotics, to reduce the amount of red cell transfusion, or to reduce stress induced hyperglycaemia, or to reduce the likelihood of the critically ill patient and/or the CIPNP-patient and/or the potential CIPNP-patient having repetitive positive EMGs, or to prevent or reduce the amount of ultimately futile intensive care to a critically ill patient and/or a CIPNP-patient and/or a potential CIPNP-patient, or to protect a critically ill patient and/or a CIPNP-patient and/or a potential CIPNP-patient from cholestasis, or to reduce the need for invasive treatment in a critically ill patient and/or a CIPNP-patient and/or a potential CIPNP-patient.

In certain embodiments, a patient in need of the compounds used in the present invention is one who is in the acute phase of stroke, and who also is incapable of auto-regulation of blood glucose. A patient is incapable of auto-regulation if that patient: (1) was previously diagnosed with insulin-dependent diabetes (IDDM) or non-insulin dependent diabetes (NIDDM), according to the definitions of the National Diabetes Data Group (Diabetes, 1979); (2) has a blood glucose level greater than 11 mmol/liter, even without a previous diagnosis of diabetes; or (3) has an abnormal glucose tolerance.

Stroke or apoplexy or cerebrovascular accident (CVA) is a cerebrovascular disease characterized by an abrupt onset of a non-convulsive and focal neurological deficit. In western countries ischemia-infarction causes stroke in about 85-90 percent of cases, whereas intracranial hemorrhages are found in the rest of the patient group. Cerebral ischemia is provoked by a reduction in blood flow lasting for several seconds. If the cessation of flow lasts for more than a few minutes, infarction of brain tissue evolves. The most common cause of cerebral ischemia and infarction are atherosclerosis with thromoboembolism and cardiogenic embolism. The ischemic stroke is characterized clinically by its mode of onset and subsequent course. The hallmark presentation is an acute onset of a hemiparesis in an individual in the atherosclerotic age group. However, any symptoms of brain dysfunction may occur. Symptoms and signs of carotid system disease often affect the distribution of the middle cerebral artery, and the patient may exhibit a contralateral hemiparesis, hemisensory deficit and hemianopsis. When the dominant hemisphere is involved, there is usually some degree of aphasia. The anterior (carotid) or posterior (vertebrobasilar) circulation may be also involved which results in more or less specific clinical symptoms.

GIP

GIP is released from intestinal endocrine K-cells into the bloodstream following ingestion of carbohydrate, protein and particularly fat. GIP's major physiological role is generally believed to be that of an incretin hormone that targets pancreatic islets to enhance insulin secretion and help reduce postprandial hyperglycemia. GIP acts through binding to specific G-protein coupled GIP receptors located on pancreatic beta-cells (Wheeler, M. B. et al., 1995, Endocrinology 136:4629-4639). GIP has been shown to stimulate beta-cell proliferation synergistically with glucose in the islet INS-1 cell line, in association with induction of MAPK and PI 3-kinase. Similarly, GIP exerts anti-apoptotic actions in studies using INS-1 beta-cells.

In certain embodiments, the compounds described herein may be taken alone or in combination with other compounds. In one embodiment, a mixture of two or more compounds of the invention may be administered to a subject in need thereof.

In yet another embodiment, one or more compounds of the invention may be administered with one or more therapeutic agents for the treatment or prevention of various diseases or conditions described herein, including, for example, diabetes, cardiovascular disease, obesity, etc. In various embodiments, combination therapies comprising a compound of the invention may refer to (1) pharmaceutical compositions that comprise one or more compounds of the invention in combination with one or more therapeutic agents (e.g., one or more therapeutic agents described herein); and (2) co-administration of one or more compounds of the invention with one or more therapeutic agents wherein the compound of the invention and therapeutic agent have not been formulated in the same composition (but may be present within the same kit or package, such as a blister pack or other multi-chamber package; connected, separately sealed containers (e.g., foil pouches) that can be separated by the user; or a kit where the compound(s) of the invention and other therapeutic agent(s) are in separate vessels). When using separate formulations, the compound of the invention may be administered at the same time, intermittently, staggered, prior to, subsequent to, or combinations thereof, relative to the administration of another therapeutic agent.

In one embodiment, a compound of the invention may be administered with one or more of the following compounds: a sirtuin activator, resveratrol, butein, fisetin, piceatannol, or quercetin. In an exemplary embodiment, a compound of the invention may be administered in combination with nicotinic acid. In another embodiment, a compound of the invention may be administered with one or more of the following compounds: nicotinamide (NAM), suranim; NF023 (a G-protein antagonist); NF279 (a purinergic receptor antagonist); Trolox (6-hydroxy-2,5,7,8,tetramethylchroman-2-carboxylic acid); (−)-epigallocatechin (hydroxy on sites 3,5,7,3′,4′, 5′); (−)-epigallocatechin gallate (Hydroxy sites 5,7,3′,4′,5′ and gallate ester on 3); cyanidin choloride (3,5,7,3′,4′-pentahydroxyflavylium chloride); delphinidin chloride (3,5,7,3′,4′,5′-hexahydroxyflavylium chloride); myricetin (cannabiscetin; 3,5,7,3′,4′,5′-hexahydroxyflavone); 3,7,3′,4′,5′-pentahydroxyflavone; gossypetin (3,5,7,8,3′,4′-hexahydroxyflavone), sirtinol; and splitomicin (see e.g., Howitz et al. (2003) Nature 425:191; Grozinger et al. (2001) J. Biol. Chem. 276:38837; Dedalov et al. (2001) PNAS 98:15113; and Hirao et al. (2003) J. Biol. Chem. 278:52773).

An aspect of the present invention is also pharmaceutical compositions containing, in combination, a compound of the invention and a pyrazole-derived antagonist for cannabinoid CB, receptors, chosen from rimonabant and N-piperidino-5-(4-bromophenyl)-1-(2,4-dichlorophenyl)-4-ethylpyrazole-3-carboxamide.

The invention also includes a compound of invention and another active principal chosen from one of the following therapeutic classes: one or more antihypertensives (e.g., an angiotensin II AT₁ receptor antagonist (e.g., losartan), alone or combined with a diuretic or with a calcium antagonist; a converting-enzyme inhibitor, alone or combined with a diuretic; a calcium antagonist; an alpha- or beta-blocker, alone or combined with a diuretic or with a calcium antagonist); a blood lipid-lowering agent or a blood cholesterol-lowering agent; an anti-diabetic agent; and an anti-obesity agent.

Converting-enzyme inhibitors are compounds such as alacepril, benazepril, captopril, cilazapril, enalapril, enalapril, fosinopril, imidapril, lisinopril, moexipril, perindopril, quinapril, ramipril, spirapril, temocapril, trandolapril or zofenopril, it being possible for each of these compounds to itself be combined with a diuretic such as hydrochlorothiazide or indapamide or with a calcium antagonist such as amlodipine, diltiazem, felodipine or verapamil.

Calcium antagonists are compounds such as amlodipine, aranidipine, benidipine, bepridil, cilnidipine, diltiazem, efonidipine hydrochloride ethanol, fasudil, felodipine, isradipine, lacidipine, lercanidipine hydrochloride, manidipine, mibefradil hydrochloride, nicardipine, nifedipine, nilvadipine, nimodipine, nisoldipine, nitrendipine, terodiline or verapamil.

Beta-blockers include non-subtype-selective beta-adrenergic antagonists and selective beta 1-adrenergic antagonists and is intended to mean a compound such as acebutolol, alprenolol, amosulalol, arotinolol, atenolol, befunolol, betaxolol, bevantolol, bisoprolol, bopindolol, bucumolol, bufetolol, bunitrolol, butofilolol, carazolol, carteolol, carvedilol, cloranolol, epanolol, esmolol, indenolol, labetalol, landiolol, levobunolol, levomoprolol, mepindolol, metipranolol, metoprolol, nadolol, nebivolol, nifenalol, nipradilol, oxprenolol, penbutolol, pindolol, propanolol, salmeterol, sotalol, talinolol, tertatolol, tilisolol, timolol, xamoterol or xibenolol.

Alpha-blockers include doxazosin, urapidil, prazosin and terazosin.

Blood lipid-lowering agents or blood cholesterol-lowering agent are compounds chosen from fibrates such as alufibrate, beclobrate, bezafibrate, ciprofibrate, clinofibrate, clofibrate, etofibrate, fenofibrate, gemfibrozil; statins (HMG-CoA reductase inhibitors) such as atorvastatin, fluvastatin sodium, lovastatin, pravastatin, rosuvastatin or simvastatin, or a compound such as acipimox, aluminium nicotinate, azacosterol, cholestyramine, colestipol, dextrothyroxine, meglutol, niceritrol, nicoclonate, nicotinic acid, probucol, beta-sitosterine, tiadenol or rimonabant.

Anti-diabetic agents include compounds belonging to one of the following classes: imidazolines, sulphonylureas, biguanidines, alpha-glucosidase inhibitors, oxadiazolidinediones, thiazolidine diones, agents acting on the ATP-dependent potassium channel of the pancreatic beta-cells, potassium channel openers (e.g., ormitiglinide), potassium channel blockers (e.g., nateglinide, BTS-67582), glucagon antagonists, GLP-1 agonists, DPP-IV (dipeptidyl peptidase-IV) inhibitors, PTPase (protein tyrosine phosphatase) inhibitors, glucokinase activators, inhibitors of hepatic enzymes involved in stimulation of gluconeogenesis and/or glycogenolysis, glucose uptake modulators, GSK-3 (glycogen synthase kinase-3) inhibitors, compounds modifying the lipid metabolism such as antihyperlipidemic agents and antilipidemic agents, compounds lowering food intake, PPAR (peroxisome proliferator-activated receptor) and RXR (retinoid X receptor) agonists, insulin sensitizers and metiglinides.

Additional suitable antidiabetic agents include insulin and insulin analogs and GLP-1 (glucagon like peptide-1) derivatives, such as those disclosed in WO 98/08871 and EP 0 792 290 (Novo Nordisk A/S), e.g., N-epsilon-B29-tetradecanoyl des (B30) human insulin, EP 0 214 826 and EP 0 705 275 (Novo Nordisk A/S), e.g., Asp-B28 human insulin, U.S. Pat. No. 5,504,188 (Eli Lilly), e.g., Lys-B28 Pro-B29 human insulin, and EP 0 368 187 (Aventis), e.g., Lantus, as well as orally active hypoglycemic agents.

Agents acting on the ATP-dependent potassium channel of the pancreatic beta-cells include potassium channel openers such as those disclosed in WO 97/26265, WO 99/03861 and WO 00/37474 (Novo Nordisk A/S), which are incorporated herein by reference.

Glucagon antagonists include those disclosed in WO 99/01423 and WO 00/39088 (Novo Nordisk A/S and Agouron Pharmaceuticals, Inc.), all of which are incorporated herein by reference.

GLP-1 agonists include those disclosed in WO 00/42026 (Novo Nordisk A/S and Agouron Pharmaceuticals, Inc.), which is incorporated herein by reference.

Glucokinase activators include those described in WO 02/08209 to Hoffmann La Roche.

PPAR (peroxisome proliferator-activated receptor) and RXR (retinoid X receptor) agonists include ALRT-268, LG-1268 and LG-1069.

Sulphonylureas include acetohexamide, carbutamide, glibomuride, gliquidone, glisoxepide, glybuzole, glymidine, metahexamide, tolbutamide, chlorpropamide, tolazamide, glibenclamide, glipizide, glimepiride, glicazide and glyburide.

Biguanides include metformin.

Meglitinides include repaglinide and senaglinide/nateglinide.

Thiazolidinediones include troglitazone, ciglitazone, pioglitazone, rosiglitazone, isaglitazone, darglitazone, englitazone, CS-011/CI-1037 or T 174 and the compounds disclosed in WO 97/41097 (e.g. 5-[[4-[3-methyl-4-oxo-3,4-dihydro-2-quinazolinyl]methoxy]phenylmethyl]thi-azolidine-2,4-dione), WO 97/41119, WO 97/41120, WO 00/41121 and WO 98/45292, which are incorporated herein by reference.

Insulin sensitizers include GI 262570, YM-440, MCC-555, JTT-501, AR-H039242, KRP-297, GW-409544, CRE-16336, AR-H049020, LY510929, MBX-102, CLX-0940, GW-501516 or the compounds disclosed in WO 99/19313 (NN622/DRF-2725), WO 00/50414, WO 00/63191, WO 00/63192, WO 00/63193, WO 00/23425, WO 00/23415, WO 00/23451, WO 00/23445, WO 00/23417, WO 00/23416, WO 00/63153, WO 00/63196, WO 00/63209, WO 00/63190 and WO 00/63189, which are incorporated herein by reference.

Alpha-glucosidase inhibitors include voglibose, emiglitate, miglitol and acarbose.

Glycogen phosphorylase inhibitors include the compounds described in WO 97/09040.

Agents acting on the ATP-dependent potassium channel of the pancreatic beta-cells include tolbutamide, glibenclamide, glipizide, glicazide, BTS-67582 and repaglinide.

Anti-obesity agents include compounds such as amfepramone, benfluorex, indanorex, mazindole, mefenorex, D-norpseudoephedrine or another antagonist for cannabinoid CB, receptors, CART (cocaine amphetamine regulated transcript) agonists, NPY (neuropeptide Y) antagonists, MC3 (melanocortin 3) agonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, beta-3 adrenergic agonists such as CL-316243, AJ-9677, GW-0604, LY362884, LY377267 or AZ-40140, MSH (melanocyte-stimulating hormone) agonists, MCH (melanocyte-concentrating hormone) antagonists, CCK (cholecystokinin) agonists, serotonin reuptake inhibitors (fluoxetine, seroxat or citalopram), norepinephrine reuptake inhibitors (e.g. sibutramine), 5HT (serotonin) agonists, bombesin agonists, galanin antagonists, growth hormone, growth factors such as prolactin or placental lactogen, growth hormone releasing compounds, TRH (thyreotropin releasing hormone) agonists, UCP 2 or 3 (uncoupling protein 2 or 3) modulators, leptin and leptin agonists, DA (dopamine) agonists (bromocriptin, doprexin), lipase/amylase inhibitors (e.g., orlistat), PPAR modulators, RXR modulators, TR-beta agonists, adrenergic CNS stimulating agents (e.g., dexamphetamine, amphetamine, benzphetamine, methamphetamine, phentermine, mazindol phendimetrazine, diethylpropion, fenfluramine or dexfenfluramine), AGRP (agouti related protein) inhibitors, H3 histamine antagonists such as those disclosed in WO 00/42023, WO 00/63208 and WO 00/64884, exendin-4, GLP-1 agonists and ciliary neurotrophic factor. Further antiobesity agents are bupropion (antidepressant), topiramate (anticonvulsant), ecopipam (dopamine D1/D5 antagonist), naltrexone (and other opioid antagonists), and peptide YY336 (Batterham et al, Nature 418, 650-654 (2002)).

In certain embodiments, the compound of the present may be administered in combination with more than one of the above-mentioned compounds, e.g., in combination with metformin and a sulphonylurea such as glyburide; a sulphonylurea and acarbose; nateglinide and metformin; acarbose and metformin; a sulfonylurea, metformin and troglitazone; insulin and a sulfonylurea; insulin and metformin; insulin, metformin and a sulfonylurea; insulin and troglitazone; insulin and lovastatin; etc.

It should be understood that any suitable combination of the compounds according to the invention with diet and/or exercise, one or more of the above-mentioned compounds and optionally one or more other active substances are considered to be within the scope of the present invention.

### Cardiovascular Disease

In another embodiment, the invention provides a method for treating and/or preventing a cardiovascular disease by administering to a subject in need thereof a compound of the invention.

Cardiovascular diseases that can be treated or prevented using the compounds of the invention include cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using compounds and methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated or prevented include those related to the retinal arterioles, the glomerular arterioles, the vasa nervorum, cardiac arterioles, and associated capillary beds of the eye, the kidney, the heart, and the central and peripheral nervous systems.

Yet other disorders that may be treated with compounds of the invention include restenosis, e.g., following coronary intervention, and disorders relating to an abnormal level of high density and low density cholesterol.

In one embodiment, a compound of the invention may be administered as part of a combination therapeutic with another cardiovascular agent including, for example, an anti-arrhythmic agent, an antihypertensive agent, a calcium channel blocker, a cardioplegic solution, a cardiotonic agent, a fibrinolytic agent, a sclerosing solution, a vasoconstrictor agent, a vasodilator agent, a nitric oxide donor, a potassium channel blocker, a sodium channel blocker, statins, or a naturiuretic agent.

In one embodiment, a compound of the invention may be administered as part of a combination therapeutic with an anti-arrhythmia agent. Anti-arrhythmia agents are often organized into four main groups according to their mechanism of action: type I, sodium channel blockade; type II, beta-adrenergic blockade; type III, repolarization prolongation; and type IV, calcium channel blockade. Type I anti-arrhythmic agents include lidocaine, moricizine, mexiletine, tocainide, procainamide, encainide, flecanide, tocainide, phenyloin, propafenone, quinidine, disopyramide, and flecainide. Type II anti-arrhythmic agents include propranolol and esmolol. Type III includes agents that act by prolonging the duration of the action potential, such as amiodarone, artilide, bretylium, clofilium, isobutilide, sotalol, azimilide, dofetilide, dronedarone, ersentilide, ibutilide, tedisamil, and trecetilide. Type IV anti-arrhythmic agents include verapamil, diltaizem, digitalis, adenosine, nickel chloride, and magnesium ions.

In another embodiment, a compound of the invention may be administered as part of a combination therapeutic with another cardiovascular agent. Examples of cardiovascular agents include vasodilators, for example, hydralazine; angiotensin converting enzyme inhibitors, for example, captopril; anti-anginal agents, for example, isosorbide nitrate, glyceryl trinitrate and pentaerythritol tetranitrate; anti-arrhythmic agents, for example, quinidine, procainaltide and lignocaine; cardioglycosides, for example, digoxin and digitoxin; calcium antagonists, for example, verapamil and nifedipine; diuretics, such as thiazides and related compounds, for example, bendrofluazide, chlorothiazide, chlorothalidone, hydrochlorothiazide and other diuretics, for example, fursemide and triamterene, and sedatives, for example, nitrazepam, flurazepam and diazepam.

Other exemplary cardiovascular agents include, for example, a cyclooxygenase inhibitor such as aspirin or indomethacin, a platelet aggregation inhibitor such as clopidogrel, ticlopidene or aspirin, fibrinogen antagonists or a diuretic such as chlorothiazide, hydrochlorothiazide, flumethiazide, hydroflumethiazide, bendroflumethiazide, methylchlorthiazide, trichloromethiazide, polythiazide or benzthiazide as well as ethacrynic acid tricrynafen, chlorthalidone, furosemide, musolimine, bumetanide, triamterene, amiloride and spironolactone and salts of such compounds, angiotensin converting enzyme inhibitors such as captopril, zofenopril, fosinopril, enalapril, ceranopril, cilazopril, delapril, pentopril, quinapril, ramipril, lisinopril, and salts of such compounds, angiotensin II antagonists such as losartan, irbesartan or valsartan, thrombolytic agents such as tissue plasminogen activator (tPA), recombinant tPA, streptokinase, urokinase, prourokinase, and anisoylated plasminogen streptokinase activator complex (APSAC, Eminase, Beecham Laboratories), or animal salivary gland plasminogen activators, calcium channel blocking agents such as verapamil, nifedipine or diltiazem, thromboxane receptor antagonists such as ifetroban, prostacyclin mimetics, or phosphodiesterase inhibitors. Such combination products if formulated as a fixed dose employ the compounds of this invention within the dose range described above and the other pharmaceutically active agent within its approved dose range.

Yet other exemplary cardiovascular agents include, for example, vasodilators, e.g., bencyclane, cinnarizine, citicoline, cyclandelate, cyclonicate, ebumamonine, phenoxezyl, flunarizine, ibudilast, ifenprodil, lomerizine, naphlole, nikamate, nosergoline, nimodipine, papaverine, pentifylline, nofedoline, vincamin, vinpocetine, vichizyl, pentoxifylline, prostacyclin derivatives (such as prostaglandin E1 and prostaglandin I2), an endothelin receptor blocking drug (such as bosentan), diltiazem, nicorandil, and nitroglycerin. Examples of the cerebral protecting drug include radical scavengers (such as edaravone, vitamin E, and vitamin C), glutamate antagonists, AMPA antagonists, kainate antagonists, NMDA antagonists, GABA agonists, growth factors, opioid antagonists, phosphatidylcholine precursors, serotonin agonists, Na⁺/Ca²⁺ channel inhibitory drugs, and K⁺ channel opening drugs. Examples of the brain metabolic stimulants include amantadine, tiapride, and gamma-aminobutyric acid. Examples of the anticoagulant include heparins (such as heparin sodium, heparin potassium, dalteparin sodium, dalteparin calcium, heparin calcium, parnaparin sodium, reviparin sodium, and danaparoid sodium), warfarin, enoxaparin, argatroban, batroxobin, and sodium citrate. Examples of the antiplatelet drug include ticlopidine hydrochloride, dipyridamole, cilostazol, ethyl icosapentate, sarpogrelate hydrochloride, dilazep hydrochloride, trapidil, a nonsteroidal antiinflammatory agent (such as aspirin), beraprostsodium, iloprost, and indobufene. Examples of the thrombolytic drug include urokinase, tissue-type plasminogen activators (such as alteplase, tisokinase, nateplase, pamiteplase, monteplase, and rateplase), and nasaruplase. Examples of the antihypertensive drug include angiotensin converting enzyme inhibitors (such as captopril, alacepril, lisinopril, imidapril, quinapril, temocapril, delapril, benazepril, cilazapril, trandolapril, enalapril, ceronapril, fosinopril, imadapril, mobertpril, perindopril, ramipril, spirapril, and randolapril), angiotensin II antagonists (such as losartan, candesartan, valsartan, eprosartan, and irbesartan), calcium channel blocking drugs (such as aranidipine, efonidipine, nicardipine, bamidipine, benidipine, manidipine, cilnidipine, nisoldipine, nitrendipine, nifedipine, nilvadipine, felodipine, amlodipine, diltiazem, bepridil, clentiazem, phendilin, galopamil, mibefradil, prenylamine, semotiadil, terodiline, verapamil, cilnidipine, elgodipine, isradipine, lacidipine, lercanidipine, nimodipine, cinnarizine, flunarizine, lidoflazine, lomerizine, bencyclane, etafenone, and perhexyline), β-adrenaline receptor blocking drugs (propranolol, pindolol, indenolol, carteolol, bunitrolol, atenolol, acebutolol, metoprolol, timolol, nipradilol, penbutolol, nadolol, tilisolol, carvedilol, bisoprolol, betaxolol, celiprolol, bopindolol, bevantolol, labetalol, alprenolol, amosulalol, arotinolol, befunolol, bucumolol, bufetolol, buferalol, buprandolol, butylidine, butofilolol, carazolol, cetamolol, cloranolol, dilevalol, epanolol, levobunolol, mepindolol, metipranolol, moprolol, nadoxolol, nevibolol, oxprenolol, practol, pronetalol, sotalol, sufinalol, talindolol, tertalol, toliprolol, xybenolol, and esmolol), α-receptor blocking drugs (such as amosulalol, prazosin, terazosin, doxazosin, bunazosin, urapidil, phentolamine, arotinolol, dapiprazole, fenspiride, indoramin, labetalol, naftopidil, nicergoline, tamsulosin, tolazoline, trimazosin, and yohimbine), sympathetic nerve inhibitors (such as clonidine, guanfacine, guanabenz, methyldopa, and reserpine), hydralazine, todralazine, budralazine, and cadralazine. Examples of the antianginal drug include nitrate drugs (such as amyl nitrite, nitroglycerin, and isosorbide), β-adrenaline receptor blocking drugs (such as propranolol, pindolol, indenolol, carteolol, bunitrolol, atenolol, acebutolol, metoprolol, timolol, nipradilol, penbutolol, nadolol, tilisolol, carvedilol, bisoprolol, betaxolol, celiprolol, bopindolol, bevantolol, labetalol, alprenolol, amosulalol, arotinolol, befunolol, bucumolol, bufetolol, buferalol, buprandolol, butylidine, butofilolol, carazolol, cetamolol, cloranolol, dilevalol, epanolol, levobunolol, mepindolol, metipranolol, moprolol, nadoxolol, nevibolol, oxprenolol, practol, pronetalol, sotalol, sufinalol, talindolol, tertalol, toliprolol, andxybenolol), calcium channel blocking drugs (such as aranidipine, efonidipine, nicardipine, bamidipine, benidipine, manidipine, cilnidipine, nisoldipine, nitrendipine, nifedipine, nilvadipine, felodipine, amlodipine, diltiazem, bepridil, clentiazem, phendiline, galopamil, mibefradil, prenylamine, semotiadil, terodiline, verapamil, cilnidipine, elgodipine, isradipine, lacidipine, lercanidipine, nimodipine, cinnarizine, flunarizine, lidoflazine, lomerizine, bencyclane, etafenone, and perhexyline), trimetazidine, dipyridamole, etafenone, dilazep, trapidil, nicorandil, enoxaparin, and aspirin. Examples of the diuretics include thiazide diuretics (such as hydrochlorothiazide, methyclothiazide, trichlormethiazide, benzylhydrochlorothiazide, and penflutizide), loop diuretics (such as furosemide, etacrynic acid, bumetanide, piretanide, azosemide, and torasemide), K⁺ sparing diuretics (spironolactone, triamterene, and potassium canrenoate), osmotic diuretics (such as isosorbide, D-mannitol, and glycerin), nonthiazide diuretics (such as meticrane, tripamide, chlorthalidone, and mefruside), and acetazolamide. Examples of the cardiotonic include digitalis formulations (such as digitoxin, digoxin, methyldigoxin, deslanoside, vesnarinone, lanatoside C, and proscillaridin), xanthine formulations (such as aminophylline, choline theophylline, diprophylline, and proxyphylline), catecholamine formulations (such as dopamine, dobutamine, and docarpamine), PDE III inhibitors (such as aminone, olprinone, and milrinone), denopamine, ubidecarenone, pimobendan, levosimendan, aminoethylsulfonic acid, vesnarinone, carperitide, and colforsin daropate. Examples of the antiarrhythmic drug include ajmaline, pirmenol, procainamide, cibenzoline, disopyramide, quinidine, aprindine, mexiletine, lidocaine, phenyloin, pilsicamide, propafenone, flecamide, atenolol, acebutolol, sotalol, propranolol, metoprolol, pindolol, amiodarone, nifekalant, diltiazem, bepridil, and verapamil. Examples of the antihyperlipidemic drug include atorvastatin, simvastatin, pravastatin sodium, fluvastatin sodium, clinofibrate, clofibrate, simfibrate, fenofibrate, bezafibrate, colestimide, and colestyramine. Examples of the immunosuppressant include azathioprine, mizoribine, cyclosporine, tacrolimus, gusperimus, and methotrexate.

### Weight Control

In another aspect, compounds of the invention may be used for treating or preventing weight gain or obesity in a subject. For example, compounds of the invention may be used, for example, to treat or prevent hereditary obesity, dietary obesity, hormone related obesity, obesity related to the administration of medication, to reduce the weight of a subject, or to reduce or prevent weight gain in a subject. A subject in need of such a treatment may be a subject who is obese, likely to become obese, overweight, or likely to become overweight. Subjects who are likely to become obese or overweight can be identified, for example, based on family history, genetics, diet, activity level, medication intake, or various combinations thereof. In certain embodiments, compounds of the invention contribute to weight loss through appetite suppression.

In yet other embodiments, compounds of the invention may be administered to subjects suffering from a variety of other diseases and conditions that may be treated or prevented by promoting weight loss in the subject. Such diseases include, for example, high blood pressure, hypertension, high blood cholesterol, dyslipidemia, type 2 diabetes, insulin resistance, glucose intolerance, hyperinsulinemia, coronary heart disease, angina pectoris, congestive heart failure, stroke, gallstones, cholescystitis and cholelithiasis, gout, osteoarthritis, obstructive sleep apnea and respiratory problems, some types of cancer (such as endometrial, breast, prostate, and colon), complications of pregnancy, poor female reproductive health (such as menstrual irregularities, infertility, irregular ovulation), bladder control problems (such as stress incontinence); uric acid nephrolithiasis; psychological disorders (such as depression, eating disorders, distorted body image, and low self esteem). Stunkard A J, Wadden T A. (Editors) Obesity: theory and therapy, Second Edition. New York: Raven Press, 1993. Finally, patients with AIDS can develop lipodystrophy or insulin resistance in response to combination therapies for AIDS.

In another embodiment, compounds of the invention may be used for inhibiting adipogenesis or fat cell differentiation, whether in vitro or in vivo. In particular, high circulating levels of insulin and/or insulin like growth factor (IGF) 1 will be prevented from recruiting preadipocytes to differentiate into adipocytes. Such methods may be used for treating or preventing obesity.

In other embodiments, compounds of the invention may be used for reducing appetite and/or increasing satiety, thereby causing weight loss or avoidance of weight gain. A subject in need of such a treatment may be a subject who is overweight, obese or a subject likely to become overweight or obese. The method may comprise administering daily or, every other day, or once a week, a dose, e.g., in the form of a pill, to a subject. The dose may be an “appetite reducing dose.”

Also provided are methods for modulating adipogenesis or fat cell differentiation, whether in vitro or in vivo. In particular, high circulating levels of insulin and/or insulin like growth factor (IGF) 1 will be prevented from recruiting preadipocytes to differentiate into adipocytes. Such methods may be used to modulate obesity. A method for stimulating adipogenesis may comprise contacting a cell with a compound of the invention.

Being obese and being overweight, although closely related, are not the same condition. Body Mass Index, or BMI, is a mathematical calculation used to determine whether a patient is overweight. BMI is calculated by dividing a person's body weight in kilograms by their height in meters squared (weight (kg)/height (m)²) or by using the conversion with pounds and inches squared (weight (lbs)/height (in)² times 704.5). A BMI of 30 or greater is considered obese and a BMI between 25 and 29.9 is considered overweight (National Institutes of Health, National Heart Lung and Blood Institute: Clinical Guidelines on the Identification, Evaluation, and Treatment of Overweight and Obesity in Adults. June 1998). The increased risks associated with obesity occur at a lower Body Mass Index (BMI) in Asians. In Asian countries, including Japan, “obesity” refers to a condition whereby a subject with at least one obesity-induced or obesity-related co-morbidity, that requires weight reduction or that would be improved by weight reduction, has a BMI greater than or equal to 25 kg/m². In Asian countries, including Japan, an “obese subject” refers to a subject with at least one obesity-induced or obesity-related co-morbidity that requires weight reduction or that would be improved by weight reduction, with a BMI greater than or equal to 25 kg/m². In Asia-Pacific, an overweight subject is a subject with a BMI of greater than 23 kg/m² to less than 25 kg/m².

Obesity is a chronic disease linked to a number of serious co-morbidities, associated not only with a social stigma, but also with decreased life span and numerous medical problems, including adverse psychological development, reproductive disorders such as polycystic ovarian disease, dermatological disorders such as infections, varicose veins, Acanthosis nigricans, and eczema, exercise intolerance, type II diabetes mellitus, insulin resistance, hypercholesterolemia, cholelithiasis, osteoarthritis, orthopedic injury, thromboembolic disease, cancer, coronary heart disease, cardiovascular disease, artherosclerosis, and hypertension (Rissanen et al., British Medical Journal, 301: 835-837 (1990)). Preventing and reversing obesity, such as through the methods disclosed herein, has a positive impact on reducing these complications.

In certain embodiments, compounds of the invention are administered in conjunction with controlling energy intake by suppressing appetite. Combining appetite suppression in its earliest stages of olfactory perception and feeding behavior with other methods of weight reduction can produce better clinical outcomes.

The compounds of the present invention are believed to be particular well-suited for the treatment of obesity as such or preventing weight gain and for the treatment of diseases or disorders where obesity is involved in the etiology. In one embodiment, the invention thus provides a method of treating metabolic syndrome, insulin resistance or elevated insulin concentrations, dyslipidemia, hypertension, obesity, type 2 diabetes, type 1 diabetes, diabetic late complications including cardiovascular diseases, cardiovascular disorders, disorders of lipid metabolism, neurodegenerative and psychiatric disorders, dysregulation of intraocular pressure including glaucoma, atherosclerosis, hypertension, coronary heart disease, gallbladder disease (e.g., gallstones), osteoarthritis, and cancer.

More specifically, such conditions include metabolic syndrome, type 2 diabetes (especially in obese patients), diabetes as a consequence of obesity, insulin resistance, hyperglycemia, prandial hyperglycemia, hyperinsulinemia, impaired glucose tolerance (IGT), impaired fasting glucose (IFG), increased hepatic glucose production, type 1 diabetes, LADA, pediatric diabetes, dyslipidemia (especially in obese patients), diabetic dyslipidemia, hyperlipidemia, hypertriglyceridemia, hyperlipoproteinemia, micro-/macroalbuminuria, nephropathy, retinopathy, neuropathy, diabetic ulcers, cardiovascular diseases, arteriosclerosis, coronary heart (artery) disease, cardiac hypertrophy, myocardial ischemia, heart insufficiency, congestive heart failure, stroke, myocardial infarction, arrythmia, decreased blood flow, erectile dysfunction (male or female), myopathy, loss of muscle tissue, muscle wasting, muscle catabolism, osteoporosis, decreased linear growth, neurodegenerative and psychiatric disorders, Alzheimer's disease, neuronal death, impaired cognitive function, depression, anxiety, eating disorders, appetite regulation, migraine, epilepsia, addiction to chemical substances, disorders of intraocular pressure, bacterial infections, and mycobacterial infections. In the present context cancer is intended to include forms such as hematological cancer, such as leukemia, acute myeloid leukemia, chronic myeloid leukemia, chronic lymphatic leukemia, myelodysplasia, multiple myeloma, and Hodgkin's disease, as well as solid tumor forms, such as fibrosarcoma, small or non-small cell long carcinoma, gastric, intestinal or colorectal cancer, prostate, endometrial, ovarian or breast cancer, brain, head or neck cancer, cancer in the urinary tract, such as kidney or bladder cancer, malignant melanoma, liver cancer, uterine and pancreatic cancer.

In a class of the embodiments of the present invention, the obesity-related disorder is selected from: obstructive sleep apnea; abnormal heart rhythms; craniopharyngioma; the Prader-Willi Syndrome; Frohlich's syndrome; GH-deficient subjects; normal variant short stature; Turner's syndrome; angina pectoris; fatty liver; cerebral infarction; cerebral thrombosis; transient ischemic attack; orthopedic disorders; arthritis deformans; lumbodynia; emmeniopathy, and other pathological conditions showing reduced metabolic activity or a decrease in resting energy expenditure as a percentage of total fat-free mass. Further examples of obesity-related disorders are metabolic syndrome, also known as syndrome X, insulin resistance syndrome, reproductive hormone abnormalities, sexual and reproductive dysfunction, such as impaired fertility, infertility, hypogonadism in males and hirsutism in females, fetal defects associated with maternal obesity, gastrointestinal motility disorders, such as obesity-related gastro-esophageal reflux, respiratory disorders, such as obesity-hypoventilation syndrome (Pickwickian syndrome), breathlessness, inflammation, such as systemic inflammation of the vasculature, hyperuricaemia, lower back pain, gout, and increased anesthetic risk. The compositions of the present invention are also useful for reducing the risk of secondary outcomes of obesity, such as reducing the risk of left ventricular hypertrophy. The compositions of the present invention are also useful to treat Alzheimer's disease.

The term “metabolic syndrome”, also known as syndrome X, is defined in the Third Report of the National Cholesterol Education Program Expert Panel on Detection, Evaluation and Treatment of High Blood Cholesterol in Adults (ATP-1). E. S. Ford et al., JAMA, vol. 287 (3), Jan. 16, 2002, pp 356-359. Briefly, a person is defined as having metabolic syndrome if the person has three or more of the following symptoms: abdominal obesity, hypertriglyceridemia, low HDL cholesterol, high blood pressure, and high fasting plasma glucose. The criteria for these are defined in ATP-III.

“Treatment” (of obesity and obesity-related disorders) refers to the administration of the compounds or combinations of the present invention to reduce or maintain the body weight of an obese subject. One outcome of treatment may be reducing the body weight of an obese subject relative to that subject's body weight immediately before the administration of the compounds or combinations of the present invention. Another outcome of treatment may be preventing body weight regain of body weight previously lost as a result of diet, exercise, or pharmacotherapy. Another outcome of treatment may be decreasing the occurrence of and/or the severity of obesity-related diseases. Another outcome of treatment may be to maintain weight loss. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

“Prevention” (of obesity and obesity-related disorders) refers to the administration of the compounds or combinations of the present invention to reduce or maintain the body weight of a subject at risk of obesity. One outcome of prevention may be reducing the body weight of a subject at risk of obesity relative to that subject's body weight immediately before the administration of the compounds or combinations of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Another outcome of prevention may be to prolong resistance to weight gain. Another outcome of prevention may be to prevent weight regain. Moreover, if treatment is commenced in already obese subjects, such treatment may prevent the occurrence, progression or severity of obesity-related disorders, such as, but not limited to, arteriosclerosis, Type II diabetes, polycystic ovarian disease, cardiovascular diseases, osteoarthritis, dermatological disorders, hypertension, insulin resistance, hypercholesterolemia, hypertriglyceridemia, and cholelithiasis.

In an exemplary embodiment, compounds of the invention may be administered as a combination therapy for treating or preventing weight gain or obesity. For example, one or more compounds of the invention may be administered in combination with one or more anti-obesity agents. Exemplary anti-obesity agents include, for example, phenylpropanolamine, ephedrine, pseudoephedrine, phentermine, a cholecystokinin-A agonist, a monoamine reuptake inhibitor (such as sibutramine), a sympathomimetic agent, a serotonergic agent (such as dexfenfluramine or fenfluramine), a dopamine agonist (such as bromocriptine), a melanocyte-stimulating hormone receptor agonist or mimetic, a melanocyte-stimulating hormone analog, a cannabinoid receptor antagonist, a melanin concentrating hormone antagonist, the OB protein (leptin), a leptin analog, a leptin receptor agonist, a galanin antagonist or a GI lipase inhibitor or decreaser (such as orlistat). Other anorectic agents include bombesin agonists, dehydroepiandrosterone or analogs thereof, glucocorticoid receptor agonists and antagonists, orexin receptor antagonists, urocortin binding protein antagonists, agonists of the glucagon-like peptide-1 receptor such as Exendin and ciliary neurotrophic factors such as Axokine.

In another embodiment, compounds of the invention may be administered to reduce drug-induced weight gain. For example, a compound of the invention may be administered as a combination therapy with medications that may stimulate appetite or cause weight gain, in particular, weight gain due to factors other than water retention. Examples of medications that may cause weight gain, include for example, diabetes treatments, including, for example, sulfonylureas (such as glipizide and glyburide), thiazolidinediones (such as pioglitazone and rosiglitazone), meglitinides, nateglinide, repaglinide, sulphonylurea medicines, and insulin; anti-depressants, including, for example, tricyclic antidepressants (such as amitriptyline and imipramine), irreversible monoamine oxidase inhibitors (MAOIs), selective serotonin reuptake inhibitors (SSRIs), bupropion, paroxetine, and mirtazapine; steroids, such as, for example, prednisone; hormone therapy; lithium carbonate; valproic acid; carbamazepine; chlorpromazine; thiothixene; beta blockers (such as propranolo); alpha blockers (such as clonidine, prazosin and terazosin); and contraceptives including oral contraceptives (birth control pills) or other contraceptives containing estrogen and/or progesterone (Depo-Provera, Norplant, Ortho), testosterone or Megestrol. In another exemplary embodiment, compounds of the invention may be administered as part of a smoking cessation program to prevent weight gain or reduce weight already gained.

### Metabolic Disorders/Diabetes

In another aspect, compounds of the invention may be used for treating or preventing a metabolic disorder, such as insulin-resistance, a pre-diabetic state, type II diabetes, and/or complications thereof. Administration of a compound of the invention may increase insulin sensitivity and/or decrease insulin levels in a subject. Alternatively, administration of a compound of the invention may improve glucose and/or insulin homeostatis. A subject in need of such a treatment may be a subject who has insulin resistance or other precursor symptom of type II diabetes, who has type II diabetes, or who is likely to develop any of these conditions. For example, the subject may be a subject having insulin resistance, e.g., having high circulating levels of insulin and/or associated conditions, such as hyperlipidemia, dyslipogenesis, hypercholesterolemia, impaired glucose tolerance, high blood glucose sugar level, other manifestations of syndrome X, hypertension, atherosclerosis and lipodystrophy.

In an exemplary embodiment, compounds of the invention may be administered as a combination therapy for treating or preventing a metabolic disorder. For example, one or more compounds of the invention may be administered in combination with one or more anti-diabetic agents. Exemplary anti-diabetic agents include, for example, an aldose reductase inhibitor, a glycogen phosphorylase inhibitor, a sorbitol dehydrogenase inhibitor, a protein tyrosine phosphatase I B inhibitor, a dipeptidyl protease inhibitor, insulin (including orally bioavailable insulin preparations), an insulin mimetic, metformin, acarbose, a peroxisome proliferator-activated receptor-γ (PPAR-γ) ligand such as troglitazone, rosaglitazone, pioglitazone or GW-1929, a sulfonylurea, glipazide, glyburide, or chlorpropamide wherein the amounts of the first and second compounds result in a therapeutic effect. Other anti-diabetic agents include a glucosidase inhibitor, a glucagon-like peptide-1 (GLP-1), insulin, a PPAR α/γ dual agonist, a meglitimide and an αP2 inhibitor. In an exemplary embodiment, an anti-diabetic agent may be a dipeptidyl peptidase IV (DP-IV or DPP-IV) inhibitor, such as, for example LAF237 from Novartis (NVP DPP728; 1-[[[2-[(5-cyanopyridin-2-yl)amino]ethyl]amino]acetyl]-2-cyano-(S)-pyrrolidine) or MK-04301 from Merck (see e.g., Hughes et al., Biochemistry 38: 11597-603 (1999)).

Compounds that are inhibitors of the dipeptidyl peptidase-IV (“DP-IV” or “DPP-IV”) enzyme are of particular interest in combination with compounds of the present invention. Particular DPP-IV inhibitors include for example Sitagliptin Phosphate, Januvia, Merck & Co., Inc. (see U.S. Pat. No. 6,699,871). Other examples include those described in WO 97/40832; WO 98/19998; U.S. Pat. No. 5,939,560; Bioorg. Med. Chem. Lett., 6(10), 1163-1166 (1996); and Bioorg. Med. Chem. Lett., 6(22), 2745-2748 (1996). The usefulness of DP-IV inhibitors in the treatment of type 2 diabetes is based on the fact that DP-IV in vivo readily inactivates glucagon like peptide-1 (GLP-1) and gastric inhibitory peptide (GIP). GLP-1 and GIP are incretins and are produced when food is consumed. The incretins stimulate production of insulin. Inhibition of DP-IV leads to decreased inactivation of the incretins, and this in turn results in increased effectiveness of the incretins in stimulating production of insulin by the pancreas. DP-IV inhibition therefore results in an increased level of serum insulin. Advantageously, since the incretins are produced by the body only when food is consumed, DP-IV inhibition is not expected to increase the level of insulin at inappropriate times, such as between meals, which can lead to excessively low blood sugar (hypoglycemia). Inhibition of DP-IV is therefore expected to increase insulin without increasing the risk of hypoglycemia, which is a dangerous side effect associated with the use of insulin secretagogues. DPP-IV also cleaves and inactivates GLP-2 and PYY, so the combination of a DPP-IV inhibitor with compounds of the present invention is expected to enhance the aforementioned biological actions of GLP-1, GIP, GLP-2 and PYY.

### 4. Assays

Yet other methods contemplated herein include screening methods for identifying compounds or agents that inhibit MTP. Assays may be conducted in a cell based or cell free format. For example, an assay may comprise incubating (or contacting) MTP with a test agent under conditions in which MTP can be modulated by an agent known to modulate MTP, and monitoring or determining the level of modulation of MTP in the presence of the test agent relative to the absence of the test agent.

The level of modulation of MTP by compounds of the invention can be determined by monitoring secretion of ApoB from cells. In one embodiment, an MTP inhibition assay comprises (i) contacting a cell with a test compound; and (ii) determining the level of ApoB secretion from the cell, where a lower level of ApoB secretion in the presence of the test compound relative to the absence of the test compound indicates that the test compound inhibits lipid transfer. The ApoB ELISA kit (ALerCHEK) may be used to determine the level of ApoB secretion. The method may further comprise determining cellular viability in the presence of test compound.

The level of modulation of MTP by compounds of the invention can also be determined by monitoring the inhibition of triglyceride transfer from donor liposomes to acceptor liposomes catalyzed by MTP. In one embodiment, an MTP inhibition assay comprises (i) contacting liposomes containing radioactively-labeled triglyceride with a test compound; and (ii) determining the level of triglyceride transfer from the donor to the acceptor liposomes, wherein a lower level of triglyceride transfer in the presence of the test compound relative to the absence of the test compound indicates that the test compound inhibits triglyceride transfer.

The level of modulation of MTP by compounds of the invention can also be determined by monitoring the reduction of fat absorption in intestines (e.g., mouse intestines). In one embodiment, an MTP inhibition assay comprises (i) feeding or otherwise administering a test compound to a subject following feeding the subject with radiolabeled triglyceride; and (ii) determining the level of radiolabeled triglyceride in the intestines of the subject, wherein a lower level of triglyceride absorption in the presence of the test compound relative to the absence of the test compound indicates that the test compound inhibits triglyceride absorption.

The level of modulation of MTP by compounds of the invention can also be determined by monitoring the lowering of triglyceride levels in blood serum. In one embodiment, an MTP inhibition assay comprises (i) feeding or otherwise administering a test compound to a subject; and (ii) determining the level of triglyceride in the blood serum, where a lower level of triglyceride in the blood serum in the presence of the test compound relative to the absence of the test compound indicates that the test compound inhibits triglyceride absorption.

The level of modulation of MTP by compounds of the invention can also be determined by monitoring the food intake of a subject (e.g., mice or dogs). In one embodiment, an MTP inhibition assay comprises (i) administering a test compound to a subject; and (ii) monitoring the food intake of the subject, where reduction in food intake is attributable to the test compound.

### 5. Pharmaceutical Compositions

The compounds described herein may be formulated in a conventional manner using one or more physiologically acceptable carriers or excipients. For example, compounds of the invention and their physiologically acceptable salts and solvates may be formulated for administration by, for example, injection (e.g. SubQ, IM, IP), inhalation or insufflation (either through the mouth or the nose) or oral, buccal, sublingual, transdermal, nasal, parenteral or rectal administration. In one embodiment, a compound of the invention may be administered locally, at the site where the target cells are present, i.e., in a specific tissue, organ, or fluid (e.g., blood, cerebrospinal fluid, etc.). Typically, compounds of the invention are administered orally.

Compounds of the invention can be formulated for a variety of modes of administration. Techniques and formulations generally may be found in Remington's Pharmaceutical Sciences, Meade Publishing Co., Easton, Pa.

For oral administration, the pharmaceutical compositions may take the form of, for example, tablets, lozenges, or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

Toxicity and therapeutic efficacy of compounds of the invention can be determined by standard pharmaceutical procedures in cell cultures or experimental animals. The LD₅₀ is the dose lethal to 50% of the population. The ED₅₀ is the dose therapeutically effective in 50% of the population. The dose ratio between toxic and therapeutic effects (LD₅₀/ED₅₀) is the therapeutic index. Compounds of the invention that exhibit large therapeutic indexes are preferred. While compounds of the invention that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may lie within a range of circulating concentrations that include the ED₅₀ with little or no toxicity. The dosage may vary within this range depending upon the dosage form employed and the route of administration utilized. For any compound, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

### 6. Kits

Also provided herein are kits, e.g., kits for therapeutic purposes or kits for compounds of the invention. A kit may comprise one or more compounds of the invention, e.g., in premeasured doses. A kit may optionally comprise devices for treating cells with the compounds of the invention and instructions for use of such compounds. Devices include syringes, stents and other devices for introducing a compound into a subject (e.g., the blood vessel of a subject) or applying it to the skin of a subject.

Another type of kit contemplated by the invention is a kit for identifying MTP inhibitor compounds. Such kits contain (1) a MTP or MTP-containing material and (2) an MTP inhibitor compound of the invention, which are in separate vessels. Such kits can be used, for example, to perform a competition-type assay to test other compounds (typically provided by the user) for MTP inhibition. In certain embodiments, these kits further comprise means for determining MTP inhibition (e.g., a compound with an appropriate indicator, such as those disclosed in the Exemplification).

In yet another embodiment, the invention provides a composition of matter comprising a compound of the invention and another therapeutic agent (e.g., an agent used in combination therapies and/or combination compositions described above) in separate dosage forms, but associated with one another. The term “associated with one another” as used herein means that the separate dosage forms are packaged together or otherwise attached to one another such that it is readily apparent that the separate dosage forms are intended to be sold and administered as part of the same regimen. The agent and the MTP inhibitor are preferably packaged together in a blister pack or other multi-chamber package, or as connected, separately sealed containers (such as foil pouches or the like) that can be separated by the user (e.g., by tearing on score lines between the two containers).

In still another embodiment, the invention provides a kit comprising in separate vessels, a) a compounds of the invention ; and b) another another therapeutic agent such as those described elsewhere in the specification.

The present invention further provides packaged pharmaceuticals. In one embodiment, the packaged pharmaceutical comprises: (i) a therapeutically effective amount of a compound of the invention that is an MTP inhibitor; and (ii) instructions and/or a label for administration of the agent for the treatment of patients with one or more of the diseases or conditions described herein. The instruction or label may be stored on an electronic medium such as CD, DVD, floppy disk, memory card, etc, which may be readable by a computer.

In certain embodiments, the compound of the invention that is an MTP inhibitor is in a container. The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In on embodiment, the container is a blister pack.

The kit may additionally comprise a memory aid of the type containing information and/or instructions for the physician, pharmacist or subject. Such memory aids include numbers printed on each chamber or division containing a dosage that corresponds with the days of the regimen which the tablets or capsules so specified should be ingested, or days of the week printed on each chamber or division, or a card which contains the same type of information. For single dose dispensers, memory aids further include a mechanical counter which indicates the number of daily doses that have been dispensed and a battery-powered micro-chip memory coupled with a liquid crystal readout and/or audible reminder signal which, for example, reads out the date that the last daily dose has been taken and/or reminds one when the next dose is to be taken. Other memory aids useful in such kits are a calendar printed on a card, as well as other variations that will be readily apparent.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

### EXEMPLIFICATION

The invention now being generally described, it will be more readily understood by reference to the following examples which are included merely for purposes of illustration of certain aspects and embodiments of the present invention, and are not intended to limit the invention in any way.

The utility of the compounds of the present invention as pharmaceutically active agents in the treatment of metabolic diseases (such as are detailed herein) in animals, particularly mammals (e.g. humans), can be demonstrated by the activity of the compounds of the present invention in conventional assays and the in vitro and in vivo assays described below. Such assays also provide a means whereby the activities of the compounds of the present invention can be compared with the activities of other known compounds. The results of these comparisons are useful for determining dosage levels.

### Example 1

### Preparation of Compounds of the Invention

Synthetic schemes for compounds of the invention are shown in Schemes 1-8 below.

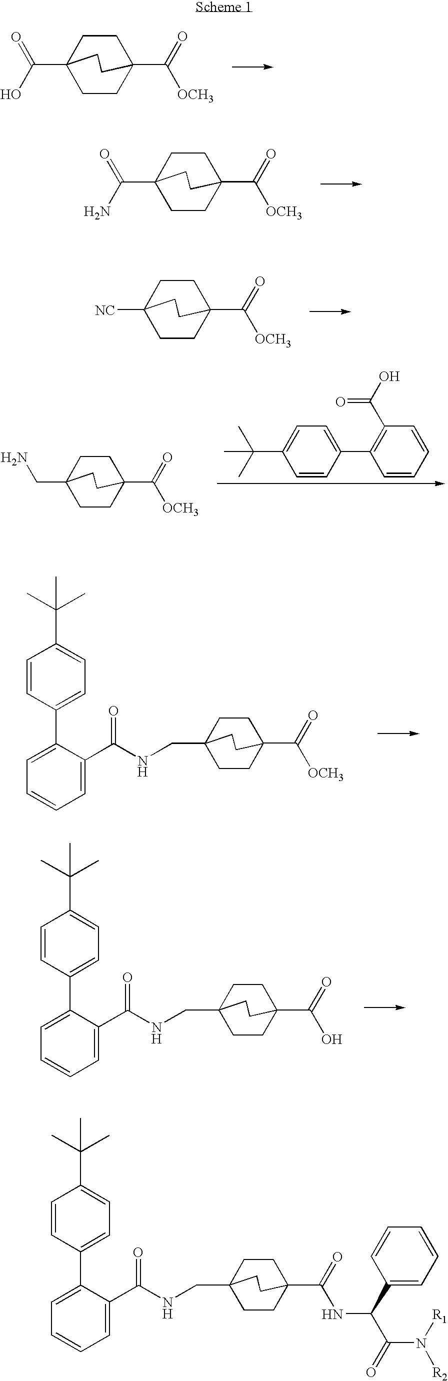

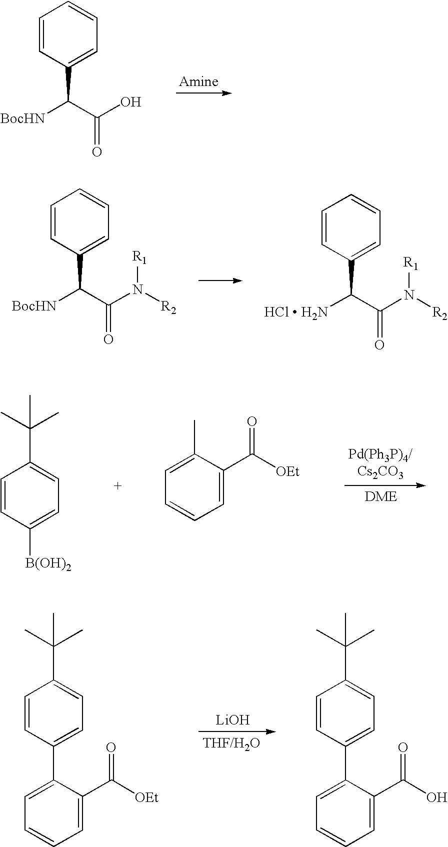

### Preparation of methyl 4-carbamoylbicyclo[2.2.2]octane-1-carboxylate

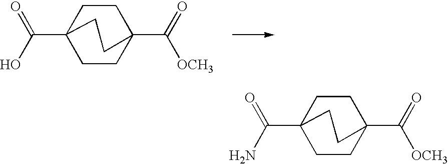

A solution of 4-(methoxycarbonyl)bicyclo[2.2.2]octane-1-carboxylic acid (2.58 g, 12.2 mmol) and triethylamine (Et₃N) (1.70 mL, 12.2 mmol) in CH₂Cl₂ (50 mL) was cooled to 0° C. and methyl chloroformate (0.96 mL, 12.2 mmol) was added rapidly with continuous stirring. After 15 min, anhydrous ammonia was bubbled through the reaction mixture for 1 h. The reaction mixture was then removed from the cooling bath and stirred at room temperature for 18 h. The suspension was filtered and the solvent was removed under reduced pressure. The resulting residue was stirred in boiling benzene (12 mL) and filtered while still hot. The filtrate was diluted with hexane (40 mL) and upon cooling, the desired compound, methyl 4-carbamoylbicyclo[2.2.2]octane-1-carboxylate, precipitated out of the solution as a white solid (1.8 g, yield: 70%). ¹H NMR (400 MHz, DMSO-d₆) δ: 1.65 (12H, m), 3.575 (3H, s), 6.699 (1H, s), 6.929 (1H, s); MS (ESI) calcd for C₁₁H₁₇NO₃ (m/z): 211.26. found: 210.9 [M−1]⁻.

### Preparation of methyl 4-cyanobicyclo[2.2.2]octane-1-carboxylate

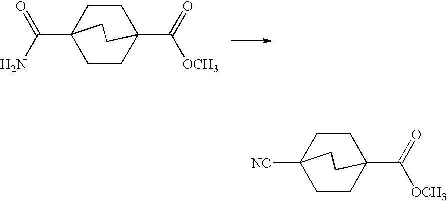

Phosphorous oxychloride (3 mL) was added to a mixture containing methyl 4-carbamoylbicyclo[2.2.2]octane-1-carboxylate (1.8 g, 8.52 mmol) in 1,2-dichloroethane (30 mL). The resulting reaction mixture was stirred under reflux for 20 min. TLC indicated that the reaction was complete (elution: petroleum ether/EtOAc=3:1, I₂ visualization). The reaction mixture was cooled to room temperature and concentrated under reduced pressure. The resulting residue was added carefully to a 2 N aqueous NaOH solution (80 mL) at 0° C. The resulting mixture was then extracted with n-hexane (200 mL×2). The combined organic layers were dried (MgSO₄) and concentrated under reduced pressure to afford methyl 4-cyanobicyclo[2.2.2]octane-1-carboxylate as a white solid (0.83 g, yield: 51%). ¹H-NMR (400 MHz, DMSO-d₆) δ: 1.74 (6H, m), 1.89 (6H, m), 3.586 (3H, s).

### Preparation of methyl 4-(aminomethyl)bicyclo[2.2.2]octane-1-carboxylate

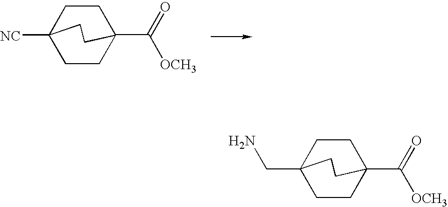

Methyl 4-cyanobicyclo[2.2.2]octane-1-carboxylate (0.83 g, 4.3 mmol) was taken up in methanol (86 mL), H₂O (35 mL) and 1 N HCl (8.6 mL) along with PtO₂.H₂O (430 mg). The resulting reaction was stirred at room temperature under 35 psi of H₂ for 5 h. TLC indicated that the reaction was complete (elution: petroleum ether/EtOAc=3:1, I₂ visualization). The reaction mixture was concentrated under reduced pressure. Saturated aqueous Na₂CO₃ solution (20 mL) was then added, and the resulting mixture was extracted with CH₂Cl₂ (50 mL×2). The combined organic layers were dried (Na₂SO₄) and concentrated under reduced pressure to afford methyl 4-(aminomethyl)bicyclo[2.2.2]octane-1-carboxylate as an oil (0.76 g, yield: 89%). ¹HNMR (400 MHz, CD₃OD) δ: 1.56 (6H, m), 1.85 (6H, m), 2.732 (2H, s), 3.654 (3H, s).

### Preparation of methyl 4-((4′-tert-butylbiphenyl-2-ylcarboxamido)methyl)-bicyclo[2.2.2]octane-1-carboxylate

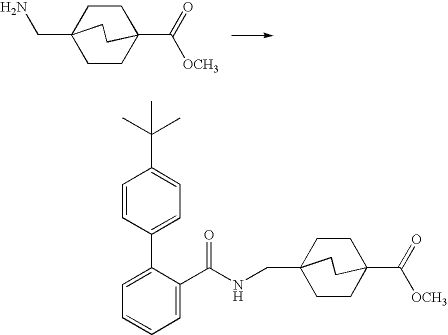

A mixture containing 4′-tert-butylbiphenyl-2-carboxylic acid (127 mg, 0.5 mmol), EDC.HCl (144 mg, 0.75 mmol) and DMAP (73 mg, 0.6 mmol) in CH₂Cl₂ (3 mL) was stirred at room temperature for 15 min. Methyl 4-(aminomethyl)bicyclo[2.2.2]octane-1-carboxylate (98.5 mg, 0.5 mmol) was then added as a solution in CH₂Cl₂ (2 mL). The resulting reaction mixture was stirred at room temperature for 18 h. It was then concentrated under reduced pressure and the resulting residue was purified by chromatography (elution: petroleum ether/EtOAc=8:1) to give methyl 4-((4′-tert-butylbiphenyl-2-ylcarboxamido)methyl)-bicyclo[2.2.2]octane-1-carboxylate as a white solid (135 mg, yield: 62%). ¹H NMR (400 MHz, DMSO-d₆) δ: 1.15 (6H, m), 1.305 (9H, s), 1.55 (6H, m), 2.80 (2H, d, J=6.4 Hz), 3.280 (2H, s), 3.533 (3H, s), 7.302-7.430 (8H, m), 7.816 (1H, t. J=6.0 Hz).

### Preparation of 4-((4′-tert-butylbiphenyl-2-ylcarboxamido)methyl)bicyclo[2.2.2]octane-1-carboxylic acid

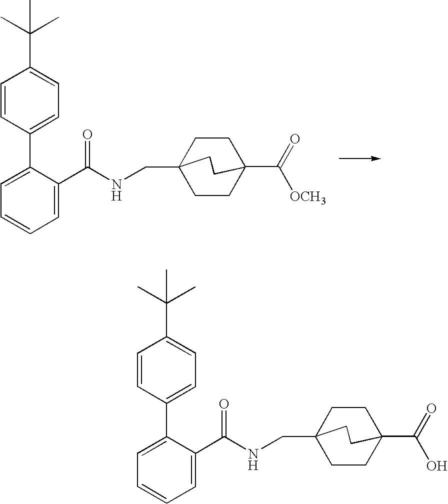

A solution of lithium hydroxide monohydrate (20 mg, 0.468 mmol) in H₂O (1 mL) was added to a solution of methyl 4-((4′-tert-butylbiphenyl-2-ylcarboxamido)methyl)-bicyclo[2.2.2]octane-1-carboxylate (135 mg, 0.312 mmol) in THF (4 mL) at room temperature. The reaction mixture was stirred at room temperature for 18 h. In order to drive the reaction to completion, an additional portion of NaOH (20 mg, 0.5 mmol) was added as a solution in H₂O (0.5 mL) and CH₃OH (2 mL). The resulting reaction mixture was stirred at 45° C. for 4 h. It was then cooled to room temperature and concentrated under reduced pressure. Water (10 mL) was added and enough 1 N HCl was added to produce a pH=2. The resulting precipitate was collected by filtration, washed with water, and dried to afford 4-((4′-tert-butylbiphenyl-2-ylcarboxamido)methyl)bicyclo[2.2.2]octane-1-carboxylic acid as a white solid (105 mg, yield: 81%). ¹H NMR (400 MHz, CDCl₃) δ: 1.12 (6H, m), 1.36 (9H, s), 1.69 (6H, m), 2.94 (2H, d, J=6.4 Hz), 5.22 (1H, t, J=6.4 Hz), 7.324-7.491 (7H, m), 7.79 (1H, dd, J=7.6 Hz, J₂=1.2 Hz).

### Preparation of (S)-4-((4′-tert-butylbiphenyl-2-ylcarboxamido)methyl)-N-(2-((4-fluorobenzyl)(methyl)amino)-2-oxo-1-phenylethyl)bicyclo[2.2.2]octane-1-carboxamide (Compound #10)

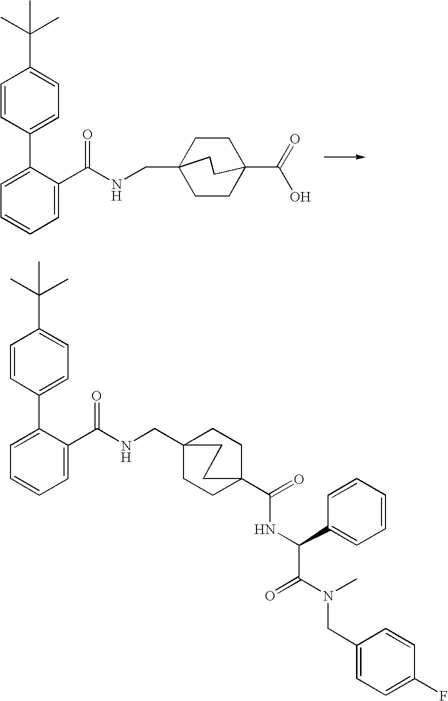

A mixture containing 4-((4′-tert-butylbiphenyl-2-ylcarboxamido)methyl)-bicyclo[2.2.2]octane-1-carboxylic acid (82 mg, 0.2 mmol), (S)-2-amino-N-(4-fluorobenzyl)-N-methyl-2-phenylacetamide hydrochloride salt (93 mg, 0.3 mmol), EDC HCl (58 mg, 0.3 mmol) and Et₃N (0.04 mL, 0.3 mmol) in CH₂Cl₂ (4 mL) was stirred at room temperature for 2 days. The reaction solution was concentrated under reduced pressure. The resulting residue was purified by chromatography (elution: CH₂Cl₂:CH₃OH=50:1) to give (S)-4-((4′-tert-butylbiphenyl-2-ylcarboxamido)methyl)-N-(2-((4-fluorobenzyl)(methyl)amino)-2-oxo-1-phenylethyl)bicyclo[2.2.2]octane-1-carboxamide as a white solid (110 mg, yield: 82%). ¹H NMR (400 MHz, CDCl₃-d) δ: 1.10 (6H, m), 1.35 (9H, S), 1.62 (6H, m), 2.800 (2H, s), 2.861 (1H, s), 2.94 (2H, d, J=6.0 Hz), 4.443˜4.702 (2H, dd, J₁=7.6 Hz, J₂=1.2 Hz), 5.189 (1H, t, J=6.0 Hz), 5.87 (1H, dd, J₁=7.6 Hz, J₂=3.2 Hz), 6.98˜7.12 (5H, m), 7.31˜7.47 (12H, m), 7.793 (1H, dd, J₁=7.6 Hz, J₂=1.6 Hz). MS (ESI) calcd for C₄₃H₄₉FN₃O₃ (m/z): 673.86. found: 674.5 [M+1]⁺, 696.5[M+23]⁺.

### Preparation of Compound #20, Compound #21, Compound #22, Compound #45, Compound #46, Compound #47, Compound #48, Compound #49, Compound #50 and Compound #58

The same procedure used in the preparation of Compound #10 was employed using the appropriate amines.

### Preparation of ethyl 4′-tert-butylbiphenyl-2-carboxylate

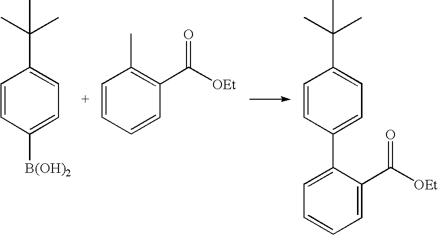

Tetrakis(triphenylphosphine)palladium(0) (1.0 g) was added to a mixture of 4-tert-butylphenylboronic acid (20.0 g, 112.3 mmol), ethyl 2-iodobenzoate (25.0 g, 90.6 mmol), and Cs₂CO₃ (73.2 g, 224.7 mmol) in DMF (250 mL) under an inert atmosphere of argon. The reaction mixture was stirred under reflux for 18 h. It was cooled to room temperature and then taken up in water (50 mL). The mixture was extracted with diethyl ether (3×300 mL). The combined organic layers were washed with brine, dried (MgSO₄) and concentrated under reduced pressure to afford crude ethyl 4′-tert-butylbiphenyl-2-carboxylate as a brown oil (25.6 g, crude yield: 100%). ¹HNMR (400 MHz, CDCl₃) δ: 7.82 (1H, m), 7.55 (1H, m), 7.44-7.40 (4H, m), 7.29-7.27 (2H, m), 4.13 (2H, q, J=7.2 Hz), 1.38 (9H, s), 0.98 (3H, t, J=7.2 Hz); MS (ESI) calcd for C₁₉H₂₂O₂ (m/z): 282.38. found: 283.4 [M+1]⁺.

### Preparation of 4′-tert-butylbiphenyl-2-carboxylic acid

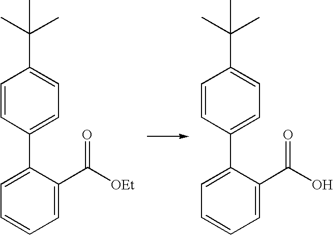

Lithium hydroxide monohydrate (11.4 g, 271.8 mmol) was added to solution containing ethyl 4′-tert-butylbiphenyl-2-carboxylate (25.6 g, 90.6 mmol) in THF (90 mL), H₂O (36 mL) and MeOH (45 mL). The resulting reaction mixture was stirred at 50° C. for 18 h. It was cooled to room temperature and washed with diethyl ether (3×50 mL). Enough 6 N HCl was then added to the aqueous layer to produce a pH=2. The resulting mixture was extracted with ethyl acetate (3×300 mL). The combined organic layers were dried (MgSO₄) and concentrated under reduced pressure to afford 4′-tert-butylbiphenyl-2-carboxylic acid as a white solid (19.562 g, yield: 85%). ¹HNMR (400 MHz, CDCl₃) δ: 7.97 (1H, d, J=7.6 Hz), 7.57 (1H, d, J=7.2 Hz), 7.44 (4H, m), 7.31-7.29 (2H, m), 1.38 (9H, s).

### Preparation of (S)-tert-butyl 2-((4-fluorobenzyl)(methyl)amino)-2-oxo-1-phenylethyl carbamate

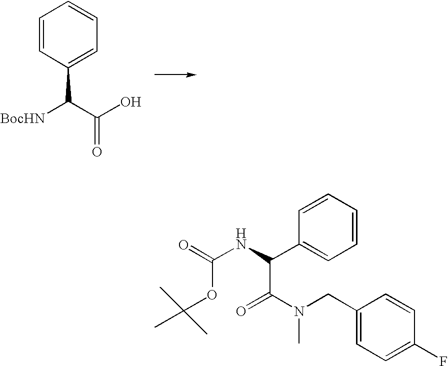

A mixture containing (S)-2-(tert-butoxycarbonylamino)-2-phenylacetic acid (4.0 g, 15.9 mmol) and 1-(4-fluorophenyl)-N-methylmethanamine (2.4 mL, 17.5 mmol) in CH₂Cl₂ (50 mL) was cooled in an ice bath under an inert atmosphere of argon. Diisopropylethylamine (DIPEA, 7.9 mL, 47.7 mmol) and PyBOP (8.9 g, 19.1 mmol) were added sequentially. The resulting reaction mixture was warmed to room temperature and stirred for 18 h. It was then concentrated under reduced pressure and the resulting residue was purified by chromatography (gradient elution: EtOAc/petroleum ether=12:1˜8:1) to afford (S)-tert-butyl 2-((4-fluorobenzyl)(methyl)amino)-2-oxo-1-phenylethylcarbamate as a yellow syrup (4.83 g, yield: 81%). ¹HNMR (400 MHz, CDCl₃) δ: 7.41-7.32 (5H, m), 7.14 (2H, m), 6.99 (2H, m), 6.05 (1H, d, J=8.0 Hz), 5.60 (1H, d, J=7.6 Hz), 4.61 (2H, m), 2.86 (1H, s), 2.81 (2H, s).

### Preparation of (S)-2-amino-N-(4-fluorobenzyl)-N-methyl-2-phenylacetamide hydrochloride salt

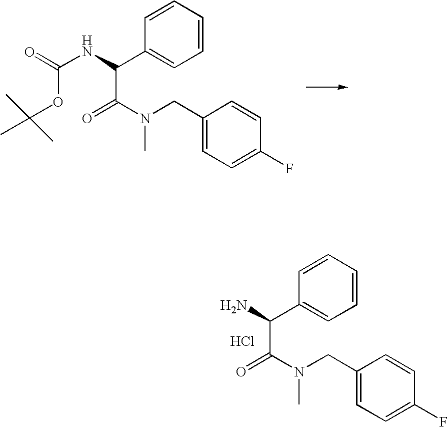

(S)-tert-Butyl 2-((4-fluorobenzyl)(methyl)amino)-2-oxo-1-phenylethylcarbamate (4.83 g, 13.0 mmol) was dissolved in an ice-cooled solution of HCl in dioxane (4N, 40 mL) under an inert atmosphere of argon. The reaction mixture was allowed to warm to room temperature and stirred for 18 h. It was concentrated under reduced pressure to afford (S)-2-amino-N-(4-fluorobenzyl)-N-methyl-2-phenylacetamide hydrochloride salt as a white solid (4.6 g, yield: 100%). ¹H-NMR (400 MHz, CDCl₃) δ: 8.80 (3H, br), 7.55-7.43 (5H, m), 7.25-7.00 (4H, m), 5.58 (1H, d, J=5.2 Hz), 4.45 (2H, m), 2.75 (3H, d, J=5.2 Hz); MS (ESI) calcd for C₁₆H₁₈ClFN₂O (m/z): 308.78. found: 271.9 [M-HCl+1]⁺.

### Preparation of (S)-2-amino-2-phenyl-1-(pyrrolidin-1-yl)ethanone hydrochloride, (S)-2-amino-1-(3,3-difluoropyrrolidin-1-yl)-2-phenylethanone hydrochloride, (S)-2-amino-1-(isoindolin-2-yl)-2-phenylethanone hydrochloride, (S)-2-amino-N-pentyl-2-phenylacetamide hydrochloride, (S)-2-amino-2-phenyl-1-(pyrrolidin-1-yl)ethanone hydrochloride, (S)-2-amino-N-butyl-N-methyl-2-phenylacetamide hydrochloride

The same procedure used in the preparation of (S)-2-amino-N-(4-fluorobenzyl)-N-methyl-2-phenylacetamide hydrochloride salt was employed using the appropriate amines.

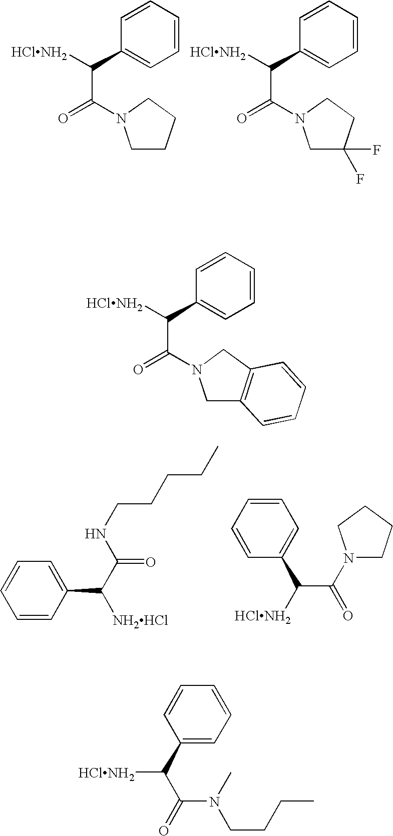

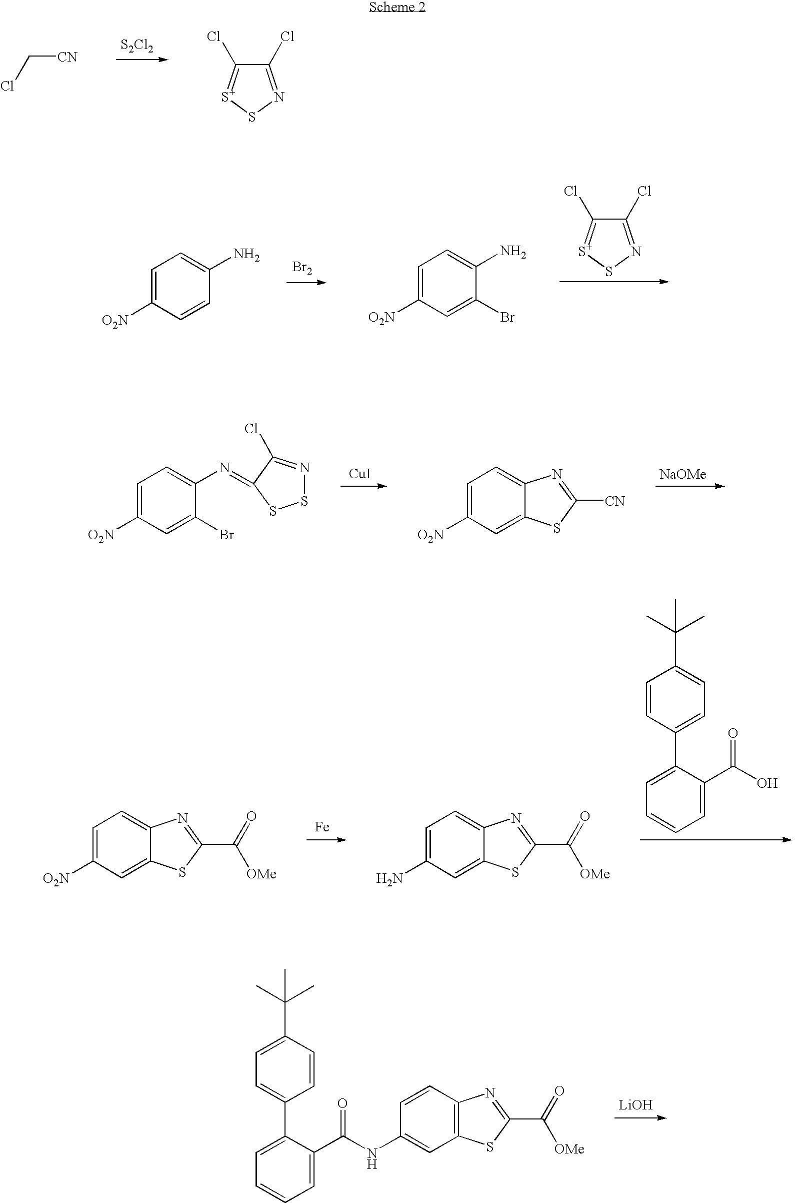

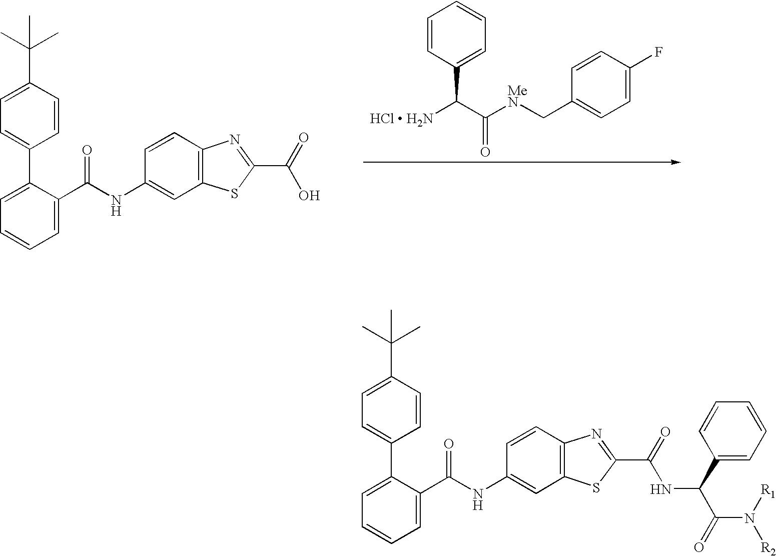

### Preparation of 4,5-dichloro-1,2,3-dithiazol-1-ium chloride (Appel's salt)

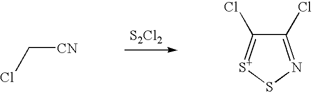

Disulfur dichloride (10 mL, 158 mmol) was added to a solution of 2-chloroacetonitrile (4.0 mL, 64 mmol) in CH₂Cl₂ (13 mL) at room temperature under an inert atmosphere of argon. The resulting mixture was stirred at room temperature for 18 h. The precipitate was collected by filtration, washed with CH₂Cl₂ copiously, and then dried to afford 4,5-dichloro-1,2,3-dithiazol-1-ium chloride (11.1 g, yield: 85%). MS (ESI) calcd for C₂Cl₃NS₂ (m/z): 208.52. found: 248.0 [M+K]⁺, 206.8 [M−2]⁻.

### Preparation of 2-bromo-4-nitroaniline

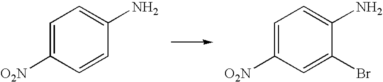

Bromine (2.05 mL, 40 mmol) was added dropwise to a solution of 4-nitroaniline (5.0 g, 36 mmol) in acetic acid (150 mL) at room temperature under an inert atmosphere of argon. The resulting reaction mixture was stirred at room temperature for 2 h. It was then quenched with dilute aqueous Na₂S₂O₃ (150 mL) and extracted with EtOAc (2×500 mL). The combined organic layers were added carefully to a saturated aqueous solution of NaHCO₃ (1 L) and stirred at room temperature for 30 min until all gas evolution had ceased. The organic layer was separated, dried (Na₂SO₄) and concentrated under reduced pressure. The resulting residue was purified by chromatography (3:1 petroleum ether/CH₂Cl₂) to afford 2-bromo-4-nitroaniline as a yellow solid (6.3 g, yield: 80%). ¹HNMR (400 MHz, CDCl₃) δ: 8.39 (1H, d, J=2.4 Hz), 8.06 (1H, d, J=2.4 Hz), 6.77 (1H, d, J=9.2 Hz).

### Preparation of (Z)-2-bromo-N-(4-chloro-5H-1,2,3-dithiazol-5-ylidene)-4-nitroaniline

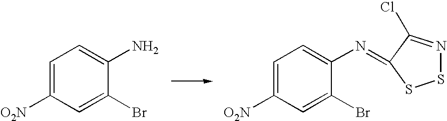

A mixture containing 4,5-dichloro-1,2,3-dithiazol-1-ium chloride (6.8 g, 5.6 mmol) and 2-bromo-4-nitroaniline (4.5 g, 24.9 mmol) in THF (100 mL) was stirred at room temperature for 18 h. The reaction mixture was concentrated under reduced pressure, and the resulting residue was purified by chromatography (elution with 4:1 petroleum ether/CH₂Cl₂) to afford (Z)-2-bromo-N-(4-chloro-5H-1,2,3-dithiazol-5-ylidene)-4-nitroaniline as an orange solid (3.5 g, yield: 40%). ¹HNMR (400 MHz, CDCl₃) δ: 8.61 (1H, d, J=2.0 Hz), 8.31 (d, J=2.4 Hz), 7.32 (1H, d, J=9.2 Hz). MS (ESI) calcd for C₈H₃BrClN₃O₂S₂ (m/z): 352.62. found: 318.1 [M-Cl]⁺, 274.1 [M-Br]⁺.

### Preparation of 6-nitrobenzo[d]thiazole-2-carbonitrile

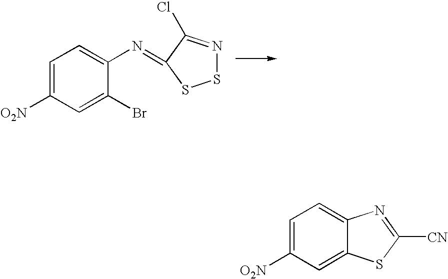

A mixture containing (Z)-2-bromo-N-(4-chloro-5H-1,2,3-dithiazol-5-ylidene)-4-nitroaniline (3.5 g, 9.9 mmol) and CuI (2.1 g, 11.0 mmol) in pyridine (32 mL) was heated in a microwave reactor at 110° C. for 40 min. After cooling to room temperature, the reaction mixture was concentrated under reduced pressure and the resulting residue was partitioned between EtOAc and water. The organic layer was separated, washed with brine, dried (MgSO₄) and concentrated under reduced pressure. The resulting residue was purified by chromatography (gradient elution using a 4:1 to 2:1 mixture of petroleum ether/CH₂Cl₂) to give 6-nitrobenzo[d]thiazole-2-carbonitrile as a yellow solid (1.4 g, yield: 70%). ¹HNMR (400 MHz, CDCl₃) δ: 8.98 (1H, s), 8.55 (1H, d, J=9.2 Hz), 8.42 (1H, d, J=9.2 Hz).

### Preparation of methyl 6-nitrobenzo[d]thiazole-2-carboxylate

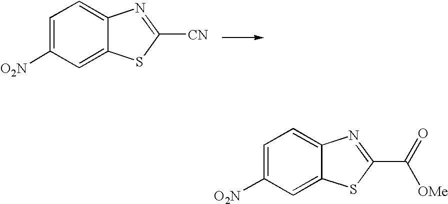

A solution of sodium methoxide in methanol (0.1 M, 6 mL) was added to a solution of 6-nitrobenzo[d]thiazole-2-carbonitrile (1.5 g, 7.3 mmol) in methanol (120 mL) and CH₂Cl₂ (10 mL) at room temperature. The resulting reaction mixture was stirred at 40° C. for 18 hrs. It was diluted with CH₂Cl₂ (10 mL), H₂O (10 mL) and 6 N HCl (0.1 mL). The resulting reaction mixture was stirred at room temperature for 18 h and concentrated under reduced pressure to afford crude methyl 6-nitrobenzo[d]thiazole-2-carboxylate as a yellow solid (2.24 g, crude yield: 129%). ¹HNMR (400 MHz, CDCl₃) δ: 8.97 (1H, s), 8.47 (1H, d, J=9.2 Hz), 8.40 (1H, d, J=9.2 Hz), 4.15 (3H, s).

### Preparation of methyl 6-aminobenzo[d]thiazole-2-carboxylate

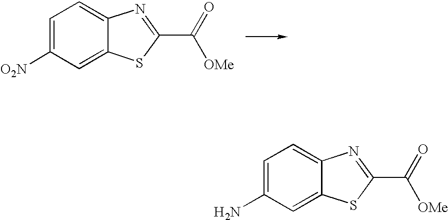

A suspension of crude methyl 6-nitrobenzo[d]thiazole-2-carboxylate (2.24 g, 9.4 mmol), NH₄Cl (4.02 g, 75.2 mmol), Fe (2.63 g, 47 mmol) in methanol (80 mL) and H₂O (20 mL) was stirred under reflux for 2 h. The reaction mixture was filtered and the filtrate was concentrated under reduced pressure. The resulting residue was partitioned between water and CH₂Cl₂. The organic layer was separated, washed with brine, dried (Na₂SO₄) and concentrated under reduced pressure. The residue was purified by chromatography (elution: CH₂Cl₂ containing 1% TEA) to afford methyl 6-aminobenzo[d]thiazole-2-carboxylate as a yellow solid (1.03 g, yield: 67%). ¹HNMR (400 MHz, CDCl₃) δ: 8.01 (1H, d, J=8.8 Hz), 7.14 (1H, J=2.4 Hz), 6.95 (1H, d, J=8.8 Hz), 4.07 (4H, s).

### Preparation of methyl 6-(4′-tert-butylbiphenyl-2-ylcarboxamido)benzo[d]thiazole-2-carboxylate

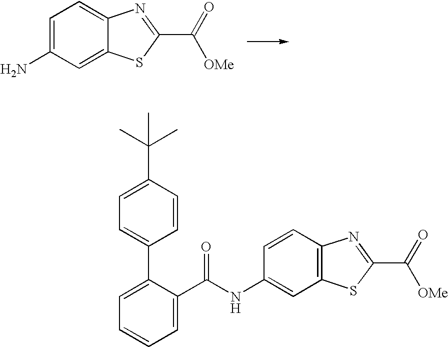

A mixture containing methyl 6-aminobenzo[d]thiazole-2-carboxylate (0.8 g, 3.84 mmol), 4′-tert-butylbiphenyl-2-carboxylic acid (1.16 g, 3.65 mmol), DMAP (508 mg, 4.16 mmol), and EDC.HCl (1.05 g, 5.47 mmol) in CH₂Cl₂ (20 mL) was stirred at room temperature for 18 h. Dilute aqueous NaHCO₃ solution was added to the reaction mixture. The organic layer was separated, washed with brine, dried (Na₂SO₄) and concentrated under reduced pressure. The resulting residue was purified by chromatography (elution: petroleum ether/EtOAc=8:1, plus 2% CH₂Cl₂) to afford methyl 6-(4′-tert-butylbiphenyl-2-ylcarboxamido)benzo[d]thiazole-2-carboxylate as a white solid (700 mg, yield: 41%). ¹H-NMR (400 MHz, CDCl₃) δ: 8.28 (1H, s), 8.01 (1H, d, J=7.6 Hz), 7.98 (1H, d, J=8.8 Hz), 7.60-7.42 (7H, m), 7.08 (1H , s), 6.46 (1H, d, J=9.2 Hz), 4.09 (3H, s).

### Preparation of 6-(4′-tert-butylbiphenyl-2-ylcarboxamido)benzo[d]thiazole-2-carboxylic acid

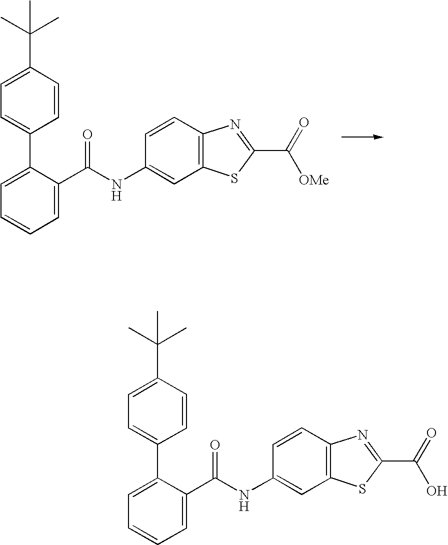

Lithium hydroxide monohydrate (68 mg) was added to a mixture of methyl 6-(4′-tert-butylbiphenyl-2-ylcarboxamido)benzo[d]thiazole-2-carboxylate (480 mg, 1.08 mmol) in methanol (5 mL), water (2 mL) and THF (2.5 mL). The reaction mixture was stirred at 40-50° C. for 18 h. The reaction mixture was concentrated under reduced pressure and the resulting residue was taken up in water (5 mL). Enough 1 N HCl was then added to produce a pH=3. The resulting white precipitate was collected by filtration and dried to give 6-(4′-tert-butylbiphenyl-2-ylcarboxamido)benzo[d]thiazole-2-carboxylic acid (320 mg, yield: 70%). ¹HNMR (400 MHz, CDCl₃) δ: 10.62 (1H, s), 8.52 (1H, s), 8.10 (1H, d, J=8.8 Hz), 7.61 (3H, m), 7.51 (2H, m), 7.40 (4H, s).

### Preparation of (S)-6-(4′-tert-butylbiphenyl-2-ylcarboxamido)-N-(2-((4-fluorobenzyl)(methyl)amino)-2-oxo-1-phenylethyl)benzo[d]thiazole-2-carboxamide (Compound #04)

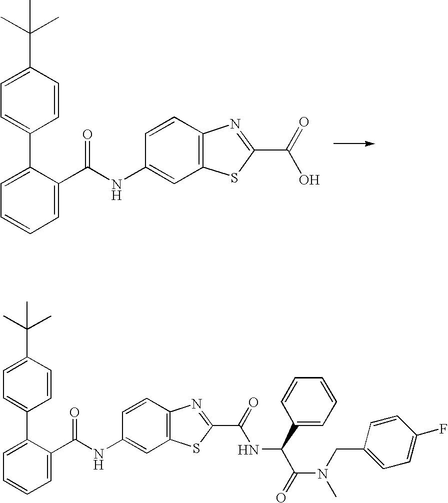

A mixture containing 6-(4′-tert-butylbiphenyl-2-ylcarboxamido)benzo[d]thiazole-2-carboxylic acid (43 mg, 0.1 mmol), (S)-2-amino-N-(4-fluorobenzyl)-N-methyl-2-phenylacetamide hydrochloride salt (46 mg, 0.15 mmol), EDC.HCl (29 mg, 0.15 mmol), and Et₃N (20 μL) in CH₂Cl₂ (2 mL) was stirred at room temperature for 18 h. The reaction mixture was concentrated under reduced pressure and the resulting residue was purified by preparative HPLC to afford (S)-6-(4′-tert-butylbiphenyl-2-ylcarboxamido)-N-(2-((4-fluorobenzyl)(methyl)-amino)-2-oxo-1-phenylethyl)benzo[d]thiazole-2-carboxamide as a white solid (14 mg). ¹HNMR (400 MHz, CDCl₃) δ: 8.83 (1H, d, J=7.6 Hz), 8.10 (1H, d, J=6.8 Hz), 8.00 (1H, d, J=6.8 Hz), 7.85 (1H, d, J=10.2 Hz), 7.83-7.34 (11H, m), 7.20 (1H, m), 7.03-6.94 (3H, m), 6.71 (1H, d, J=7.6 Hz), 6.05 (1H, d, J=11.6 Hz), 4.66 (2H, m), 2.94 (3H, d), 1.37 (9H, s); MS (ESI) calcd for C₄₁H₃₇FN₄O₃S (m/z): 684.26. found: 686.4 [M+2]⁺.

### Preparation of Compound #05, Compound #06, Compound #07, Compound #08, Compound #14, Compound #23, Compound #34, Compound #35

The same procedure used in the preparation of Compound #04 was employed using the appropriate amines.

### Preparation of Compound #76, Compound #77, Compound #78, Compound #79, Compound #80 and Compound #84

The general procedure outlined in the preparation of Compound #04 was employed using the appropriate acid component. Some of these acids are commercially available and some could be prepared according to the general procedure outlined in J. Med. Chem. 2001, p. 4677 and WO2006/113910.

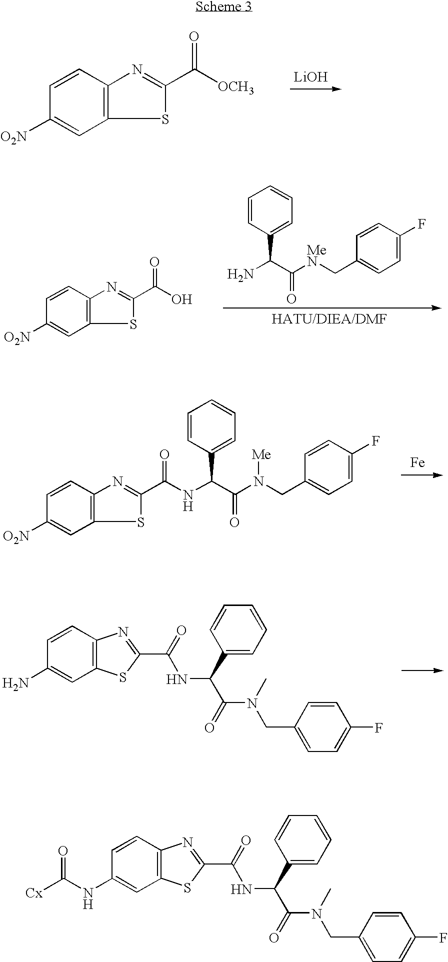

### Preparation of 6-nitrobenzo[d]thiazole-2-carboxylic acid

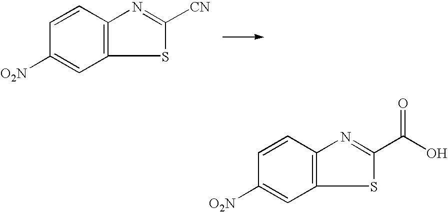

A solution of sodium methoxide in MeOH (0.1 M, 140 mL) was added to a solution of 6-nitrobenzo[d]thiazole-2-carbonitrile (35 g, 170.6 mmol) in MeOH (3 L) and CH₂Cl₂ (1 L) under an inert atmosphere of argon. The resulting reaction mixture was stirred at room temperature for 2 days. The reaction mixture was concentrated under reduced pressure and the resulting residue was taken up in CH₂Cl₂ (2 L) and 0.5 N HCl (1 L). The mixture was stirred vigorously for 1 h and the two layers were separated. The organic layer was dried (Na₂SO₄) and concentrated under reduced pressure to afford methyl 6-nitrobenzo[d]thiazole-2-carbonitrile as a yellow solid (30 g, yield: 74%).

A solution containing methyl 6-nitrobenzo[d]thiazole-2-carbonitrile (5 g, 21.0 mmol) in dioxane (300 mL) was cooled to 0° C. A solution of ice-cooled NaOH (300 mL, 1 M) was then added. The resulting reaction mixture was stirred at 0° C. for 15 min and ice-cooled 1 N HCl (400 mL) was added. The resulting mixture was immediately extracted with EtOAc (3×300 mL). The combined organic layers were washed with brine, dried (Na₂SO₄) and concentrated under reduced pressure to afford 6-nitrobenzo[d]thiazole-2-carboxylic acid as a yellow solid (4.5 g, yield: 96%). ¹HNMR (400 MHz, DMSO-d₆) δ: 9.077 (1H, s), 8.33 (1H, dd, J=8.8 Hz), 8.20 (1H, d, J=8.8 Hz); MS (ESI) calcd for C₈H₄N₂O₄S (m/z): 224.19. found: 224.9 [M+1]⁺.

### Preparation of (S)—N-(2-((4-fluorobenzyl)(methyl)amino)-2-oxo-1-phenylethyl)-6-nitrobenzo[d]thiazole-2-carboxamide

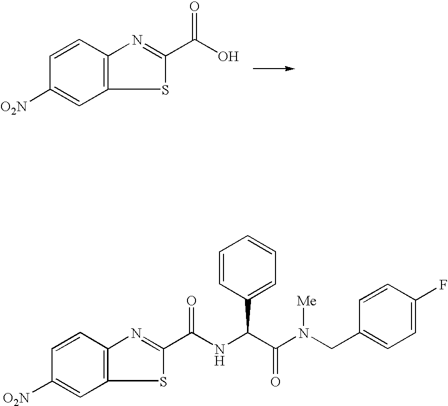

A mixture containing 6-nitrobenzo[d]thiazole-2-carboxylic acid (500 mg, 2.23 mmol), (S)-2-amino-N-(4-fluorobenzyl)-N-methyl-2-phenylacetamide (910.7 mg, 3.34 mmol), HATU (1.69 g, 4.46 mmol) and DIPEA (0.74 mmol) in DMF (5 mL) was stirred at room temperature for 18 h. The reaction mixture was diluted with water (50 mL) and the resulting precipitate was collected. The collected solids were further extracted with ethyl acetate (100 mL), dried (MgSO₄) and concentrated under reduced pressure. The resulting residue was purified by chromatography (elution: 3:1 mixture of petroleum ether/EtOAc) to afford (S)—N-(2-((4-fluorobenzyl)(methyl)amino)-2-oxo-1-phenylethyl)-6-nitrobenzo[d]thiazole-2-carboxamide as a yellow solid (780 mg, yield: 36%). ¹HNMR (400 MHz, CDCl₃) δ: 8.908 (1H, s), 8.904 (1H, s), 8.43 (1H, dd, J₁=8.8 Hz, J₂=2.0 Hz), 8.24 (1H, d, J=8.8 Hz), 7.55 (2H, m), 7.42 (3H, m), 7.20 (1H, dd, J₁=8.4 Hz, J₂=5.2 Hz), 7.02 (2H, d, J=8.4 Hz), 6.03 (1H, d, J=7.2 Hz), 4.67 (2H, q, J=7.6 Hz), 2.96 (2H, s), 2.87 (1H, s).

### Preparation of (S)-6-amino-N-(2-((4-fluorobenzyl)(methyl)amino)-2-oxo-1-phenylethyl)benzo[d]thiazole-2-carboxamide

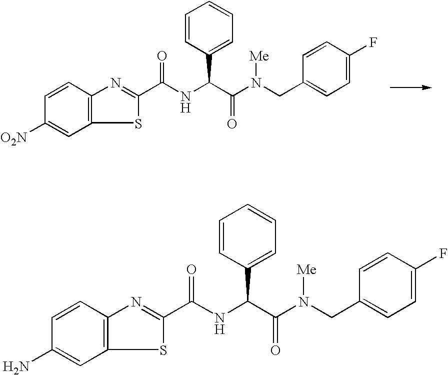

Iron powder (222 mg, 3.97 mmol) and NH₄Cl (338 mg, 6.32 mmol) were added to a suspension of (S)—N-(2-((4-fluorobenzyl)(methyl)amino)-2-oxo-1-phenylethyl)-6-nitrobenzo[d]thiazole-2-carboxamide (380 mg, 0.79 mmol) in methanol (8 mL) and H₂O (2 mL). The resulting reaction mixture was stirred under reflux for 2 h. Upon cooling to room temperature, the reaction mixture was filtered and the filtrate was concentrated under reduced pressure. The resulting residue was purified by chromatography (elution: petroleum ether/EtOAc=1:1) to afford (S)-6-amino-N-(2-((4-fluorobenzyl)(methyl)amino)-2-oxo-1-phenylethyl)benzo[d]thiazole-2-carboxamide as a yellow solid (287 mg, yield: 81%). ¹H NMR (400 MHz, CDCl₃) δ: 13.2 (1H, m), 6.864-7.861 (12H, m), 6.035-6.091 (1H, m), 4.573-4.689 (2H, m), 3.953 (2H, s), 2.94 (1H, s), 2.87 (2H, s).

### Preparation of ethyl 2-(imidazo[2,1-b]thiazol-6-yl)benzoate

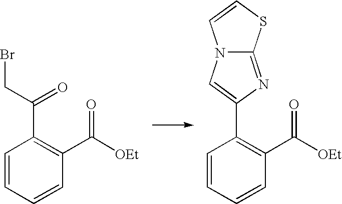

A mixture containing ethyl 2-(2-bromoacetyl)benzoate (400 mg, 1.48 mmol) and thiazol-2-amine (220 mg, 2.2 mmol) in EtOH (6 mL) was stirred under reflux for 18 h. The reaction mixture was cooled to room temperature and diluted with water. The resulting mixture was extracted with ethyl acetate. The combined organic layers were dried (MgSO₄) and concentrated under reduced pressure to afford ethyl 2-(imidazo[2,1-b]thiazol-6-yl)benzoate as a tan oil (354 mg, yield: 88%). ¹H NMR (400 MHz, CDCl₃) δ: 7.76 (1H, d, J=7.6), 7.71 (1H, t, J=7.6 Hz), 7.53 (1H, t, J=7.6 Hz), 7.46 (1H, d, J=4.4 Hz), 7.40 (1H, t, J=8.0 Hz), 6.84 (1H, d, J=4.0 Hz), 4.34 (2H, q, J=7.2 Hz), 1.27 (3H, t, J=7.2 Hz).

### Preparation of 2-(imidazo[2,1-b]thiazol-6-yl)benzoic acid

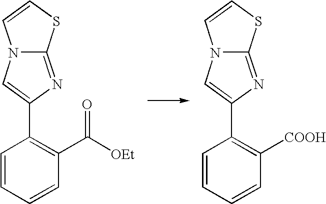

A solution of ethyl 2-(imidazo[2,1-b]thiazol-6-yl)benzoate (100 mg, 0.36 mmol) in 6 N HCl (2 mL) was stirred under reflux for 4 h. The reaction mixture was cooled to room temperature. The precipitated product was collected by filtration and dried to afford 2-(imidazo[2,1-b]thiazol-6-yl)benzoic acid as a tan solid (43 mg, yield: 49%). ¹H NMR (400 MHz, DMSO-d₆) δ: 8.14 (2H, m), 7.77 (1H, d, J=7.2 Hz), 7.70 (1H, d, J=7.6 Hz), 7.64 (1H, t, J=7.6 Hz), 7.54 (2H, m); MS (ESI) calcd for C₁₂H₈N₂O₂S (m/z): 244.27. found: 244.8 [M+1]⁺.

### Preparation of (S)—N-(2-((4-fluorobenzyl)(methyl)amino)-2-oxo-1-phenylethyl)-6-(2-(imidazo[2,1-b]thiazol-6-yl)benzamido)benzo[d]thiazole-2-carboxamide (Compound #24)

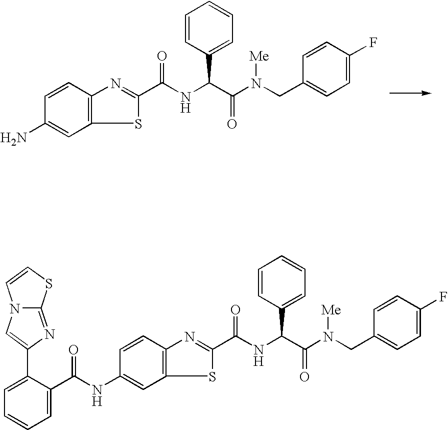

A mixture containing (S)-6-amino-N-(2-((4-fluorobenzyl)(methyl)amino)-2-oxo-1-phenylethyl)benzo[d]thiazole-2-carboxamide (50 mg, 0.11 mmol), 2-(imidazo[2,1-b]thiazol-6-yl)benzoic acid (68 mg, 0.28 mmol), HATU (168 mg, 0.44 mmol) and DIPEA (0.1 mL, 0.66 mmol) in DMF (3 mL) was stirred at room temperature for 18 h and at 50° C. for an additional 4 h. The reaction mixture was diluted with water (5 mL) and saturated aqueous NaHCO₃ (5 mL). The resulting precipitate was collected by filtration and further purified by preparative TLC to afford (S)—N-(2-((4-fluorobenzyl)(methyl)amino)-2-oxo-1-phenylethyl)-6-(2-(imidazo[2,1-b]thiazol-6-yl)benzamido)benzo[d]thiazole-2-carboxamide as a yellow solid (47 mg, yield: 63%). ¹H NMR (400 MHz, CDCl₃) δ: 9.81 (1H, s), 8.83 (1H, d, J=6.8 Hz), 8.67 (1H, s), 7.98 (1H, d, J=9.2 Hz), 7.89 (1H, d, J=7.2 Hz), 7.66 (2H, m), 7.51 (4H, m), 7.39 (5H, m), 7.19 (1H, q, J=7.6 Hz), 7.00 (3H, m), 6.88 (1H, d, J=4.4 Hz), 6.04 (1H, q, J=7.6 Hz), 4.63 (2H, q, J=7.6 Hz), 2.91 (1H, s), 2.84 (2H, s); MS (ESI) calcd for C₃₆H₂₇FN₆O₃S₂ (m/z): 674.16. found: 675.2 [M+1]⁺, 676.3 [M+2]⁺.

### Preparation of Compound #25, Compound #28, Compound #29, Compound #30, Compound #31, Compound #32, Compound #38, Compound #39, Compound #40, Compound #43, Compound #44, Compound #51, Compound #52, Compound #53, Compound #61, Compound #62 and Compound #63

The same procedure used in the preparation of Compound #24 was employed using the appropriate acids.

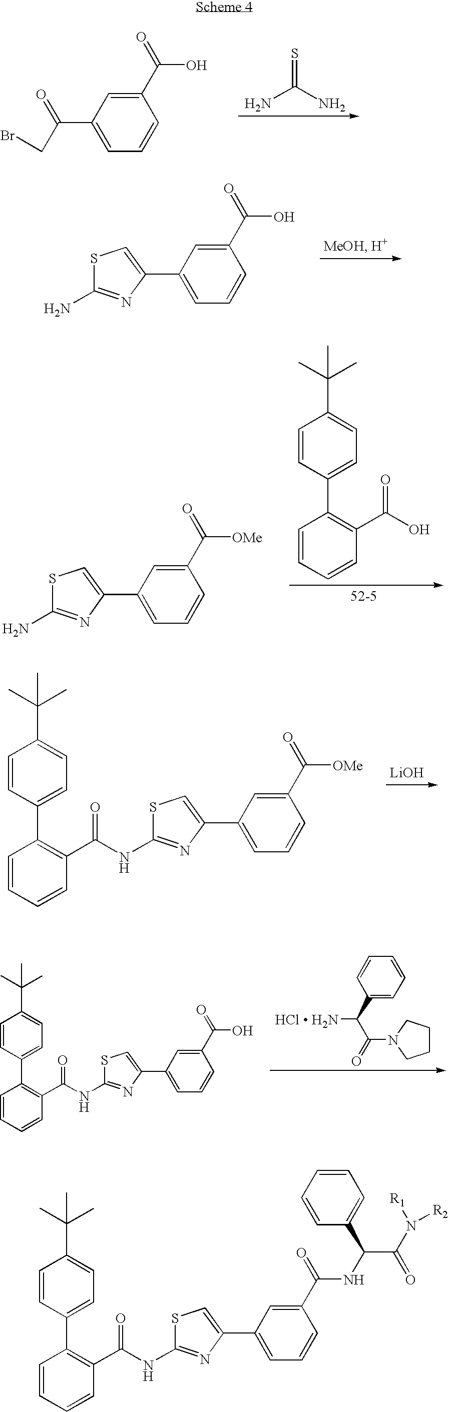

### Preparation of 3-(2-aminothiazol-4-yl)benzoic acid

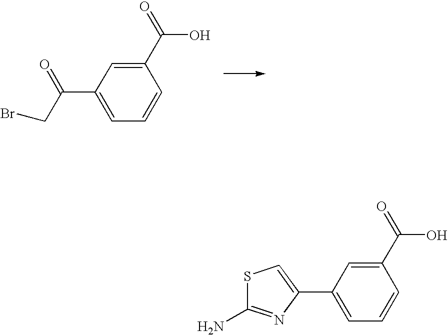

A mixture containing 3-(2-bromoacetyl)benzoic acid (2.43 g, 10 mmol) and thiourea (1.14 g, 15 mmol) in EtOH (20 mL) was stirred under reflux for 18 h. The reaction mixture was cooled to room temperature and diluted with water (40 mL). The precipitated solids were collected by filtration and dried to afford compound 3-(2-aminothiazol-4-yl)benzoic acid as a white solid (1.9 g, yield: 86%).

### Preparation of methyl 3-(2-aminothiazol-4-yl)benzoate

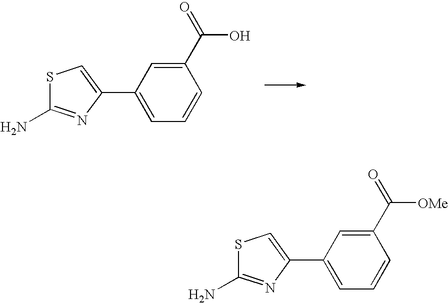

Thionyl chloride (1.23 mL, 17 mmol) was added dropwise at 0° C. to a solution of compound 3-(2-aminothiazol-4-yl)benzoic acid (1.87 g, 8.5 mmol) in methanol (20 mL). The resulting reaction mixture was then stirred under reflux for 18 h. It was then cooled to room temperature and concentrated under reduced pressure. The resulting residue was partitioned between saturated aqueous NaHCO₃ solution (50 mL) and CH₂Cl₂ (100 mL). The organic layer was separated and the aqueous layer was further extracted with CH₂Cl₂ (2×100 mL). The combined organic layers were dried (Na₂SO₄) and concentrated under reduced pressure to afford methyl 3-(2-aminothiazol-4-yl)benzoate as a white solid (1.94 g, yield: 97%). ¹HNMR (400 MHz, DMSO-d₆) δ: 3.876 (3H, s), 7.171 (1H, s), 7.182 (2H, s), 7.520 (1H, dd, J₁=J₂=8.0 Hz), 7.846 (1H, d, J=8.0 Hz), 8.063 (1H, d, J=8.0 Hz), 8.431 (1H, s).

### Preparation of methyl 3-(2-(4′-tert-butylbiphenyl-2-ylcarboxamido)thiazol-4-yl)benzoate

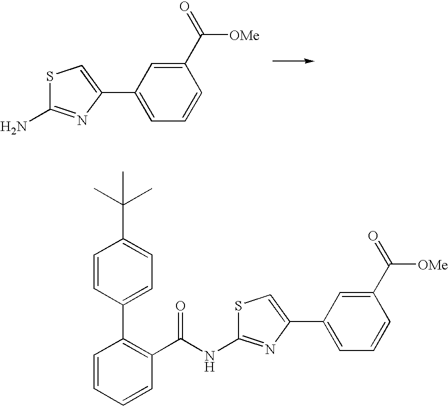

A mixture containing methyl 3-(2-aminothiazol-4-yl)benzoate (60 mg, 0.25 mmol), 4′-tert-butylbiphenyl-2-carboxylic acid (56 mg, 0.22 mmol), DMAP (32 mg, 0.26 mmol) and EDC.HCl (64 mg, 33 mmol) in CH₂Cl₂ (4 mL) was stirred at room temperature for 18 h. The reaction solution was concentrated under reduced pressure and the resulting residue was purified by chromatography (elution: CH₂Cl₂) to afford methyl 3-(2-(4′-tert-butylbiphenyl-2-ylcarboxamido)thiazol-4-yl)benzoate as a white solid (30 mg, yield: 29%). ¹H NMR (400 MHz, DMSO-d₆) δ: 1.254 (9H, s), 3.876 (3H, s), 7.324 (2H, d, J=8.0 Hz), 7.412 (2H, d, J=8.0 Hz), 7.491 (2H, m), 7.587˜7.652 (3H, m), 7.808 (1H, s), 7.911 (1H, d, J=7.2 Hz), 8.161 (1H, d, J=7.6 Hz), 8.515 (1H, s).

### Preparation of 3-(2-(4′-tert-butylbiphenyl-2-ylcarboxamido)thiazol-4-yl)benzoic acid

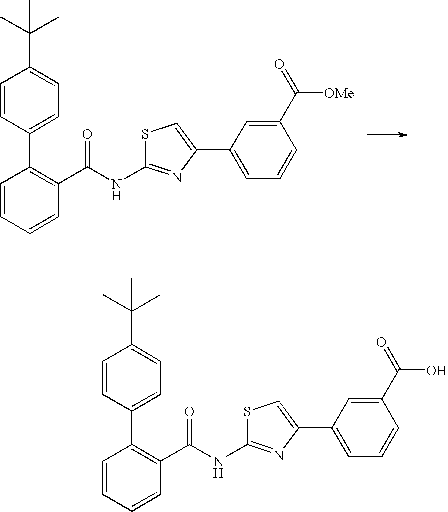

Lithium hydroxide monohydrate (232 mg, 5.532 mmol) was added to a mixture of 3-(2-(4′-tert-butylbiphenyl-2-ylcarboxamido)thiazol-4-yl)benzoate (869 mg, 1.845 mmol) in THF (10 mL), H₂O (4 mL) and MeOH (6 mL). The resulting reaction mixture was stirred at 50° C. for 18 h. It was then cooled to room temperature and concentrated under reduced pressure. The resulting residue was taken up in water and washed with Et₂O. Enough 6 N HCl was then added to produce pH=3. The precipitate was collected by filtration, washed with petroleum ether and dried to afford 3-(2-(4′-tert-butylbiphenyl-2-ylcarboxamido)thiazol-4-yl)benzoic acid as a white solid (777 mg, yield: 92%). ¹HNMR (400 MHz, DMSO-d₆) δ: 12.619 (1H, s), 7.309-8.501 (13H, m), 1.251 (9H, s).

### Preparation of (S)-4′-tert-butyl-N-(4-(3-(2-oxo-1-phenyl-2-(pyrrolidin-1-yl)ethylcarbamoyl)phenyl)thiazol-2-yl)biphenyl-2-carboxamide (Compound #12)

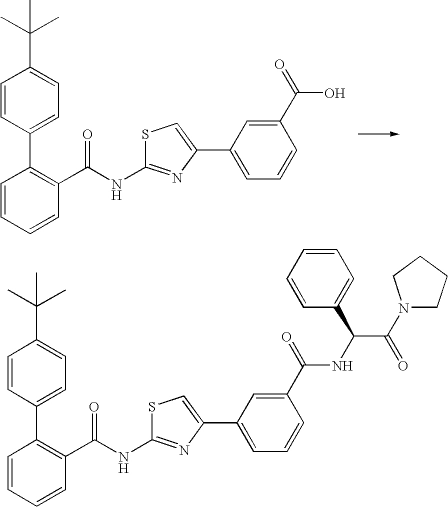

A mixture containing 3-(2-(4′-tert-butylbiphenyl-2-ylcarboxamido)thiazol-4-yl)benzoic acid (100 mg, 0.219 mmol) and (S)-2-amino-2-phenyl-1-(pyrrolidin-1-yl)ethanone hydrochloride salt (64 mg, 0.263 mmol) in CH₂Cl₂ (3 mL) was stirred at room temperature. Triethylamine (0.046 mL, 0.329 mmol) and EDC.HCl (63 mg, 0.329 mmol) were then added sequentially. The resulting reaction mixture was stirred at room temperature for 18 h. It was concentrated under reduced pressure and the resulting residue was purified by preparative TLC (elution: petroleum ether/EtOAc=2:1 plus 5% CH₂Cl₂) to afford (S)-4′-tert-butyl-N-(4-(3-(2-oxo-1-phenyl-2-(pyrrolidin-1-yl)ethylcarbamoyl)phenyl)thiazol-2-yl)biphenyl-2-carboxamide as a white solid (36 mg, yield: 26%). ¹HNMR (400 MHz, CDCl₃): a: 10.275 (1H, s), 6.835-8.178 (18H, m), 5.958 (1H, d, J=8.0 Hz), 3.115-3.616 (4H, m), 1.740-1.939 (4H, m), 1.213 (9H, s); MS (ESI) calcd for C₃₉H₃₈N₄O₃S (m/z): 642.81. found: 643.5 [M+1]⁺.

### Preparation of Compound #11, Compound #13, Compound #26 and Compound #27

The same procedure used in the preparation of Compound #12 was employed using the appropriate amines.

### Preparation of Compound #87, Compound #88, Compound #89, Compound #90, Compound #94, Compound #95, Compound #97, Compound #98 and Compound #99

The general procedure outlined in the preparation of Compound #12 was employed using the appropriate acid component. Some of these acids are commercially available and some could be prepared according to the general procedure outlined in J. Med. Chem. 2001, p. 4677 and WO2006/113910.

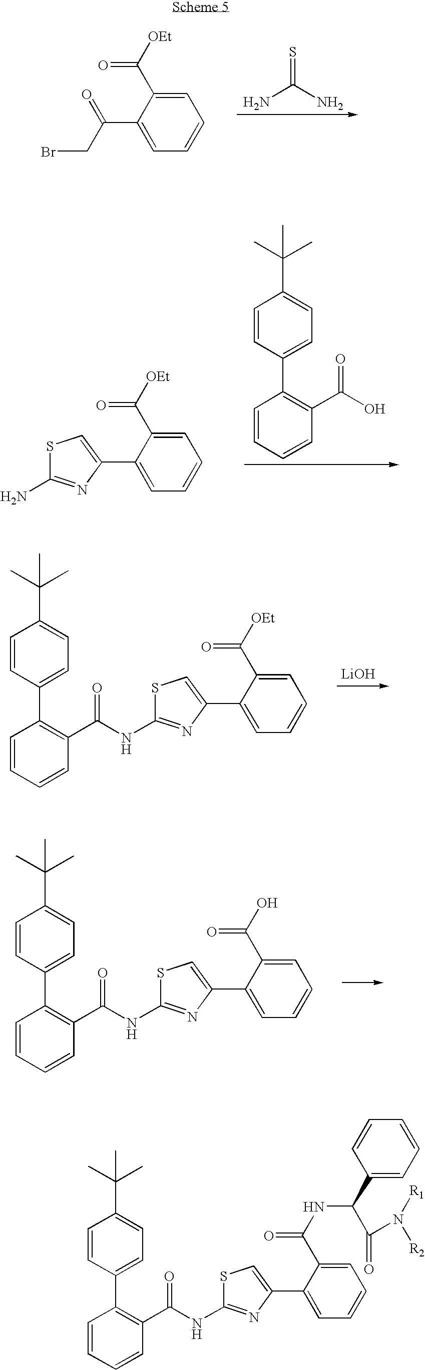

### Preparation of ethyl 2-(2-aminothiazol-4-yl)benzoate

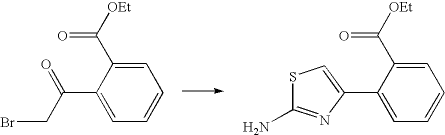

A mixture containing ethyl 2-(2-bromoacetyl)benzoate (2.71 g, 10 mmol) and thiourea (1.14 g, 15 mmol) in EtOH (20 mL) was stirred under reflux for 18 h. The reaction mixture was cooled to room temperature and diluted with water (30 mL). Enough saturated aqueous NaHCO₃ was then added to produce a pH=8. After stirring for 30 min, the precipitate was collected by filtration, washed with water and dried to afford ethyl 2-(2-aminothiazol-4-yl)benzoate as a white solid (2.4 g, yield: 97%). ¹HNMR (400 MHz, DMSO-d₆) δ: 1.120 (3H, t, J=7.2 Hz), 4.166 (2H, t, J=7.2 Hz), 6.722 (1H, s), 6.974 (2H, s), 7.390 (1H, d, J=7.6 Hz), 7.587˜7.652 (2H, dd, J₁=J₂=8.0 Hz), 7.614 (1H, d, J=8.0 Hz).

### Preparation of ethyl 2-(2-(4′-tert-butylbiphenyl-2-ylcarboxamido)thiazol-4-yl)benzoate

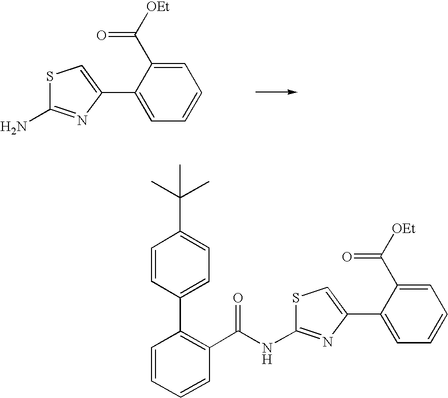

A mixture containing ethyl 2-(2-aminothiazol-4-yl)benzoate (1.3 g, 5.24 mmol), 4′-tert-butylbiphenyl-2-carboxylic acid (1.29 g, 4.36 mmol), DMAP (0.64 g, 5.24 mmol) and EDC.HCl (1.26 g, 6.54 mmol) in CH₂Cl₂ (20 mL) was stirred at room temperature for 18 h. The reaction solution was concentrated and purified by chromotography (elution:petroleum ether:CH₂Cl₂=1:2) to afford ethyl 2-(2-(4′-tert-butylbiphenyl-2-ylcarboxamido)thiazol-4-yl)benzoate as a white solid (1.36 g, yield: 64%). ¹H-NMR (400 MHz, DMSO-d₆) δ: 1.007 (3H, t, J=7.2 Hz), 1.276 (9H, s), 4.107 (2H, m), 7.325 (2H, d, J=8.4 Hz), 7.362 (1H, s), 7.403 (2H, d, J=8.0 Hz), 7.475 (3H, m), 7.560˜7.616 (4H, m), 7.641 (1H, d, J=8.0 Hz), 12.678 (1H, s).

### Preparation of 2-(2-(4′-tert-butylbiphenyl-2-ylcarboxamido)thiazol-4-yl)benzoic acid

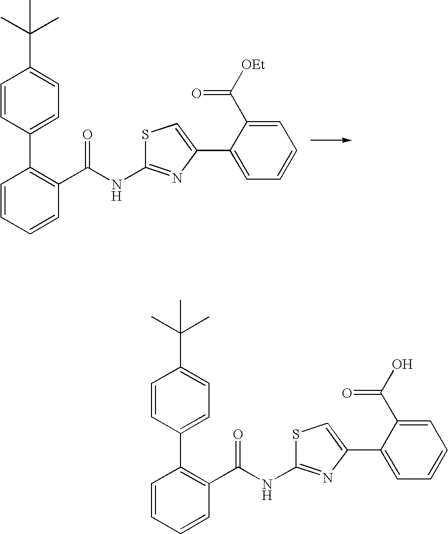

A solution of lithium hydroxide monohydrate (32 mg, 0.75 mmol.) in water (2 mL) was added to a mixture of ethyl 2-(2-(4′-tert-butylbiphenyl-2-ylcarboxamido)thiazol-4-yl)benzoate (242 mg, 0.5 mmol) in CH₃OH (2 mL) and THF (2 mL). The resulting reaction mixture was then stirred at room temperature for 18 h. In order to drive the hydrolysis to completion, another portion of lithium hydroxide monohydrate (40 mg) in water (2 mL) was added and the resulting reaction mixture was stirred at 50° C. for an additional 6 h. The reaction mixture was cooled to room temperature and concentrated under reduced pressure. Enough 10% aqueous HCl solution was added to produce a pH=2. The resulting mixture was extracted with CH₂Cl₂ (30 mL×2). The combined organic layers were dried (Na₂SO₄) and concentrated under reduced pressure to afford 2-(2-(4′-tert-butylbiphenyl-2-ylcarboxamido)thiazol-4-yl)benzoic acid as a white solid (0.23 g, yield: 99%). ¹H NMR (400 MHz, DMSO-d₆) δ: 1.288 (9H, s), 7.233 (1H, s), 7.330 (2H, d, J=8.4 Hz), 7.417˜7.492 (5H, m), 7.544˜7.613 (3H, m), 7.613 (1H, s), 7.656 (1H, d, J=8.0 Hz), 12.645 (1H, s), 12.675 (1H, s).

### Preparation of (S)-4′-tert-butyl-N-(4-(2-(2-oxo-1-phenyl-2-(pyrrolidin-1-yl)ethylcarbamoyl)phenyl)thiazol-2-yl)biphenyl-2-carboxamide (Compound #16)

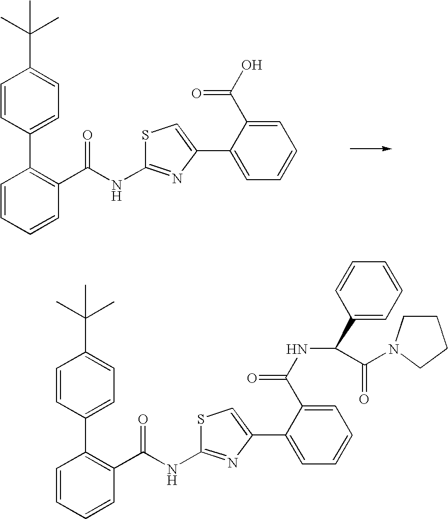

A mixture containing 2-(2-(4′-tert-butylbiphenyl-2-ylcarboxamido)thiazol-4-yl)benzoic acid (60 mg, 0.131 mmol), (S)-2-amino-2-phenyl-1-(pyrrolidin-1-yl)ethanone hydrochloride salt (40 mg, 0.197 mmol), HATU (100 mg, 0.262 mmol) and DIPEA (0.05 mL, 0.262 mmol) in DMF (1 mL) was stirred at room temperature for 18 h. The reaction mixture was then diluted with water. The resulting precipitate was collected by filtration and purified by preparative TLC (elution: petroleum ether:EtOAc=1:1 plus 5% CH₂Cl₂) to afford (S)-4′-tert-butyl-N-(4-(2-(2-oxo-1-phenyl-2-(pyrrolidin-1-yl)ethylcarbamoyl)phenyl)thiazol-2-yl)biphenyl-2-carboxamide as a yellow solid (38 mg, yield: 26%). ¹H NMR (400 MHz, CDCl₃): δ: 9.619 (1H, s), 6.781-8.249 (18H, m), 5.775 (1H, d, J=7.2 Hz), 3.044-3.652 (4H, m), 1.743-1.941 (4H, m), 1.321 (9H, s); MS (ESI) calcd for C₃₉H₃₈N₄O₃S (m/z): 642.81. found: 643.6 [M+1]⁺.

### Preparation of Compound #15 and Compound #17

The same procedure used in the preparation of Compound #16 was employed using the appropriate amines.

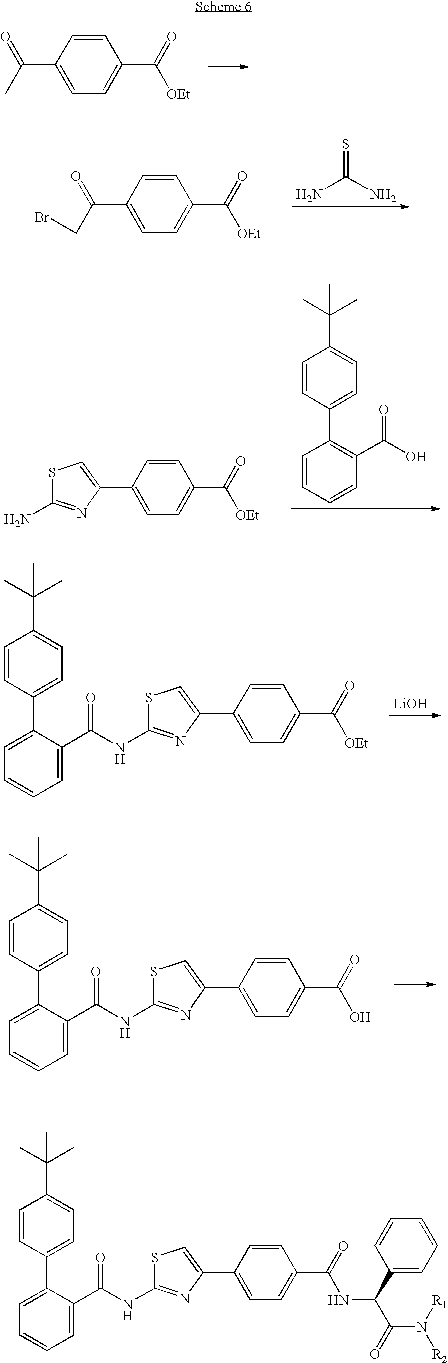

### Preparation of ethyl 4-(2-bromoacetyl)benzoate

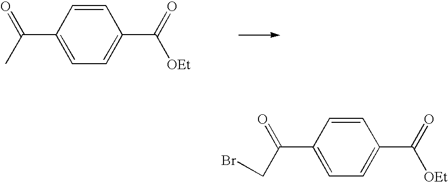

A mixture containing ethyl 4-acetylbenzoate (502 mg, 2.61 mmol), AlCl₃ (50 mg, 0.37 mmol) in anhydrous ether (5 mL) was cooled in an ice bath under an inert atmosphere of argon. Bromine (0.17 mL, 3.31 mmol) was then added as a solution in ether (15 mL) over a period of 10 min. The reaction mixture was slowly allowed to warm to room temperature and stirred for 18 h. The reaction mixture was carefully poured into saturated aqueous NaHCO₃ solution and stirred for 30 min. The organic layer was separated and washed successively with dilute aqueous NaHCO₃ and H₂O. It was then dried (Na₂SO₄) and concentrated under reduced pressure to afford crude ethyl 4-(2-bromoacetyl)benzoate as a white solid (650 mg). ¹H NMR (400 MHz, CDCl₃) δ: 1.45 (3H, t, J=7.2 Hz), 4.44 (2H, q, J=7.2 Hz), 8.06-8.00 (5H, m).

### Preparation of ethyl 4-(2-aminothiazol-4-yl)benzoate

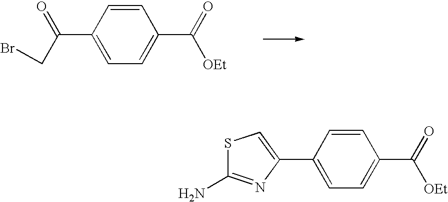

A mixture containing ethyl 4-(2-bromoacetyl)benzoate (374 mg, 1.38 mmol) and thiourea (126 mg, 1.66 mmol) in EtOH (15 mL) was stirred at room temperature for 18 h. The precipitated solids were collected by filtration and dried to afford ethyl 4-(2-aminothiazol-4-yl)benzoate as a white solid (342 mg, yield: 100%). ¹H NMR (400 MHz, CDCl₃) δ: 1.33 (3H, t, J=7.2 Hz), 4.32 (2H, q, J=7.2 Hz), 7.34 (1H, m), 7.90 (2H, dd, J=5.6 Hz, J₂=1.6 Hz), 7.99 (2H, t, J=8.0 Hz).

### Preparation of ethyl 4-(2-(4′-tert-butylbiphenyl-2-ylcarboxamido)thiazol-4-yl)benzoate

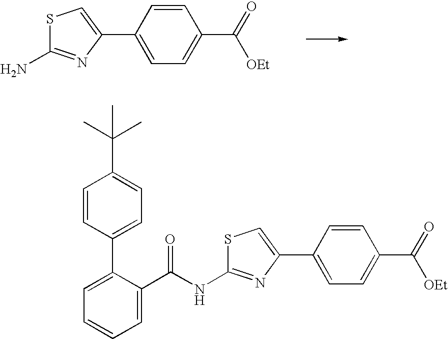

A mixture containing ethyl 4-(2-aminothiazol-4-yl)benzoate (340 mg, 1.37 mmol), 4′-tert-butylbiphenyl-2-carboxylic acid (731 mg, 2.88 mmol), DMAP (419 mg, 3.43 mmol), and EDC.HCl (659 mg, 3.43 mmol) in CH₂Cl₂ (10 mL) was stirred at room temperature for 40 h. The reaction mixture was diluted with water the organic layer was separated, dried (Na₂SO₄) and concentrated under reduced pressure. The resulting residue was purified by chromatography (elution: 1:1 petroleum ether/EtOAc) to afford ethyl 4-(2-(4′-tert-butylbiphenyl-2-ylcarboxamido)thiazol-4-yl)benzoate as a white solid (230 mg, yield: 35%). ¹H NMR (400 MHz, CDCl₃) δ: 1.35 (9H, s), 1.42 (3H, t, J=7.2 Hz), 4.40 (2H, q, J=7.2 Hz), 7.24 (1H, s), 7.37 (2H, d, J=22.8 Hz), 7.49 (4H, m), 7.57 (1H, m), 7.76 (2H, d, J=8.4 Hz), 7.95 (1H, d, J=7.2 Hz), 8.02 (2H, d, J=8.8 Hz), 8.75 (1H, s).

### Preparation of 4-(2-(4′-tert-butylbiphenyl-2-ylcarboxamido)thiazol-4-yl)benzoic acid

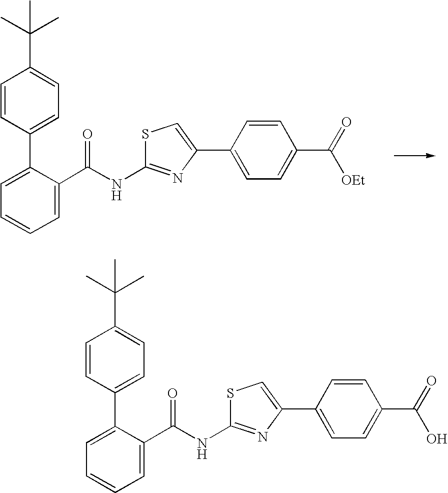

Lithium hydroxide monohydrate (40 mg, 0.95 mmol) was added to a mixture of ethyl 4-(2-(4′-tert-butylbiphenyl-2-ylcarboxamido)thiazol-4-yl)benzoate (230 mg, 0.47 mmol) in methanol (2 mL), water (0.5 mL) and THF (1 mL). The reaction mixture was stirred at 50° C. for 24 h, allowed to cool to room temperature and concentrated under reduced pressure. The resulting residue was taken up in water and enough 1 N HCl was added to produce a pH=2. The precipitate was collected by filtration and dried to afford 4-(2-(4′-tert-butylbiphenyl-2-ylcarboxamido)thiazol-4-yl)benzoic acid as a white solid (195 mg, yield: 90%). ¹H NMR (400 MHz, DMSO-d₆) δ: 1.26 (9H, s), 7.32 (2H, d, J=8.4 Hz), 7.42 (2H, d, J=8.4 Hz), 7.49 (2H, m), 7.63 (2H, m), 7.83 (1H, s), 7.99 (4H, s), 12.75 (1H, s).

### Preparation of (S)-4′-tert-butyl-N-(4-(4-(2-oxo-1-phenyl-2-(pyrrolidin-1-yl)ethylcarbamoyl)phenyl)thiazol-2-yl)biphenyl-2-carboxamide (Compound #36)

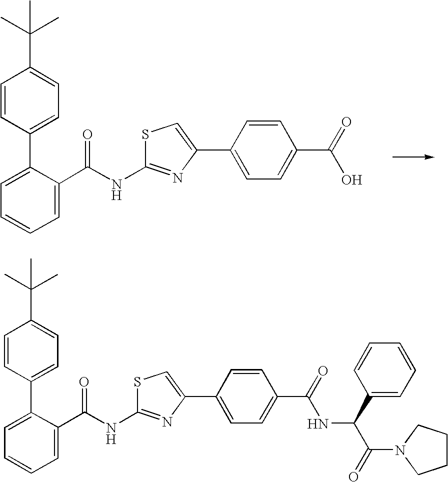

A mixture containing 4-(2-(4′-tert-butylbiphenyl-2-ylcarboxamido)thiazol-4-yl)benzoic acid (50 mg, 0.11 mmol), (S)-2-(chloroamino)-2-phenyl-1-(pyrrolidin-1-yl)ethanone (40 mg, 0.165 mmol), HATU (84 mg, 0.22 mmol), and DIPEA (0.11 mL, 0.66 mmol) in DMF (3 mL) was stirred room temperature for 18 h. The reaction mixture was then diluted with water. The resulting precipitate was collected by filtration and purified by preparative TLC to afford (S)-4′-tert-butyl-N-(4-(4-(2-oxo-1-phenyl-2-(pyrrolidin-1-yl)ethylcarbamoyl)phenyl)thiazol-2-yl)biphenyl-2-carboxamide as a white solid (61 mg, yield: 87%). ¹H NMR (400 MHz, CDCl₃) δ: 1.34 (9H, s), 1.85 (4H, m), 3.14 (1H, m), 3.48 (1H, m), 3.61 (2H, m), 5.87 (1H, d, J=6.8 Hz), 7.19 (1H, s), 7.36 (5H, m), 7.55 (7H, m), 7.68 (2H, d, J=8.4 Hz), 7.75 (2H, d, J=8.0 Hz). 7.93 (2H, m), 8.93 (1H, s); MS (ESI) calcd for C₃₉H₃₈N₄O₃S (m/z): 642.27. found: 643.6 [M+1]⁺, 644.6 [M+2]⁺.

### Preparation of Compound #37

The same procedure used in the preparation of Compound #36, Compound #81, Compound #82, Compound #83, Compound #91, Compound #92 and Compound #93 was employed using the appropriate amine.

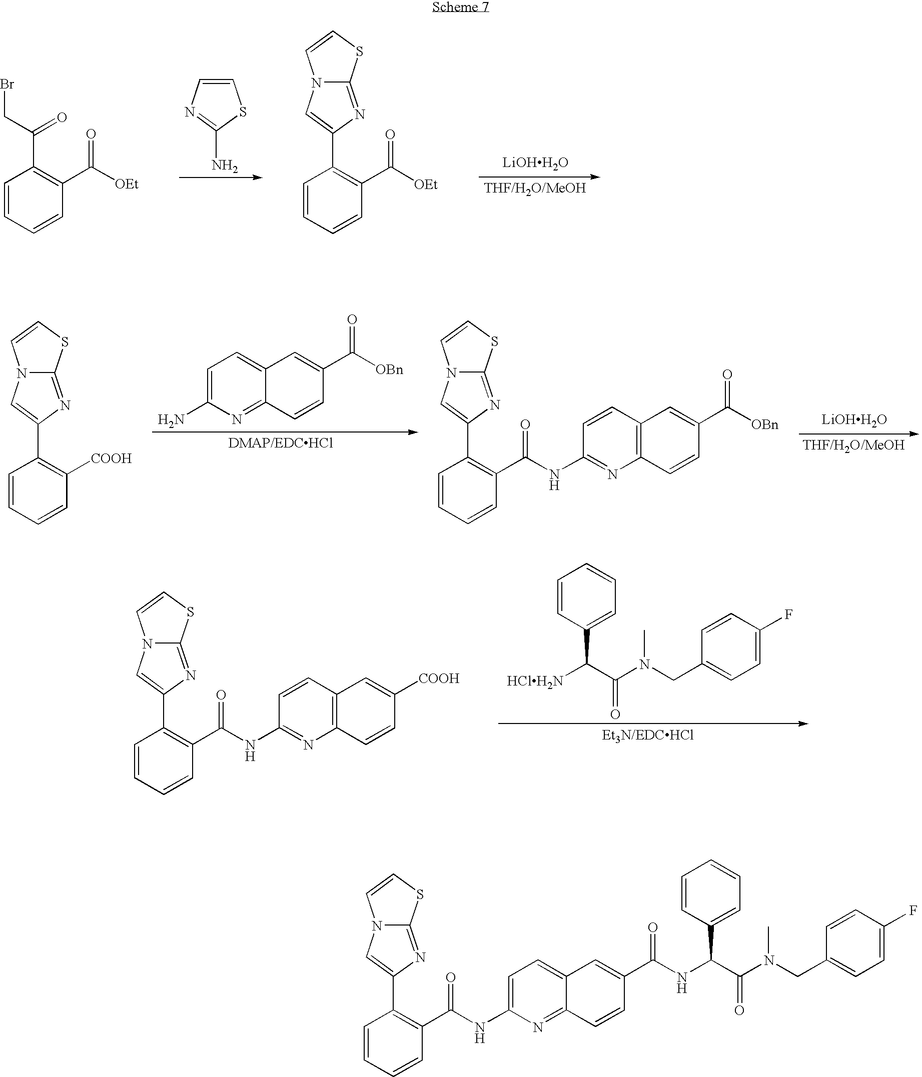

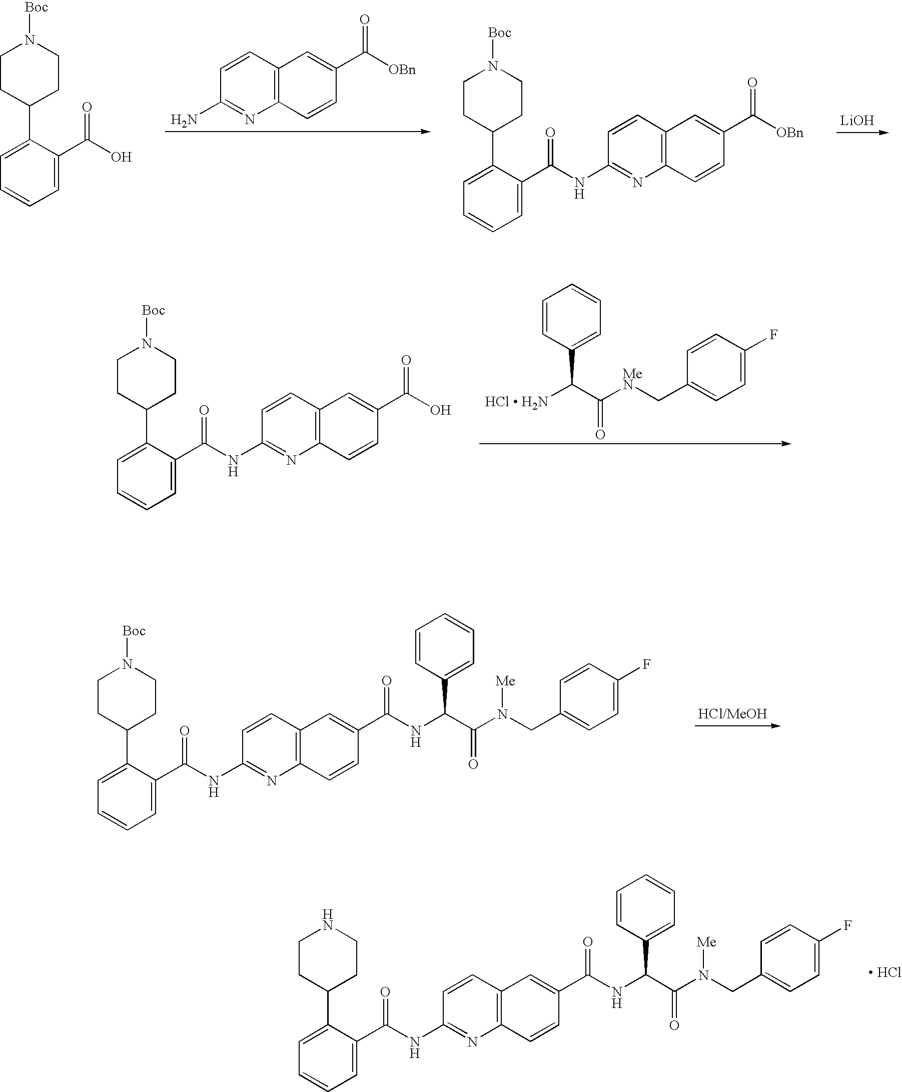

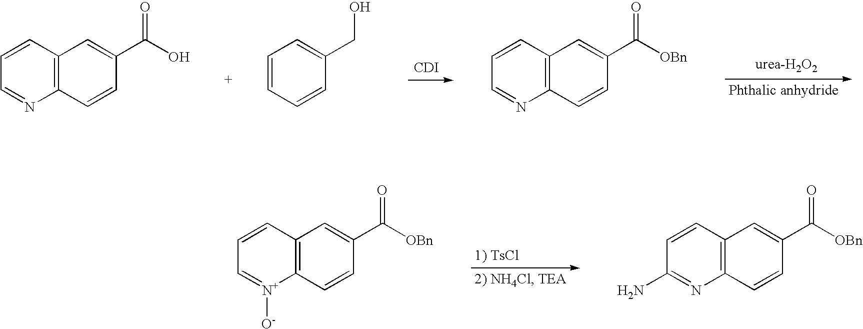

### Preparation of benzyl 2-(2-(imidazo[2,1-b]thiazol-6-yl)benzamido)quinoline-6-carboxylate

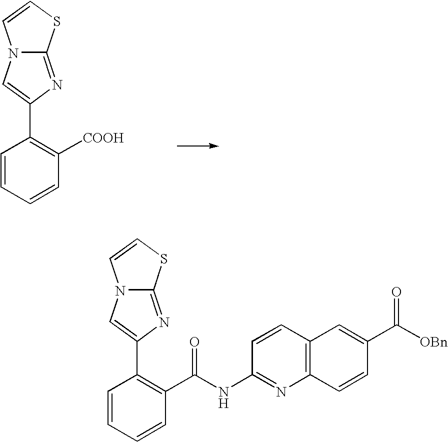

Benzyl 2-aminoquinoline-6-carboxylate was prepared according to the procedure outlined in WO 2005/080373 A1. A mixture containing 2-(imidazo[2,1-b]thiazol-6-yl)benzoic acid (40 mg, 0.16 mmol), benzyl 2-aminoquinoline-6-carboxylate (55 mg, 0.19 mmol), DMAP (22 mg, 0.18 mmol) and EDC.HCl (173 mg, 0.90 mmol) in CH₂Cl₂ (2 mL) was stirred at room temperature for 18 h. It was diluted with saturated aqueous NaHCO₃ and the aqueous phase was separated and further extracted with CH₂Cl₂. The combined organic layers were washed with brine, dried (MgSO₄) and concentrated under reduced pressure. The resulting residue was purified by chromatography to afford benzyl 2-(2-(imidazo[2,1-b]thiazol-6-yl)benzamido)quinoline-6-carboxylate as a tan solid (44 mg, yield: 26%). ¹H NMR (400 MHz, CDCl₃) δ: 9.55 (1H, s), 8.65 (1H, d), 8.58 (1H, s), 8.29 (2H, m), 7.81 (3H, t, J=4.4 Hz), 7.59-7.40 (8H, m), 6.84 (1H, d, J=4.4 Hz), 5.45 (2H, s); MS (ESI) calcd for C₂₉H₂₀N₄O₃S (m/z): 504.56. found: 505.2 [M+1]⁺.

### Preparation of 2-(2-(imidazo[2,1-b]thiazol-6-yl)benzamido)quinoline-6-carboxylic acid

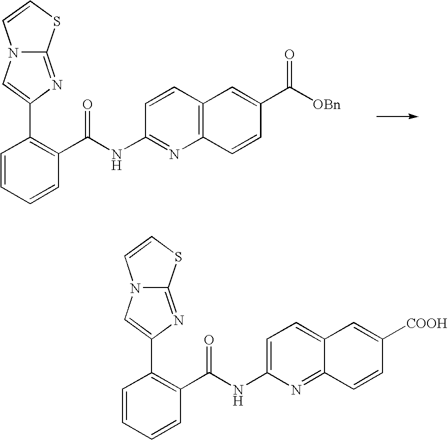

Lithium hydroxide monohydrate (12 mg, 0.27 mmol) was added to a solution of benzyl 2-(2-(imidazo[2,1-b]thiazol-6-yl)benzamido)quinoline-6-carboxylate (90 mg, 0.18 mmol) in THF (2 mL), H₂O (1 mL) and concentrated under reduced pressure. The resulting residue was taken up in water, and enough 6 N HCl was added to produce a pH=3. The precipitate was collected by filtration, washed with Et₂O and dried to give 2-(2-(imidazo[2,1-b]thiazol-6-yl)benzamido)quinoline-6-carboxylic acid as a yellow solid (46 mg, yield: 62%). ¹H NMR (400 MHz, DMSO) a: 8.60 (2H, m), 8.37 (1H, d, J=7.6 Hz), 8.18 (1H, d, J=7.6 Hz), 8.10 (1H, s), 8.01 (1H, d, J=4.4 Hz), 7.85 (2H, m), 7.64 (2H, m), 7.51 (1H, t, J=7.6 Hz), 7.37 (1H, d, J=4.4 Hz).

### Preparation of (S)—N-(2-((4-fluorobenzyl)(methyl)amino)-2-oxo-1-phenylethyl)-2-(2-(imidazo[2,1-b]thiazol-6-yl)benzamido)quinoline-6-carboxamide (Compound #03)

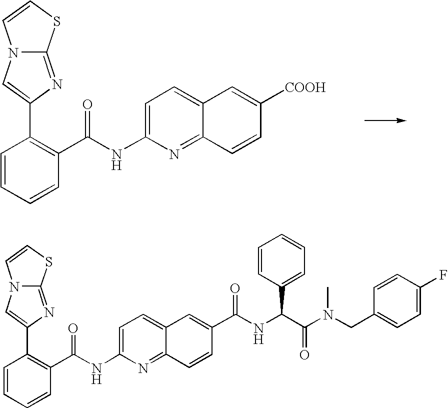

Triethylamine (0.024 mL, 0.17 mmol) and EDC.HCl (33 mg, 0.17 mmol) were added sequentially to a mixture containing 2-(2-(imidazo[2,1-b]thiazol-6-yl)benzamido)quinoline-6-carboxylic acid (46 mg, 0.11 mmol) and (S)-2-amino-N-(4-fluorobenzyl)-N-methyl-2-phenylacetamide hydrochloride salt (52 mg, 0.17 mmol) in CH₂Cl₂ (3 mL). The reaction mixture was stirred at room temperature for 3 h and concentrated under reduced pressure. The resulting residue was purified by chromatography to afford (S)—N-(2-((4-fluorobenzyl)(methyl)amino)-2-oxo-1-phenylethyl)-2-(2-(imidazo[2,1-b]thiazol-6-yl)benzamido)quinoline-6-carboxamide as a white solid (45 mg, yield: 61%). ¹H NMR (400 MHz, CDCl₃): δ: 9.38 (1H, s), 8.62 (1H, d, J=8.8 Hz), 8.24 (2H, m), 8.06 (1H, m), 7.98 (1H, d, J=7.6 Hz), 7.83 (3H, m), 7.79 (1H, s), 7.58 (3H, m), 7.56-7.35 (6H, m), 7.18 (2H, m), 7.03-6.81 (3H, m), 6.81 (1H, s), 6.13 (1H, d, J=7.2 Hz), 4.71 (2H, m), 2.94 (1H, s), 2.91 (2H, s); MS (ESI) calcd for C₃₈H₂₉FN₆O₃S (m/z): 668.2. found: 669.4 [M+1]⁺.

### Preparation of (S)-tert-butyl 4-(2-(6-(2-((4-fluorobenzyl)(methyl)amino)-2-oxo-1-phenylethylcarbamoyl)quinolin-2-ylcarbamoyl)phenyl)piperidine-1-carboxylate and

### (S)-tert-butyl 3-(2-(6-((S)-2-((4-fluorobenzyl)(methyl)amino)-2-oxo-1-phenylethylcarbamoyl)quinolin-2-ylcarbamoyl)phenyl)pyrrolidine-1-carboxylate

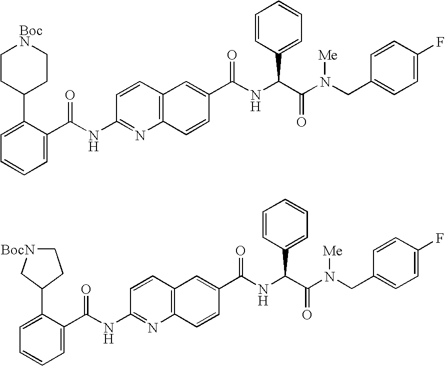

(S)-tert-Butyl 4-(2-(6-(2-((4-fluorobenzyl)(methyl)amino)-2-oxo-1-phenylethylcarbamoyl)quinolin-2-ylcarbamoyl)phenyl)piperidine-1-carboxylate and (S)-tert-butyl 3-(2-(6-((S)-2-((4-fluorobenzyl)(methyl)amino)-2-oxo-1-phenylethylcarbamoyl)quinolin-2-ylcarbamoyl)phenyl)pyrrolidine-1-carboxylate were prepared according to the procedure outlined in the synthesis of Compound #03, using the appropriate ortho-substituted 2-(1-(tert-butoxycarbonyl)piperidin-4-yl)benzoic acid and 2-(1-(tert-butoxycarbonyl)pyrrolidin-3-yl)benzoic acid as the starting materials.

### Preparation of (S)—N-(2-((4-fluorobenzyl)(methyl)amino)-2-oxo-1-phenylethyl)-2-(2-(piperidin-4-yl)benzamido)quinoline-6-carboxamide hydrochloride (Compound #01)

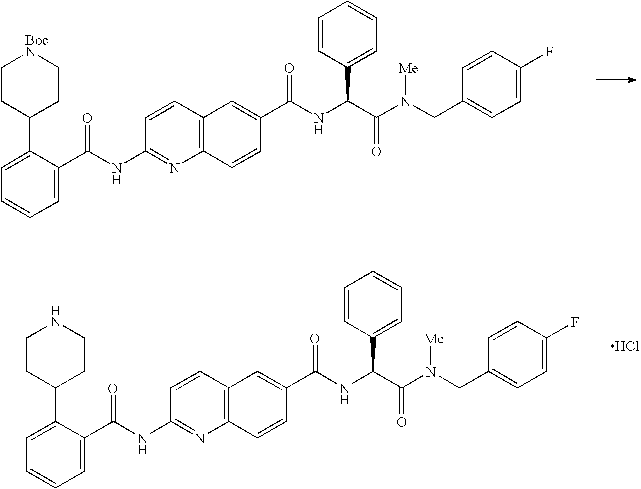

A solution of HCl in MeOH (4M, 5 mL) was added to mixture of (S)-tert-butyl 4-(2-(6-(2-((4-fluorobenzyl)(methyl)amino)-2-oxo-1-phenylethylcarbamoyl)quinolin-2-ylcarbamoyl)phenyl)piperidine-1-carboxylate (535 mg, 0.733 mmol) in CH₂Cl₂ (5 mL). The resulting reaction mixture was stirred at room temperature for 2 h and concentrated under reduced pressure to afford (S)—N-(2-((4-fluorobenzyl)(methyl)amino)-2-oxo-1-phenylethyl)-2-(2-(piperidin-4-yl)benzamido)quinoline-6-carboxamide hydrochloride salt as a light yellow solid (455 mg, yield: 93%). ¹H NMR (400 MHz, DMSO-d₆) δ: 11.570 (1H, s); 9.196 (2H, d, J=8.0 Hz), 9.142 (1H, d, J=7.2 Hz), 9.023 (2H, m), 8.615 (1H, dd, J=10.8 Hz), 8.398 (1H, d, J=8.8 Hz), 8.224 (1H, d, J=8.0 Hz), 7.932 (1H, d, J=7.2 Hz), 7.568 (4H, m), 7.438 (5H, m), 7.271 (2H, m), 7.171 (2H, m), 6.185 (1H, d, J=7.2 Hz), 4.741 (2H, m), 3.387 (4H, m), 2.989 (4H, m), 2.778 (1H, s), 2.044 (4H, m); MS (ESI) calcd for C₃₈H₃₇ClFN₅O₃ (m/z): 665.26. found: 631.4 [M-HCl+1]⁺.

### Preparation of N—((S)-2-((4-fluorobenzyl)(methyl)amino)-2-oxo-1-phenylethyl)-2-(2-(pyrrolidin-3-yl)benzamido)quinoline-6-carboxamide hydrochloride (Compound #02)

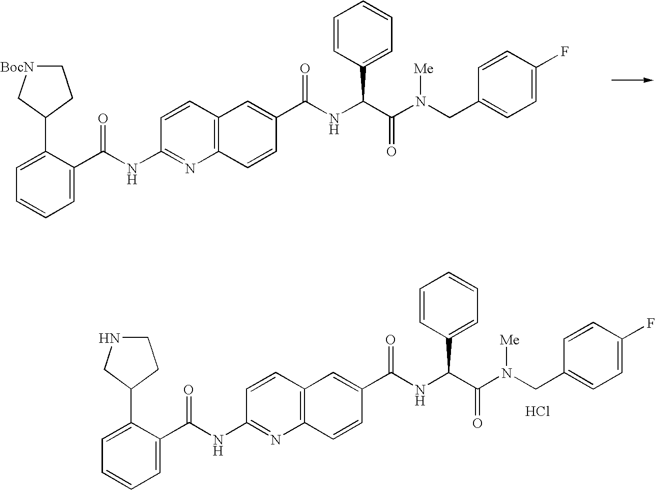

N—((S)-2-((4-Fluorobenzyl)(methyl)amino)-2-oxo-1-phenylethyl)-2-(2-(pyrrolidin-3-yl)benzamido)quinoline-6-carboxamide hydrochloride was prepared according to the procedure outlined in the synthesis of Compound #01 using the appropriate starting material.

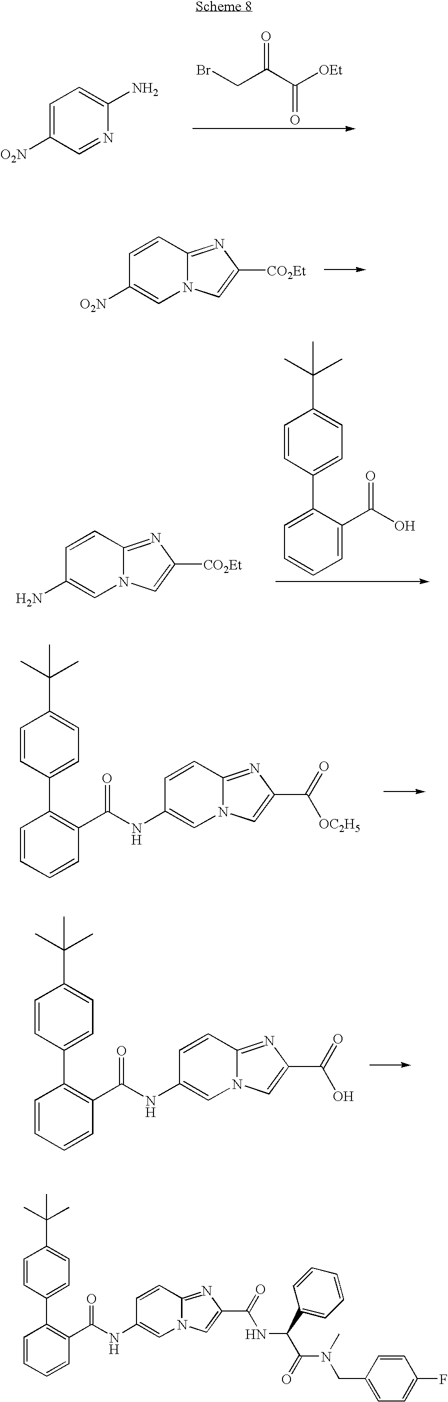

### Preparation of ethyl 6-nitroimidazo[1,2-a]pyridine-2-carboxylate

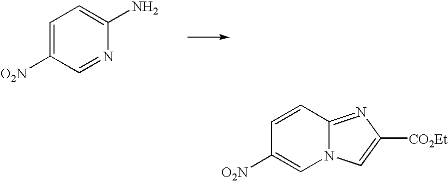

A solution of 5-nitropyridin-2-amine (2.0 g, 14.38 mmol) and ethyl bromopyruvate (3.36 g, 17.25 mmol) in ethanol (EtOH) (20 mL) was stirred under reflux for 18 h. The reaction mixture was cooled to room temperature. The precipitated solids were collected by filtration and then suspended in ethyl acetate. The organic layer was washed with saturated aqueous NaHCO₃ solution, dried (Na₂SO₄) and concentrated under reduced pressure to afford ethyl 6-nitroimidazo[1,2-a]pyridine-2-carboxylate as a brown solid (1.137 g, yield: 34%). ¹H NMR (400 MHz, CDCl₃) δ: 9.320 (1H, s), 8.402 (1H, s), 8.065 (1H, dd, J=10.0 Hz), 7.905 (1H, d, J=10.0 Hz), 4.479-4.533 (2H, q, J=7.2 Hz), 1.469 (3H, t, J=7.2 Hz).

### Preparation of ethyl 6-aminoimidazo[1,2-a]pyridine-2-carboxylate

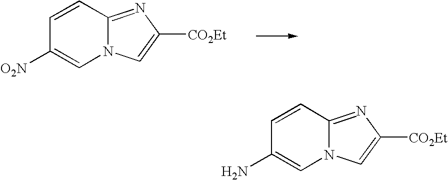

Ethyl 6-nitroimidazo[1,2-a]pyridine-2-carboxylate (1.137 g, 4.83 mmol) and 10% Pd on C (200 mg) were suspended in EtOH (4 mL). The reaction mixture was stirred at room temperature under 1 atm of hydrogen for 18 h. It was filtered through a pad of Celite and the filtrate was concentrated under reduced pressure. The resulting residue was purified by chromatography to afford ethyl 6-aminoimidazo[1,2-a]pyridine-2-carboxylate as a green solid (478 mg, yield: 48%). ¹H NMR (400 MHz, CDCl₃): δ: 8.026 (1H, s), 7.571 (2H, m), 6.911 (1H, dd, J=9.6 Hz, J₂=2.0 Hz), 4.475 (2H, q, J=7.2 Hz), 1.438 (3H, t, J=7.2 Hz).

### Preparation of ethyl 6-(4′-tert-butylbiphenyl-2-ylcarboxamido)imidazo[1,2-a]pyridine-2-carboxylate

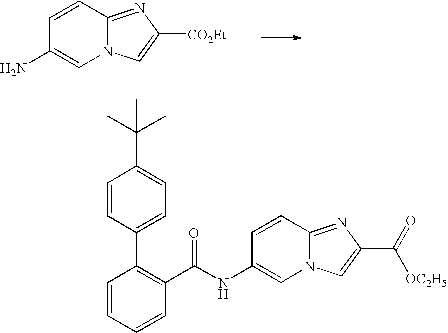

A mixture containing 4′-tert-butylbiphenyl-2-carboxylic acid (539 mg, 2.12 mmol), ethyl 6-aminoimidazo[1,2-a]pyridine-2-carboxylate (478 mg, 2.33 mmol), DMAP (295 mg, 2.42 mmol) and EDC.HCl (611 mg, 3.18 mmol) in CH₂Cl₂ (10 mL) was stirred at room temperature for 18 h. The reaction mixture was diluted with saturated aqueous NaHCO₃ solution. The aqueous layer was separated and further extracted with CH₂Cl₂. The combined organic layers were washed with brine, dried (MgSO₄) and concentrated under reduced pressure. The resulting residue was purified by chromatography (elution: petroleum ether/EtOAc=1:2 plus 5% CH₂Cl₂) to give ethyl 6-(4′-tert-butylbiphenyl-2-ylcarboxamido)imidazo[1,2-a]pyridine-2-carboxylate as a green solid (652 mg, yield: 70%). ¹H NMR (400 MHz, CDCl₃): δ: 9.116 (1H, s), 8.141 (1H, s), 7.982 (1H, dd, J=7.6 Hz), 7.379-7.621 (8H, m), 6.799 (1H, s), 5.965 (1H, dd, J₁=9.6 Hz, J₂=2.0 Hz), 4.483 (2H, q, J=7.2 Hz), 1.442 (3H, t, J=7.2 Hz), 1.368 (9H, s).

### Preparation of 6-(4′-tert-butylbiphenyl-2-ylcarboxamido)imidazo[1,2-a]pyridine-2-carboxylic acid

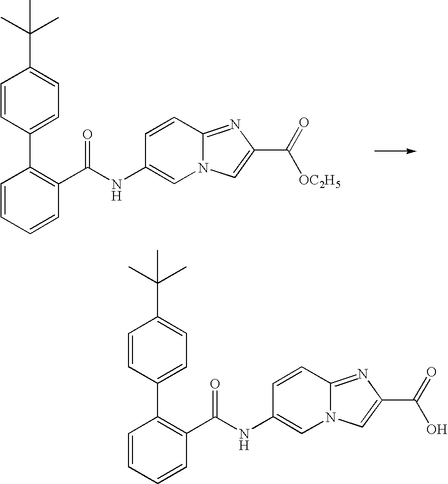

Lithium hydroxide monohydrate (186 mg, 4.43 mmol) was added to a mixture containing ethyl 6-(4′-tert-butylbiphenyl-2-ylcarboxamido)imidazo[1,2-a]pyridine-2-carboxylate (652 mg, 1.48 mmol) in THF (4 mL), H₂O (1.5 mL) and MeOH (2 mL). The reaction mixture was stirred at 50° for 18 h. It was cooled to room temperature and concentrated under reduced pressure. The resulting residue was taken up in water, and enough 6 N HCl was added to produce a pH=4. The precipitated solids were collected by filtration and dried to afford 6-(4′-tert-butylbiphenyl-2-ylcarboxamido)imidazo[1,2-a]pyridine-2-carboxylic acid as a tan solid (532 mg, yield: 87%). ¹H NMR (400 MHz, DMSO-d₆): δ: 10.666 (1H, s), 9.546 (1H, s), 8.296 (1H, s), 7.801 (2H, s), 7.513 (1H, d, J=6.8 Hz), 7.380 (1H, dd, J=7.2 Hz), 7.264 (3H, m), 7.174 (4H, s), 1.099 (9H, s).

### Preparation of (S)-6-(4′-tert-butylbiphenyl-2-ylcarboxamido)-N-(2-((4-fluorobenzyl)(methyl)amino)-2-oxo-1-phenylethyl)imidazo[1,2-a]pyridine-2-carboxamide (Compound #33)

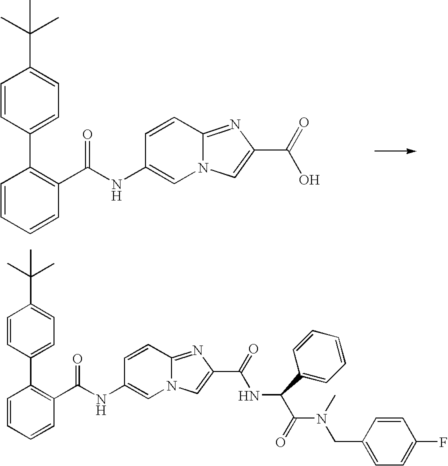

A mixture containing 6-(4′-tert-butylbiphenyl-2-ylcarboxamido)imidazo[1,2-a]pyridine-2-carboxylic acid (80 mg, 0.193 mmol), (S)-2-amino-N-(4-fluorobenzyl)-N-methyl-2-phenylacetamide hydrochloride (90 mg, 0.290 mmol), HATU (147 mg, 0.386 mmol) and DIPEA (0.07 mL, 0.386 mmol) in DMF (2 mL) was stirred at room temperature for 18 h. The reaction mixture was diluted with water, and the precipitated solids were collected by filtration. This residue was purified by preparative TLC (elution: CH₂Cl₂/MeOH=20:1) to afford (S)-6-(4′-tert-butylbiphenyl-2-ylcarboxamido)-N-(2-((4-fluorobenzyl)(methyl)amino)-2-oxo-1-phenylethyl)imidazo[1,2-a]pyridine-2-carboxamide as an off-white solid (50 mg, 39%). ¹H NMR (400 MHz, CDCl₃): δ: 9.004 (1H, s), 8.648 (1H, m), 8.037 (1H, d, J=7.2 Hz), 7.963 (1H, d, J=7.6 Hz), 7.308-7.599 (12H, m), 6.858-7.190 (5H, m), 6.136 (1H, m), 5.994 (1H, d, J=1.6 Hz), 4.658 (2H, m), 2.904 (3H, t, J=4.4 Hz), 1.368 (9H, s); MS (ESI) calcd for C₄₁H₃₉FN₅O₃ (m/z): 667.77. found: 668.3[M−1]⁺.

### Preparation of 6-(2-(3-methylimidazo[2,1-b]thiazol-6-yl)benzamido)benzo[d]thiazole-2-carboxylic acid

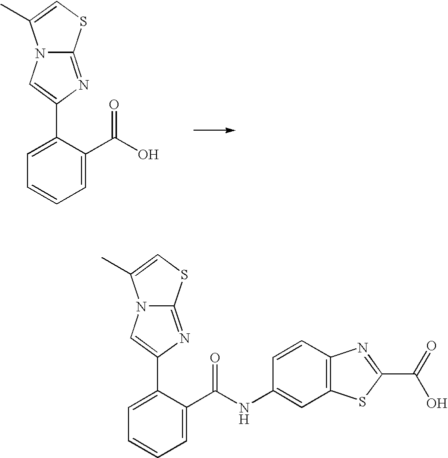

2-(3-Methylimidazo[2,1-b]thiazol-6-yl)benzoic acid was prepared according to the procedure outlined in the preparation of 2-(imidazo[2,1-b]thiazol-6-yl)benzoic acid using 4-methylthiazol-2-amine instead of thiazol-2-amine. In a typical run, 2-(3-methylimidazo[2,1-b]thiazol-6-yl)benzoic acid (126 mg, 0.488 mmol) and methyl 6-aminobenzo[d]thiazole-2-carboxylate (101 mg, 0.488 mmol) were taken up in 3 mL of DMF along with HATU (278 mg, 0.732 mmol) and DIPEA (0.17 mL, 0.98 mmol). The reaction mixture was stirred at room temperature for 18 h. It was then diluted with EtOAc (25 mL) and washed with water (4×4 mL). The organic layer was dried (Na₂SO₄) and concentrated under reduced pressure to afford crude methyl 6-(2-(3-methylimidazo[2,1-b]thiazol-6-yl)benzamido)benzo[d]thiazole-2-carboxylate. This crude material was dissolved in 3 mL of water and 3 mL of MeOH along with lithium hydroxide monohydrate (35 mg, 1.46 mmol). The reaction mixture was stirred at 50° C. for 3 h and then concentrated under reduced pressure. The aqueous layer was washed with CH₂Cl₂ (5 mL). Enough 6 N HCl was added to produce a pH=2. The resulting mixture was extracted with CH₂Cl₂. The combined organic layers were dried (Na₂SO₄) and concentrated under reduced pressure to afford 152 mg of 6-(2-(3-methylimidazo[2,1-b]thiazol-6-yl)benzamido)benzo[d]thiazole-2-carboxylic acid. MS (ESI) calcd for C₂₁H₁₄N₄O₃S₂ (m/z): 434.49. found: 435 (M+1)⁺.

### Preparation of (S)—N-(2-((4-fluorobenzyl)(methyl)amino)-2-oxo-1-phenylethyl)-6-(2-(3-methylimidazo[2,1-b]thiazol-6-yl)benzamido)benzo[d]thiazole-2-carboxamide (Compound #64)

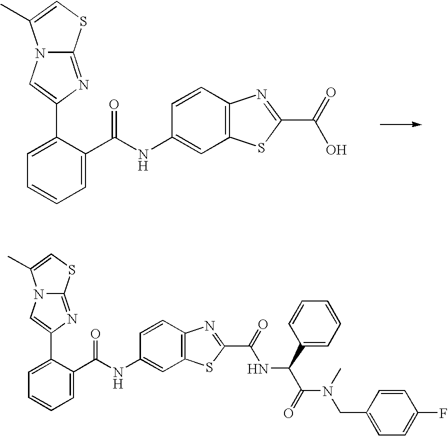

6-(2-(3-Methylimidazo[2,1-b]thiazol-6-yl)benzamido)benzo[d]thiazole-2-carboxylic acid (152 mg, 0.35 mmol) was taken up in 3 mL of DMF along with (S)-2-amino-N-(4-fluorobenzyl)-N-methyl-2-phenylacetamide hydrochloride (108 mg, 0.35 mmol), HATU (200 mg, 0.53 mmol) and DIPEA (0.12 mL, 0.7 mmol). The reaction mixture was stirred at room temperature for 18 h. It was then diluted with EtOAc (25 mL) and washed with water (5 mL). The organic layer was dried (Na₂SO₄) and concentrated under reduced pressure. The resulting residue was purified by preparative HPLC to afford 20 mg of (S)—N-(2-((4-fluorobenzyl)(methyl)amino)-2-oxo-1-phenylethyl)-6-(2-(3-methylimidazo[2,1-b]thiazol-6-yl)benzamido)benzo[d]thiazole-2-carboxamide. MS (ESI) calcd for C₃₇H₂₉FN₆O₃S₂ (m/z): 688.79. found: 689.1 (M+1)⁺.

### Preparation of 4′-tert-butyl-6-methylbiphenyl-2-carboxylic acid

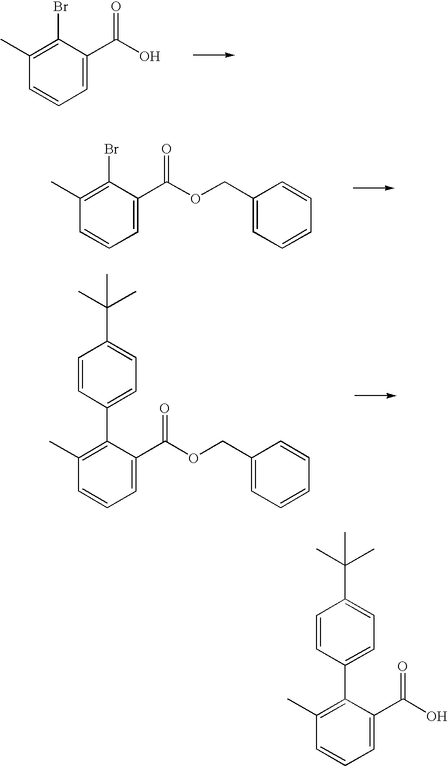

### Step 1: Preparation of benzyl 2-bromo-3-methylbenzoate

In a typical run, 2-bromo-3-methylbenzoic acid (1.06 g, 4.93 mmol) was dissolved in DMF (10 mL). Anhydrous K₂CO₃ (1.02 g, 7.40 mmol) and benzyl chloride (636.5 mg, 5.03 mmol) were added at room temperature. The resulting reaction mixture was stirred at room temperature for 19 h. It was then partitioned between EtOAc and H₂O. The organic layer was washed with H₂O, brine, dried (MgSO₄), and concentrated under reduced pressure to afford benzyl 2-bromo-3-methylbenzoate as a light yellow oil (1.52 g, yield: 100%). ¹H NMR (400 MHz, CDCl₃): δ 7.49-7.25 (8H, m), 5.39 (2H, s), 2.17 (2H, s).

### Step 2: Preparation of benzyl 4′-tert-butyl-6-methylbiphenyl-2-carboxylate

Benzyl 2-bromo-3-methylbenzoate (1.52 g, 4.98 mmol) and 4-tert-butylphenylboronic acid (1.07 g, 5.98 mmol) were taken up in DMF (16 mL) along with K₃PO₄.3H₂O (1.99 g, 7.47 mmol) and Pd(PPh₃)₄ (120 mg, 0.10 mmol). The resulting reaction mixture was stirred at 100° C. for 15 h. The mixture was partitioned between EtOAc and H₂O. The organic layer was washed successively with H₂O, brine, dried (MgSO₄), and concentrated under reduced pressure. The resulting residue was purified by column chromatography to afford benzyl 4′-tert-butyl-6-methylbiphenyl-2-carboxylate as a light green oil (1.599 g, yield: 90%). ¹H NMR (400 MHz, CDCl₃): δ 7.65-7.03 (12H, m), 4.98 (2H, s), 2.14 (2H, s), 1.37 (9H, s).

### Step 3: Preparation of 4′-tert-butyl-6-methylbiphenyl-2-carboxylic acid

A solution of 1 N NaOH (17 mL) was added to a mixture of benzyl 4′-tert-butyl-6-methylbiphenyl-2-carboxylate (1.73 g, 4.826 mmol) in MeOH (17 mL). The reaction mixture was stirred under reflux for 16 h. Upon cooling to room temperature, the reaction mixture was concentrated under reduced pressure. The resulting residue was taken up in water and enough 1 N HCl was added to produce a pH=3. The precipitated solids were collected by filtration, washed with H₂O, and dried to afford 4′-tert-butyl-6-methylbiphenyl-2-carboxylic acid as a colorless solid (895 mg, yield: 69%). ¹H NMR (400 MHz, CDCl₃): δ 7.78 (1H, d, J=8.0 Hz), 7.43-7.32 (4H, m), 7.10 (2H, dd, J₁=6.4 Hz, J₂=2.0 Hz), 2.10 (3H, s), 1.37 (9H, s).

### Preparation of Compound #41, Compound #42, Compound #65, Compound #69, Compound #70, Compound #71, Compound #72, Compound #73, Compound #74, Compound #75 and Compound #96

The same procedure outlined in the preparation of Compound #04 was used, employing 4′-tert-butyl-6-methylbiphenyl-2-carboxylic acid and the appropriate amine components.

### Preparation of 2-bromo-4-tert-butylthiazole

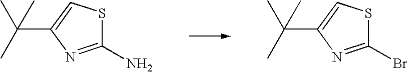

A mixture containing concentrated phosphoric acid (85%, 50 ml) and concentrated nitric acid (70%, 20 ml) was cooled to 0° C. 4-tert-Butyl-thiazol-2-ylamine (3.12 g, 20.0 mmol) was added at 0° C. in a single portion. This was followed by dropwise addition of a solution of NaNO₂ (1.38 g, 20 mmol) in water (10 ml) over a period of 30 min. The resulting reaction mixture was stirred at 0° C. for 1 h. This mixture was added dropwise to a suspension of NaBr (20.0 g) and CuBr (5.8 g) in water (20 ml) at 0° C. and then stirred at the same temperature until all gas evolution had ceased. Enough 10% aqueous KOH was added to the mixture to produce a pH=10. The product was separated from the aqueous mixture by steam distillation. The distillate was further extracted with ether. The combined organic layers were dried (Na₂SO₄) and concentrated under reduced pressure. The resulting residue was purified by chromatography (elution=petroleum ether) to afford 2-bromo-4-tert-butylthiazole as a colorless oil (1.3 g, yield=31%). ¹H NMR (400 MHz, CDCl₃) δ: 6.82 (1H, s), 1.32 (9H, s).

### Preparation of methyl 2-(4-tert-butylthiazol-2-yl)benzoate

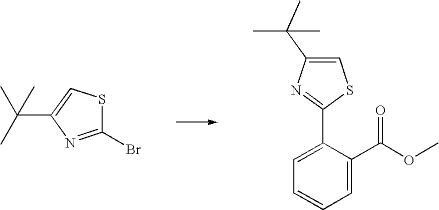

In a typical run, 2-bromo-4-tert-butylthiazole (1.258 g, 5.66 mmol) was taken up in DMF (10 mL) along with 2-(methoxycarbonyl)phenylboronic acid (1.223 g, 6.80 mmol), tetrakis(triphenylphosphine)palladium(0) (196 mg, 0.17 mmol) and Cs₂CO₃ (4.42 g, 13.59 mmol). The resulting reaction mixture was stirred at 160° C. in a microwave reactor for 20 min. The reaction mixture was cooled to room temperature and diluted with water (20 mL). The aqueous mixture was extracted with EtOAc (3×100 mL). The combined organic layers were washed with brine, dried (Na₂SO₄) and concentrated under reduced pressure. The resulting residue was purified by chromatography (elution with a 10:1 mixture of petroleum ether/EtOAc) to afford methyl 2-(4-tert-butylthiazol-2-yl)benzoate as a colorless oil (320 mg, yield: 20%). ¹H NMR (400 MHz, CDCl₃) δ 7.67 (1H, dd, J₁=4.0, J₂=2.4 Hz), 7.47-7.53 (4H, m), 6.96 (1H, s), 3.82 (3H, s), 1.39 (9H, s).

### Preparation of 2-(4-tert-butylthiazol-2-yl)benzoic acid

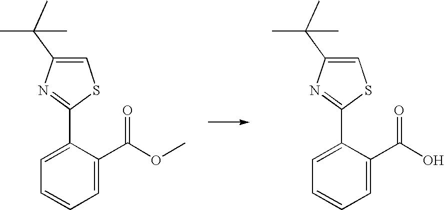

A mixture containing methyl 2-(4-tert-butylthiazol-2-yl)benzoate (320 mg) in 6 N HCl (5 mL) was stirred under reflux for 12 h. Upon cooling to room temperature, the mixture was extracted with EtOAc (3×20 mL). The combined organic layers were washed with brine, dried (Na₂SO₄) and concentrated under reduced pressure to afford 2-(4-tert-butylthiazol-2-yl)benzoic acid as a light yellow solid (280 mg, yield: 92%). ¹H NMR (400 MHz, CDCl₃) δ 12.80 (1H, s), 7.54-7.70 (4H, m), 7.34 (1H, s), 1.31 (9H, s).

### Preparation of (S)-6-(2-(4-tert-butylthiazol-2-yl)benzamido)-N-(2-((4-fluoro benzyl)(methyl)amino)-2-oxo-1-phenylethyl)benzo[d]thiazole-2-carboxamide (Compound #66)

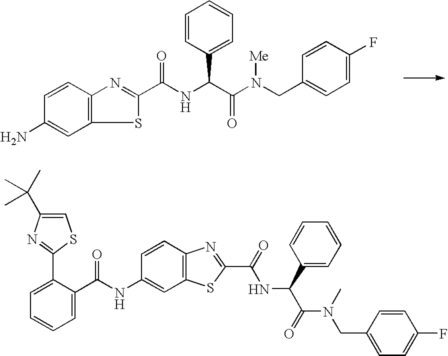

In a typical run, 2-(4-tert-butylthiazol-2-yl)benzoic acid (57 mg, 0.217 mmol), was taken up in DMF (2 mL) along with HATU (221 mg, 0.58 mmol), DIPEA (112 mg, 0.87 mmol) and (S)-6-amino-N-(2-((4-fluorobenzyl)(methyl)amino)-2-oxo-1-phenylethyl)benzo[d]thiazole-2-carboxamide (65 mg, 0.145 mmol). The mixture was stirred at room temperature for 18 h and then diluted with water (10 mL). The aqueous mixture was extracted with EtOAc (3×20 mL). The combined organic layers were washed with brine (2×20 mL), dried (Na₂SO₄) and concentrated under reduced pressure. The resulting residue was purified by chromatography (elution with a 2:1 mixture of petroleum ether/EtOAc) to afford (S)-6-(2-(4-tert-butylthiazol-2-yl)benzamido)-N-(2-((4-fluorobenzyl)(methyl)amino)-2-oxo-1-phenylethyl)benzo[d]thiazole-2-carboxamide as a white solid (30 mg, yield: 29%). ¹H NMR (400 MHz, CDCl₃) δ 8.86 (1H, d, J=7.2 Hz), 8.63 (1H, s), 8.29 (1H, s), 7.99 (1H, d, J=8.4 Hz), 7.75 (2H, m), 7.55 (4H, m), 7.37 (4H, m), 7.19 (2H, q, J=6.4 Hz), 6.98 (4H, m), 6.06 (1H, q, J=8.0 Hz), 4.63 (2H, q, J=7.6 Hz), 2.95 (1H, s), 2.87 (2H, d), 1.17 (9H, s); MS (ESI) calcd for C₃₈H₃₄FN₅O₃S₂ (m/z): 691.84. found: 692.3 [M+1]⁺.

### Preparation of Compound #100 was done in a similar manner coupling 2-(4-tert-butylthiazol-2-yl)benzoic acid with the corresponding amine as done for Compound #67

### Preparation of 4′-isopropylbiphenyl-2-carboxylic acid

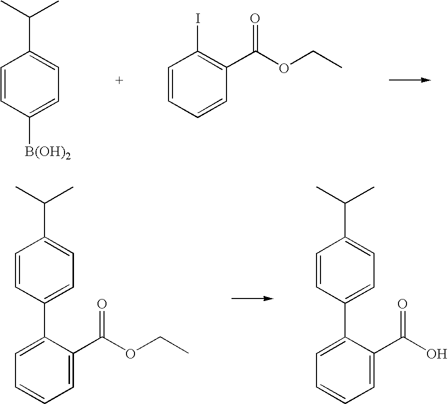

In a typical run, 4-isopropylphenylboronic acid (200 mg, 1.22 mmol) was taken up in 4 mL of DMF along with ethyl 2-iodobenzoate, [1,1′-bis(diphenylphosphino)-ferrocene]dichloropalladium (II) complex with CH₂Cl₂ (149 mg, 15 mol %) and anhydrous Na₂CO₃ (194 mg, 1.83 mmol). The reaction mixture was heated in a microwave reactor at 140° C. for 45 min. It was then cooled to room temperature and diluted with EtOAc. The organic layer was washed with water, dried (Na₂SO₄) and concentrated under reduced pressure to afford crude ethyl 4′-isopropylbiphenyl-2-carboxylate. This crude material was taken up in 15 mL of MeOH and 5 mL of water along with lithium hydroxide monohydrate (175 mg, 7.3 mmol). The reaction mixture was stirred at 50° C. for 18 h. It was then cooled to room temperature, diluted with water (5 mL) and concentrated under reduced pressure to remove most of the methanol. The aqueous layer was washed with CH₂Cl₂ (3×5 mL). Enough 6 N HCl was then added to the aqueous layer to produce a pH=2. The resulting mixture was extracted with CH₂Cl₂. The combined organic layers were dried (Na₂SO₄) and concentrated under reduced pressure to afford 4′-isopropylbiphenyl-2-carboxylic acid (120 mg, yield: 41%). MS (ESI) calcd for C₁₆H₁₆O₂ (m/z): 240.12. found: 241 (M+1)⁺.

### Preparation of 6-(4′-isopropylbiphenyl-2-ylcarboxamido)benzo[d]thiazole-2-carboxylic acid

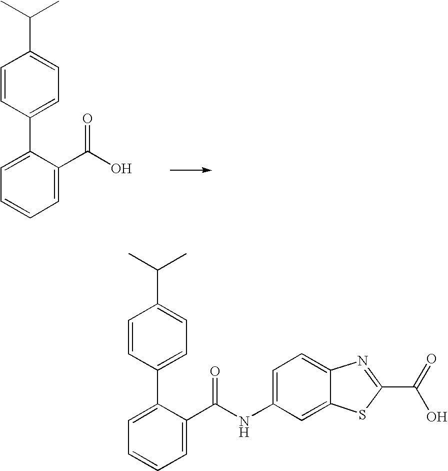

4′-Isopropylbiphenyl-2-carboxylic acid (120 mg, 0.5 mmol) was taken up in 3 mL of DMF along with methyl 6-aminobenzo[d]thiazole-2-carboxylate (104 mg, 0.5 mmol), HATU (285 mg, 0.75 mmol), and DIPEA (0.18 mL, 1 mmol). The reaction mixture was stirred at room temperature for 18 h. It was then diluted with EtOAc and washed with water. The organic layer was dried (Na₂SO₄) and concentrated under reduced pressure to afford crude methyl 6-(4′-isopropylbiphenyl-2-ylcarboxamido)benzo[d]thiazole-2-carboxylate. This material was taken up in a solution containing 4 mL of THF, 4 mL of water and lithium hydroxide monohydrate (35 mg, 1.46 mmol). The resulting reaction mixture was stirred at room temperature for 18 h. It was then diluted with water (5 mL) and concentrated to remove most of the volatile solvent. The aqueous layer was washed with CH₂Cl₂ (5 mL) and enough 6 N HCl was then added to produce a pH=2. The resulting mixture was extracted with CH₂Cl₂. The combined organic layers were dried (Na₂SO₄) and concentrated under reduced pressure to afford 6-(4′-isopropylbiphenyl-2-ylcarboxamido)benzo[d]thiazole-2-carboxylic acid (125 mg, yield: 60%). MS (ESI) calcd for C₂₄H₂₀N₂O₃S (m/z): 416.12. found: 417 (M+1)⁺.

### Preparation of ethyl 4-(4-aminophenyl)thiazole-2-carboxylate

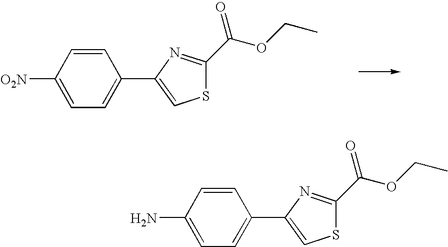

In a typical run, ethyl 4-(4-nitrophenyl)thiazole-2-carboxylate (500 mg, 1.80 mmol) was dissolved in 50 mL of EtOH and 70 mL of EtOAc. Palladium catalyst (10% Pd on C, 50 mg) was then added and the resulting reaction mixture was stirred at room temperature under 1 atm of hydrogen for 18 h. The reaction mixture was filtered through a pad of Celite and the filtrate was concentrated under reduced pressure to afford essentially quantitative yield of ethyl 4-(4-aminophenyl)thiazole-2-carboxylate. MS (ESI) calcd for C₁₂H₁₂N₂O₂S (m/z): 248.06. found: 249 (M+1)⁺.

### Preparation of 4-(4-(4′-tert-butylbiphenyl-2-ylcarboxamido)phenyl)thiazole-2-carboxylic acid

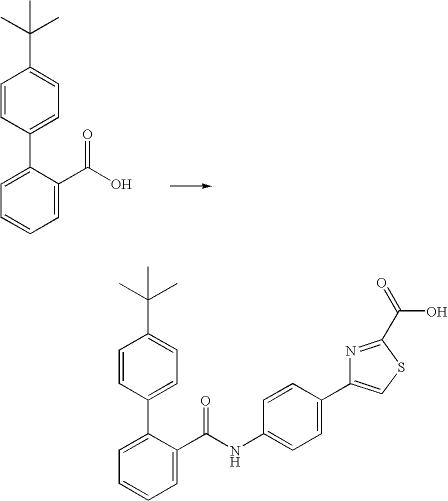

Ethyl 4-(4-aminophenyl)thiazole-2-carboxylate (120 mg, 0.484 mmol) was taken up in 3 mL of DMF along with 4′-tert-butylbiphenyl-2-carboxylic acid (123 mg, 0.484 mmol), HATU (275 mg, 0.726 mmol), and DIPEA (0.17 mL, 0.968 mmol). The reaction mixture was stirred at room temperature for 18 h. It was then diluted with EtOAc (25 mL) and washed with water (3×4 mL). The organic layer was dried (Na₂SO₄) and concentrated under reduced pressure to afford crude ethyl 4-(4-(4′-tert-butylbiphenyl-2-ylcarboxamido)phenyl)thiazole-2-carboxylate. This material was taken up in a solution containing 4 mL of THF, 4 mL of water and lithium hydroxide monohydrate (35 mg, 1.45 mmol). The resulting reaction mixture was stirred at room temperature for 18 h and then concentrated under reduced pressure. Enough 6 N HCl was added to the resulting aqueous layer to produce a pH=2. This aqueous mixture was extracted with CH₂Cl₂ (3×25 mL). The combined organic layers were dried (Na₂SO₄) and concentrated under reduced pressure to afford 4-(4-(4′-tert-butylbiphenyl-2-ylcarboxamido)phenyl)thiazole-2-carboxylic acid (210 mg, yield: 95%). MS (ESI) calcd for C₂₇H₂₄N₂O₃S (m/z): 456.15. found: 457 (M+1)⁺.

### Preparation of (S)—N-(2-((4-fluorobenzyl)(methyl)amino)-2-oxo-1-phenylethyl)-6-(4′-isopropylbiphenyl-2-ylcarboxamido)benzo[d]thiazole-2-carboxamide (Compound #54)

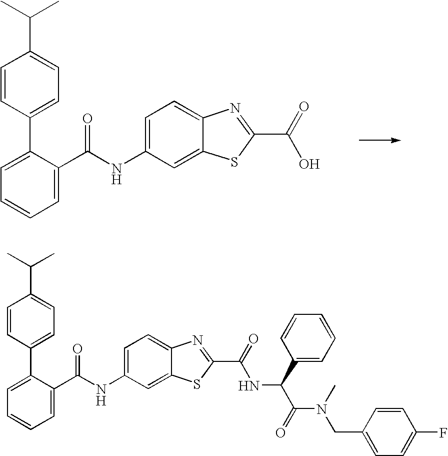

In a typical run, 6-(4′-isopropylbiphenyl-2-ylcarboxamido)benzo[d]thiazole-2-carboxylic acid (60 mg, 0.144 mmol) was taken up in 4 mL of DMF along with (S)-2-amino-N-(4-fluorobenzyl)-N-methyl-2-phenylacetamide hydrochloride (45 mg, 0.144 mmol), HATU (82 mg, 0.216 mmol) and DIPEA (50 μL, 0.288 mmol). The reaction mixture was stirred at room temperature for 18 h. It was then diluted with EtOAc (20 mL) and washed with water (3 mL). The organic layer was dried (Na₂SO₄) and concentrated under reduced pressure. The resulting residue was purified by preparative HPLC to afford 12 mg of (S)—N-(2-((4-fluorobenzyl)(methyl)amino)-2-oxo-1-phenylethyl)-6-(4′-isopropylbiphenyl-2-ylcarboxamido)benzo[d]thiazole-2-carboxamide as a light yellow solid. ¹H NMR (300 MHz, DMSO-d₆) δ 10.6 (1H, br s), 8.9 (1H, d, J=7.6 Hz), 8.6 (1H, br s), 8.1 (1H, d, J=7.6 Hz), 7.0-7.8 (18H, m), 6.1 (1H, d, J=8 Hz), 4.6 (2H, br s), 2.9 (3H, s), 1.1 (6H, m). MS (ESI) calcd for C₄₀H₃₅FN₄O₃S (m/z): 670.24. found: 671 (M+1)⁺.

### Preparation of (S)-6-(4′-isopropylbiphenyl-2-ylcarboxamido)-N-(2-oxo-1-phenyl-2-(pyrrolidin-1-yl)ethyl)benzo[d]thiazole-2-carboxamide, (S)-4-(4-(4′-tert-butylbiphenyl-2-ylcarboxamido)phenyl)-N-(2-((4-fluorobenzyl)(methyl)amino)-2-oxo-1-phenylethyl)thiazole-2-carboxamide and (S)-4-(4-(4′-tert-butylbiphenyl-2-ylcarboxamido)phenyl)-N-(2-oxo-1-phenyl-2-(pyrrolidin-1-yl)ethyl)thiazole-2-carboxamide (Compound #55, Compound #56 and Compound #57)

Each compound was prepared according to the general procedure outlined above in the synthesis of (S)—N-(2-((4-fluorobenzyl)(methyl)amino)-2-oxo-1-phenylethyl)-6-(4′-isopropylbiphenyl-2-ylcarboxamido)benzo[d]thiazole-2-carboxamide using the appropriate acid and amine components.

### Preparation of methyl 4-(benzyloxycarbonylamino)bicyclo[2.2.2]octane-1-carboxylate

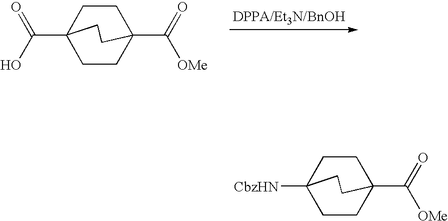

A mixture containing 4-(methoxycarbonyl)bicyclo[2.2.2]octane-1-carboxylic acid (100 mg, 0.471 mmol), diphenyl phosphoryl azide (130 mg, 0.471 mmol) and Et₃N (0.07 mL, 0.500 mmol) in toluene (4 mL) was stirred at room temperature for 2 h. It was then stirred under reflux for 2 h. At this point, benzyl alcohol (1 mL, 1.0 mmol) was added. The resulting reaction mixture was stirred under reflux for an additional 16 h. The reaction mixture was cooled to room temperature and washed successively with a 10% aqueous citric acid, saturated aqueous solution of NaHCO₃ and brine. The organic layer was dried (Na₂SO₄) and concentrated under reduced pressure to afford methyl 4-(benzyloxycarbonylamino)-bicyclo[2.2.2]octane-1-carboxylate as a thick, light brown oil (162 mg, essentially quantitative yield). ¹H NMR (400 MHz, DMSO-d₆) δ: 7.29-7.35 (5H, m), 4.95 (2H, s), 3.56 (3H, d, J=5.6 Hz), 1.76 (12H, t, J=17.2 Hz); MS (ESI) calcd for C₁₈H₂₃NO₄ (m/z): 317.38. found: 316.1 [M−1]⁻.

### Preparation of methyl 4-aminobicyclo[2.2.2]octane-1-carboxylate

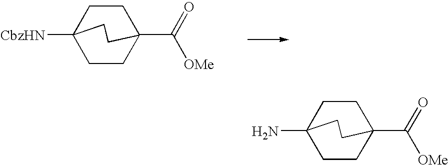

In a typical run, methyl 4-(benzyloxycarbonylamino)bicyclo[2.2.2]octane-1-carboxylate (2.14 g, 14.1 mmol) was taken up in MeOH (50 mL) along with 10% Pd on C (300 mg) and flushed thoroughly with nitrogen. The reaction mixture was stirred at room temperature under 1 atm of hydrogen for 2 days. The mixture was filtered through a pad of Celite and the filtrate was concentrated under reduced pressure. The crude product was taken up in CH₂Cl₂ and extracted with dilute 1 N HCl. The combined aqueous layers were neutralized with NaHCO₃ and then extracted with CH₂Cl₂. The combined organic layers were dried (Na₂SO₄) and concentrated under reduced pressure to afford methyl 4-aminobicyclo[2.2.2]octane-1-carboxylate as an oil (298 mg, yield: 12%). ¹H NMR (400 MHz, DMSO): δ 3.59-3.54 (3H, m), 1.74-1.68 (6H, m), 1.39-1.34 (6H, m).

### Preparation of methyl 4-(4′-tert-butylbiphenyl-2-ylcarboxamido)bicyclo[2.2.2]octane-1-carboxylate

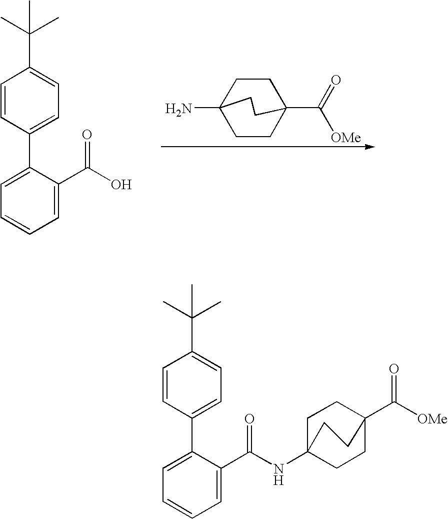

A mixture containing methyl 4-aminobicyclo[2.2.2]octane-1-carboxylate (298 mg, 1.63 mmol), 4′-tert-butylbiphenyl-2-carboxylic acid (414 mg, 1.63 mmol), DMAP (227 mg, 1.86 mmol) and EDC.HCl (470 mg, 2.45 mmol) in CH₂Cl₂ (5 mL) was stirred at room temperature for 18 h. The reaction mixture was concentrated under reduced pressure and the resulting residue was purified by chromatography (elution=16:1 petroleum ether/EtOAc) to afford methyl 4-(4′-tert-butylbiphenyl-2-ylcarboxamido)bicyclo[2.2.2]octane-1-carboxylate as an off-white solid (186 mg, yield: 27%). ¹H NMR (400 MHz, CDCl₃): δ 7.74 (1H, d, J=6.4 Hz), 7.48-7.39 (4H, m), 7.35-7.31 (3H, m), 4.77 (1H, s), 3.62 (3H, s), 1.79-1.75 (6H, m), 1.62-1.58 (6H, m), 1.36 (9H, s).

### Preparation of 4-(4′-tert-butylbiphenyl-2-ylcarboxamido)bicyclo[2.2.2]octane-1-carboxylic acid

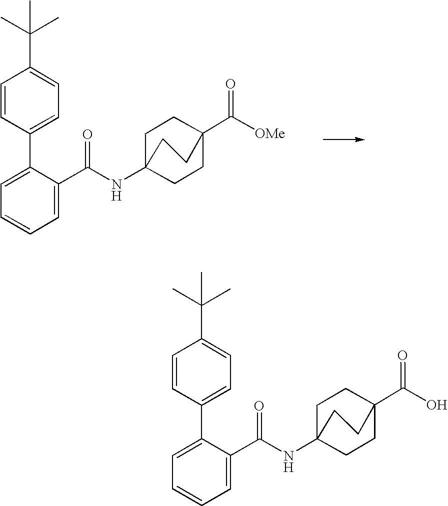

Sodium hydroxide (27 mg, 0.66 mmol) was added to a mixture containing methyl 4-(4′-tert-butylbiphenyl-2-ylcarboxamido)bicyclo[2.2.2]octane-1-carboxylate (186 mg, 0.43 mmol) in MeOH (4 mL) and H₂O (1 mL). The resulting reaction mixture was stirred at 50° C. for 5 h and then concentrated under reduced pressure. The residue was taken up in water and enough 6 N HCl was added to produce a pH=3. The precipitated solids were collected by filtration and dried to afford 4-(4′-tert-butylbiphenyl-2-ylcarboxamido)bicyclo[2.2.2]octane-1-carboxylic acid as a light yellow solid (121 mg, yield: 67%). ¹H NMR (400 MHz, DMSO-d₆): δ 12.01 (1H, s), 7.39-7.44 (3H, m), 7.30-7.37 (5H, m), 1.16 (12H, s), 1.31 (9H, s).

### Preparation of (S)-4-(4′-tert-butylbiphenyl-2-ylcarboxamido)-N-(2-oxo-1-phenyl-2-(pyrrolidin-1-yl)ethyl)bicyclo[2.2.2]octane-1-carboxamide (Compound #67)

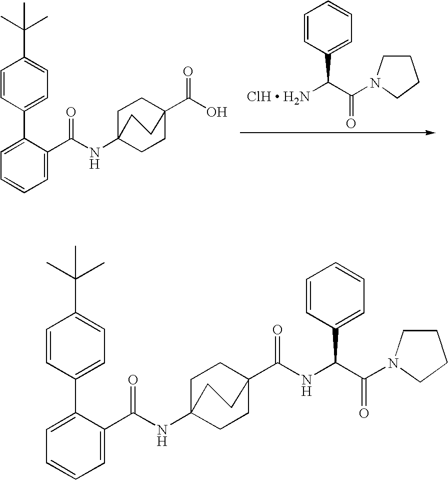

In a typical run, a mixture containing 4-(4′-tert-butylbiphenyl-2-ylcarboxamido)bicyclo[2.2.2]octane-1-carboxylic acid (60 mg, 0.15 mmol), (S)-2-amino-2-phenyl-1-(pyrrolidin-1-yl)ethanone hydrochloride (54 mg, 0.22 mmol), HATU (114 mg, 0.30 mmol), and DIPEA (0.15 mL, 0.9 mmol) in DMF (2 mL) was stirred at room temperature for 18 h. The reaction mixture was concentrated under reduced pressure and the resulting residue was purified by preparative TLC (elution=1:1 petroleum ether/EtOAc) to afford (S)-4-(4′-tert-butylbiphenyl-2-ylcarboxamido)-N-(2-oxo-1-phenyl-2-(pyrrolidin-1-yl)ethyl)-bicyclo[2.2.2]octane-1-carboxamide as a white solid (16 mg, yield: 18%). ¹H NMR (400 MHz, CDCl₃): δ 7.74 (1H, d, J=1.2 Hz), 7.13-7.72 (12H, m), 7.12 (1H, d, J=7.2 Hz), 5.55 (1H, d, J=7.2 Hz), 4.76 (1H, s), 3.52 (2H, m), 3.44 (1H, m), 3.30 (1H, m), 1.876 (4H, m), 1.70-1.74 (6H, m), 1.57-1.61 (6H, m), 1.36 (9H, s); MS (ESI) calcd for C₃₉H₄₅N₃O₃ (m/z): 591.78. found: 590.5 [M−1]⁻.

### Preparation of Compound #103, Compound #104, Compound #105, Compound #106, Compound #107, Compound #108, Compound #109, Compound #110, Compound #111, Compound #112, Compound #113, Compound #114, Compound #115, and Compound #116

The general procedure outlined in the preparation of either Compound #10 or Compound #67 was employed using the appropriate acid component. Some of these acids are commercially available and some could be prepared according to the general procedure outlined in J. Med. Chem. 2001, p. 4677 and WO2006/113910.

### Preparation of (S)-4-(4′-tert-butylbiphenyl-2-ylcarboxamido)-N-(2-((4-fluorobenzyl)(methyl)amino)-2-oxo-1-phenylethyl)bicyclo[2.2.2]octane-1-carboxamide (Compound #68)

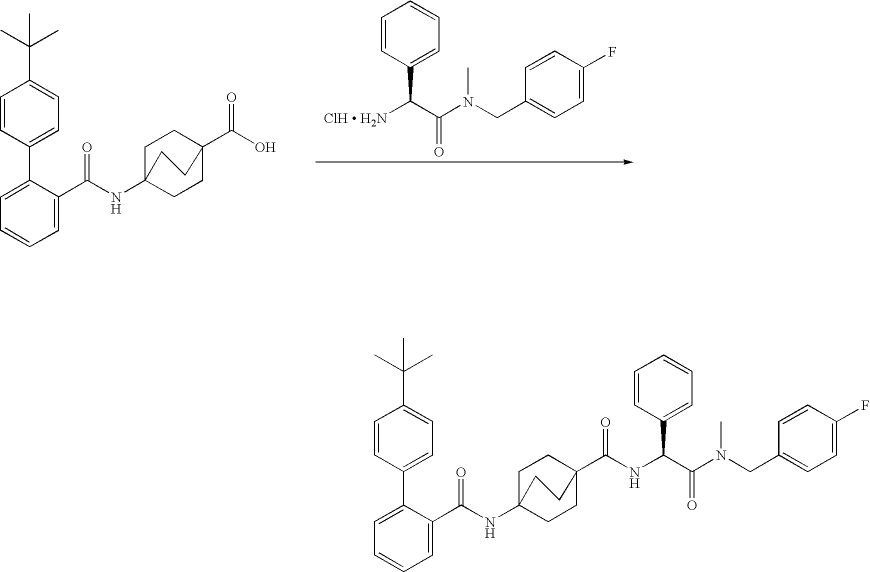

A mixture containing (S)-2-amino-N-(4-fluorobenzyl)-N-methyl-2-phenylacetamide hydrochloride (68 mg, 0.22 mmol), 4-(4′-tert-butylbiphenyl-2-ylcarboxamido)bicyclo[2.2.2]octane-1-carboxylic acid (60 mg, 0.15 mmol), HATU (114 mg, 0.30 mmol), and DIPEA (0.15 mL, 0.9 mmol) in DMF (2 mL) was stirred at room temperature for 18 h. The reaction mixture was concentrated under reduced pressure and the resulting residue was purified by preparative TLC (elution=1:1 petroleum ether/EtOAc) to afford (S)-4-(4′-tert-butylbiphenyl-2-ylcarboxamido)-N-(2-((4-fluorobenzyl)(methyl)amino)-2-oxo-1-phenylethyl)bicyclo[2.2.2]octane-1-carboxamide as a white solid (10 mg, yield: 10%). ¹H NMR (400 MHz, CDCl₃): δ: 7.73 (1H, d, J=7.2 Hz), 7.294-7.473 (11H, m), 7.11 (2H, m), 6.95 (2H, m), 6.83 (1H, t, J=14 Hz), 4.71 (2H, t, J=14.8 Hz), 2.94 (1H, s), 2.81 (2H, s), 1.73 (6H, m), 1.59 (6H, m), 1.35 (9H, s); MS (ESI) calcd for C₄₂H₄₆FN₃O₃ (m/z): 659.83. found: 660.5 [M+1]⁺.

### Preparation of Compound #101 and Compound #102

The same procedure used in the preparation of Compound #68 was employed using the appropriate amine.

### Example 2

### ApoB Secretion Inhibition

Abbreviations used:
BSA=Bovine serum albumin

### DMEM=Dulbecco's Modified Eagles Medium

DMSO=Dimethyl sulfoxide
EDTA=Ethylenediaminetetraacetic acid
ELISA=specific enzyme-linked immunosorbent assay

### EtOH=Ethanol

FBS=Fetal bovine serum
g=gram
H₂SO₄=sulfuric acid
HCl=Hydrochloric acid
μl=microliters
ml=milliliter
OA=Oleic acid
nm=nanometers
PBS=phosphate-buffered saline

### Pen/strep=Penicillin-Streptomycin

rt=room temperature
TMB=tetramethylbenzidine
Tris=2-Amino-2-(hydroxymethyl)-1,3-propanediol

The ability of the compounds of the present invention to inhibit the secretion of ApoB can be determined using the following cell-based assay, which measures the secretion of ApoB by HepG2 cells. ApoB has two possible metabolic fates: entrance into the lipoprotein assembly pathway within the lumen of the endoplasmic reticulum (ER), or degradation in the cytoplasm by the ubiquitin-dependent proteasome. The destiny of ApoB is determined by the relative availability of individual lipids, which is controlled by MTP (Davis, Biochim. Biophys. Acta, 1999, 1440, 1-31). It has been demonstrated that MTP inhibitors decrease apoB secretion (Haghpassand et al., 1996, J Lipid Res., 37, 1468-1480; Gordon et al., Proc. Natl. Acad. Sci. USA, 1996, 93, 11991-11995; Gordon et al, J. Biol. Chem., 1996, 271, 33047-33053).

HepG2 cells (ATCC, HB-8065) are seeded in 96-well plates in DMEM with 10% FBS+pen/strep at a density of 1×10⁵ cells/well (100 μl /well). Twenty-four hours after seeding, the plate is aspirated and the media changed to DMEM with 1% BSA and pen/strep, and the cells are serum-starved for 24 hours. After 24 hour serum-starvation, the plate is aspirated again and the cells are then treated for 24 hours with or without compound in DMEM with 1% BSA, 0.3 mM OA (OA complexed to BSA at a 2:1 ratio) and pen/strep. The final concentration of DMSO in the assay (from compound and control) is 0.1%. After 24 hour treatment, conditioned media is collected in an empty 96-well plate.

Cellular viability is measured using the cell permeable dye, AlamarBlue™. This dye undergoes electron reduction in viable cells but not dead cells. The reduced dye product gives a fluorescent signal which can be monitored with a fluorescence plate reader (excitation 545 nm and emission 575 nm). The amount of fluorescence generated in a given well is proportional to the number of viable cells. After collection, the 96-well plate is re-fed with 100 μl media (DMEM with 10% FBS) and 10 μl Alamar Blue™ per well, incubated for 2 hours and then read in a fluorescence plate reader.

ApoB secretion from HepG2 cells is quantitated by testing the conditioned media at 1:10 dilution using an ApoB ELISA kit (ALerCHEK) according to the manufacturer's instructions. The ELISA assay for ApoB is performed as follows: a polyclonal antibody against human ApoB (Chemicon) is diluted 1:1000 in carbonate-bicarbonate buffer and 100 μl are added to each well of a 96-well plate (NUNC). After 5 hours incubation at room temperature, the antibody solution is removed and wells are washed four times with PBS/0.05% TweenD 20 (Cayman Chemical Co.). Non-specific sites on the plastic plate are blocked by incubating wells for 1 to 1.5 hours in a solution of 0.5% BSA and 0.1% TweenD 20 made in PBS. One hundred microliters of a 1:20 dilution of growth medium from the HepG2 cells (made in 0.004% TweenD 20/1% BSA in PBS) is added to each well and incubated for 3 hours at room temperature. Wells are aspirated and washed four times (0.05% TweenD 20 in PBS) prior to adding 100 μl of a 1:1000 dilution (5 μg/ml) of the secondary antibody, mouse anti-human ApoB (Chemicon). After 2 hours incubation at room temperature, this solution is aspirated and the wells are again washed 4 times as above. One hundred microliters of peroxidase-conjugated affinity purified goat anti-mouse IgG (H+L) (Jackson ImmunoResearch Laboratories) (1:10,000 dilution in PBS/1% BSA/0.1% TweenD 20) is then added to each well and incubated for 1 hour at room temperature. After aspirating, the wells are washed 4 times as above and 50 μl of 1-step Ultra TMB ELISA reagent (Pierce) is added to each well and incubated for 5 minutes. The reaction is stopped by the addition of 50 μl of 2 NH₂SO₄ and absorbance of each well is read at 450 nm. Percent inhibition is calculated using absorbance from vehicle-treated supernatants minus the absorbance from media alone as the total or 100% value, and from these data IC₅₀ values (i.e., the concentration of the test compound that achieves a half-maximal inhibition of ApoB secretion) are determined.

The effect of the test compound Naringenin (Reference Compound #1) demonstrated a 50% inhibition of ApoB secretion at 200 μM. Results for compounds of the instant invention are summarized in the tables below.

As a counterscreen, the effects of test compounds on the secretion of ApoA1 by HepG2 cells was determined using the same conditioned media as used for the ApoB measurements. There was no non-specific inhibition of ApoA secretion by any compounds. Specifically, a commercially available ApoA1 Elisa kit was used (AlerCHEK Inc., Portland Me., Cat. #A70101) was used according to the manufacture's instructions. In general, all of the compounds tested to date had no or limited effect on ApoA1 secretion up to 10 uM.

### HepG2 ApoB Secretion Inhibition—Alternative Procedure

HepG2cells are grown in DMEM with 10% FBS in 96-well culture plates in a humidified atmosphere containing 5% carbon dioxide until the cells are approximately 70% confluent. Test compounds are dissolved at 10 mM in DMSO. From this stock, the initial dose concentration is prepared in 70% EtOH and subsequent serial dilutions made in 70% EtOH with DMSO at a concentration equivalent to the initial dilution. Dilutions of test compounds are prepared at 100× the desired final concentration and are added in triplicate to separate wells of a 96-well culture plate containing HepG2 cells. Forty hours later, growth medium is collected and assayed by ELISA for ApoB as described.

### Example 3

### In Vitro Inhibition of MTP

This assay utilizes donor and acceptor liposomes as described in Athar et al. (A simple, rapid, and sensitive fluorescence assay for microsomal triglyceride transfer protein. J Lipid Res. 2004, 45(4):764-72). This assay is commercially available (Cat. #D400, Chylos Inc., New York).

Liposomes are pipetted in 5 μl aliquots into the wells of a black fluorescence microtiter plate, followed by addition of 5 μl of water and an incubation of 2-5 min to allow the samples to equilibrate to rt. Next, 5 μl of compound dissolved in DMSO is added to the vesicles. The reaction is initated by the addition of 5 μl MTP, followed by incubation for 30 min at room temperature. Fluorescence units (FU) are measured using excitation and emission wavelengths of 460-470 nm and 530-550 nm, respectively.

% Transfer in Test Samples: (Test FU−Blank FU)/(Total FU−Blank FU)×100

The amounts of sample can be increased and water decreased proportionately to measure low activities. Volumes can be proportionately increased for different spectrofluorometers. Time of incubation can be increased to measure low activity in different samples. In such situations, samples are prepared just prior to measuring fluorescence units. For this purpose, isopropanol is added to substrate vesicles about 5 min prior to fluorescence measurement as the fluorescence readings in isopropanol decrease with time. Naringenin, Reference Compound #1, was used to validate the assay and demonstrated >90% inhibition at 200 μM. Results for compounds of the instant invention are summarized in the tables below.

An alternative procedure may be used to identify the ability of compounds to inhibit triglyceride transfer catalyzed by canine MTP. The assay measures the transfer rate of ¹⁴C-labeled triglyceride from a donor liposome to an acceptor liposome (labeled with ³H).

Canine liposomes can be isolated from canine liver in the following manner: frozen canine liver is thawed on ice and rinsed several times with 0.25 M sucrose. A 50% liver homogenate (w/v) is made in 0.25 M sucrose. The homogenate is diluted 1:1 with 0.25 M sucrose, and centrifuged at 10,000 g at 4° C. for 20 minutes. The supernatant is saved and set aside. The pellet is re-suspended in a minimal volume of 0.25 M sucrose and centrifuged at 10,000 g for 20 minutes at 4° C. The supernatants are combined and centrifuged at 105,000 g for 75 minutes at 4° C. The supernatant is discarded and the resulting microsomal pellet is re-suspended in a minimum volume of 0.25 M sucrose and diluted to 3 ml/gram liver weight in 0.15 M Tris-HCl, pH 8.0. The resulting suspension is divided into 12 tubes and centrifuged at 105,000 g for 75 minutes. The resulting liposomal pellets can be stored at −80° C. until needed.

MTP can be isolated in the following manner: the liposomal pellet is thawed and re-suspended in 12 ml/tube of cold 50 mM Tris-HCl, 50 mM KCl, 2 mM MgCl₂, pH 7.4, followed by the slow addition of 1.2 ml of a 0.54% deoxycholate, pH 7.4 solution. After a 30-minute incubation on ice with gentle mixing, the solution is centrifuged at 105,000 g for 75 minutes at 4° C. The supernatant, containing soluble MTP, is dialyzed for 2-3 days with 5 changes of assay buffer (15 mM Tris-HCl, 40 mM NaCl, 1 mM EDTA, 0.02% NaN₃, pH 7.4).

Donor liposomes are created by adding 447 mM egg phosphatidylcholine 68/20 ml), 83 mM bovine heart cardiolipin (169/20 ml) and 0.91 mM [¹⁴C]triolein (110 Ci/mol) (20/20 ml). The lipids are available in chloroform and are first dried under nitrogen and then hydrated in assay buffer to the volume needed. To create liposomes, lipids are sonicated for ˜7 min. Lipids are centrifuged at 105,000 g for 2 h and liposomes are harvested by removing the top -80% of supernatant into separate tube. Acceptor liposomes are created by adding 1.33 mM egg phosphatidylcholine (404/40 ml), 2.6 mM triolein (100/40 ml) and 0.5 nM [³H]egg phosphatidylcholine (50 Ci/mol) (10/40 ml). The lipids are available in chloroform and are first dried under nitrogen and then hydrated in assay buffer to the volume needed. To create liposomes, lipids are sonicated for ˜20 min. Lipids are centrifuged at 105,000 g for 2 h and are harvested by removing the top ˜80% of supernatant into separate tube.

Appropriately diluted drug or control samples in 100 μl assay buffer containing 5% BSA are added to reaction tubes containing assay buffer, 50 μl donor liposomes, 10 μl acceptor liposomes, and partially purified canine liver MTP. The tubes are vortexed and incubated on a tube shaker for 1 hour at 37° C. to allow lipid transfer reaction to occur. Donor liposomes are precipitated by adding 300 μl of a 50% (w/v) DEAE cellulose suspension in assay buffer to each tube. The tubes are centrifuged at ˜1000 rpm to pellet resin. Four hundred microliters of supernatant is transferred into a scintillation vial with scintillation fluid and DPM counts for both [³H] and [¹⁴C] are determined. Triolein transfer is calculated by comparing the amount of [¹⁴C] and [³H] remaining in the supernatant to [¹⁴C] and [³H] in the original donor and acceptor liposomes, respectively, using the following equation: % Triolen Transfer=([¹⁴C] supernatant/[¹⁴C] donor)×([³H] acceptor/[³H] supernatant)×100. IC₅₀ values are calculated using standard methods and first order kinetic calculations.

### Example 4

### Fat Absorption Inhibition

Healthy female CF1 mice (Charles River) weighing 18-20 grams upon arrival can be employed as test subjects. The mice are housed in groups of 10 in standard caging, and are allowed to acclimate for one week prior to testing. Mice are fasted overnight in a separate procedure room prior to testing. Each treatment group typically consists of 5 mice.

The test compounds are preferably provided as a powder in a glass vial. The dosing solution (0.10 ml/25 g body weight) administered by oral gavage consists of an emulsion of Miglyol E 812 (20%), Cremophor™ (5%) and water (75%). The emulsion is prepared as follows: an appropriate volume of Miglyol™ (Condea Vista Co.) is first added to the test compound, and the vial is vortexed for approximately 1 minute. The appropriate volume of Cremophor™ is then added, and the vial is again vortexed as before. The appropriate volume of water is added, and an emulsion is formed by vortexing and briefly sonicating.

Hamster liquid diet (Bioserve) (dose volume 0.5 ml/25 g body weight) is prepared by adding (for every 10 ml needed) 2.5 g liquid diet powder, 10 mL water and 5 microcuries glycerol-3H-trioleate to a laboratory blender. The mixture is then blended at high speed for approximately 1 min. The liquid diet is stored at 4° C. until needed. Sample tubes are weighed (Falcon 15 ml polypropylene conical). Three milliliters of 2.5 N KOH is added to each tube.

Following overnight fasting, each mouse is dosed (see above volumes) with test compound followed immediately by liquid diet. Positive and negative control groups (vehicle) are included in each assay. The positive control is Reference Compound #2, N-(2,2,2-trifluoroethyl)-9-(4-(4-(4′-(trifluoromethyl)biphenyl-2-ylcarboxamido)piperidin-1-yl)butyl)-9H-fluorene-9-carboxamide, a known potent MTP inhibitor (Wetterau et al., Science, 282:751, 1998):

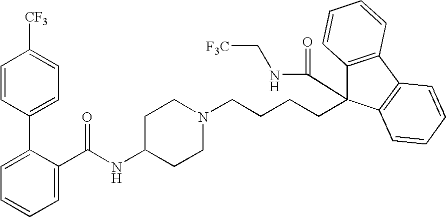

One scintillation vial is sham-dosed every 30 mice in order to determine the activity of the initial bolus. At two hours post dose the mice are euthanized by carbon dioxide inhalation, the abdominal cavity opened, and the small intestines removed and placed in the KOH conical tube. Each tube is then weighed. Tubes containing intestines are then placed in a 75° C. water bath for 1.5-2 hours. Following saponification, the tubes are vortexed and 200 μl saponate placed in a 20 ml liquid scintillation vial. Samples are decolorized by adding 200 μl of 30% (w/w) hydrogen peroxide followed by a 30 minute incubation. Each sample is neutralized by the addition of 200 μl of 3 N HCl. Ten milliliters of Ready Safe (Beckman) liquid scintillation fluid are added and the samples are counted on a scintillation system (Beckman Coulter).

The calculations are carried out as follows:

weight of saponate=weight of tube (KOH+intestine)−weight of empty tube−saponate fraction=0.22/saponate weight (density of the saponate=1.1 g/mL)−total DPM for the entire intestine=disintegration per minute (DPM) of sample/saponate fraction.

The initial bolus DPM is calculated by averaging the counts from the sham-dosed scintillation vials. The fraction of bolus recovered from the intestine (percent recovery)=total DPM/bolus count. Percent recovery from each test group=average of percent recovery from each mouse.

To compare efficacy of test compounds, an ED₂₅ for intestinal fat absorption is calculated. The (average) percent triglyceride recovery (percent unabsorbed and remaining in the intestine) of the vehicle control group is adjusted to equal 0%, and the (average) percent recovery of the compound control group is adjusted to equal 100%.

The same calculations are applied to the percent recovery values obtained for test compounds and an adjusted percent recovery is obtained (% recovery of the test sample−% recovery of vehicle control group/(% recovery of positive control group−% recovery of vehicle control group)). An ED₅₀ is then calculated by plotting a graph of compound concentration vs. adjusted percent recovery.

### Example 5

### Serum Triglyceride Lowering

Healthy female CF1 mice (Charles River) weighing 18-20 grams upon arrival are employed as test subjects. The mice are housed in groups of 10 in standard caging, and are allowed to acclimate for one week prior to testing. Mice are fasted overnight in a separate procedure room prior to testing. Each treatment group typically consists of 10 mice.

The test compound is preferably provided as a powder in a glass vial. The dosing solution (0.250 ml/25 g body weight) administered by oral gavage can be an emulsion of Miglyole 812 (40%), Cremophor (10%) and water (50%). An appropriate volume of Miglyole (Condea Vista Co.) is first added to the test compound, and the vial vortexed for approximately 1 minute. Next, the appropriate volume of Cremophor is added, and the vial again vortexed as previously. The appropriate volume of water is then added and an emulsion is formed by vortexing and briefly sonicating.

Following overnight fasting, each mouse is dosed (see above volumes) with test compound. At 1 hour post dose, the mice are euthanized by carbon dioxide inhalation and blood collected for triglyceride quantitation. Serum triglyceride values are quantitated using a colorimetric endpoint assay (Wako Triglyceride E kit) on a SpectraMax 250 plate reader with Softmax Pro software. All samples are run in duplicate.

For comparison of triglyceride values, the percent change from control is calculated. The average triglyceride value of the test compound group is divided by the average triglyceride value of the vehicle group, multiplied by 100 and then subtracted from 100%. The ED₂₅ value is then calculated by plotting a graph of compound concentration versus percent change from control. The relative values of the ED₂₅ for triglyceride lowering and the ED25 for inhibition of intestinal fat absorption are used as a means to compare selectivity of the test compounds.

### Example 6

### Murine or Canine Food Intake

C57BL/6 male mice, 18-22 grams (Wilmington, Mass. Charles River Labs), 10 mice per group, are fasted overnight (16 hrs). Mice are dosed PO at indicated dose (10 animals vehicle and 10 animals receive 100 mg/kg of test compound) and a preweighed amount of food is given to animals. Food is weighed every hour up to 6 hrs. Blood is collected at 2 to 6 hours.

Results demonstrated a nearly 50% drop in food intake of animals dosed with Compound C1 (pictured in Table 5) compared to control out through 6 hours.

Alternatively, healthy, young adult (1 to 3 years of age) male and female beagles weighing 13-19 kg at the start of the treatment period can be employed as test subjects.

The test compound is provided as a powder. The dosing solution, administered by oral gavage, can be provided employing a Miglyol/cremaphor/water 20/5/75 solution as the test vehicle. Miglyol™ (Condea Vista Co.). The dosing solution is prepared at 0.5 to 2 mg/mL activity so that 0.5 mL is delivered per kg of body weight at dosages of 0.25 to 1 mg/kg. Following a seven-day acclimation period, a four-to seven-day evaluation study can be effected.

The study consists of three groups of animals containing 2 male and 2 female dogs each. Each group of four animals is randomly assigned to receive 0.25, 0.5 or 1 mg/kg test compound. On Days 0 to 3 or 6, each dog receives the dosing solution administered as a single dose at Time 0 on each dosing day via a feeding tube. This is followed by a 10 ml water rinse to ensure total delivery of dosing solution. Each test animal is permitted ad libitum access to water and dry food each day during the study and approximately 0.5 to 1 hour post-dose.

Reduction in food intake is quantitated by weighing individual food bowls each day prior to feeding and at the end of each 24-hour consumption period during the acclimation period and again during the treatment period. The difference between the weight of the full bowl prior to feeding and the weight of the bowl and amount of food remaining at the end of the 24-hour consumption period represents the reduction in food intake attributable to the test compound.

A modified mouse food intake assay was also developed. Lean six week old C57BU6 male mice (Charles River Labs) were fed a high fat diet (60% calories from fat; Research Diets) for either 1 week or for 12 weeks prior to dosing. Mice were dosed once a day for three days at 4:30 pm (prior to dark cycle) at 10 mg/kg using a 79.75% PEG-400/0.25% Polysorbate 80/20% water formulation (100 ul/20 gr volume). On the fourth day, mice were dosed one last time in the morning and sacrificed at either 0.5, 2 or 6 hours (3 animals per time point). Daily food intake and body weight was taken over the course of the study. Final blood draw was used to determine serum drug levels, PYY, triglycerides, ALT and AST levels.

### Example 7

### Murine DIO Diabetes Models

A mouse diet induced obesity (DIO) model is used to evaluate the in vivo effects of compounds of the present invention. Six week old C57BL/6 male mice (Charles River Labs) are fed a high fat diet (60% calories from fat; Research Diets) for approximately 6 weeks until their body weight reaches ˜40 g. Test compounds are administered once daily via oral gavage at doses between 10 mg/kg, 3 mg/kg, 1 mg/kg and lower to determine efficacious threshholds. The vehicle used is 79.75% PEG-400/0.25% Polysorbate 80/20% water. A DPP-IV inhibitor (Sitagliptin Phosphate, Januvia, Merck & Co., Inc.) is also dosed alone or in combination with compounds of the present invention at 0.3 mg/kg, 1.0 mg/kg or 3.0 mg/kg. Individual mouse body weights are measured twice weekly. Every other week throughout the study, mice from each group are bled via the tail vein for determination of blood glucose and blood plasma insulin. After 1 and 3 weeks of dosing, a fasted blood glucose measure is taken. Statistical analysis is completed using the JMP program (Version 6). Data are analyzed by a one way ANOVA with comparison to control using a Dunnett's Test. A p value <0.05 indicates a significant difference between groups.

A second mouse diabetes model is also used, the ob/ob mouse. Ob/ob mice are a genetically manipulated knockout mouse that lacks leptin, an important satiety protein that neurologically signals the end of hunger. The leptin mutation is homozygous and a heterozygous strain is included as a control (Ob/+); this group should not show increased weight or blood glucose, aside from the normal effects of being on a high fat diet.

### Example 8

### Exemplary Compounds and Results

Several compounds have been tested in the assays described above. The results are as shown in Tables 1-6 below.

TABLE 1

| | | gMTP IC₅₀ | gMTP IC₅₀ |
|---|---|---|---|
| | | Triacylglycerol | ApoB Secretion |
| Compound | | Transfer Assay | Assay |
| ID | Structure | (Example 3) | (Example 2) |
| Referencecompound 1(Naringenin) | 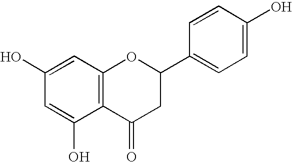 | >90% inhibitionat 200 μM | 200 μM |
| Referencecompound 2 | 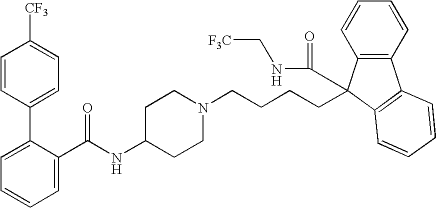 | 0.5 nM* | 0.8 nM* |
| Referencecompound 3 | 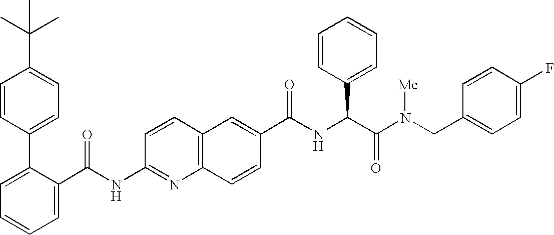 | 2.8 nM | 1.6 nM |

*As referenced in Wetterau et al., Science 282: 751, 1998.

For Tables 2 through 6, IC₅₀ data is shown for the Tri-acylglycerol (TAG) Transfer Assay (Example 3) and the Apo B Secretion Assay (Example 2), respectively. For both assays, IC₅₀ data is reported as A for values less than 100 nM, B for values between 101 nM and 1.0 uM, and C for values greater than 1.01 uM.

In Table 2, Amines A1-A5 correspond to the —NR₁R₂ moiety of the compound pictured.

In Table 3, Amines A6-A10 correspond to the —NR₁R₂ moiety of the compound pictured.

In Table 4, Amines A11-A16 correspond to the —NR₁R₂ moiety of the compound pictured.

In Table 5, Acids C1-C5 form the RC(O)— moiety of the compound pictured (the acid hydroxyl groups are lost).

TABLE 2

| | Amine | | | | |
|---|---|---|---|---|---|
| Compound | 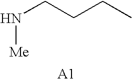 | 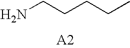 | 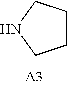 | 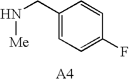 | 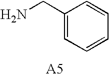 |
| 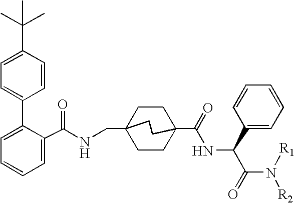 | | | —/A | A/B | |

TABLE 3

| | Amine | | | | |
|---|---|---|---|---|---|
| Compound | 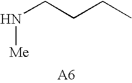 | 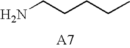 | 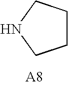 | 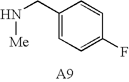 | 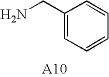 |
| 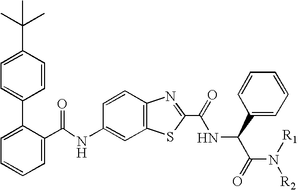 | —/A | —/A | A/A | A/A | —/A |

TABLE 4

| | Amine | | |
|---|---|---|---|
| Compound | 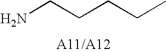 | 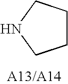 | 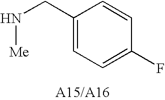 |
| 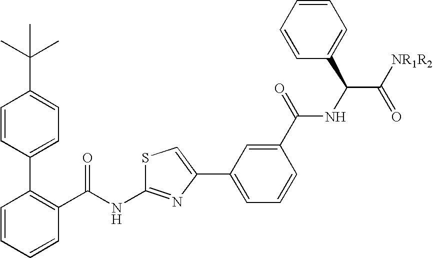 | —/B | —/A | —/B |
| 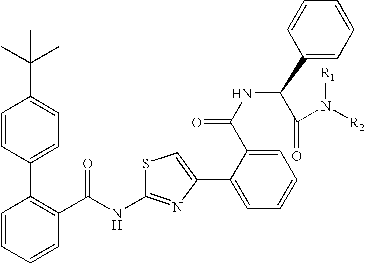 | —/C | —/C | —/C |

TABLE 5

| | Acid | | |
|---|---|---|---|
| | 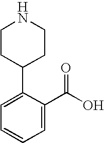 | 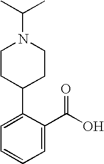 | 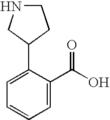 |
| Compound | C1 | C2 | C3 |
| 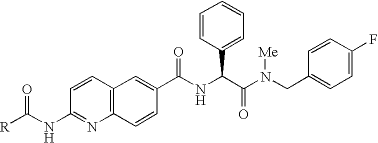 | C/C | | B/C |

| | Acid | |
|---|---|---|
| | 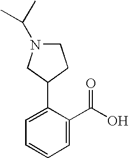 | 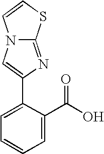 |
| Compound | C4 | C5 |
| 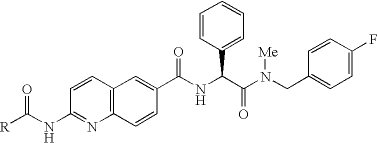 | | A/A |

### Example 8

### Activities of Additional Compounds

For the compounds listed in Table 6, the ApoB and Enzyme assay correspond to the assays described in Examples 2 and 3, respectively.

TABLE 6

| | | | IC50 | IC50 |
|---|---|---|---|---|
| | | | ApoB | Enzyme |
| Comp. | | | Assay | Assay |
| No. | [M + H]+ | Structure | (nM) | (nM) |
| 1 | 631.4 | 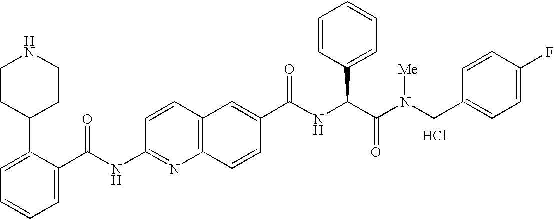 | 6000 | >1000 |
| 2 | 616.4 | 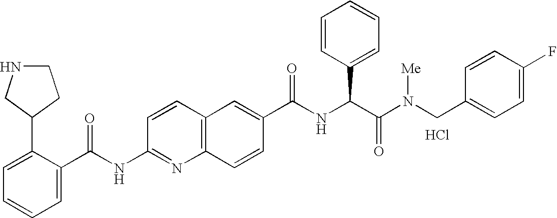 | 3500 | 530 |
| 3 | 669 | 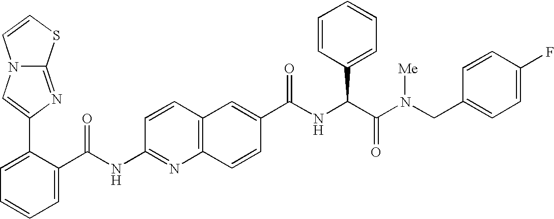 | 8.0 | 10.9 |
| 4 | 686.4 | 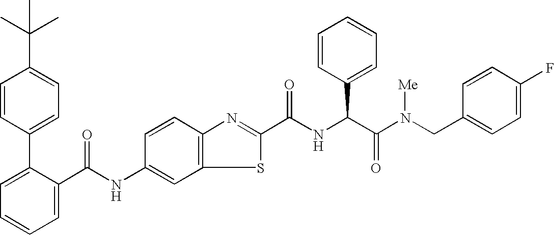 | 3.0 | 13.9/3.0 |
| 5 | 633.5 | 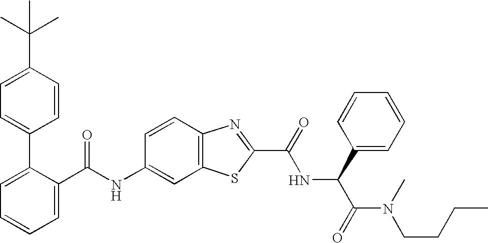 | 6.5 | 9.5 |
| 6 | 651.5 | 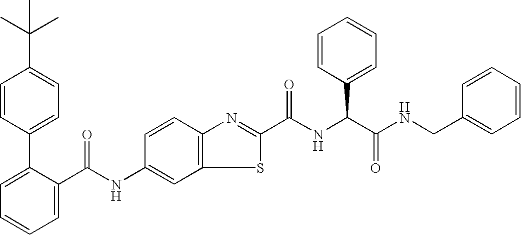 | 4.0 | |
| 7 | [M − 1]−616.0 | 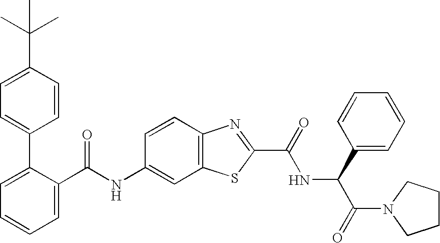 | 1.0 | 2.6 |
| 8 | [M − 1]−631.2 | 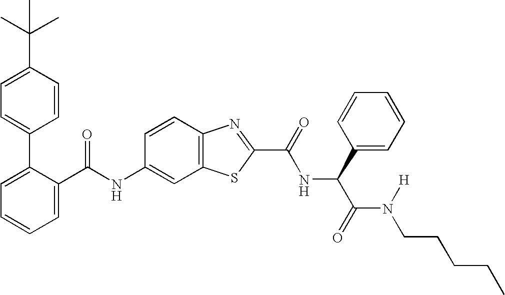 | 3.1 | 8.4 |
| 10 | 674.5 | 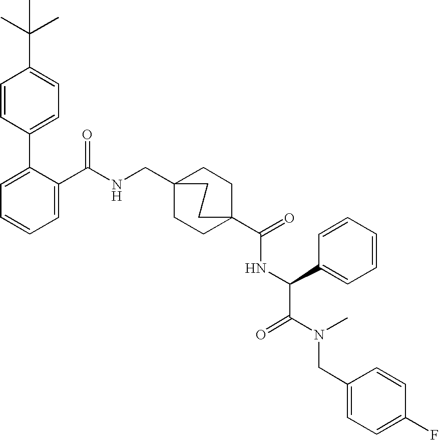 | 54 | 33 |
| 11 | 659.6 | 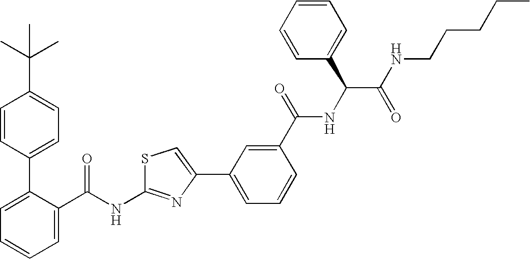 | 296 | |
| 12 | 643.5 | 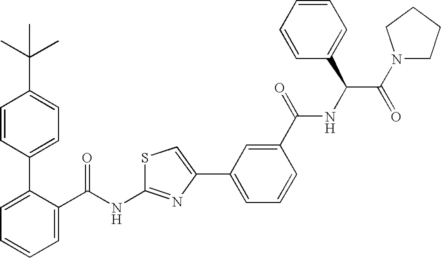 | 41 | 2 |
| 13 | [M − 1]−709.2 | 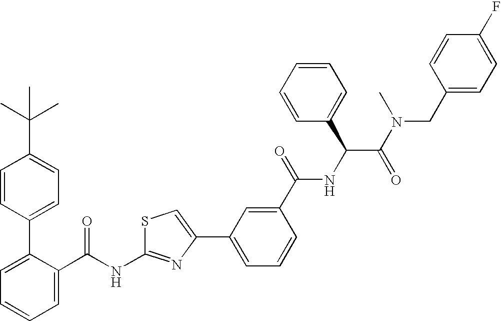 | 238 | |
| 14 | [M − 1]−651.0 | 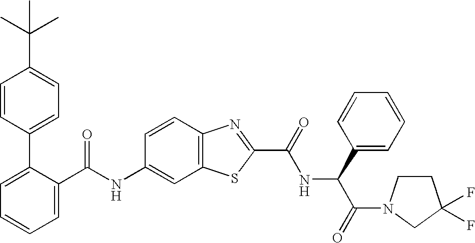 | 8.8 | 5.2 |
| 15 | 659.5 | 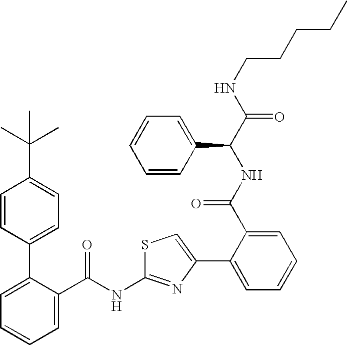 | >10000 | |
| 16 | 643.6 | 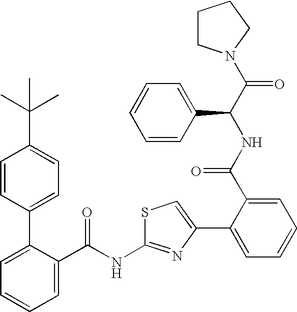 | 1100 | |
| 17 | [M − 1]−709.3 | 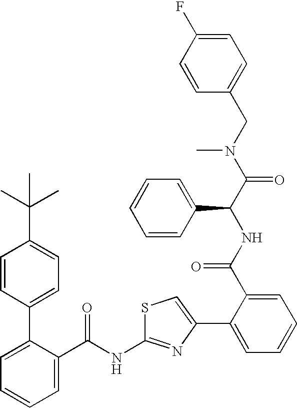 | >10000 | |
| 18 | [M − 1]−430.0 | 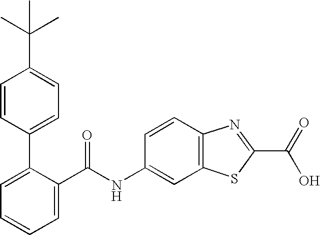 | 2700 | |
| 19 | 449 | 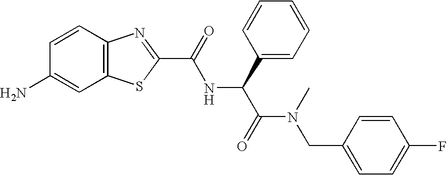 | >10000 | |
| 20 | [M − 1]−604.4 | 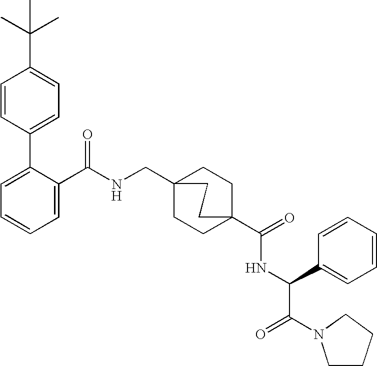 | 95 | |
| 21 | 642.5 | 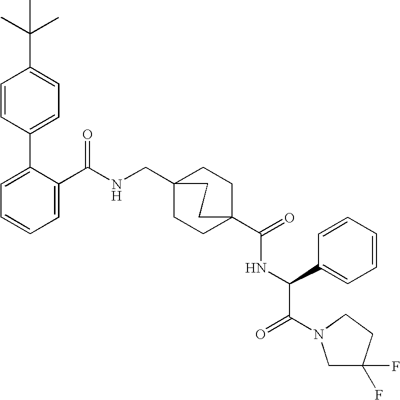 | 190 | |
| 22 | 654.5 | 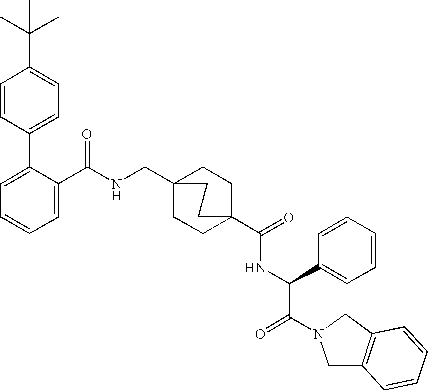 | 253 | |
| 23 | 665 | 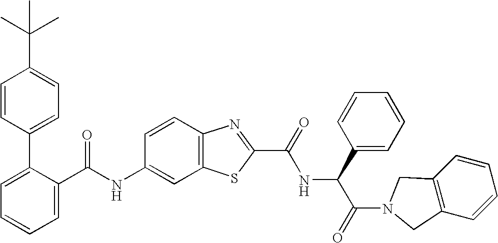 | 21.6 | |
| 24 | 675.2 | 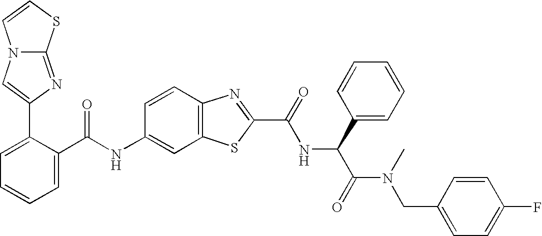 | 23.7 | 15 |
| 25 | [M − 1]−633.2 | 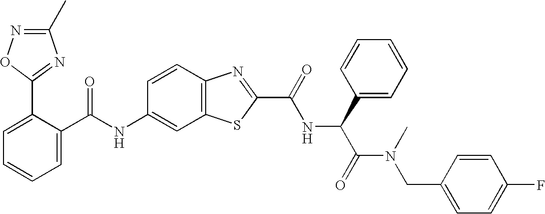 | 59.4 | |
| 26 | [M − 1]−677.1 | 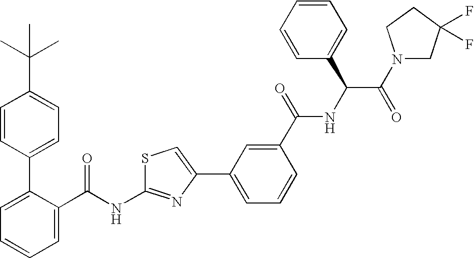 | 77.2 | |
| 27 | [M − 1]−689.0 | 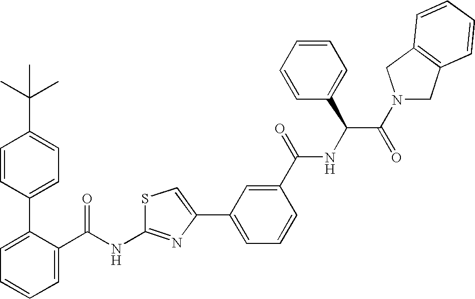 | 318 | |
| 28 | 630 | 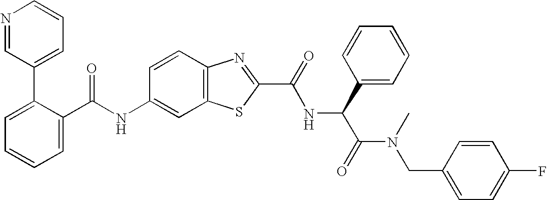 | 157.7 | |
| 29 | 621 | 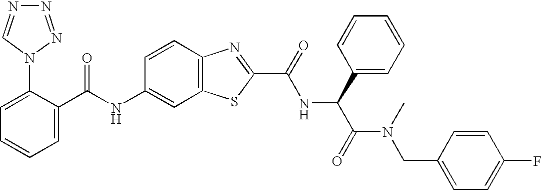 | 4200 | |
| 30 | 618.5 | 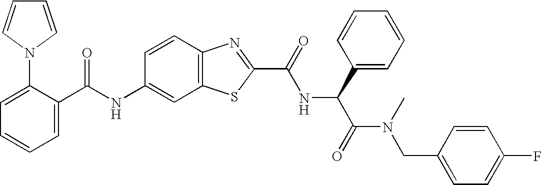 | 31 | 45 |
| 31 | 619.4 | 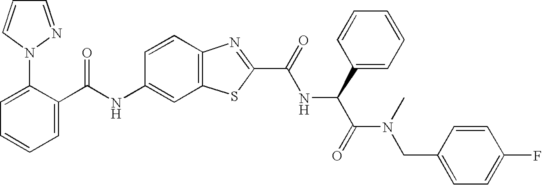 | 93.1 | |
| 32 | 697.1 | 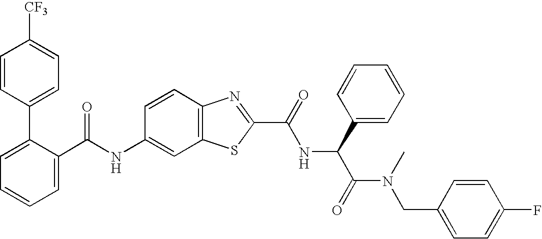 | 17.3 | 145.2 |
| 33 | 668.3 | 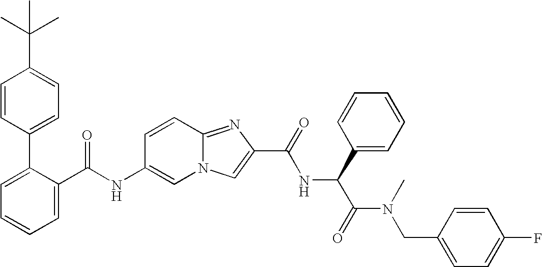 | 6.4 | 7.1 |
| 34 | 617.5 | 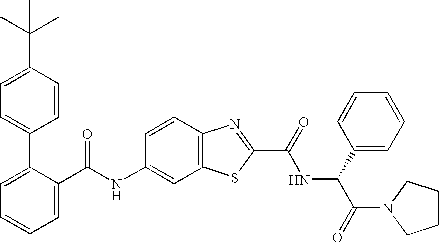 | 34.8 | |
| 35 | [M − H]−683.3 | 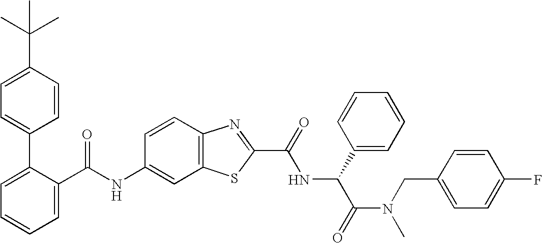 | 9.8 | |
| 36 | 643.6 | 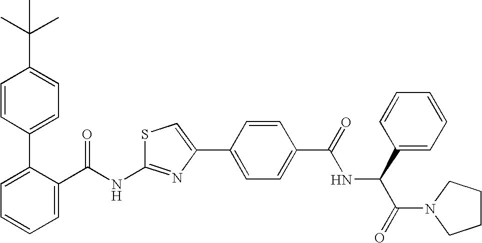 | 6.7 | 5.7 |
| 37 | 711.6 | 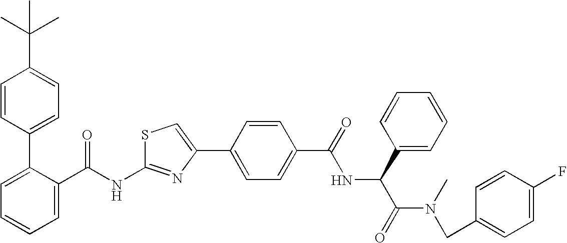 | 35.3 | |
| 38 | 712.4 | 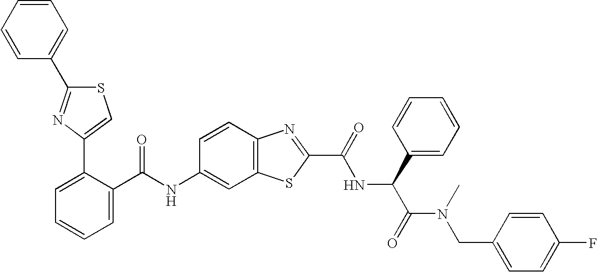 | 22.4 | 24 |
| 39 | 669.5 | 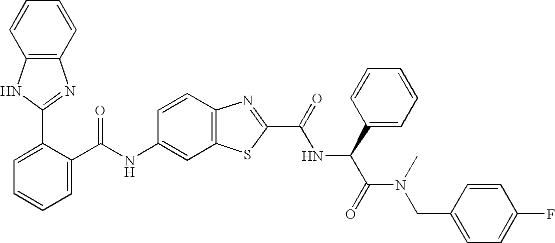 | 256.3 | |
| 40 | 669 | 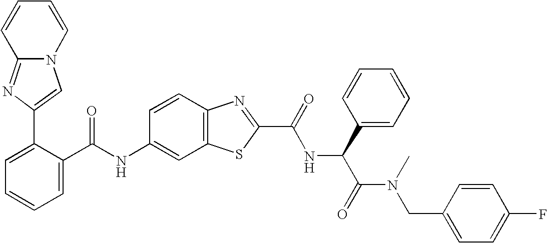 | 4.2 | 13 |
| 41 | 699 | 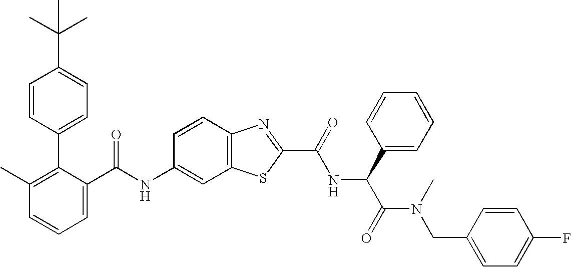 | 2.9 | 4.7 |
| 42 | 657 | 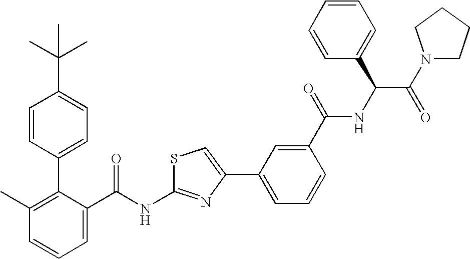 | 8.5 | 4.5 |
| 43 | 607 | 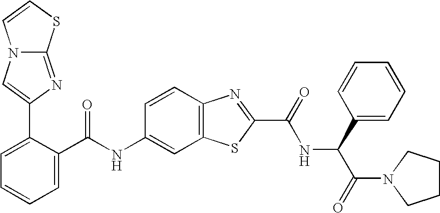 | 105 | |
| 44 | 621 | 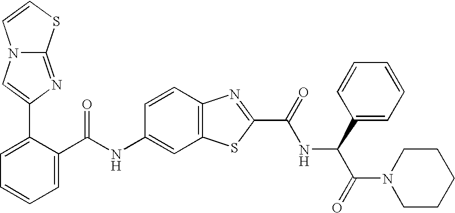 | 1607 | |
| 45 | 620.6 | 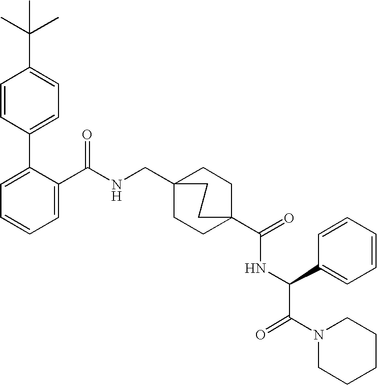 | 98.2 | |
| 46 | 620.6 | 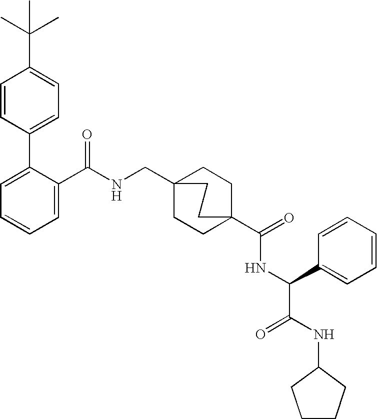 | 150.9 | |
| 47 | 634.7 | 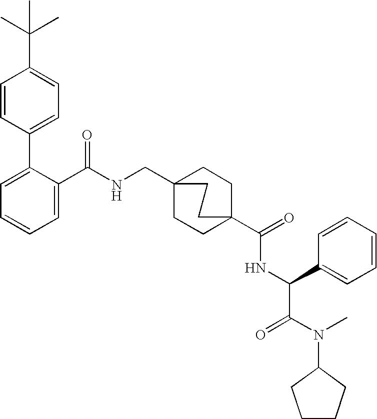 | 78.8 | |
| 48 | 634.6 | 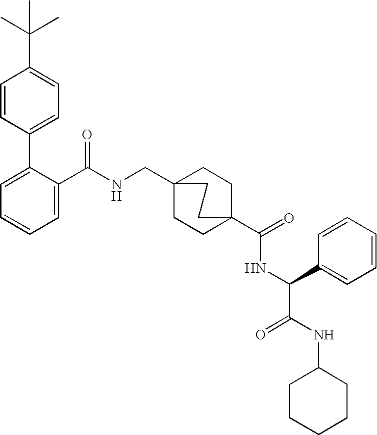 | 230 | |
| 49 | 606.7 | 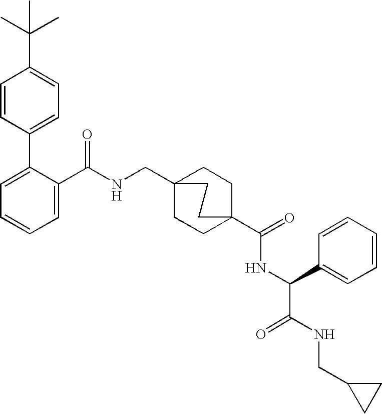 | 60.3 | |
| 50 | 608.6 | 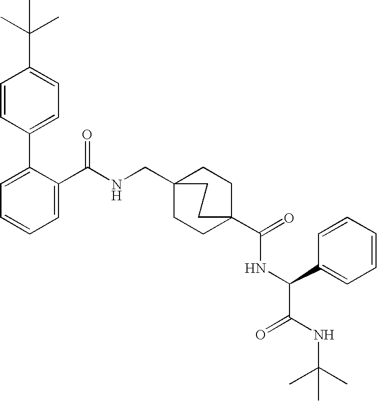 | 165 | |
| 51 | 635.3 | 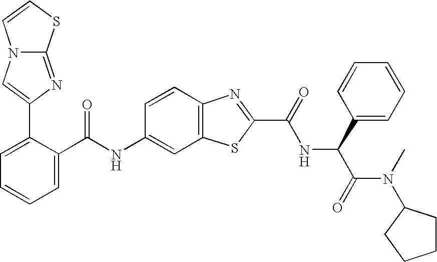 | 872 | |
| 52 | 609.3 | 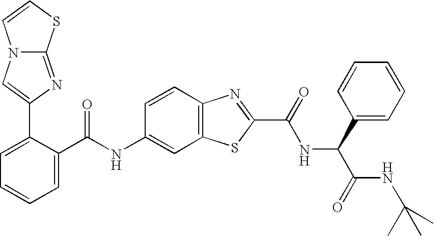 | 248 | |
| 53 | 623.4 | 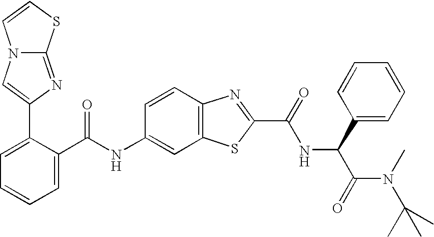 | 4179 | |
| 54 | 671 | 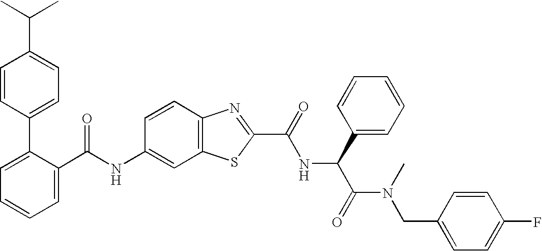 | 6.2 | 15.3/11.2 |
| 55 | 603 | 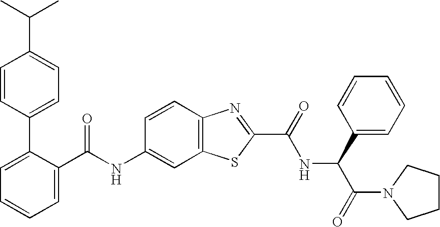 | 6.7 | 8.1/3.9 |
| 56 | 711 | 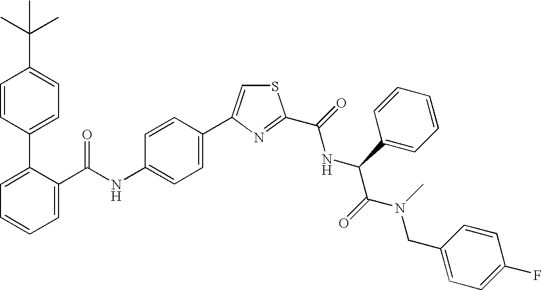 | 846 | |
| 57 | 643 | 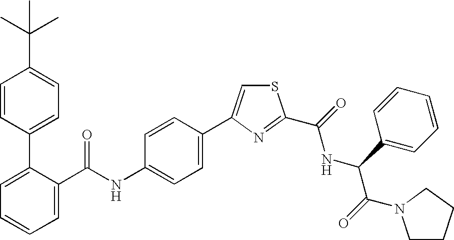 | 272 | |
| 58 | 648 | 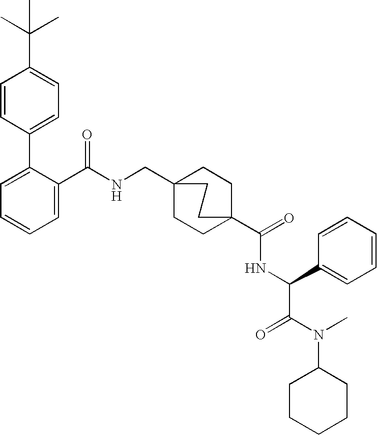 | 300 | |
| 61 | 649 | 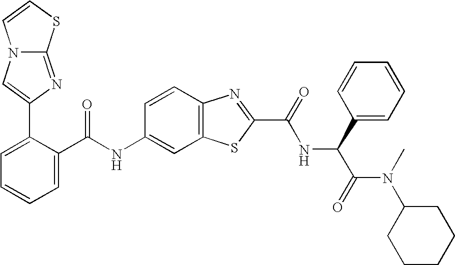 | 2105 | |
| 62 | 607.3 | 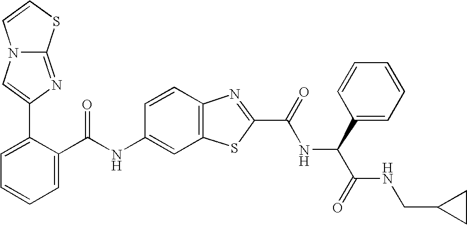 | 286 | |
| 63 | 635.4 | 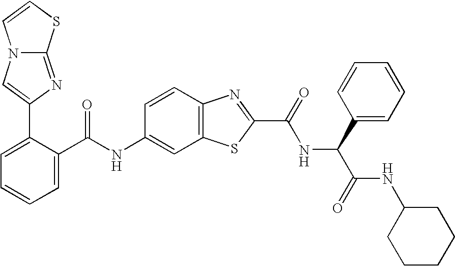 | 82 | |
| 64 | 689.1 | 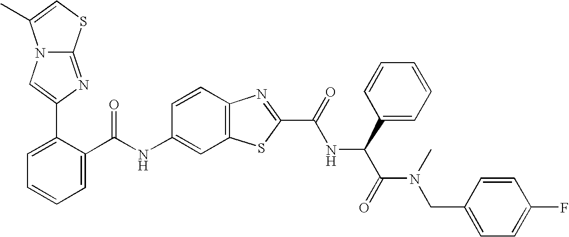 | 6.9 | 95.8 |
| 65 | 620.5 | 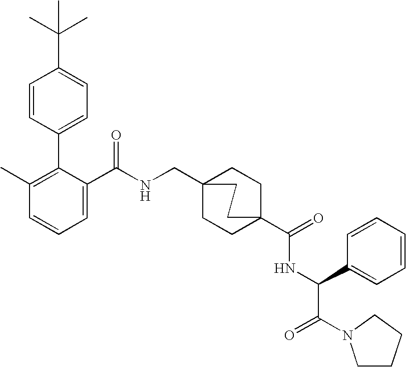 | 5.2/4.5 | |
| 66 | 692.3 | 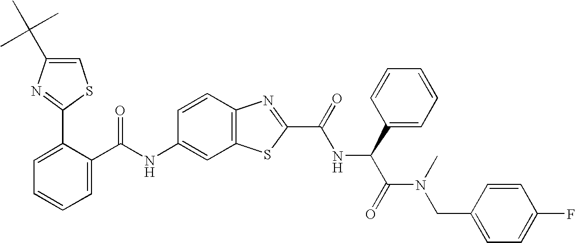 | 4.0 | 4.6 |
| 67 | 592.5 | 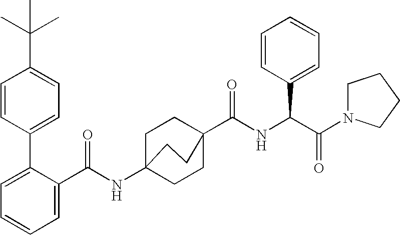 | 1.8 | 2.4 |
| 68 | 660.5 | 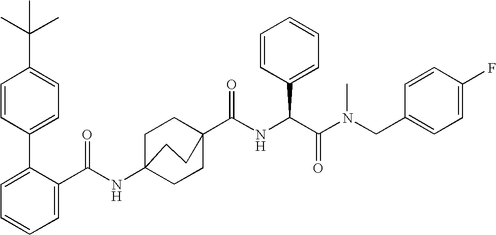 | 5.6 | 9.9 |
| 69 | 620 | 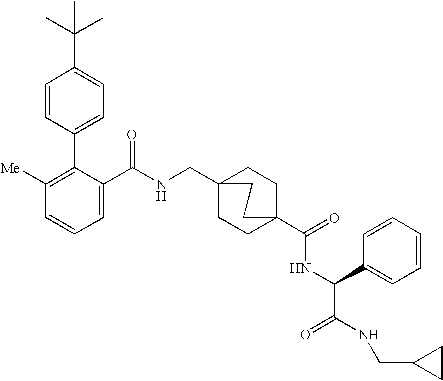 | 8.9 | |
| 70 | 622.6 | 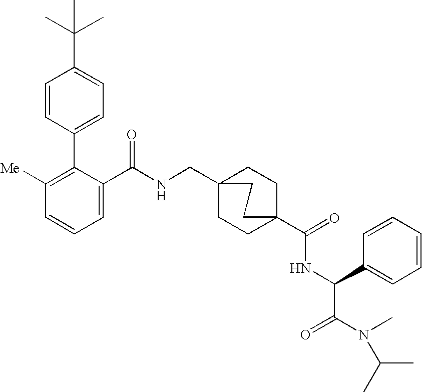 | 13.8 | |
| 71 | 622 | 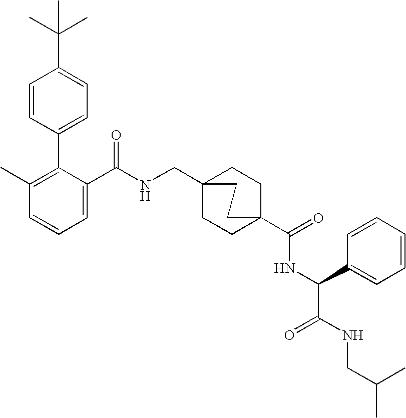 | 14.2 | |
| 72 | 636 | 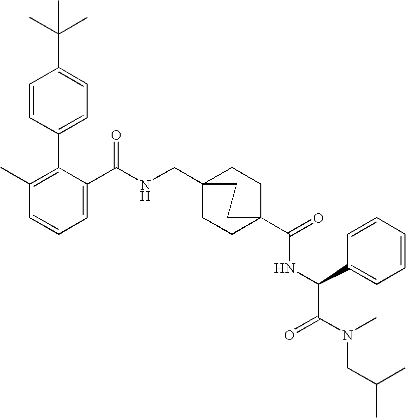 | 21.8 | |
| 73 | 622 | 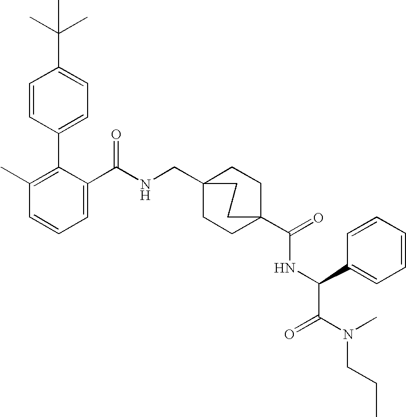 | 11.8 | 3.5 |
| 74 | 636 | 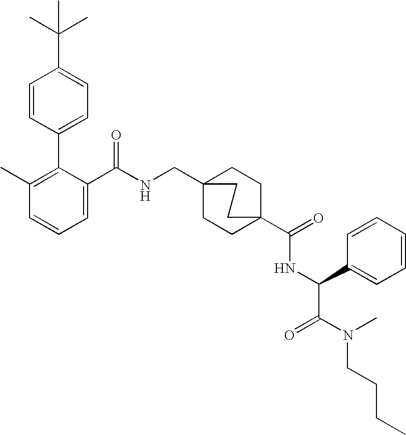 | 19.8 | |
| 75 | 688 | 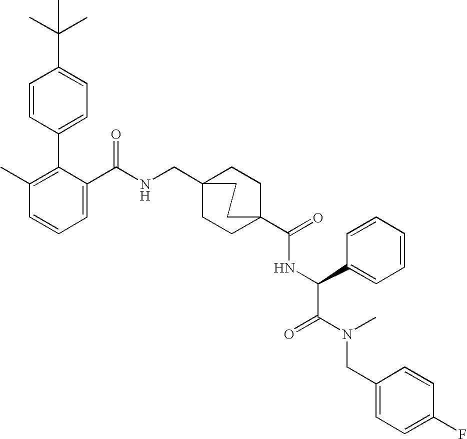 | 5.9 | 56.2 |
| 76 | 701 | 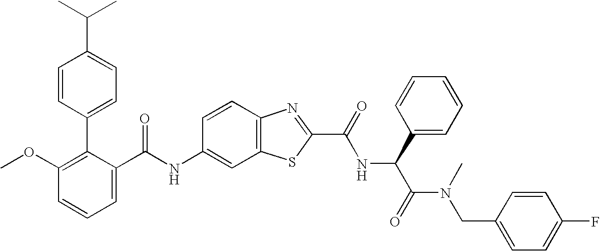 | 2.8 | |
| 77 | 647 | 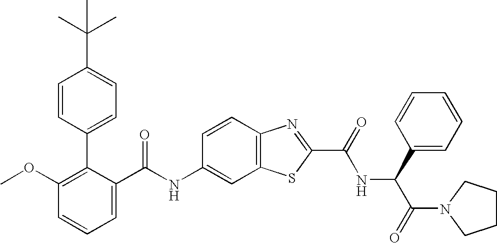 | 1.2 | 3.8 |
| 78 | 715 | 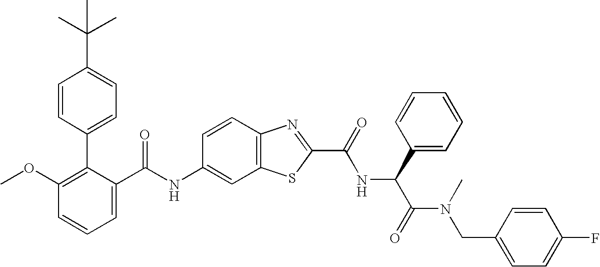 | 2.4 | 20.5/7.9 |
| 79 | 659 | 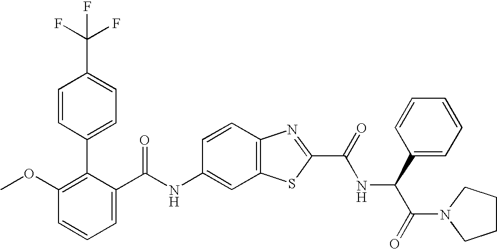 | 2.7 | 3.1 |
| 80 | 727 | 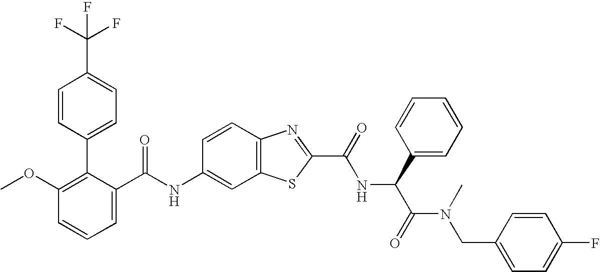 | 1.6 | |
| 81 | 643 | 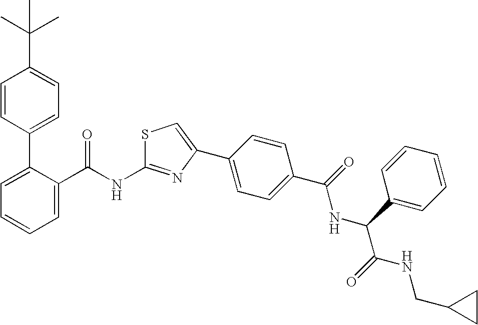 | 61.1 | |
| 82 | 645 | 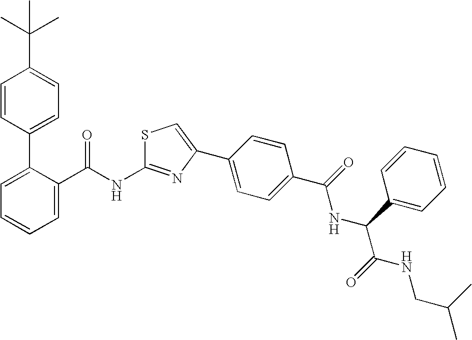 | 197.2 | |
| 83 | 657 | 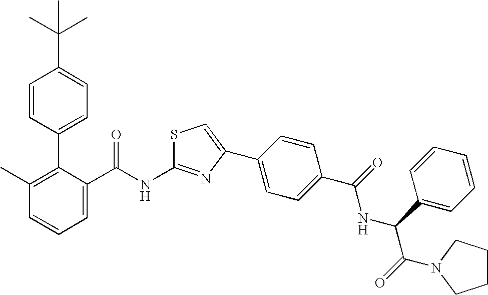 | 13.2 | 10.2 |
| 84 | 633 | 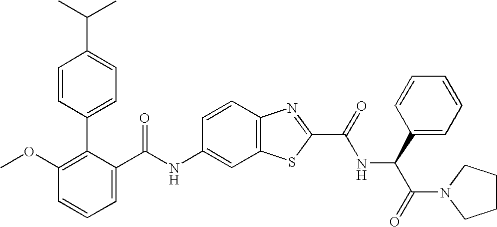 | 1.1 | 3.6 |
| 85 | 617 | 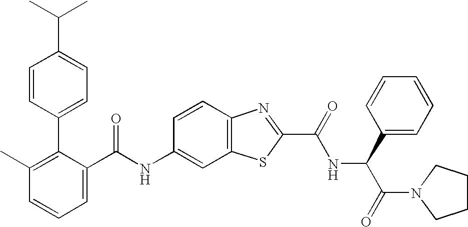 | 2.3 | 5.1 |
| 86 | 685 | 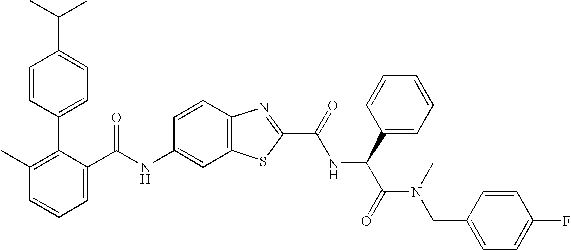 | 1.5 | 9.9/5.2 |
| 87 | 673 | 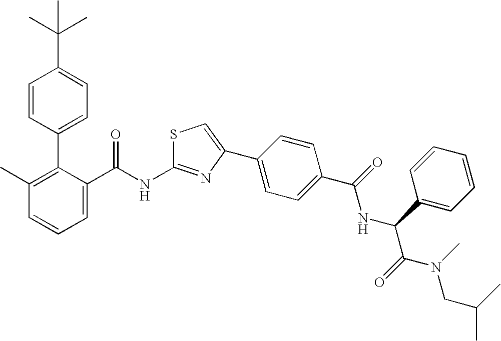 | 338.6 | |
| 88 | 659 | 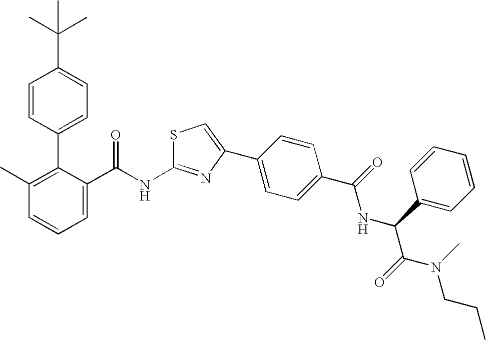 | 81.3 | |
| 89 | 643 | 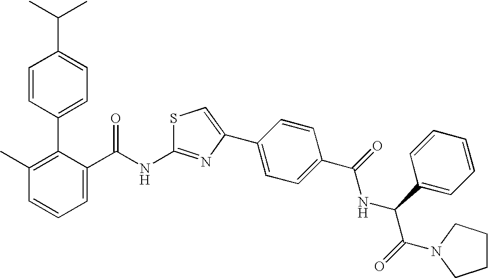 | 8.8 | |
| 90 | 659 | 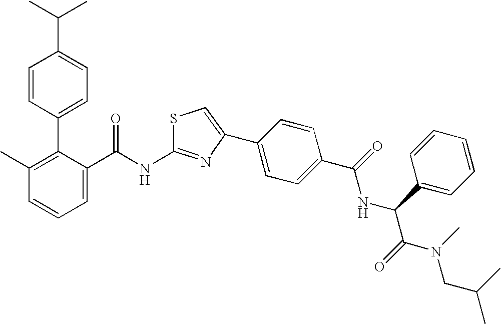 | 115 | |
| 91 | 659 | 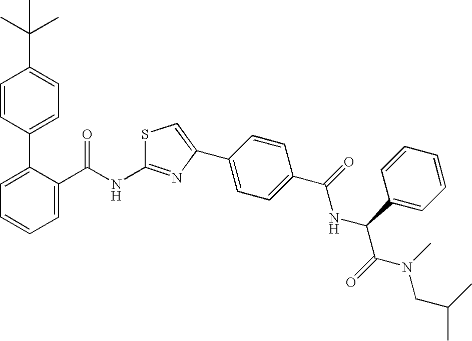 | 421.5 | |
| 92 | 645 | 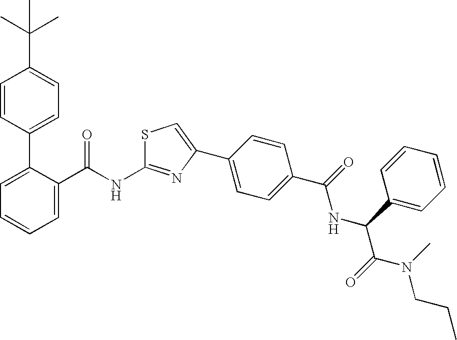 | 110.2 | |
| 93 | 645 | 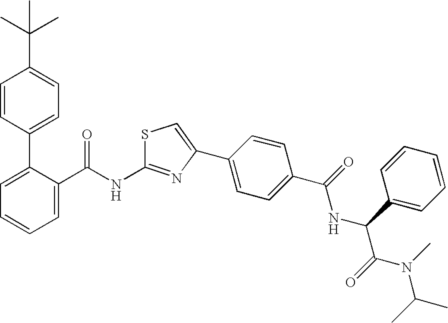 | 89.7 | |
| 94 | 629 | 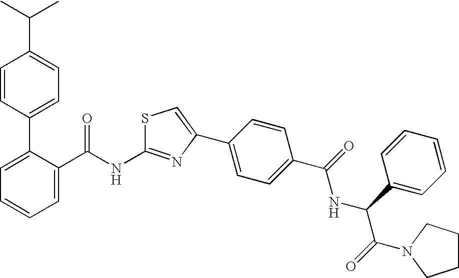 | 18.5 | |
| 95 | 631 | 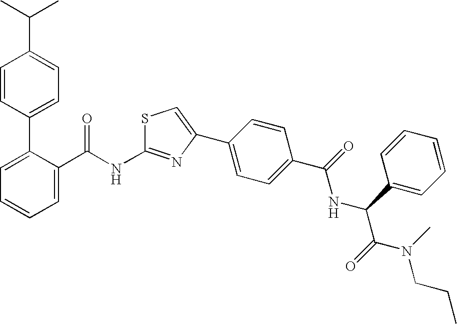 | 228.4 | |
| 96 | 606 | 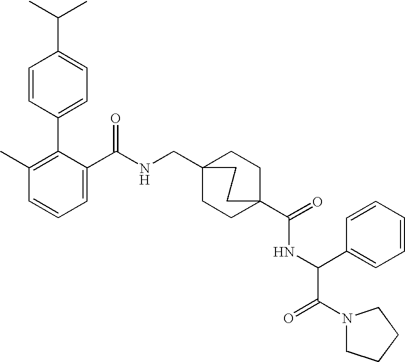 | 11 | |
| 97 | 645 | 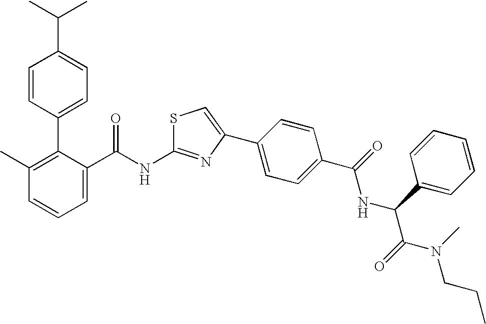 | 143.3 | |
| 98 | 645 | 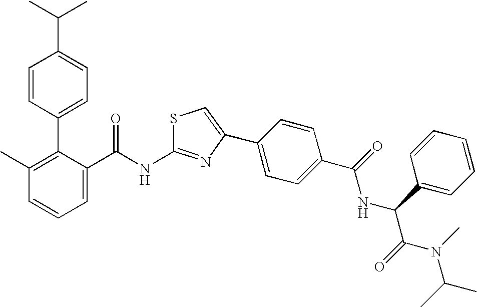 | 49.7 | |
| 99 | 631 | 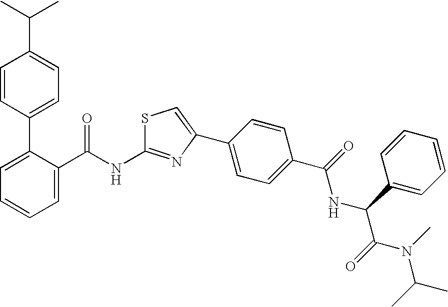 | 120.6 | |
| 100 | 613 | 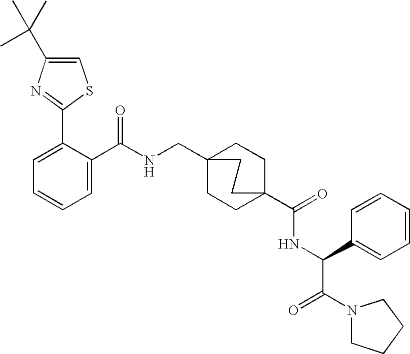 | 112.4 | |
| 101 | 608 | 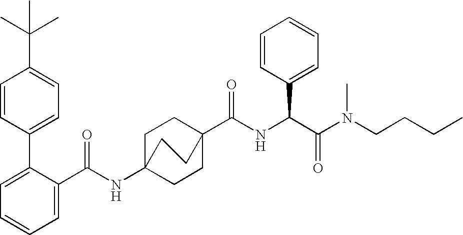 | 11.7 | |
| 102 | 608 | 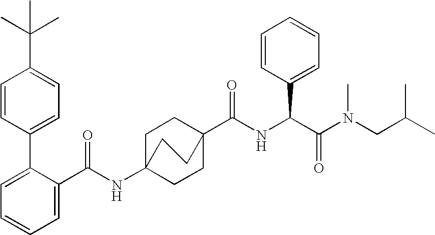 | 34.1 | |
| 103 | 594 | 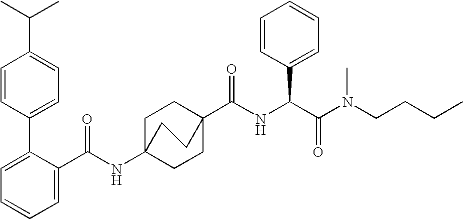 | 26.1 | |
| 104 | 578 | 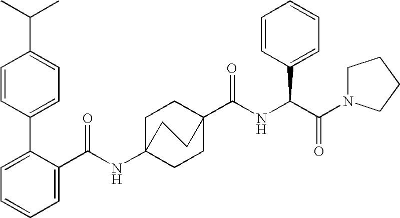 | 3.5 | |
| 105 | 646 | 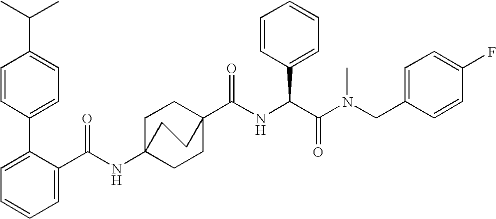 | 13.4 | |
| 106 | 594 | 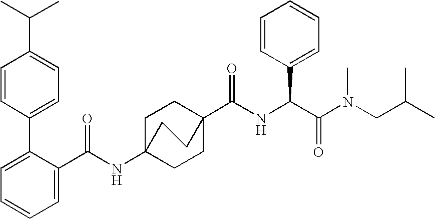 | 40.2 | |
| 107 | 606 | 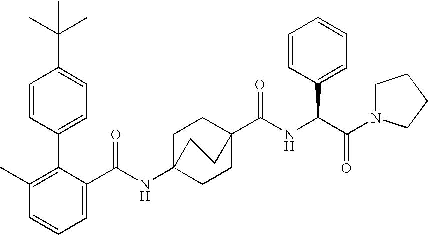 | 1.7/1.5 | |
| 108 | 674 | 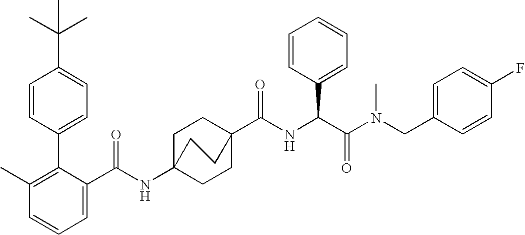 | 1.2 | |
| 109 | 622 | 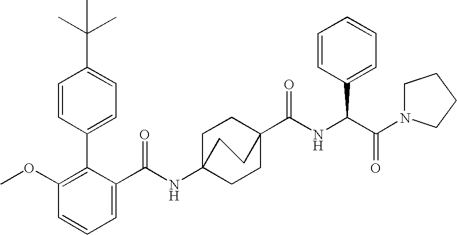 | 8.2 | |
| 110 | 690 | 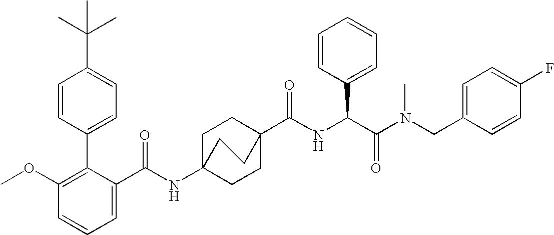 | 14.6 | |
| 111 | 676 | 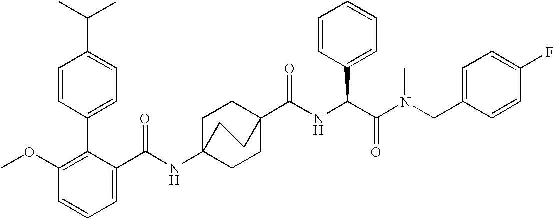 | 109.6 | |
| 112 | 608 | 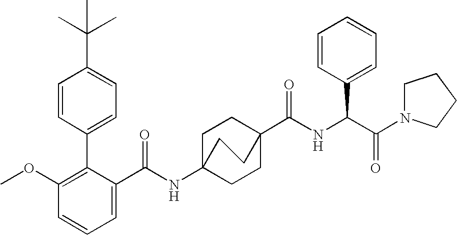 | 26.7 | |
| 113 | 592 | 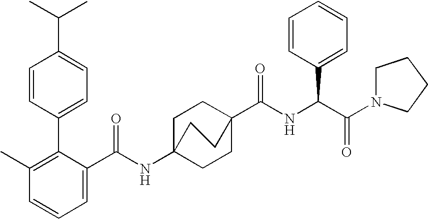 | 4.4 | |
| 114 | 660 | 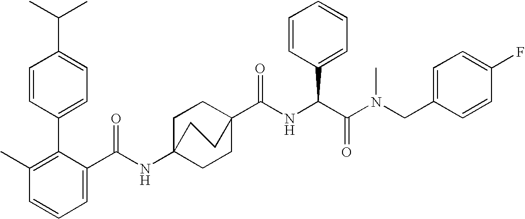 | 3.5 | |
| 115 | 624 | 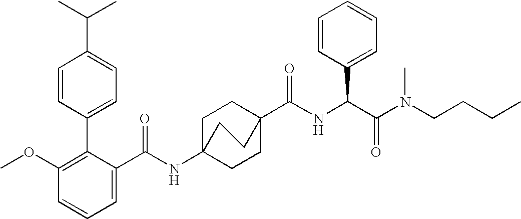 | 99.3 | |
| 116 | 624 | 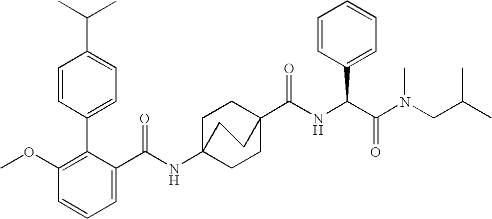 | 247.3 | |

### EQUIVALENTS

The present invention provides among other things MTP-inhibiting compounds and methods of use thereof. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATION BY REFERENCE

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

1. A compound of formula (I): 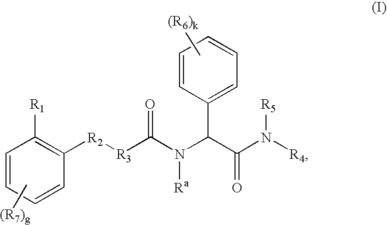
or a pharmaceutically acceptable salt thereof, wherein:
R₁ is selected from phenyl, non-aromatic heterocyclyl, or partially or fully aromatic heterocyclic;
R₂ is selected from —C(O)—N(R^{a})— or —CH(R^{a})—N(R^{a})—;
R₃ is -L₁-R₂₀-L₂-;
R₄ is selected from H, (C₁-C₆)alkyl, (C₃-C₈)cycloalkyl, —C(O)R₁₅, —C(S)R₁₅, —(CR^{a}R^{b})_{q}O(C₁-C₆ alkyl), —(CR^{a}R^{b})_{q}S(C₁-C₆ alkyl), —(CR^{a}R^{b})ᵣC(O)R₁₅, —(CR^{a}R^{b})ᵣR₁₅ or —SO₂R₁₅;
R₅ is selected from (C₁-C₆)alkyl, (C₂-C₆)alkenyl, (C₂-C₆)alkynyl, —(CR^{a}R^{b})_{q}O(C₁-C₆ alkyl), —(CR^{a}R^{b})_{q}S(C₁-C₆ alkyl); (C₃-C₈)cycloalkyl, —C(O)R₁₅, —C(S)R₁₅, —(CR^{a}R^{b})ᵣC(O)R₁₅, —(CR^{a}R^{b})ᵣC(S)R₁₅, —(CR^{a}R^{b})ᵣR₁₅, —SO₂R₁₅, phenyl, pyridyl, phenyl-Z₁- or pyridyl-Z₁-;
or R₄ and R₅ taken together with the nitrogen atom to which they are attached form a 4-10 membered heterocyclyl, wherein:
each R^{a} and R^{b} is independently H or (C₁-C₆)alkyl;
L₁ is a direct bond or —CH₂—;
L₂ is a direct bond, —CH₂—, —S—, or —O—, wherein at least one of L₁ and L₂ is a direct bond;
R₂₀ is a carbocyclic or heterocyclic ring;
Z₁ is —SO₂— or —(OR^{a}R^{b})ᵥ—;
each R₁₅ is independently H, (C₁-C₆)alkyl, or (C₃-C₈)cycloalkyl;
k is an integer from 0 to 5;
g is an integer from 0 to 4;
each j is independently 0, 1 or 2;
each q is independently an integer from 0 to 6;
each r is independently an integer from 1 to 5;
each v is independently an integer from 1 to 6; and
R₆ is selected from halo, C₁-C₄ alkyl, or O—C₁-C₄ alkyl; and
R₇ is selected from R₆ or —X—R₁₆, wherein:
X is selected from a bond, —O—, —S—, —N(R^{a})—, —C(O)N(R^{a})—, or —N(R^{a})C(O)—; and
R₁₆ is selected from C₁-C₄ alkyl, —CH₂C(O)N(R₁₇)(R₁₈), cycloalkyl, or —(CH₂)ⱼ-heterocyclyl, wherein R₁₇ and R₁₈ are independently selected from hydrogen, alkyl, carbocyclyl, heterocyclyl, heteroalkyl, aminocarbonyl, alkylcarbonyl, alkoxycarbonyl, arylcarbonyl, or aryloxycarbonyl;
wherein any alkyl, alkenyl, alkynyl or cyclic moiety of the compounds is optionally substituted with one or more suitable substituents;
provided that:
a) when R₂ is —C(O)—N(R^{a})— and R₃ is quinolinyl R₁ is not phenyl, pyridyl, naphthyl, benzofuranyl, benzodioxolyl, tetrahydroquinoline or dihydrobenzofuranyl;
b) when R₁ is phenyl or pyridyl and R₂ is —C(O)—N(R^{a})—; R₃ is not benzofuranyl, benzo[b]thienyl, indolyl, pyrrolyl, phenyl, pyridyl or phenyl-CH₂; and
c) when R₂ is —C(O)—N(R^{a})— and R₃ is benzofuranyl, benzo[b]thienyl, or indolyl; R₅ is not pyridyl, -Z₁-pyridyl, phenyl, or -Z₁-phenyl.

2. The compound of claim 1, wherein g is 0 or 1 and k is 0 or 1.

3. The compound of claim 2, wherein R₂₀ is a monocyclic ring.

4. The compound of claim 3, wherein R₂₀ is a monocyclic aromatic ring.

5. The compound of claim 2, wherein R₂₀ is a bicyclic ring.

6. The compound of claim 5, wherein R₂₀ is a bicyclic ring wherein at least one ring is aromatic.

7. The compound of claim 2, wherein R₃ is selected from -bicyclic heteroaryl-, -(bicyclic partially or fully aromatic heterocyclic)-CH₂—, —CH₂-(bicyclic partially or fully aromatic heterocyclic)-, -(bicyclic partially or fully aromatic heterocyclic)-S—, -(bicyclic partially or fully aromatic heterocyclic)-O—, -bicyclic aryl-, -(bicyclic aryl)-CH₂—, —CH₂-(bicyclic aryl)-, -(bicyclic aryl)-O—, -(bicyclic aryl)-S—, -(bicyclic non-aromatic carbocyclic)-, —CH₂-(bicyclic non-aromatic carbocyclic)-, -(bicyclic non-aromatic carbocyclic)-CH₂, -(bicyclic non-aromatic carbocyclic)-O—, -(bicyclic non-aromatic carbocyclic)-S—, -(bicyclic non-aromatic heterocyclyl)-, —CH₂-(bicyclic non-aromatic heterocyclyl)-, -(bicyclic non-aromatic heterocyclyl)-CH₂—, -(bicyclic non-aromatic heterocyclyl)-S—, -(bicyclic non-aromatic heterocyclyl)-O—, -phenyl-S—, -phenyl-O—, -phenyl-CH₂—, -phenyl-, -(monocyclic non-aromatic heterocyclyl)-, —CH₂-(monocyclic non-aromatic heterocyclyl)-, -(monocyclic non-aromatic heterocyclyl)-CH₂—, -(monocyclic non-aromatic heterocyclyl)-S—, -(monocyclic non-aromatic heterocyclyl)-O—, -monocyclic non-aromatic carbocyclic-, —CH₂-(monocyclic non-aromatic carbocyclic)-, -(monocyclic non-aromatic carbocyclic)-CH₂—, -(monocyclic non-aromatic carbocyclic)-S—, or -(monocyclic non-aromatic carbocyclic)-O—.

8. The compound of claim 2, wherein:
R₃ is selected from -bicyclic partially or fully aromatic heterocyclic-, -(bicyclic partially or fully aromatic heterocyclic)-CH₂—, -bicyclic aryl-, -(bicyclic aryl)-CH₂—, —CH₂-(bicyclic non-aromatic carbocyclic)-, —CH₂-(bicyclic non-aromatic heterocyclyl)-, -(bicyclic non-aromatic carbocyclic)-, -(bicyclic non-aromatic heterocyclyl)-, -phenyl-S—, -phenyl-O—, -phenyl-CH₂—, -phenyl-, -(monocyclic non-aromatic heterocyclyl)-CH₂—, -monocyclic non-aromatic carbocyclic-, or -(monocyclic non-aromatic carbocyclic)-CH₂—;
R₄ is selected from H, (C₁-C₆)alkyl, (C₃-C₈)cycloalkyl, —C(O)R₁₅, —C(S)R₁₅, —(CR^{a}R^{b})_{q}O(C₁-C₆ alkyl), —(CR^{a}R^{b})_{q}S(C₁-C₆ alkyl), —(CR^{a}R^{b})ᵣC(O)R₁₅, —(CR^{a}R^{b})ᵣR₁₅ or —SO₂R₁₅;
R₅ is selected from (C₁-C₆)alkyl, (C₂-C₆)alkenyl, (C₂-C₆)alkynyl, —(CR^{a}R^{b})_{q}O(C₁-C₆ alkyl), (CR^{a}R^{b})_{q}S(C₁-C₆ alkyl); (C₃-C₈)cycloalkyl, —C(O)R₁₅, —C(S)R₁₅, —(CR^{a}R^{b})ᵣC(O)R₁₅, —(CR^{a}R^{b})ᵣC(O)R₁₅, —(CR^{a}R^{b})ᵣR₁₅, —SO₂R₁₅, phenyl, pyridyl, phenyl-Z₁- or pyridyl-Z₁-, wherein said phenyl or pyridyl is optionally substituted with one to five independently selected R₁₂;
or R₄ and R₅ taken together with the nitrogen atom to which they are attached form a 4-10 membered monocyclic heterocyclyl, wherein:
R₁₂ is selected from halo, cyano, nitro, azido, amino, hydroxy, (C₁-C₆)alkyl, (C₂-C₆)alkoxy, methoxy, (C₁-C₆)alkoxy(C₁-C₆)alkyl, mono-, di- or tri-halo(C₂-C₆)alkyl), perfluoro(C₂-C₄)alkyl, trifluoromethyl, trifluoromethyl(C₁-C₅)alkyl, mono-, di- or tri-halo(C₂-C₆)alkoxy, trifluoromethyl(C₁-C₅)alkoxy, (C₁-C₆)alkylthio, hydroxy(C₁-C₆)alkyl, (C₃-C₈)cycloalkyl(CR^{a}R^{b})_{q}—, (C₂-C₆)alkenyl, (C₂-C₆)alkynyl, (C₁-C₆)alkylamino-, (C₁-C₆)dialkylamino, amino(C₁-C₆)alkyl-, —(CR^{a}R^{b})_{q}NR^{a}R₁₄, —C(O)NR^{a}R₁₄, —NR₁₄C(O)R₁₅, —NR₁₄OR₁₅, —CH═NOR₁₅, —NR₁₄C(O)OR₁₅, —NR₁₄S(O)ⱼR₁₅, —C(O))R₁₅, —C(S)R₁₅, —C(O)OR₁₅, —OC(O)R₁₅, —SO₂NR^{a}R₁₄, —S(O)ⱼR₁₅, or —(CR^{a}R^{b})_{q}S(O)ⱼR₁₅;
each R₁₄ is independently H, (C₁-C₆)alkyl, (C₃-C₈)cycloalkyl, —C(O)R₁₅, —C(S)R₁₅, —(CR^{a}R^{b})ₜO(C₁-C₆ alkyl), —(CR^{a}R^{b})ₜS(C₁-C₆ alkyl), —(CR^{a}R^{b})ᵣC(O)R₁₅, —(CR^{a}R^{b})ₜR₁₅ or —SO₂R₁₅;
each R₁₅ is independently , (C₁-C₆)alkyl or (C₃-C₈)cycloalkyl, wherein the alkyl moieties of the foregoing R₁₅ groups are independently optionally substituted with 1 to 3 substituents independently selected from C₁-C₆ alkyl, C₁-C₆ alkoxy, amino, hydroxy, halo, cyano, nitro, trifluoromethyl and trifluoromethoxy;
each j is independently 0, 1 or 2;
each q is independently an integer from 0 to 6;
each r is independently an integer from 1 to 5;
each t is independently an integer from 1 to 6; and
each v is independently an integer from 1 to 6;
wherein any alkyl, alkenyl, alkynyl or cyclic moieties of R₁, R₂, R₃, R₄, R₅, R₆, or R₇ grounps are independently optionally substituted with 1 to 3 substituents independently selected from halo, cyano, nitro, trifluoromethyl, trifluoromethoxy, azido, —OR₁₅, —C(O)R₁₅, —C(O)OR₁₅, —OC(O)R₁₅, —NR₁₄C(O)R₁₅, C(O)NR^{a}R₁₄, NR^{a}R₁₄, and —NR₁₄OR¹⁵, C₁-C₆ alkyl, C₂-C₆ alkenyl, and C₂-C₆ alkynyl.

9. A compound of formula (II): 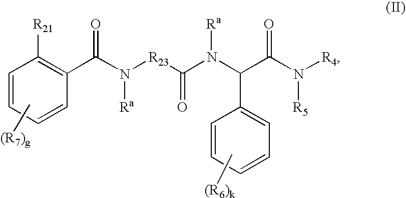
or a pharmaceutically acceptable salt thereof, wherein:
R₂₁ is selected from non-aromatic heterocyclyl; monocyclic heteroaryl, wherein said monocyclic heteroaryl comprises either: (i) at least one ring heteroatom selected from O or S, or (ii) at least two ring atoms independently selected from O, N or S; or bicyclic heteroaryl, wherein said bicyclic heteroaryl comprises a ring heteroatom selected from N or S;
R₂₃ is quinolinyl;
R₄ is selected from H, (C₁-C₆)alkyl, (C₃-C₈)cycloalkyl, —C(O)R₁₅, —C(S)R₁₅, —(CR^{a}R^{b})_{q}O(C₁-C₆ alkyl), —(CR^{a}R^{b})_{q}S(C₁-C₆ alkyl), —(CR^{a}R^{b})ᵣC(O)R₁₅, —(CR^{a}R^{b})ᵣR₁₅ or —SO₂R₁₅;
R₅ is selected from (C₁-C₆)alkyl, (C₂-C₆)alkenyl, (C₂-C₆)alkynyl, —(CR^{a}R^{b})_{q}O(C₁-C₆ alkyl), (CR^{a}R^{b})_{q}S(C₁-C₆ alkyl), (C₃-C₈)cycloalkyl, —C(O)R₁₅, —C(S)R₁₅, —(CR^{a}R^{b})ᵣC(O)R₁₅, —(CR^{a}R^{b})ᵣC(S)R₁₅, —(CR^{a}R^{b})ᵣR₁₅, —SO₂R₁₅, phenyl, pyridyl, phenyl-Z₁- or pyridyl-Z₁-;
or R₄ and R₅ taken together with the nitrogen atom to which they are attached form a 4-10 membered heterocyclyl;
each R^{a} and R^{b} is independently H or (C₁-C₆)alkyl;
Z₁ is —SO₂— or —(CR^{a}R^{b})ᵥ;
each R₁₅ is independently H, (C₁-C₆)alkyl, or (C₃-C₈)cycloalkyl;
k is an integer from 0 to 5;
g is an integer from 0 to 4;
each j is 0, 1 or 2;
each q is independently an integer from 0 to 6;
each r is independently an integer from 1 to 5;
each v is independently an integer from 1 to 6;
R₆ is selected from halo, C₁-C₄ alkyl, or O—C₁-C₄ alkyl; and
R₇ is selected from R₆ or —X—R₁₆, wherein:
X is selected from a bond, —O—, —S—, —N(R^{a})—, —C(O)N(R^{a})—, or —N(R^{a})C(O)—; and
R₁₆ is selected from C₁-C₄ alkyl, —CH₂C(O)N(R₁₇)(R₁₈), cycloalkyl, or —(CH₂)ⱼ-heterocyclyl, wherein R₁₇ and R₁₈ are independently selected from hydrogen, alkyl, carbocyclyl, heterocyclyl, heteroalkyl, aminocarbonyl, alkylcarbonyl, alkoxycarbonyl, arylcarbonyl, or aryloxycarbonyl,
wherein any alkyl, alkenyl, alkynyl or cyclic moiety of the compounds is optionally substituted with one or more suitable substituents.

10-11. (canceled)

12. A compound of formula (III): 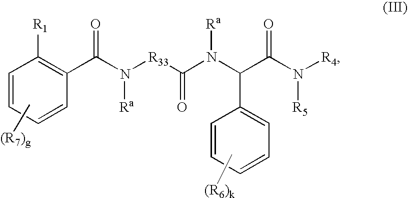
or a pharmaceutically acceptable salt thereof, wherein:
R₁ is selected from phenyl, a non-aromatic heterocyclyl, or partially or fully aromatic heterocyclic;
R₃₃ is selected from a bicyclic heterocycle comprising at least one aromatic ring and comprising at least two ring heteroatoms independently selected from N, O or S; -(bicyclic heteroaryl)-CH₂—; -bicyclic aryl-; -(bicyclic aryl)-CH₂—; —CH₂-(bicyclic non-aromatic carbocyclic)-; -(bicyclic non-aromatic carbocyclic)-; -(bicyclic non-aromatic carbocyclic)-CH₂; —CH₂-(bicyclic non-aromatic heterocyclyl)-; -(bicyclic non-aromatic heterocyclyl)-CH₂—; -phenyl-S—; -phenyl-O—; -(monocyclic saturated heterocyclyl)-CH₂—; -monocyclic non-aromatic carbocyclic-; or -(monocyclic non-aromatic carbocyclic)-CH₂—,
R₄ is selected from H, (C₁-C₆)alkyl, (C₃-C₈)cycloalkyl, —C(O)R₁₅, —C(S)R₁₅, —(CR^{a}R^{b})_{q}O(C₁-C₆ alkyl), —(CR^{a}R^{b})_{q}S(C₁-C₆ alkyl), —(CR^{a}R^{b})ᵣC(O)R₁₅, —(CR^{a}R^{b})ᵣR₁₅ or —SO₂R₁₅;
R₅ is selected from (C₁-C₆)alkyl, (C₂-C₆)alkenyl, (C₂-C₆)alkynyl, —(CR^{a}R^{b})_{q}O(C₁-C₆ alkyl), —(CR^{a}R^{b})_{q}S(C₁-C₆ alkyl); (C₃-C₈)cycloalkyl, —C(O)R₁₅, —C(S)R₁₅, —(CR^{a}R^{b})ᵣC(O)R₁₅, —(CR^{a}R^{b})ᵣC(S)R₁₅, —(CR^{a}R^{b})ᵣR₁₅, —SO₂R₁₅, phenyl, pyridyl, phenyl-Z₁- or pyridyl-Z₁-;
or R₄ and R₅ taken together with the nitrogen atom to which they are attached form a 4-10 membered heterocyclyl, wherein:
each R^{a} and R^{b} is independently H or (C₁-C₆)alkyl;
Z₁ is —SO₂— or —(CR^{a}R^{b})ᵥ—;
each R₁₅ is independently H, (C₁-C₆)alkyl, or (C₃-C₈)cycloalkyl;
k is an integer from 0 to 5;
g is an integer from 0 to 4;
each j is independently 0, 1 or 2;
each q is independently an integer from 0 to 6;
each r is independently an integer from 1 to 5;
each v is independently an integer from 1 to 6; and
R₆ is selected from halo, C₁-C₄ alkyl, or O—C₁-C₄ alkyl; and
R₇ is selected from R₆, or —X—R₁₆, wherein:
X is selected from a bond, —O—, —S—, —N(R^{a})—, —C(O)N(R^{a})—, or —N(Rₐ)C(O)—; and
R₁₆ is selected from C₁-C₄ alkyl, —CH₂C(O)N(R₁₇)(R₁₈), cycloalkyl, or —(CH₂)ⱼ-heterocyclyl, wherein R₁₇ and R₁₈ are independently selected from hydrogen, alkyl, carbocyclyl, heterocyclyl, heteroalkyl, aminocarbonyl, alkylcarbonyl, alkoxycarbonyl, arylcarbonyl, or aryloxycarbonyl,
wherein any alkyl, alkenyl, alkynyl or cyclic moiety of the compounds is optionally substituted with one or more suitable substituents.

13-14. (canceled)

15. A compound of formula (IV): 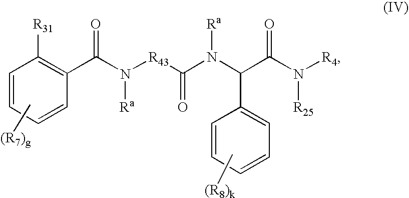
or a pharmaceutically acceptable salt thereof, wherein:
R₃₁ is selected from non-aromatic heterocyclyl, or partially or fully aromatic heterocyclic, wherein when said partially or fully aromatic heterocyclic is monocyclic it comprises either (i) at least one ring heteroatom selected from O or S, or (ii) at least two ring atoms independently selected from O, N or S;
R₄₃ is selected from benzofuranyl, dihydrobenzofuranyl, tetrahydrobenzofuranyl, benzothienyl, dihydrobenzothienyl, tetrahydrobenzothienyl, indolyl, dihydroindolyl or tetrahydroindolyl;
each of R₄ and R₂₅ is independently selected from (C₁-C₆)alkyl, (C₂-C₆)alkenyl, (C₂-C₆)alkynyl, —(CR^{a}R^{b})_{q}O(C₁-C₆ alkyl), (CR^{a}R^{b})_{q}S(C₁-C₆ allyl); (C₃-C₈)cycloalkyl, —C(O)R₁₅, —C(S)R₁₅, —(CR^{a}R^{b})ᵣC(O)R₁₅, —(CR^{a}R^{b})ᵣC(S)R₁₅, —(CR^{a}R^{b})ᵣR₁₅, or —SO₂R₁₅;
or R₄ and R₂₅ taken together with the nitrogen atom to which they are attached together form a 4-10 membered heterocyclyl, wherein:
each R^{a} and R^{b} is independently H or (C₁-C₆)alkyl;
each R₁₅ is independently H, (C₁-C₆)alkyl, or (C₃-C₈)cycloalkyl;
k is air integer from 0 to 5;
g is an integer from 0 to 4;
each j is independently 0, 1 or 2;
each q is independently an integer from 0 to 6;
each r is independently an integer from 1 to 5; and
R₆ is selected from halo, C₁-C₄ alkyl, or O—C₁-C₄ alkyl; and
R₇ is selected from R₆, or —X—R₁₆, wherein:
X is selected from a bond, —O—, —S—, —N(R^{a})—, —C(O)N(R^{a})—, or —N(R^{a})C(O)—; and
R₁₆ is selected from C₁-C₄ alkyl, —CH₂C(O)N(R₁₇)(R₁₈), cycloalkyl, or —(CH₂)ⱼ-heterocyclyl, wherein R₁₇ and R₁₈ are independently selected from hydrogen, alkyl, carbocyclyl, heterocyclyl, heteroalkyl, aminocarbonyl, alkylcarbonyl, alkoxycarbonyl, arylcarbonyl, or aryloxycarbonyl,
wherein any alkyl, alkenyl, alkynyl or cyclic moiety of the compounds is optionally substituted with one or more suitable substituents.

16-17. (canceled)

18. A compound of formula (V): 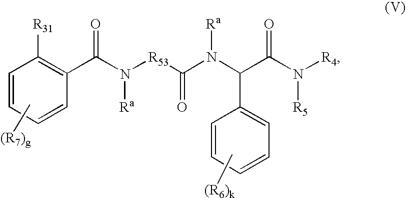
or a pharmaceutically acceptable salt thereof, wherein:
R₃₁ is selected from non-aromatic heterocyclyl, or partially or fully aromatic heterocyclic, wherein when said partially or fully aromatic heterocyclic is monocyclic it comprises either (i) at least one ring heteroatom selected from O or S, or (ii) at least two ring atoms independently selected from O, N or S;
R₅₃ is selected from phenyl or —CH₂-(phenyl)-;
R₄ is selected from H, (C₁-C₆)alkyl, (C₃-C₈)cycloalkyl, —C(O)R₁₅, —C(S)R₁₅, —(CR^{a}R^{b})_{q}O(C₁-C₆ alkyl), —(CR^{a}R^{b})_{q}S(C₁-C₆ alkyl), —(CR^{a}R^{b})ᵣC(O)R₁₅, —(CR^{a}R^{b})ᵣR₁₅ or —SO₂R₁₅;
R₅ is selected from (C₁-C₆)alkyl, (C₂-C₆)alkenyl, (C₂-C₆)alkynyl, —(CR^{a}R^{b})_{q}O(C₁-C₆ alkyl), —(CR^{a}R^{b})_{q}S(C₁-C₆ alkyl); (C₃-C₈)cycloalkyl, —C(O)R₁₅, —C(S)R₁₅, —C(CR^{a}R^{b})ᵣC(O)R₁₅, —(CR^{a}R^{b})ᵣC(S)R₁₅, —(CR^{a}R^{b})ᵣR₁₅, —SO₂R₁₅, phenyl, pyridyl, phenyl-Z₁- or pyridyl-Z₁-;
or R₄ and R⁵ taken together with the nitrogen atom to which they are attached form a 4-10 membered heterocyclyl, wherein:
each R^{a} and R^{b} is independently H or (C₁-C₆)alkyl;
Z₁ is —SO₂— or —(CR^{a}R^{b})ᵥ—;
each R₁₅ is independently H, (C₁-C₆)alkyl, or (C₃-C₈)cycloalkyl;
k is an integer from 0 to 5;
g is an integer from 0 to 4;
each j is independently 0, 1 or 2;
each q is independently an integer from 0 to 6;
each r is independently an integer from 1 to 5;
each v is independently an integer from 1 to 6; and
R₆ is selected from halo, C₁-C₄ alkyl, or O—C₁-C₄ alkyl; and
R₇ is selected from R₆ or —X—R₁₆, wherein:
X is selected from a bond, —O—, —S—, —N(R^{a})—, —C(O)N(R^{a})—, or —N(O)C(O)—; and
R₁₆ is selected from C₁-C₄ alkyl, —CH₂C(O)N(R₁₇)(R₁₈), cycloalkyl, or —(CH₂)ⱼ-heterocyclyl, wherein R₁₇ and R₁₈ are independently selected from hydrogen, alkyl, carbocyclyl, heterocyclyl, heteroalkyl, aminocarbonyl, alkylcarbonyl, alkoxycarbonyl, arylcarbonyl, or aryloxycarbonyl,
wherein any alkyl, alkenyl, alkynyl or cyclic moiety of the compounds is optionally substituted with one or more suitable substituents.

19-20. (canceled)

21. A compound of formula (VI): 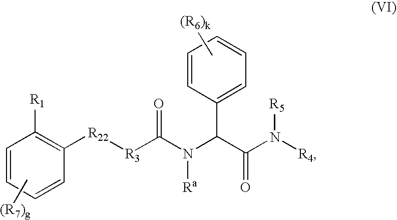
or a pharmaceutically acceptable salt thereof, wherein:
R₁ is selected from phenyl, non-aromatic heterocyclyl, or partially or fully aromatic heterocyclyl;
R₂₂ is —CH(R^{a})—N(R^{a})—;
R₃ is selected from -bicyclic partially or fully aromatic heterocyclyl-, -(bicyclic partially or fully aromatic heterocyclyl)-CH₂—, -bicyclic aryl-, -(bicyclic aryl)-CH₂—, —CH₂-(bicyclic non-aromatic cycloalkyl), —CH₂-(bicyclic non-aromatic heterocyclyl), -phenyl-S—, -phenyl-O—, -phenyl-CH₂—, -phenyl-, -(monocyclic non-aromatic heterocyclyl)-CH₂—, -monocyclic non-aromatic cycloalkyl-, or -(monocyclic non-aromatic cycloalkyl)-CH₂—;
R₄ is selected from H, (C₁-C₆)alkyl, (C₃-C₈)cycloalkyl, —C(O)R₁₅, —C(S)R₁₅, —(CR^{a}R^{b})_{q}O(C₁-C₆ alkyl), —(CR^{a}R^{b})_{q}S(C₁-C₆ alkyl), —(CR^{a}R^{b})ᵣC(O)R₁₅, —(CR^{a}R^{b})₁₅ or —SO₂R₁₅;
R₅ is selected from (C₁-C₆)alkyl, (C₂-C₆)alkenyl, (C₂-C₆)alkynyl, —(CR^{a}R^{b})_{q}O(C₁-C₆ alkyl), —(CR^{a}R^{b})_{q}S(C₁-C₆ alkyl); (C₃-C₈)cycloalkyl, —C(O)R₁₅, —C(S)R₁₅, —(CR^{a}R^{b})ᵣC(O)R₁₅, —(CR^{a}R^{b})ᵣC(S)R₁₅, —(CR^{a}R^{b})ᵣR₁₅, —SO₂R₁₅, phenyl, pyridyl, phenyl-Z₁- or pyridyl-Z₁-;
or R₄ and R₅ taken together with the nitrogen atom to which they are attached form a 4-10 membered heterocyclyl, wherein:
each R^{a} and R^{b} is independently H or (C₁-C₆)alkyl;
Z₁ is —SO₂— or —(CR^{a}R^{b})ᵥ—;
each R₁₅ is independently H, (C₁-C₆)alkyl, or (C₃-C₈)cycloalkyl;
k is an integer from 0 to 5;
g is an integer from 0 to 4;
each j is independently 0, 1 or 2;
each q is independently an integer from 0 to 6;
each r is independently an integer from 1 to 5;
each v is independently an integer from 1 to 6; and
R₆ is selected from halo, C₁-C₄ alkyl, or O—C₁-C₄ alkyl; and
R₇ is selected from R₆, or —X—R₁₆, wherein:
X is selected from a bond, —O—, —S—, —N(R^{a})—, —C(O)N(R^{a})—, or —N(R^{a})C(O)—; and
R₁₆ is selected from C₁-C₄ alkyl, —CH₂C(O)N(R₁₇)(R₁₈), cycloalkyl, or —(CH₂)-heterocyclyl, wherein R₁₇ and R₁₈ are independently selected from hydrogen, alkyl, carbocyclyl, heterocyclyl, heteroalkyl, aminocarbonyl, alkylcarbonyl, alkoxycarbonyl, arylcarbonyl, or aryloxycarbonyl,
wherein any alkyl, alkenyl, alkynyl or cyclic moiety of the compounds is optionally substituted with one or more suitable substituents.

22-23. (canceled)

24. The compound according to claim 2, wherein R₁ is phenyl.

25. The compound according to claim 24, wherein the phenyl is optionally substituted with C₁-C₆ alkyl, —O—(C₁-C₆ alkyl) or —S—(C₁-C₆ alkyl).

26. The compound according to claim 25, wherein R₁ is 4-t-butylphenyl.

27. The compound according to claim 2, wherein R₃ is quinolinyl.

28. The compound according to claim 27, wherein R₃ is 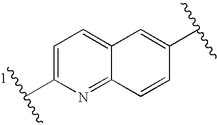
and is oriented such that R₃ is bound to R₂ through the bond indicated by “1”.

29. The compound according to claim 2, wherein R₃ is selected from: 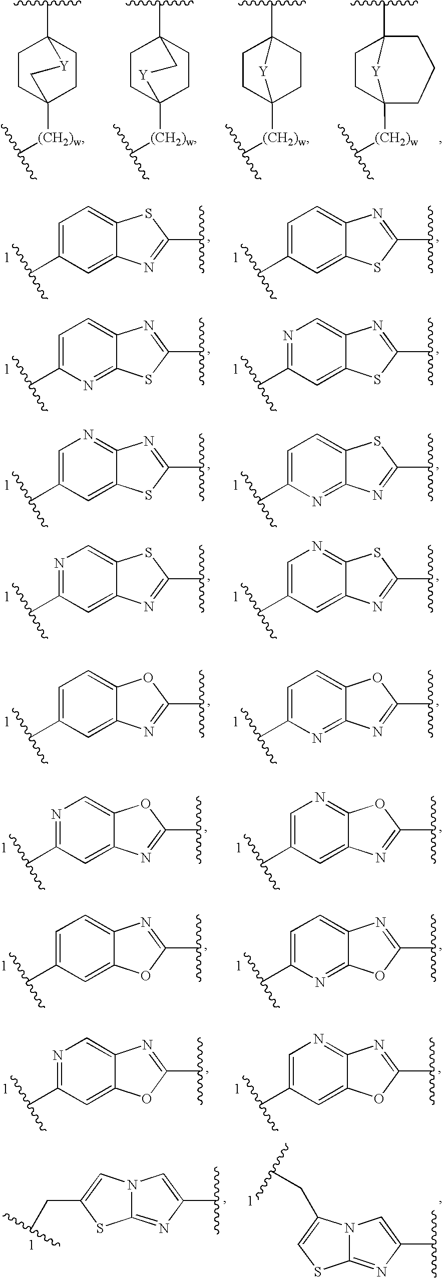 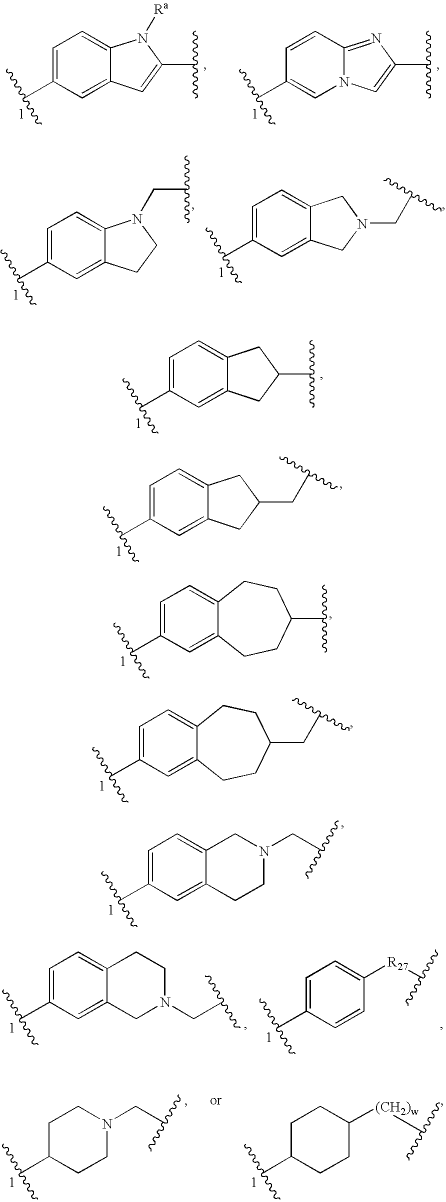
wherein:
Y is selected from —S—, —O—, —N— or —CH₂—;
R₂₇ is selected from a direct bond, —S—, —O—, or —CH₂—;
w is 0 or 1; and
said R₃ is bound to R₂ through the bond indicated by “1” or through either bond if no bond is labeled by “1”.

30. The compound according to claim 29, wherein R₃ is selected from: 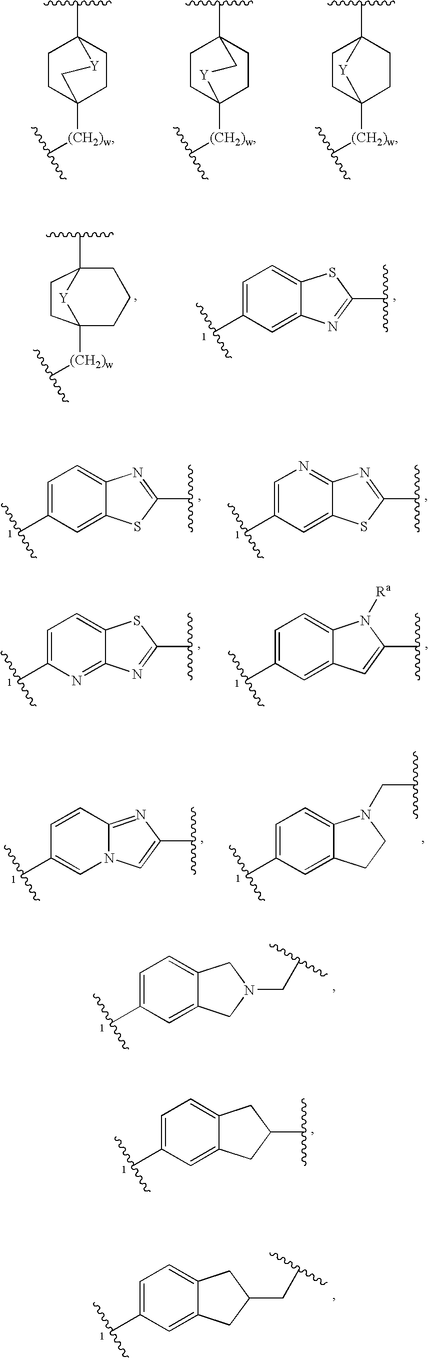 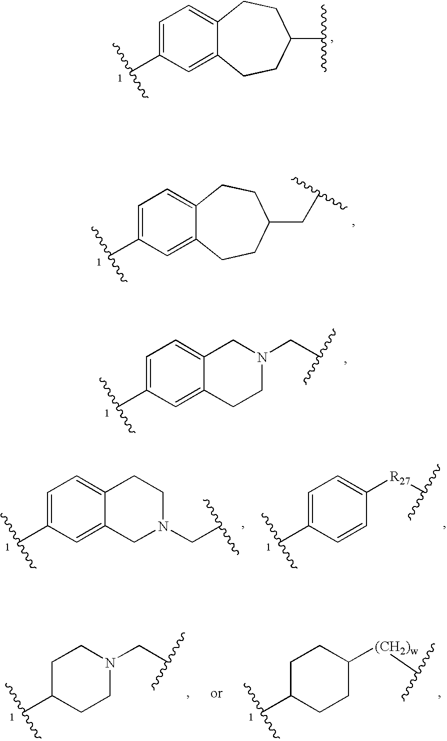
wherein:
Y is selected from —S—, —O—, —N— or —CH₂—;
R₂₇ is selected from a direct bond, —S—, —O—, or —CH₂—;
w is 0 or 1; and
said R₃ is bound to R₂ through the bond indicated by “1” or through either bond if no bond is labeled by “1”.

31. The compound according to claim 29, wherein R₃ is

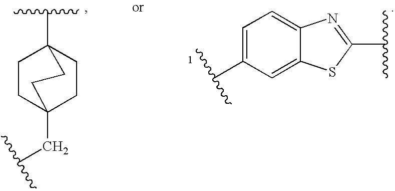

32. The compound according to claim 1, wherein R₁ is substituted with a t-butyl or trifluromethyl.

33. The compound according to claim 1, wherein R₁ is selected from: 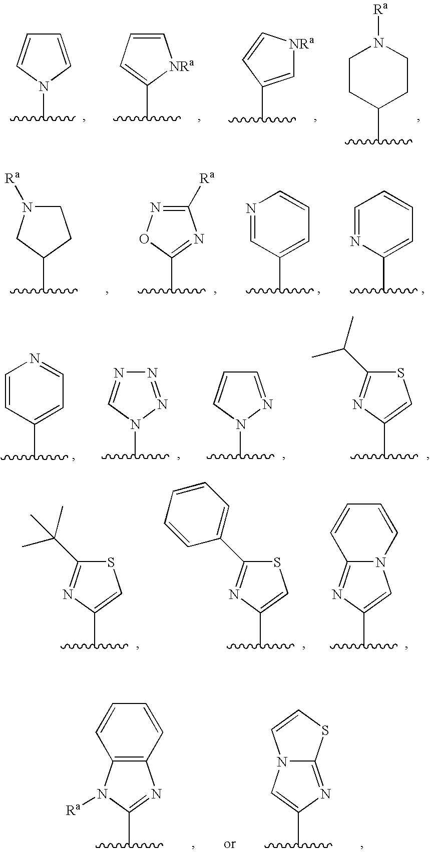
wherein any ring carbon is optionally substituted with a C₁-C₄ straight or branched alkyl group.

34. The compound according to claim 33, wherein R₁ is selected from:

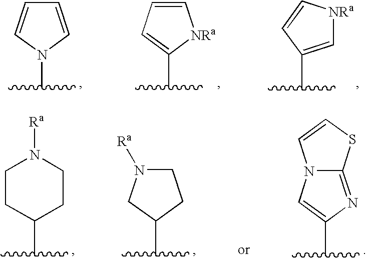

35. The compound according to claim 2, wherein R₂ is —CH(CH₃)—NH—.

36. The compound according to claim 2, wherein R₄ is hydrogen or (C₁-C₆)alkyl; and R₅ is (C₁-C₆)alkyl or —(CR^{a}R^{b})ᵥ-phenyl, wherein said phenyl is optionally substituted with a C₁-C₄ alkyl or halo; or wherein R₄ and R₅ taken together with the nitrogen atom to which they are attached form a 5- to 7-membered monocyclic saturated heterocyclyl moiety.

37. The compound according to claim 36, wherein N(R₄)(R₅) is selected from

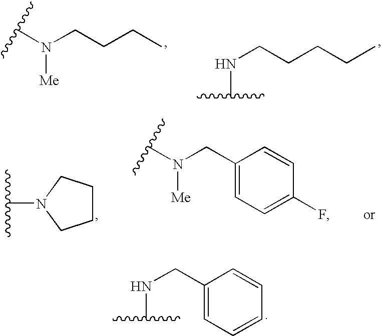

38-39. (canceled)

40. The compound according to claim 37, wherein said compound is selected from: 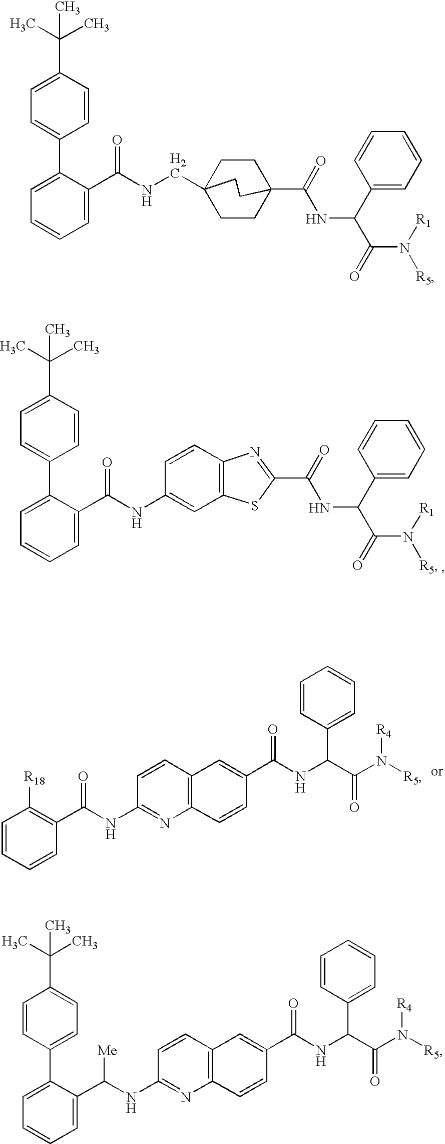
wherein:
R₁₈ is selected from

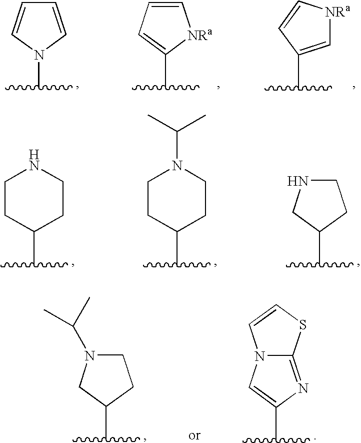

41. A composition comprising a compound of claim 1, wherein the composition is pyrogen-free.

42. A pharmaceutical composition comprising a pharmaceutically acceptable carrier or diluent and a compound of any of claims 1-8.

43. The pharmaceutical composition of claim 42, wherein the pharmaceutical composition is suitable for oral administration or administration to the gastrointestinal tract.

44. A packaged pharmaceutical comprising a compound of claim 1 and instructions for using said compound to treat a disorder or condition.

45. A method of treating a subject in need of microsomal triglyceride transfer protein (MTP) inhibition, comprising administering to the subject a therapeutically effective amount of a compound of claim 1.

46-48. (canceled)

49. A method of treating a subject at risk of having atherosclerosis, comprising administering to the subject a therapeutically effective amount of a compound of claim 1.

50. (canceled)

51. A method of treating a subject suffering from disorder associated with lipid metabolism, comprising administering to the subject a therapeutically effective amount of a compound of claim 1.

52. A method of treating a subject at risk of developing restenosis, comprising administering to the subject a therapeutically effective amount of a compound of claim 1.

53. A method of treating a subject at risk, of having myocardial infarction or a stroke, comprising administering to the subject a therapeutically effective amount of a compound of claim 1.

54-55. (canceled)
